SEQUENCE LISTING (573 sequences)

SEQ ID NO: 1 - protein, 15 aa, Artificial Sequence
  EPO glycopeptide
EAISPPDAASAAPLR

SEQ ID NO: 2 - protein, 30 aa, Artificial Sequence
  30mer peptide covering aa 81-110 of FXYD5
TDGPLVTDPETHKSTKAAHPTDDTTTLSER

SEQ ID NO: 3 - protein, 20 aa, Artificial Sequence
  20mer 3Tn glycopeptide
AHGVTSAPDTRPAPGSTAPP

SEQ ID NO: 4 - protein, 30 aa, Artificial Sequence
  30mer OTS8 5Tn glyco-peptide
KAPLVPTQRERGTKPPLEELSTSATSDHDH

SEQ ID NO: 5 - DNA, 38 bp, Artificial Sequence
  DNA primer
ctcgatatcgaattcgagggcctatttcccatgattcc

SEQ ID NO: 6 - DNA, 92 bp, Artificial Sequence
  DNA primer
cgaattaacggtaccaaaaaaagcaccgactcggtgccactttttcaagttgataacggactagccttattttaacttgctatttctagctc

SEQ ID NO: 7 - DNA, 72 bp, Artificial Sequence
  DNA primer
  misc_feature (29)..(48): n is a, c, g, or t
tttaacttgctatttctagctctaaaacnnnnnnnnnnnnnnnnnnnnggtgtttcgtcctttccacaagat

SEQ ID NO: 8 - DNA, 20 bp, Artificial Sequence
  DNA primer
tcccactgtacactcacaat

SEQ ID NO: 9 - DNA, 20 bp, Artificial Sequence
  DNA primer
gtgaaacgtgatcaccacgc

SEQ ID NO: 10 - DNA, 20 bp, Artificial Sequence
  DNA primer
tatggaagtaaccataaccg

SEQ ID NO: 11 - DNA, 20 bp, Artificial Sequence
  DNA primer
aacagtggcctatatcttcg

SEQ ID NO: 12 - DNA, 20 bp, Artificial Sequence
  DNA primer
gatgacttcgattactggac

SEQ ID NO: 13 - DNA, 20 bp, Artificial Sequence
  DNA primer
gaagagcaagtggacccccc

SEQ ID NO: 14 - DNA, 20 bp, Artificial Sequence
  DNA primer
atgcccaaccgaggcggcaa

SEQ ID NO: 15 - DNA, 20 bp, Artificial Sequence
  DNA primer
gtagtctcgcgtgtcgggga

SEQ ID NO: 16 - DNA, 20 bp, Artificial Sequence
  DNA primer
cgcactgtcgcggatccgag

SEQ ID NO: 17 - DNA, 20 bp, Artificial Sequence
  DNA primer
ctctctcagcatcggtcatg

SEQ ID NO: 18 - DNA, 20 bp, Artificial Sequence
  DNA primer
tatgcttatcagtgaccgct

SEQ ID NO: 19 - DNA, 20 bp, Artificial Sequence
  DNA primer
ttcttctagcaggatatccg

SEQ ID NO: 20 - DNA, 20 bp, Artificial Sequence
  DNA primer
ttaatacgtgcccgtcttcg

SEQ ID NO: 21 - DNA, 20 bp, Artificial Sequence
  DNA primer
cttacaggactacacgcggg

SEQ ID NO: 22 - DNA, 20 bp, Artificial Sequence
  DNA primer
gctggctgaggtcgtccacg

SEQ ID NO: 23 - DNA, 20 bp, Artificial Sequence
  DNA primer
gtaatggcgggtgtcccgga

SEQ ID NO: 24 - DNA, 20 bp, Artificial Sequence
  DNA primer
agttcacattgacctcgcag

SEQ ID NO: 25 - DNA, 20 bp, Artificial Sequence
  DNA primer
tgagatagaagagtacccgc

SEQ ID NO: 26 - DNA, 20 bp, Artificial Sequence
  DNA primer
aggttggccacctcggcgcg

SEQ ID NO: 27 - DNA, 20 bp, Artificial Sequence
  DNA primer
atactctgttcacctcacag

SEQ ID NO: 28 - DNA, 20 bp, Artificial Sequence
  DNA primer
acaacactttgttacaacgc

SEQ ID NO: 29 - DNA, 20 bp, Artificial Sequence
  DNA primer
ccaagtagctttgacgtgtt

SEQ ID NO: 30 - DNA, 20 bp, Artificial Sequence
  DNA primer
caacactttgttacaacgct

SEQ ID NO: 31 - DNA, 20 bp, Artificial Sequence
  DNA primer
gtaggtgatgatgctcatgg

SEQ ID NO: 32 - DNA, 20 bp, Artificial Sequence
  DNA primer
gagctccaacactatctggt

SEQ ID NO: 33 - DNA, 20 bp, Artificial Sequence
  DNA primer
cttcgaggcggtcggctggt

SEQ ID NO: 34 - DNA, 20 bp, Artificial Sequence
  DNA primer
gagggacacatgggccttcg

SEQ ID NO: 35 - DNA, 20 bp, Artificial Sequence
  DNA primer
gcaacgccgccgacaaggag

SEQ ID NO: 36 - DNA, 20 bp, Artificial Sequence
  DNA primer
agggcgactccaaggaaacg

SEQ ID NO: 37 - DNA, 20 bp, Artificial Sequence
  DNA primer
gagcaggacgacgacgacaa

SEQ ID NO: 38 - DNA, 20 bp, Artificial Sequence
  DNA primer
gctgggcctcaaggaccgcg

SEQ ID NO: 39 - DNA, 20 bp, Artificial Sequence
  DNA primer
ttatttcagtgcgacgggcc

SEQ ID NO: 40 - DNA, 20 bp, Artificial Sequence
  DNA primer
tcttgcttatttcagtgcga

SEQ ID NO: 41 - DNA, 20 bp, Artificial Sequence
  DNA primer
cttcttccataagagtaagg

SEQ ID NO: 42 - DNA, 20 bp, Artificial Sequence
  DNA primer
ggatatccgccttactctta

SEQ ID NO: 43 - DNA, 20 bp, Artificial Sequence
  DNA primer
ggtggcctgtggcaatcggc

SEQ ID NO: 44 - DNA, 20 bp, Artificial Sequence
  DNA primer
cgatggtgttggcgtcgccg

SEQ ID NO: 45 - DNA, 20 bp, Artificial Sequence
  DNA primer
ggagagtgccttcgagtgcg

SEQ ID NO: 46 - DNA, 20 bp, Artificial Sequence
  DNA primer
cttgaaagactcgagtgtgt

SEQ ID NO: 47 - DNA, 20 bp, Artificial Sequence
  DNA primer
tagctgggcagcaacgggct

SEQ ID NO: 48 - DNA, 20 bp, Artificial Sequence
  DNA primer
cttccaaggtcaaaggacga

SEQ ID NO: 49 - DNA, 20 bp, Artificial Sequence
  DNA primer
ctcttgaaccgtaagtagtc

SEQ ID NO: 50 - DNA, 20 bp, Artificial Sequence
  DNA primer
acttgagaactcgctattca

SEQ ID NO: 51 - DNA, 20 bp, Artificial Sequence
  DNA primer
cacaccaagcatgtttggca

SEQ ID NO: 52 - DNA, 20 bp, Artificial Sequence
  DNA primer
ctcatcatcgcgtcagacga

SEQ ID NO: 53 - DNA, 20 bp, Artificial Sequence
  DNA primer
ggtgctgagaattcgcggct

SEQ ID NO: 54 - DNA, 20 bp, Artificial Sequence
  DNA primer
ctgcccgagggcatccgccc

SEQ ID NO: 55 - DNA, 20 bp, Artificial Sequence
  DNA primer
gtgttcctactactgctcca

SEQ ID NO: 56 - DNA, 20 bp, Artificial Sequence
  DNA primer
catagattcgcacaaccctg

SEQ ID NO: 57 - DNA, 20 bp, Artificial Sequence
  DNA primer
cagtgagaccgacggccggg

SEQ ID NO: 58 - DNA, 20 bp, Artificial Sequence
  DNA primer
gtctgtcacctgcaaatccg

SEQ ID NO: 59 - DNA, 20 bp, Artificial Sequence
  DNA primer
agtgaccaccgggaaatcga

SEQ ID NO: 60 - DNA, 20 bp, Artificial Sequence
  DNA primer
atgttgttcttagcataaga

SEQ ID NO: 61 - DNA, 20 bp, Artificial Sequence
  DNA primer
ttgggtgtatcactagctgc

SEQ ID NO: 62 - DNA, 20 bp, Artificial Sequence
  DNA primer
tgtatcctcaagcagcaccc

SEQ ID NO: 63 - DNA, 20 bp, Artificial Sequence
  DNA primer
agctgggtacaggctcagcg

SEQ ID NO: 64 - DNA, 20 bp, Artificial Sequence
  DNA primer
acggtgtcagagaagcacca

SEQ ID NO: 65 - DNA, 20 bp, Artificial Sequence
  DNA primer
gagcccccgccagccatacg

SEQ ID NO: 66 - DNA, 20 bp, Artificial Sequence
  DNA primer
gtatccatagtgagttcgaa

SEQ ID NO: 67 - DNA, 20 bp, Artificial Sequence
  DNA primer
tgtgtgtgagacgacacgca

SEQ ID NO: 68 - DNA, 20 bp, Artificial Sequence
  DNA primer
ctagtgtacagcagcctcgg

SEQ ID NO: 69 - DNA, 20 bp, Artificial Sequence
  DNA primer
tcttccattacggctccctg

SEQ ID NO: 70 - DNA, 20 bp, Artificial Sequence
  DNA primer
gcattcttctcagtagagct

SEQ ID NO: 71 - DNA, 20 bp, Artificial Sequence
  DNA primer
tccaagtcgatggtcttgaa

SEQ ID NO: 72 - DNA, 20 bp, Artificial Sequence
  DNA primer
gtggctgtcaaaccagtcgg

SEQ ID NO: 73 - DNA, 20 bp, Artificial Sequence
  DNA primer
gattctagcccacttgcgaa

SEQ ID NO: 74 - DNA, 20 bp, Artificial Sequence
  DNA primer
ttacccgcttcttatcactc

SEQ ID NO: 75 - DNA, 20 bp, Artificial Sequence
  DNA primer
atttgagcacaggtatagcg

SEQ ID NO: 76 - DNA, 20 bp, Artificial Sequence
  DNA primer
tgagaatcactgtcgtatta

SEQ ID NO: 77 - DNA, 20 bp, Artificial Sequence
  DNA primer
tgacctgctccctctcaacg

SEQ ID NO: 78 - DNA, 20 bp, Artificial Sequence
  DNA primer
gataaaggatcccaagcaag

SEQ ID NO: 79 - DNA, 20 bp, Artificial Sequence
  DNA primer
cgtgtgcaagtagaagtgcg

SEQ ID NO: 80 - DNA, 20 bp, Artificial Sequence
  DNA primer
gctaggatgtcccgtctctt

SEQ ID NO: 81 - DNA, 20 bp, Artificial Sequence
  DNA primer
aaagaagcgggtgaaaagcg

SEQ ID NO: 82 - DNA, 20 bp, Artificial Sequence
  DNA primer
tcggagttccgcttgcagct

SEQ ID NO: 83 - DNA, 20 bp, Artificial Sequence
  DNA primer
ggagtctccacaccgctgca

SEQ ID NO: 84 - DNA, 20 bp, Artificial Sequence
  DNA primer
agtagaggatgacggccacg

SEQ ID NO: 85 - DNA, 20 bp, Artificial Sequence
  DNA primer
tccctgatctcggccaaata

SEQ ID NO: 86 - DNA, 20 bp, Artificial Sequence
  DNA primer
acccacgaagtagttactgg

SEQ ID NO: 87 - DNA, 20 bp, Artificial Sequence
  DNA primer
ttcggagtgcttatgccaag

SEQ ID NO: 88 - DNA, 20 bp, Artificial Sequence
  DNA primer
ctctttatggtacaagctcg

SEQ ID NO: 89 - DNA, 20 bp, Artificial Sequence
  DNA primer
catcgccaccagcatgagcg

SEQ ID NO: 90 - DNA, 20 bp, Artificial Sequence
  DNA primer
gttccagtatgcctcgggag

SEQ ID NO: 91 - DNA, 20 bp, Artificial Sequence
  DNA primer
gcagccaccggcgatccccg

SEQ ID NO: 92 - DNA, 20 bp, Artificial Sequence
  DNA primer
gtatccttggaacagcctga

SEQ ID NO: 93 - DNA, 20 bp, Artificial Sequence
  DNA primer
ctcacaatgtgattatcgat

SEQ ID NO: 94 - DNA, 20 bp, Artificial Sequence
  DNA primer
ctcttaagcacacctcagcg

SEQ ID NO: 95 - DNA, 20 bp, Artificial Sequence
  DNA primer
ccaatacttgattaaccaca

SEQ ID NO: 96 - DNA, 20 bp, Artificial Sequence
  DNA primer
gcggggaccttggcgcctgg

SEQ ID NO: 97 - DNA, 20 bp, Artificial Sequence
  DNA primer
gctcctgcagaaactgacca

SEQ ID NO: 98 - DNA, 20 bp, Artificial Sequence
  DNA primer
ggagtgtgagcagtgctgac

SEQ ID NO: 99 - DNA, 20 bp, Artificial Sequence
  DNA primer
gcttattgctgtcaagtcgg

SEQ ID NO: 100 - DNA, 20 bp, Artificial Sequence
  DNA primer
ccctcagtcagcgctctcga

SEQ ID NO: 101 - DNA, 20 bp, Artificial Sequence
  DNA primer
gttcggcgtccagcaacggt

SEQ ID NO: 102 - DNA, 20 bp, Artificial Sequence
  DNA primer
tcctcggccgccttgctggg

SEQ ID NO: 103 - DNA, 20 bp, Artificial Sequence
  DNA primer
ctttgtcttggtatactaca

SEQ ID NO: 104 - DNA, 20 bp, Artificial Sequence
  DNA primer
ggagagcctcaagcgctcca

SEQ ID NO: 105 - DNA, 20 bp, Artificial Sequence
  DNA primer
tgtggcagctaggtagcgat

SEQ ID NO: 106 - DNA, 20 bp, Artificial Sequence
  DNA primer
ggtatttccactgttaacag

SEQ ID NO: 107 - DNA, 20 bp, Artificial Sequence
  DNA primer
gctgtcatgactccagcgta

SEQ ID NO: 108 - DNA, 20 bp, Artificial Sequence
  DNA primer
ccaccaaggtaccttctgtg

SEQ ID NO: 109 - DNA, 20 bp, Artificial Sequence
  DNA primer
cagggtgatgcggaataccg

SEQ ID NO: 110 - DNA, 20 bp, Artificial Sequence
  DNA primer
agtgctagcctcaacatcaa

SEQ ID NO: 111 - DNA, 20 bp, Artificial Sequence
  DNA primer
tgtccgtagcaggatcagga

SEQ ID NO: 112 - DNA, 20 bp, Artificial Sequence
  DNA primer
cctccacgcctgcggacgcg

SEQ ID NO: 113 - DNA, 20 bp, Artificial Sequence
  DNA primer
ggcagtggaacctgtcaccg

SEQ ID NO: 114 - DNA, 20 bp, Artificial Sequence
  DNA primer
ggacccattagggtacacag

SEQ ID NO: 115 - DNA, 20 bp, Artificial Sequence
  DNA primer
tagcgggtgcaggtgtcgct

SEQ ID NO: 116 - DNA, 20 bp, Artificial Sequence
  DNA primer
accttgctgttttatatagg

SEQ ID NO: 117 - DNA, 20 bp, Artificial Sequence
  DNA primer
gtgaacggtccgttgtgaga

SEQ ID NO: 118 - DNA, 20 bp, Artificial Sequence
  DNA primer
gggaccaccagagcataatg

SEQ ID NO: 119 - DNA, 20 bp, Artificial Sequence
  DNA primer
cgcgcagctctgggacgccg

SEQ ID NO: 120 - DNA, 20 bp, Artificial Sequence
  DNA primer
aaagctgctaaaccgtacct

SEQ ID NO: 121 - DNA, 20 bp, Artificial Sequence
  DNA primer
ggaaggcttcaacctgcgca

SEQ ID NO: 122 - DNA, 20 bp, Artificial Sequence
  DNA primer
gatgaagcggtcatactcca

SEQ ID NO: 123 - DNA, 20 bp, Artificial Sequence
  DNA primer
gcgcaagcggtggaaagccc

SEQ ID NO: 124 - DNA, 20 bp, Artificial Sequence
  DNA primer
gccaccctggaccctctcgg

SEQ ID NO: 125 - DNA, 20 bp, Artificial Sequence
  DNA primer
gaggatctaactcctttccg

SEQ ID NO: 126 - DNA, 20 bp, Artificial Sequence
  DNA primer
aattgttttagcttgacaac

SEQ ID NO: 127 - DNA, 20 bp, Artificial Sequence
  DNA primer
aacaatctctgcgaagcaca

SEQ ID NO: 128 - DNA, 20 bp, Artificial Sequence
  DNA primer
ttcgcagagattgttcttgc

SEQ ID NO: 129 - DNA, 20 bp, Artificial Sequence
  DNA primer
acccgagctctggatccacc

SEQ ID NO: 130 - DNA, 20 bp, Artificial Sequence
  DNA primer
gcagtgagcgcagcgcgacg

SEQ ID NO: 131 - DNA, 20 bp, Artificial Sequence
  DNA primer
tcagccacctaacagttgcc

SEQ ID NO: 132 - DNA, 20 bp, Artificial Sequence
  DNA primer
cctgtgacatacactttccg

SEQ ID NO: 133 - DNA, 20 bp, Artificial Sequence
  DNA primer
ggaagcttgcagtggtcccg

SEQ ID NO: 134 - DNA, 20 bp, Artificial Sequence
  DNA primer
tttcccccacgtctgccgga

SEQ ID NO: 135 - DNA, 20 bp, Artificial Sequence
  DNA primer
cttcgagttcgtgctcaagg

SEQ ID NO: 136 - DNA, 20 bp, Artificial Sequence
  DNA primer
cggacgacgactccttcgcg

SEQ ID NO: 137 - DNA, 20 bp, Artificial Sequence
  DNA primer
agaagccccagtagaggcgg

SEQ ID NO: 138 - DNA, 20 bp, Artificial Sequence
  DNA primer
gtttagccagctgatgaagg

SEQ ID NO: 139 - DNA, 20 bp, Artificial Sequence
  DNA primer
catcagctggctaaacaaga

SEQ ID NO: 140 - DNA, 20 bp, Artificial Sequence
  DNA primer
caaacgtactgcggtaacaa

SEQ ID NO: 141 - DNA, 20 bp, Artificial Sequence
  DNA primer
cgttctatcacattgtagtg

SEQ ID NO: 142 - DNA, 20 bp, Artificial Sequence
  DNA primer
gaaacgtgataagaagcacc

SEQ ID NO: 143 - DNA, 20 bp, Artificial Sequence
  DNA primer
accgggatgtgtgcgtagcg

SEQ ID NO: 144 - DNA, 20 bp, Artificial Sequence
  DNA primer
ccgtggactgggtacccaaa

SEQ ID NO: 145 - DNA, 20 bp, Artificial Sequence
  DNA primer
tagtcgtcaggtgtccaccg

SEQ ID NO: 146 - DNA, 20 bp, Artificial Sequence
  DNA primer
atagcaggtagcttcatcaa

SEQ ID NO: 147 - DNA, 20 bp, Artificial Sequence
  DNA primer
actgttcaggggtcacccga

SEQ ID NO: 148 - DNA, 20 bp, Artificial Sequence
  DNA primer
gcagccatagggttaaaaac

SEQ ID NO: 149 - DNA, 20 bp, Artificial Sequence
  DNA primer
ggtcaaatagatgacgtagg

SEQ ID NO: 150 - DNA, 20 bp, Artificial Sequence
  DNA primer
actgctccaggatttctcgg

SEQ ID NO: 151 - DNA, 20 bp, Artificial Sequence
  DNA primer
cgttgggcagccggtagacg

SEQ ID NO: 152 - DNA, 20 bp, Artificial Sequence
  DNA primer
tgccatcgtgggcaactcgg

SEQ ID NO: 153 - DNA, 20 bp, Artificial Sequence
  DNA primer
gatgagcgataaaatcagca

SEQ ID NO: 154 - DNA, 20 bp, Artificial Sequence
  DNA primer
agatgcgctccattaggaag

SEQ ID NO: 155 - DNA, 20 bp, Artificial Sequence
  DNA primer
atacaggatctgttgcagca

SEQ ID NO: 156 - DNA, 20 bp, Artificial Sequence
  DNA primer
ggagcgtcctcagcgctgcg

SEQ ID NO: 157 - DNA, 20 bp, Artificial Sequence
  DNA primer
acaacagcaacttcgcaccc

SEQ ID NO: 158 - DNA, 20 bp, Artificial Sequence
  DNA primer
gacagttcagcagggcgacg

SEQ ID NO: 159 - DNA, 20 bp, Artificial Sequence
  DNA primer
ggtacccctccttccccgtg

SEQ ID NO: 160 - DNA, 20 bp, Artificial Sequence
  DNA primer
gtattatcaagccctcctac

SEQ ID NO: 161 - DNA, 20 bp, Artificial Sequence
  DNA primer
ggaacaccacacgctccagc

SEQ ID NO: 162 - DNA, 20 bp, Artificial Sequence
  DNA primer
cagtgaagtagagtaaccga

SEQ ID NO: 163 - DNA, 20 bp, Artificial Sequence
  DNA primer
gtacatcaaaggagaccgtc

SEQ ID NO: 164 - DNA, 20 bp, Artificial Sequence
  DNA primer
tctcggctaaagatcatgcc

SEQ ID NO: 165 - DNA, 20 bp, Artificial Sequence
  DNA primer
gtagaacctggagccctcga

SEQ ID NO: 166 - DNA, 20 bp, Artificial Sequence
  DNA primer
tcggctggcagcctaacaac

SEQ ID NO: 167 - DNA, 20 bp, Artificial Sequence
  DNA primer
gtagaagcgagagccctcaa

SEQ ID NO: 168 - DNA, 20 bp, Artificial Sequence
  DNA primer
cagctaccagtaataatacg

SEQ ID NO: 169 - DNA, 20 bp, Artificial Sequence
  DNA primer
ggtggggaacgagctgtgcg

SEQ ID NO: 170 - DNA, 20 bp, Artificial Sequence
  DNA primer
gttggcgcccatcgtctccg

SEQ ID NO: 171 - DNA, 20 bp, Artificial Sequence
  DNA primer
gcaacctattcttctctaac

SEQ ID NO: 172 - DNA, 20 bp, Artificial Sequence
  DNA primer
gtttgcagctatgtcgacat

SEQ ID NO: 173 - DNA, 20 bp, Artificial Sequence
  DNA primer
actgaggatcgactacccga

SEQ ID NO: 174 - DNA, 20 bp, Artificial Sequence
  DNA primer
cctggaggtgcgcatgcgcg

SEQ ID NO: 175 - DNA, 20 bp, Artificial Sequence
  DNA primer
gcaccaggacggtgacacgg

SEQ ID NO: 176 - DNA, 20 bp, Artificial Sequence
  DNA primer
gagtctatcccgtctagccg

SEQ ID NO: 177 - DNA, 20 bp, Artificial Sequence
  DNA primer
ctgaatatagaaatagcggg

SEQ ID NO: 178 - DNA, 20 bp, Artificial Sequence
  DNA primer
gatttgactacgactttgaa

SEQ ID NO: 179 - DNA, 20 bp, Artificial Sequence
  DNA primer
gctcttcatctctatagggg

SEQ ID NO: 180 - DNA, 20 bp, Artificial Sequence
  DNA primer
tcagagtgtgatacacactg

SEQ ID NO: 181 - DNA, 20 bp, Artificial Sequence
  DNA primer
gctctccgacatgcagtaga

SEQ ID NO: 182 - DNA, 20 bp, Artificial Sequence
  DNA primer
gctattgatggcagccatag

SEQ ID NO: 183 - DNA, 20 bp, Artificial Sequence
  DNA primer
acatttcagtgagcgctaca

SEQ ID NO: 184 - DNA, 20 bp, Artificial Sequence
  DNA primer
ataggctgtcctttgtcgac

SEQ ID NO: 185 - DNA, 20 bp, Artificial Sequence
  DNA primer
ttacactgtaccgcagtgaa

SEQ ID NO: 186 - DNA, 20 bp, Artificial Sequence
  DNA primer
ccgacagctgacccgagatg

SEQ ID NO: 187 - DNA, 20 bp, Artificial Sequence
  DNA primer
gctatctgctcgcgatccag

SEQ ID NO: 188 - DNA, 20 bp, Artificial Sequence
  DNA primer
cttcattagcccatagaggc

SEQ ID NO: 189 - DNA, 20 bp, Artificial Sequence
  DNA primer
ccgtgtccaggacaatgacg

SEQ ID NO: 190 - DNA, 20 bp, Artificial Sequence
  DNA primer
ccagagctccgagatgtcag

SEQ ID NO: 191 - DNA, 20 bp, Artificial Sequence
  DNA primer
ggcctgatgcactcacaaca

SEQ ID NO: 192 - DNA, 20 bp, Artificial Sequence
  DNA primer
aggaacgtgaccttccgctg

SEQ ID NO: 193 - DNA, 20 bp, Artificial Sequence
  DNA primer
gacctgccagtcatagcaca

SEQ ID NO: 194 - DNA, 20 bp, Artificial Sequence
  DNA primer
gcctgaaccatgccattgga

SEQ ID NO: 195 - DNA, 20 bp, Artificial Sequence
  DNA primer
actgctggacagtttctccg

SEQ ID NO: 196 - DNA, 20 bp, Artificial Sequence
  DNA primer
gctgcgtgcaaaacacacaa

SEQ ID NO: 197 - DNA, 20 bp, Artificial Sequence
  DNA primer
agccgcaagtaacgacaccc

SEQ ID NO: 198 - DNA, 20 bp, Artificial Sequence
  DNA primer
gcaatcggtgaccggcgtag

SEQ ID NO: 199 - DNA, 20 bp, Artificial Sequence
  DNA primer
ctaagaggccaagtgtaagt

SEQ ID NO: 200 - DNA, 20 bp, Artificial Sequence
  DNA primer
cggcagatcctacttacact

SEQ ID NO: 201 - DNA, 20 bp, Artificial Sequence
  DNA primer
gcttttacctgaatttaggg

SEQ ID NO: 202 - DNA, 20 bp, Artificial Sequence
  DNA primer
tccggggcgccccaaggcag

SEQ ID NO: 203 - DNA, 20 bp, Artificial Sequence
  DNA primer
gtctgcaccctgttcatcat

SEQ ID NO: 204 - DNA, 20 bp, Artificial Sequence
  DNA primer
gcacgttgtgggagagccca

SEQ ID NO: 205 - DNA, 20 bp, Artificial Sequence
  DNA primer
acttcagggtgaactggtag

SEQ ID NO: 206 - DNA, 20 bp, Artificial Sequence
  DNA primer
ctacggaacaggagaccaaa

SEQ ID NO: 207 - DNA, 20 bp, Artificial Sequence
  DNA primer
aatcttggcagcagactcta

SEQ ID NO: 208 - DNA, 20 bp, Artificial Sequence
  DNA primer
aatgtgccctcccagacaat

SEQ ID NO: 209 - DNA, 20 bp, Artificial Sequence
  DNA primer
actagactcttgcctgtcag

SEQ ID NO: 210 - DNA, 20 bp, Artificial Sequence
  DNA primer
ttaggatctacccctttcag

SEQ ID NO: 211 - DNA, 20 bp, Artificial Sequence
  DNA primer
ctactatcatgcaatattgg

SEQ ID NO: 212 - DNA, 20 bp, Artificial Sequence
  DNA primer
ttcgcagctcggctccggga

SEQ ID NO: 213 - DNA, 20 bp, Artificial Sequence
  DNA primer
ggcagtcaccgacttggacg

SEQ ID NO: 214 - DNA, 20 bp, Artificial Sequence
  DNA primer
cggggtctcgggccacttcg

SEQ ID NO: 215 - DNA, 20 bp, Artificial Sequence
  DNA primer
tggtactgttaaagatccct

SEQ ID NO: 216 - DNA, 20 bp, Artificial Sequence
  DNA primer
gggcatggttgccaaccatc

SEQ ID NO: 217 - DNA, 20 bp, Artificial Sequence
  DNA primer
tgtgatagctcatcttttag

SEQ ID NO: 218 - DNA, 20 bp, Artificial Sequence
  DNA primer
catggtcgacatgttccgcg

SEQ ID NO: 219 - DNA, 20 bp, Artificial Sequence
  DNA primer
tgaaaaggctaacctaccct

SEQ ID NO: 220 - DNA, 20 bp, Artificial Sequence
  DNA primer
ggtggtggatggcaaccgcc

SEQ ID NO: 221 - DNA, 20 bp, Artificial Sequence
  DNA primer
gacgaaggcgaaggtgacag

SEQ ID NO: 222 - DNA, 20 bp, Artificial Sequence
  DNA primer
actgtaagaagaatgcttcg

SEQ ID NO: 223 - DNA, 20 bp, Artificial Sequence
  DNA primer
agttcttcgacttcccactg

SEQ ID NO: 224 - DNA, 20 bp, Artificial Sequence
  DNA primer
ggagcagtacaaacctttat

SEQ ID NO: 225 - DNA, 20 bp, Artificial Sequence
  DNA primer
gaccagggcacctttggcgt

SEQ ID NO: 226 - DNA, 20 bp, Artificial Sequence
  DNA primer
gagagtccaacagtgaagct

SEQ ID NO: 227 - DNA, 20 bp, Artificial Sequence
  DNA primer
gggaagggactttcgacagg

SEQ ID NO: 228 - DNA, 20 bp, Artificial Sequence
  DNA primer
gtctgccccttcagcccgca

SEQ ID NO: 229 - DNA, 20 bp, Artificial Sequence
  DNA primer
ggtggctacggaccacaaca

SEQ ID NO: 230 - DNA, 20 bp, Artificial Sequence
  DNA primer
ataacacgtcaactgtgctg

SEQ ID NO: 231 - DNA, 20 bp, Artificial Sequence
  DNA primer
gaagagtttgtaccattccg

SEQ ID NO: 232 - DNA, 20 bp, Artificial Sequence
  DNA primer
actacgttcagattcgagaa

SEQ ID NO: 233 - DNA, 20 bp, Artificial Sequence
  DNA primer
ccaccttcctaattgacctc

SEQ ID NO: 234 - DNA, 20 bp, Artificial Sequence
  DNA primer
gtagaaagtcagcttgtccg

SEQ ID NO: 235 - DNA, 20 bp, Artificial Sequence
  DNA primer
cagtccacaacgctgaaccg

SEQ ID NO: 236 - DNA, 20 bp, Artificial Sequence
  DNA primer
gcagtttccaagctcaacac

SEQ ID NO: 237 - DNA, 20 bp, Artificial Sequence
  DNA primer
ggctgtgtaccacatctaag

SEQ ID NO: 238 - DNA, 20 bp, Artificial Sequence
  DNA primer
acactctctcgggttagccc

SEQ ID NO: 239 - DNA, 20 bp, Artificial Sequence
  DNA primer
ccgtacccataatatatgca

SEQ ID NO: 240 - DNA, 20 bp, Artificial Sequence
  DNA primer
ttttcgatttccataagcat

SEQ ID NO: 241 - DNA, 20 bp, Artificial Sequence
  DNA primer
aagtgcggaatggagccggg

SEQ ID NO: 242 - DNA, 20 bp, Artificial Sequence
  DNA primer
ttcgcagctttatcttgccg

SEQ ID NO: 243 - DNA, 20 bp, Artificial Sequence
  DNA primer
taccttgtacttcaacaccc

SEQ ID NO: 244 - DNA, 20 bp, Artificial Sequence
  DNA primer
tcaacctgtgactaacacca

SEQ ID NO: 245 - DNA, 20 bp, Artificial Sequence
  DNA primer
ggagctcttccaggaatcgc

SEQ ID NO: 246 - DNA, 20 bp, Artificial Sequence
  DNA primer
agtccctaaaagccaatggg

SEQ ID NO: 247 - DNA, 20 bp, Artificial Sequence
  DNA primer
ggcaggacggggagtagtag

SEQ ID NO: 248 - DNA, 20 bp, Artificial Sequence
  DNA primer
gcagaattcccggcaggacg

SEQ ID NO: 249 - DNA, 20 bp, Artificial Sequence
  DNA primer
acttgccattacctcgcaga

SEQ ID NO: 250 - DNA, 20 bp, Artificial Sequence
  DNA primer
gaagacgccatagaaaacca

SEQ ID NO: 251 - DNA, 20 bp, Artificial Sequence
  DNA primer
cccctccgtgacgaagcgcg

SEQ ID NO: 252 - DNA, 20 bp, Artificial Sequence
  DNA primer
actttccaaagagctcgctg

SEQ ID NO: 253 - DNA, 20 bp, Artificial Sequence
  DNA primer
tacagtactttacctcgtgt

SEQ ID NO: 254 - DNA, 20 bp, Artificial Sequence
  DNA primer
caggaataaaggcccacacg

SEQ ID NO: 255 - DNA, 20 bp, Artificial Sequence
  DNA primer
aacttacttttgaggccaat

SEQ ID NO: 256 - DNA, 20 bp, Artificial Sequence
  DNA primer
cacctggtgccactgccggc

SEQ ID NO: 257 - DNA, 20 bp, Artificial Sequence
  DNA primer
cggcacgttctggagctgcg

SEQ ID NO: 258 - DNA, 20 bp, Artificial Sequence
  DNA primer
catcttctatgaccagcgcc

SEQ ID NO: 259 - DNA, 20 bp, Artificial Sequence
  DNA primer
agttggtccacaaagcctga

SEQ ID NO: 260 - DNA, 20 bp, Artificial Sequence
  DNA primer
aggctttgtggaccaactcg

SEQ ID NO: 261 - DNA, 20 bp, Artificial Sequence
  DNA primer
aactgttgagacccttacgg

SEQ ID NO: 262 - DNA, 20 bp, Artificial Sequence
  DNA primer
gcgtagcatctgtagcagct

SEQ ID NO: 263 - DNA, 20 bp, Artificial Sequence
  DNA primer
gcaacaactggatctgaaga

SEQ ID NO: 264 - DNA, 20 bp, Artificial Sequence
  DNA primer
gcacatagcccgtctgcgga

SEQ ID NO: 265 - DNA, 20 bp, Artificial Sequence
  DNA primer
gttgaagctcttccgcacct

SEQ ID NO: 266 - DNA, 20 bp, Artificial Sequence
  DNA primer
aggtgcggaagagcttcaac

SEQ ID NO: 267 - DNA, 20 bp, Artificial Sequence
  DNA primer
agcgcgaagaagtagtcgcg

SEQ ID NO: 268 - DNA, 20 bp, Artificial Sequence
  DNA primer
tgacggaccaggagaagcgg

SEQ ID NO: 269 - DNA, 20 bp, Artificial Sequence
  DNA primer
tctccacgcccacgatgccg

SEQ ID NO: 270 - DNA, 20 bp, Artificial Sequence
  DNA primer
cgcgaagtagtagtcgcggg

SEQ ID NO: 271 - DNA, 20 bp, Artificial Sequence
  DNA primer
cgagtgtgaaatgcaggtgc

SEQ ID NO: 272 - DNA, 20 bp, Artificial Sequence
  DNA primer
agcaaagtagtagtctcgtg

SEQ ID NO: 273 - DNA, 20 bp, Artificial Sequence
  DNA primer
acgagactactactttgctc

SEQ ID NO: 274 - DNA, 20 bp, Artificial Sequence
  DNA primer
cattgagcagtcacatcgct

SEQ ID NO: 275 - DNA, 20 bp, Artificial Sequence
  DNA primer
ccagcgatgtgactgctcaa

SEQ ID NO: 276 - DNA, 20 bp, Artificial Sequence
  DNA primer
agagaaggaaagtcaatcgt

SEQ ID NO: 277 - DNA, 20 bp, Artificial Sequence
  DNA primer
aatgacggtcattttcaaag

SEQ ID NO: 278 - DNA, 20 bp, Artificial Sequence
  DNA primer
tctccctttccaatgttgag

SEQ ID NO: 279 - DNA, 20 bp, Artificial Sequence
  DNA primer
ctcacctctcaacattggaa

SEQ ID NO: 280 - DNA, 20 bp, Artificial Sequence
  DNA primer
agccagtctaaggggcggcg

SEQ ID NO: 281 - DNA, 20 bp, Artificial Sequence
  DNA primer
cagactttggatcctccccg

SEQ ID NO: 282 - DNA, 20 bp, Artificial Sequence
  DNA primer
gcagctccgggaaaaggtgc

SEQ ID NO: 283 - DNA, 20 bp, Artificial Sequence
  DNA primer
gagctgtgacatagatcgcc

SEQ ID NO: 284 - DNA, 20 bp, Artificial Sequence
  DNA primer
tgtaccactgagtagccagc

SEQ ID NO: 285 - DNA, 20 bp, Artificial Sequence
  DNA primer
gctggctactcagtggtaca

SEQ ID NO: 286 - DNA, 20 bp, Artificial Sequence
  DNA primer
tgtcttgcagcggttactag

SEQ ID NO: 287 - DNA, 20 bp, Artificial Sequence
  DNA primer
acttatcagcataccatgaa

SEQ ID NO: 288 - DNA, 20 bp, Artificial Sequence
  DNA primer
cataccatgaacggaaattc

SEQ ID NO: 289 - DNA, 20 bp, Artificial Sequence
  DNA primer
ccatttaaacagggttaatt

SEQ ID NO: 290 - DNA, 20 bp, Artificial Sequence
  DNA primer
ggagtggaaaactctccagc

SEQ ID NO: 291 - DNA, 20 bp, Artificial Sequence
  DNA primer
gaagtcttatgatttaactc

SEQ ID NO: 292 - DNA, 20 bp, Artificial Sequence
  DNA primer
gatgtcccgtaggatctcca

SEQ ID NO: 293 - DNA, 20 bp, Artificial Sequence
  DNA primer
agctaatcatgtcccagcgg

SEQ ID NO: 294 - DNA, 20 bp, Artificial Sequence
  DNA primer
caacatgaactacacggccg

SEQ ID NO: 295 - DNA, 20 bp, Artificial Sequence
  DNA primer
gcaatgctaggcagacctgg

SEQ ID NO: 296 - DNA, 20 bp, Artificial Sequence
  DNA primer
tgacgagcttgcttccacaa

SEQ ID NO: 297 - DNA, 20 bp, Artificial Sequence
  DNA primer
gcagaacgaggggatggttg

SEQ ID NO: 298 - DNA, 20 bp, Artificial Sequence
  DNA primer
tgttcaggctggctagacgg

SEQ ID NO: 299 - DNA, 20 bp, Artificial Sequence
  DNA primer
ttttcttaaacgactataca

SEQ ID NO: 300 - DNA, 20 bp, Artificial Sequence
  DNA primer
ggccccaattgactggatag

SEQ ID NO: 301 - DNA, 20 bp, Artificial Sequence
  DNA primer
ttgtactatgccaccagccg

SEQ ID NO: 302 - DNA, 20 bp, Artificial Sequence
  DNA primer
ccgaggcactgacatccgca

SEQ ID NO: 303 - DNA, 20 bp, Artificial Sequence
  DNA primer
gatctatcaccagacctgca

SEQ ID NO: 304 - DNA, 20 bp, Artificial Sequence
  DNA primer
agcagttgtaaatgcaacga

SEQ ID NO: 305 - DNA, 20 bp, Artificial Sequence
  DNA primer
tcttcatgtcgatggagtgc

SEQ ID NO: 306 - DNA, 20 bp, Artificial Sequence
  DNA primer
gtaggtgagtcccatatgct

SEQ ID NO: 307 - DNA, 20 bp, Artificial Sequence
  DNA primer
ggctgccattctttacagaa

SEQ ID NO: 308 - DNA, 20 bp, Artificial Sequence
  DNA primer
agagtcttcttagaacctgc

SEQ ID NO: 309 - DNA, 20 bp, Artificial Sequence
  DNA primer
agactcttcccggttgatcg

SEQ ID NO: 310 - DNA, 20 bp, Artificial Sequence
  DNA primer
gtgcggtgccgcagcaatgg

SEQ ID NO: 311 - DNA, 20 bp, Artificial Sequence
  DNA primer
gcggcgctcacaattgccac

SEQ ID NO: 312 - DNA, 20 bp, Artificial Sequence
  DNA primer
tgcgagtcccttactatgtg

SEQ ID NO: 313 - DNA, 20 bp, Artificial Sequence
  DNA primer
gtgtacatacagaagctact

SEQ ID NO: 314 - DNA, 20 bp, Artificial Sequence
  DNA primer
cgttgatagccatgactgga

SEQ ID NO: 315 - DNA, 20 bp, Artificial Sequence
  DNA primer
ggccattcagtgcagctctt

SEQ ID NO: 316 - DNA, 20 bp, Artificial Sequence
  DNA primer
tgatcactccaattgacacc

SEQ ID NO: 317 - DNA, 20 bp, Artificial Sequence
  DNA primer
ggcttgtacctggtgtcaat

SEQ ID NO: 318 - DNA, 20 bp, Artificial Sequence
  DNA primer
ctgccatttggatctttgga

SEQ ID NO: 319 - DNA, 20 bp, Artificial Sequence
  DNA primer
gcaggaatggcgcagctaga

SEQ ID NO: 320 - DNA, 20 bp, Artificial Sequence
  DNA primer
gctagagggttactgtttct

SEQ ID NO: 321 - DNA, 20 bp, Artificial Sequence
  DNA primer
tgtagggctctcgcagcgcc

SEQ ID NO: 322 - DNA, 20 bp, Artificial Sequence
  DNA primer
gttccacatacaatgagccc

SEQ ID NO: 323 - DNA, 20 bp, Artificial Sequence
  DNA primer
gcagcagtctgattccccaa

SEQ ID NO: 324 - DNA, 20 bp, Artificial Sequence
  DNA primer
ccatactgcaatgctggtgg

SEQ ID NO: 325 - DNA, 20 bp, Artificial Sequence
  DNA primer
agttccccggagtcgtcccc

SEQ ID NO: 326 - DNA, 20 bp, Artificial Sequence
  DNA primer
ctgcgatcaccactggcccg

SEQ ID NO: 327 - DNA, 20 bp, Artificial Sequence
  DNA primer
gcgaaagcacgtaaaccgcg

SEQ ID NO: 328 - DNA, 20 bp, Artificial Sequence
  DNA primer
accctgtacaaaatgcataa

SEQ ID NO: 329 - DNA, 20 bp, Artificial Sequence
  DNA primer
aatggcccgaaagaggcaag

SEQ ID NO: 330 - DNA, 20 bp, Artificial Sequence
  DNA primer
gctccgaaatggcccgaaag

SEQ ID NO: 331 - DNA, 20 bp, Artificial Sequence
  DNA primer
gtgcgttctcgttctagctg

SEQ ID NO: 332 - DNA, 20 bp, Artificial Sequence
  DNA primer
gaagcactacccatattcgc

SEQ ID NO: 333 - DNA, 20 bp, Artificial Sequence
  DNA primer
aagatactgagagactcccg

SEQ ID NO: 334 - DNA, 20 bp, Artificial Sequence
  DNA primer
gtagtcctcgcaggtctcgg

SEQ ID NO: 335 - DNA, 20 bp, Artificial Sequence
  DNA primer
ttctcgcgctcgttgtaggt

SEQ ID NO: 336 - DNA, 20 bp, Artificial Sequence
  DNA primer
accagcagccacacgatgag

SEQ ID NO: 337 - DNA, 20 bp, Artificial Sequence
  DNA primer
cgaggccgagtcccaccacc

SEQ ID NO: 338 - DNA, 20 bp, Artificial Sequence
  DNA primer
tgatcaggctaacggcgacg

SEQ ID NO: 339 - DNA, 20 bp, Artificial Sequence
  DNA primer
cttcacctactacaccgcct

SEQ ID NO: 340 - DNA, 20 bp, Artificial Sequence
  DNA primer
gccctgaccacgggagcctt

SEQ ID NO: 341 - DNA, 20 bp, Artificial Sequence
  DNA primer
gcggacaccagcaagtaggc

SEQ ID NO: 342 - DNA, 20 bp, Artificial Sequence
  DNA primer
gcctacttgctggtgtccgc

SEQ ID NO: 343 - DNA, 20 bp, Artificial Sequence
  DNA primer
agttggtgaaatagaccatg

SEQ ID NO: 344 - DNA, 20 bp, Artificial Sequence
  DNA primer
gaagggcttgggcaccacaa

SEQ ID NO: 345 - DNA, 20 bp, Artificial Sequence
  DNA primer
gatgcaggccaagtatcggg

SEQ ID NO: 346 - DNA, 20 bp, Artificial Sequence
  DNA primer
ccccactgagacatgctctg

SEQ ID NO: 347 - DNA, 20 bp, Artificial Sequence
  DNA primer
ggtctggctccagtctcgct

SEQ ID NO: 348 - DNA, 20 bp, Artificial Sequence
  DNA primer
cgttagccacagcaggttga

SEQ ID NO: 349 - DNA, 20 bp, Artificial Sequence
  DNA primer
agaggcaagcttcacacgca

SEQ ID NO: 350 - DNA, 20 bp, Artificial Sequence
  DNA primer
gtagctctccacatttcgag

SEQ ID NO: 351 - DNA, 20 bp, Artificial Sequence
  DNA primer
agtaggtcctcagagcgcgt

SEQ ID NO: 352 - DNA, 20 bp, Artificial Sequence
  DNA primer
ctatgattgtcagggccaac

SEQ ID NO: 353 - DNA, 20 bp, Artificial Sequence
  DNA primer
gacaatcatagccagcacct

SEQ ID NO: 354 - DNA, 20 bp, Artificial Sequence
  DNA primer
ctacctcaccaagcatgacg

SEQ ID NO: 355 - DNA, 20 bp, Artificial Sequence
  DNA primer
gcgcaaaccgcgccaagcaa

SEQ ID NO: 356 - DNA, 20 bp, Artificial Sequence
  DNA primer
tccagcagcactaaggtgcg

SEQ ID NO: 357 - DNA, 20 bp, Artificial Sequence
  DNA primer
aatgagtctcccgcacgttg

SEQ ID NO: 358 - DNA, 20 bp, Artificial Sequence
  DNA primer
cggtagtgtctgtcatttcg

SEQ ID NO: 359 - DNA, 20 bp, Artificial Sequence
  DNA primer
gtaaccgaactgcagcgccc

SEQ ID NO: 360 - DNA, 20 bp, Artificial Sequence
  DNA primer
gttcggttactgtctcctcg

SEQ ID NO: 361 - DNA, 20 bp, Artificial Sequence
  DNA primer
cacgccgtaggcaagcgggg

SEQ ID NO: 362 - DNA, 20 bp, Artificial Sequence
  DNA primer
aggaagggaaagctcccggt

SEQ ID NO: 363 - DNA, 20 bp, Artificial Sequence
  DNA primer
ctgctctgcatccagctcgg

SEQ ID NO: 364 - DNA, 20 bp, Artificial Sequence
  DNA primer
tcttctccgcaggatgatca

SEQ ID NO: 365 - DNA, 20 bp, Artificial Sequence
  DNA primer
gcccagcatgttggcgaaga

SEQ ID NO: 366 - DNA, 20 bp, Artificial Sequence
  DNA primer
gccatcttcgccaacatgct

SEQ ID NO: 367 - DNA, 20 bp, Artificial Sequence
  DNA primer
gccagcaccgatccactcag

SEQ ID NO: 368 - DNA, 20 bp, Artificial Sequence
  DNA primer
ccacgagtatcggtcccata

SEQ ID NO: 369 - DNA, 20 bp, Artificial Sequence
  DNA primer
ccgatactcgtggtgcgccg

SEQ ID NO: 370 - DNA, 20 bp, Artificial Sequence
  DNA primer
cgtggtagaggggctcaaac

SEQ ID NO: 371 - DNA, 20 bp, Artificial Sequence
  DNA primer
tcttgccctgggtgaagcgg

SEQ ID NO: 372 - DNA, 20 bp, Artificial Sequence
  DNA primer
gcctagcatgacccgccggt

SEQ ID NO: 373 - DNA, 20 bp, Artificial Sequence
  DNA primer
aaggagtgcggaggtccaaa

SEQ ID NO: 374 - DNA, 20 bp, Artificial Sequence
  DNA primer
tggtgtacgtgttcaccacg

SEQ ID NO: 375 - DNA, 20 bp, Artificial Sequence
  DNA primer
gctattcaaccagaatcccg

SEQ ID NO: 376 - DNA, 20 bp, Artificial Sequence
  DNA primer
gttggcatctgctagagctt

SEQ ID NO: 377 - DNA, 20 bp, Artificial Sequence
  DNA primer
ggagccgcccagaccggccg

SEQ ID NO: 378 - DNA, 20 bp, Artificial Sequence
  DNA primer
atgagcagcacgtggcgccg

SEQ ID NO: 379 - DNA, 20 bp, Artificial Sequence
  DNA primer
ccttcaagcagagcaccgcc

SEQ ID NO: 380 - DNA, 20 bp, Artificial Sequence
  DNA primer
gctcatgtcgcacaagaaga

SEQ ID NO: 381 - DNA, 20 bp, Artificial Sequence
  DNA primer
aagaggctggactgtctccg

SEQ ID NO: 382 - DNA, 20 bp, Artificial Sequence
  DNA primer
actgtcttgctggagaaccg

SEQ ID NO: 383 - DNA, 20 bp, Artificial Sequence
  DNA primer
tcttcatcatctcccggcca

SEQ ID NO: 384 - DNA, 20 bp, Artificial Sequence
  DNA primer
catgccacgcgggctccatc

SEQ ID NO: 385 - DNA, 20 bp, Artificial Sequence
  DNA primer
cgtgccacgcgggctccatt

SEQ ID NO: 386 - DNA, 20 bp, Artificial Sequence
  DNA primer
cacagccatgtgcagcgttg

SEQ ID NO: 387 - DNA, 20 bp, Artificial Sequence
  DNA primer
cctgtccgacctcttccagt

SEQ ID NO: 388 - DNA, 20 bp, Artificial Sequence
  DNA primer
gtcccctccgtattggacgg

SEQ ID NO: 389 - DNA, 20 bp, Artificial Sequence
  DNA primer
cggttcccaagcaacctcag

SEQ ID NO: 390 - DNA, 20 bp, Artificial Sequence
  DNA primer
gctggttaaagagttcgccc

SEQ ID NO: 391 - DNA, 20 bp, Artificial Sequence
  DNA primer
ccctgcgacctggaacaatg

SEQ ID NO: 392 - DNA, 20 bp, Artificial Sequence
  DNA primer
tgcagctccgaacagcagga

SEQ ID NO: 393 - DNA, 20 bp, Artificial Sequence
  DNA primer
gctggggggcgagctccgta

SEQ ID NO: 394 - DNA, 20 bp, Artificial Sequence
  DNA primer
tcaattctgagagatctact

SEQ ID NO: 395 - DNA, 20 bp, Artificial Sequence
  DNA primer
gaccagtcattcacaaggag

SEQ ID NO: 396 - DNA, 20 bp, Artificial Sequence
  DNA primer
aagctttaagtaagtccaca

SEQ ID NO: 397 - DNA, 20 bp, Artificial Sequence
  DNA primer
aggcgctccatgtagaccag

SEQ ID NO: 398 - DNA, 20 bp, Artificial Sequence
  DNA primer
actcatcagaaacgtctgca

SEQ ID NO: 399 - DNA, 20 bp, Artificial Sequence
  DNA primer
tattcggtccaggacaaact

SEQ ID NO: 400 - DNA, 20 bp, Artificial Sequence
  DNA primer
ctgccggtgcaccttcgcca

SEQ ID NO: 401 - DNA, 20 bp, Artificial Sequence
  DNA primer
cctgtagccgccactcacgc

SEQ ID NO: 402 - DNA, 20 bp, Artificial Sequence
  DNA primer
tcttgcgcggagatggcgcg

SEQ ID NO: 403 - DNA, 20 bp, Artificial Sequence
  DNA primer
ctgctgctcatgatcgagcg

SEQ ID NO: 404 - DNA, 20 bp, Artificial Sequence
  DNA primer
gctgtgccctcgcggccggg

SEQ ID NO: 405 - DNA, 20 bp, Artificial Sequence
  DNA primer
ctgtccgcacaccgcccgca

SEQ ID NO: 406 - DNA, 20 bp, Artificial Sequence
  DNA primer
tgcatacagctgttacccga

SEQ ID NO: 407 - DNA, 20 bp, Artificial Sequence
  DNA primer
ttattatcagtccaaaaacg

SEQ ID NO: 408 - DNA, 20 bp, Artificial Sequence
  DNA primer
ggtttttgcgcttcaaaaag

SEQ ID NO: 409 - DNA, 20 bp, Artificial Sequence
  DNA primer
gatccgggccaacggctcgg

SEQ ID NO: 410 - DNA, 20 bp, Artificial Sequence
  DNA primer
aagaacacgatgttgcgccg

SEQ ID NO: 411 - DNA, 20 bp, Artificial Sequence
  DNA primer
gcggaacaggatgttgagca

SEQ ID NO: 412 - DNA, 20 bp, Artificial Sequence
  DNA primer
aacatgatgttggtgaccgg

SEQ ID NO: 413 - DNA, 20 bp, Artificial Sequence
  DNA primer
cgagacccacaacctgtccg

SEQ ID NO: 414 - DNA, 20 bp, Artificial Sequence
  DNA primer
tagcgcgccaggaagagcca

SEQ ID NO: 415 - DNA, 20 bp, Artificial Sequence
  DNA primer
gtcatgtgcttggggcgcgg

SEQ ID NO: 416 - DNA, 20 bp, Artificial Sequence
  DNA primer
tgccgagcgccacaacctga

SEQ ID NO: 417 - DNA, 20 bp, Artificial Sequence
  DNA primer
acttcgtgcacccggccacg

SEQ ID NO: 418 - DNA, 20 bp, Artificial Sequence
  DNA primer
ttgggtagccaaactggtag

SEQ ID NO: 419 - DNA, 20 bp, Artificial Sequence
  DNA primer
gtaaaaggctaccgcccaca

SEQ ID NO: 420 - DNA, 20 bp, Artificial Sequence
  DNA primer
tgaacctcatgtggtgacag

SEQ ID NO: 421 - DNA, 20 bp, Artificial Sequence
  DNA primer
aattgagcagcgacatacaa

SEQ ID NO: 422 - DNA, 20 bp, Artificial Sequence
  DNA primer
tctaaagtggcatcttgccg

SEQ ID NO: 423 - DNA, 20 bp, Artificial Sequence
  DNA primer
gcaagatgccactttagatg

SEQ ID NO: 424 - DNA, 20 bp, Artificial Sequence
  DNA primer
aggagcttctgcggaaagcg

SEQ ID NO: 425 - DNA, 20 bp, Artificial Sequence
  DNA primer
atcatcatcggcgtgcgcaa

SEQ ID NO: 426 - DNA, 20 bp, Artificial Sequence
  DNA primer
gaagtggacctcgttctccg

SEQ ID NO: 427 - DNA, 20 bp, Artificial Sequence
  DNA primer
cagcgtgatctggctctcga

SEQ ID NO: 428 - DNA, 20 bp, Artificial Sequence
  DNA primer
gacatgttgaagatgcgtcg

SEQ ID NO: 429 - DNA, 20 bp, Artificial Sequence
  DNA primer
cgtgtaatcagagatggcac

SEQ ID NO: 430 - DNA, 20 bp, Artificial Sequence
  DNA primer
cgtcctggccggaggcccga

SEQ ID NO: 431 - DNA, 20 bp, Artificial Sequence
  DNA primer
gctggcggtgtggccggcgg

SEQ ID NO: 432 - DNA, 20 bp, Artificial Sequence
  DNA primer
gcctcctcgccgtcgtcgcg

SEQ ID NO: 433 - DNA, 20 bp, Artificial Sequence
  DNA primer
gcgagcttcctcctcaccgg

SEQ ID NO: 434 - DNA, 20 bp, Artificial Sequence
  DNA primer
atagagccagacgcagagca

SEQ ID NO: 435 - DNA, 20 bp, Artificial Sequence
  DNA primer
atgttcctgtactcgtgcgc

SEQ ID NO: 436 - DNA, 20 bp, Artificial Sequence
  DNA primer
cttcttctccccatagtcgg

SEQ ID NO: 437 - DNA, 20 bp, Artificial Sequence
  DNA primer
ggatcgcctccagcagcgcg

SEQ ID NO: 438 - DNA, 20 bp, Artificial Sequence
  DNA primer
cgtgcacccggacgtgcggg

SEQ ID NO: 439 - DNA, 20 bp, Artificial Sequence
  DNA primer
cacccagtcgaccttcaatg

SEQ ID NO: 440 - DNA, 20 bp, Artificial Sequence
  DNA primer
cgcgccctgcagtttaagcg

SEQ ID NO: 441 - DNA, 20 bp, Artificial Sequence
  DNA primer
cctgctgcacgagttccgga

SEQ ID NO: 442 - DNA, 20 bp, Artificial Sequence
  DNA primer
tcgcgtcacgaagtagctgg

SEQ ID NO: 443 - DNA, 20 bp, Artificial Sequence
  DNA primer
gacatggcgtggatgcggcg

SEQ ID NO: 444 - DNA, 20 bp, Artificial Sequence
  DNA primer
ggacacgaagctgatcgtgg

SEQ ID NO: 445 - DNA, 20 bp, Artificial Sequence
  DNA primer
cgctcaggtagcgggacacg

SEQ ID NO: 446 - DNA, 20 bp, Artificial Sequence
  DNA primer
atgcaacgacgtcttccacg

SEQ ID NO: 447 - DNA, 20 bp, Artificial Sequence
  DNA primer
ggagctgccgccctgctacg

SEQ ID NO: 448 - DNA, 20 bp, Artificial Sequence
  DNA primer
ggaggacgatcacggcaaat

SEQ ID NO: 449 - DNA, 20 bp, Artificial Sequence
  DNA primer
ggtgccggacccgtaccgct

SEQ ID NO: 450 - DNA, 20 bp, Artificial Sequence
  DNA primer
ccgcgggtgaaattgtagcg

SEQ ID NO: 451 - DNA, 20 bp, Artificial Sequence
  DNA primer
cctccacatccagaagacgg

SEQ ID NO: 452 - DNA, 20 bp, Artificial Sequence
  DNA primer
cctggtgaagaacatccggc

SEQ ID NO: 453 - DNA, 20 bp, Artificial Sequence
  DNA primer
tctccttcttgccaggccgg

SEQ ID NO: 454 - DNA, 20 bp, Artificial Sequence
  DNA primer
acgctcactgacaagggccg

SEQ ID NO: 455 - DNA, 20 bp, Artificial Sequence
  DNA primer
cgtccaggttgacatacttg

SEQ ID NO: 456 - DNA, 20 bp, Artificial Sequence
  DNA primer
gtactgtgtggcctacggcg

SEQ ID NO: 457 - DNA, 20 bp, Artificial Sequence
  DNA primer
acattgactgataataccca

SEQ ID NO: 458 - DNA, 20 bp, Artificial Sequence
  DNA primer
actgctagaccggtactgcg

SEQ ID NO: 459 - DNA, 20 bp, Artificial Sequence
  DNA primer
ctactgagcgcccagctcaa

SEQ ID NO: 460 - DNA, 20 bp, Artificial Sequence
  DNA primer
tctgcttactacctgtacag

SEQ ID NO: 461 - DNA, 20 bp, Artificial Sequence
  DNA primer
gttgatatggtaggtgttgg

SEQ ID NO: 462 - DNA, 20 bp, Artificial Sequence
  DNA primer
ctacaaatactaggactgtg

SEQ ID NO: 463 - DNA, 20 bp, Artificial Sequence
  DNA primer
gtagacccctgagtgatgtg

SEQ ID NO: 464 - DNA, 20 bp, Artificial Sequence
  DNA primer
acatcactcaggggtctacc

SEQ ID NO: 465 - DNA, 20 bp, Artificial Sequence
  DNA primer
acattcagctgtatgcagct

SEQ ID NO: 466 - DNA, 20 bp, Artificial Sequence
  DNA primer
tgttgtacaccacctggctt

SEQ ID NO: 467 - DNA, 20 bp, Artificial Sequence
  DNA primer
ctaccctgttgtacaccacc

SEQ ID NO: 468 - DNA, 20 bp, Artificial Sequence
  DNA primer
agaatcttgtcggagaagca

SEQ ID NO: 469 - DNA, 20 bp, Artificial Sequence
  DNA primer
tggctagtagagaatgcacc

SEQ ID NO: 470 - DNA, 20 bp, Artificial Sequence
  DNA primer
ggaaatgtacgagtattcca

SEQ ID NO: 471 - DNA, 20 bp, Artificial Sequence
  DNA primer
gtattccaaggtccgctcat

SEQ ID NO: 472 - DNA, 20 bp, Artificial Sequence
  DNA primer
aagccactactcgaacgccg

SEQ ID NO: 473 - DNA, 20 bp, Artificial Sequence
  DNA primer
taacaactatatgaaccacg

SEQ ID NO: 474 - DNA, 20 bp, Artificial Sequence
  DNA primer
ttgaagcccccaacaacagg

SEQ ID NO: 475 - DNA, 20 bp, Artificial Sequence
  DNA primer
tacatcagctgccaaccggg

SEQ ID NO: 476 - DNA, 20 bp, Artificial Sequence
  DNA primer
aatgatgactggcttgcggg

SEQ ID NO: 477 - DNA, 20 bp, Artificial Sequence
  DNA primer
cactgggctgcaatctccca

SEQ ID NO: 478 - DNA, 20 bp, Artificial Sequence
  DNA primer
cctgagcttaggagtccccg

SEQ ID NO: 479 - DNA, 20 bp, Artificial Sequence
  DNA primer
tggctgatccaccaatagcc

SEQ ID NO: 480 - DNA, 20 bp, Artificial Sequence
  DNA primer
gaacttctccgtcaggcgga

SEQ ID NO: 481 - DNA, 20 bp, Artificial Sequence
  DNA primer
tgggggcacgaagtccaccg

SEQ ID NO: 482 - DNA, 20 bp, Artificial Sequence
  DNA primer
tgcccttttctcgcggatgg

SEQ ID NO: 483 - DNA, 20 bp, Artificial Sequence
  DNA primer
gtcaacattcgatttattgg

SEQ ID NO: 484 - DNA, 20 bp, Artificial Sequence
  DNA primer
gcggtgatcgagcctgagca

SEQ ID NO: 485 - DNA, 20 bp, Artificial Sequence
  DNA primer
gctataagcgttatgcaatg

SEQ ID NO: 486 - DNA, 20 bp, Artificial Sequence
  DNA primer
ggtcagcttgaaattgtgac

SEQ ID NO: 487 - DNA, 20 bp, Artificial Sequence
  DNA primer
ggagctgccgtttgacaacg

SEQ ID NO: 488 - DNA, 20 bp, Artificial Sequence
  DNA primer
atgtcatcatggcaaagttt

SEQ ID NO: 489 - DNA, 20 bp, Artificial Sequence
  DNA primer
taaacaacgtcaatcggcat

SEQ ID NO: 490 - DNA, 20 bp, Artificial Sequence
  DNA primer
gctgctcacccaaacgcgtt

SEQ ID NO: 491 - DNA, 20 bp, Artificial Sequence
  DNA primer
tagcaagtcgtcgtcgcggg

SEQ ID NO: 492 - DNA, 23 bp, Artificial Sequence
  DNA primer
ggggccactagggacaggattgg

SEQ ID NO: 493 - DNA, 30 bp, Artificial Sequence
  DNA primer
accccacagtggggccactagggacaggat

SEQ ID NO: 494 - DNA, 39 bp, Artificial Sequence
  DNA primer
tcccgaggcatactggaaccgagagcaagagaagctgaa

SEQ ID NO: 495 - DNA, 41 bp, Artificial Sequence
  DNA primer
tggaagccttctgattgcatgcctcggtggaaggtagggtg

SEQ ID NO: 496 - DNA, 42 bp, Artificial Sequence
  DNA primer
ctgtctgtacttcatctatgtggccccaggcatcggtaagca

SEQ ID NO: 497 - DNA, 39 bp, Artificial Sequence
  DNA primer
gtcggcggcatcctgctgctctccaagcagcactaccgg

SEQ ID NO: 498 - DNA, 37 bp, Artificial Sequence
  DNA primer
tacctgtttccactacttttttagggtcctggttgct

SEQ ID NO: 499 - DNA, 40 bp, Artificial Sequence
  DNA primer
aacttcgtcagccccccgcagtacccggactacggcggtt

SEQ ID NO: 500 - DNA, 43 bp, Artificial Sequence
  DNA primer
ccccaaccaggtagtagaaggctgttgttcagatatggctgtt

SEQ ID NO: 501 - DNA, 36 bp, Artificial Sequence
  DNA primer
ctggatgcccattgtgagtgtacagtgggatggctg

SEQ ID NO: 502 - DNA, 38 bp, Artificial Sequence
  DNA primer
gaccttaccccatgaccgatgctgagagagtggatcag

SEQ ID NO: 503 - DNA, 39 bp, Artificial Sequence
  DNA primer
gaccgcttgggctaccacagagatgtgccagacacaagg

SEQ ID NO: 504 - DNA, 39 bp, Artificial Sequence
  DNA primer
cttgagacatccccggatatcctgctagaagaagtgatc

SEQ ID NO: 505 - DNA, 38 bp, Artificial Sequence
  DNA primer
agcctttgctggcaagaataaaggaagacaggtaagaa

SEQ ID NO: 506 - DNA, 43 bp, Artificial Sequence
  DNA primer
accttcacctacatcgagtctgcctcggagctcagagggggtg

SEQ ID NO: 507 - DNA, 42 bp, Artificial Sequence
  DNA primer
gtcggccctactcaggaccgtggtcaggtgaggccaggagat

SEQ ID NO: 508 - DNA, 36 bp, Artificial Sequence
  DNA primer
ttcaacaaaccttctccttatggaagtaaccataac

SEQ ID NO: 509 - DNA, 43 bp, Artificial Sequence
  DNA primer
ttcatgcctccgcaggagccggccgtgccagggagctggggtc

SEQ ID NO: 510 - DNA, 38 bp, Artificial Sequence
  DNA primer
ccagtaatcgaagtcatcaatgataaggatatgaggta

SEQ ID NO: 511 - DNA, 39 bp, Artificial Sequence
  DNA primer
ggccaccacaggaccccaatgcccctggggcagatggaa

SEQ ID NO: 512 - DNA, 39 bp, Artificial Sequence
  DNA primer
cctgtggtaccatggcctcatgttgaaggagtagaagtg

SEQ ID NO: 513 - DNA, 41 bp, Artificial Sequence
  DNA primer
catccccgacacgcgagactacaggtgggatgaaccaggct

SEQ ID NO: 514 - DNA, 36 bp, Artificial Sequence
  DNA primer
agcccgcactgtcgcggatccgagaggaccggcgtc

SEQ ID NO: 515 - DNA, 43 bp, Artificial Sequence
  DNA primer
ccgagtggctgccgcccatgctgcagcgggtgaaggaggtgag

SEQ ID NO: 516 - DNA, 36 bp, Artificial Sequence
  DNA primer
agcgtcatcctctgtttccatgatgaggcctggtcc

SEQ ID NO: 517 - DNA, 37 bp, Artificial Sequence
  DNA primer
ctggtgttcctggacagccactgtgaggtgaacagag

SEQ ID NO: 518 - DNA, 42 bp, Artificial Sequence
  DNA primer
tccggacccgtctcctgggggcatctatggccagaggagaag

SEQ ID NO: 519 - DNA, 43 bp, Artificial Sequence
  DNA primer
ccctcttccctttgcagccatcaagctggatggggtggatgtc

SEQ ID NO: 520 - DNA, 38 bp, Artificial Sequence
  DNA primer
taccgcagccgcgtctatcaggtgctccggctctcgac

SEQ ID NO: 521 - DNA, 38 bp, Artificial Sequence
  DNA primer
aacatcgcccgcttttcttcaaggggaatgactacctg

SEQ ID NO: 522 - DNA, 42 bp, Artificial Sequence
  DNA primer
gtcttcctggaccacttcccgcccggcggccggcaggacggc

SEQ ID NO: 523 - DNA, 43 bp, Artificial Sequence
  DNA primer
ctttgtcttggtatactacatggcaaaatgggaaaggtaagga

SEQ ID NO: 524 - DNA, 42 bp, Artificial Sequence
  DNA primer
atcctggacctcagcaaaaggtacatcaaggcactggcagaa

SEQ ID NO: 525 - DNA, 39 bp, Artificial Sequence
  DNA primer
ctcgaggaccctggctggagagacagggcaggtaaggtc

SEQ ID NO: 526 - DNA, 42 bp, Artificial Sequence
  DNA primer
ctgctcctcacagtgcttacaggtgaggggcacgaggtgggg

SEQ ID NO: 527 - DNA, 42 bp, Artificial Sequence
  DNA primer
aacatcctctttgattcctggattaagggacaccaggacgtc

SEQ ID NO: 528 - DNA, 39 bp, Artificial Sequence
  DNA primer
ggcttcccagcccccgcccccaacaggccacgggatgac

SEQ ID NO: 529 - DNA, 39 bp, Artificial Sequence
  DNA primer
taccacttctaccatagcaggacgattaaaaggtcagtt

SEQ ID NO: 530 - DNA, 38 bp, Artificial Sequence
  DNA primer
ctactccccagcctcagctcccgaggtaggtgctgggg

SEQ ID NO: 531 - DNA, 35 bp, Artificial Sequence
  DNA primer
agccaaggctctgctgaggagcctgggcagccagg

SEQ ID NO: 532 - DNA, 42 bp, Artificial Sequence
  DNA primer
atgatcctggtgcccttcaagaccatcgacttggagtgggtg

SEQ ID NO: 533 - DNA, 39 bp, Artificial Sequence
  DNA primer
cacaaagacgacccaaggaaatgggggccagaccaggaa

SEQ ID NO: 534 - DNA, 34 bp, Artificial Sequence
  DNA primer
agccaacacaaagccccgtatggctggcgggggc

SEQ ID NO: 535 - DNA, 38 bp, Artificial Sequence
  DNA primer
gacctgcagctcctcgtcttcatgtttccaggtatgtg

SEQ ID NO: 536 - DNA, 40 bp, Artificial Sequence
  DNA primer
tgctgcaagcttatgctttcttgcagtatctgagagaccg

SEQ ID NO: 537 - DNA, 41 bp, Artificial Sequence
  DNA primer
ttctcctgctgctgctgctctgcatccagctcgggggagga

SEQ ID NO: 538 - DNA, 40 bp, Artificial Sequence
  DNA primer
ggacgccggagacccttctctccccatcaggtctgtggct

SEQ ID NO: 539 - DNA, 42 bp, Artificial Sequence; identical to SEQ ID NO: 532
  DNA primer

SEQ ID NO: 540 - DNA, 44 bp, Artificial Sequence
  DNA primer
ccctacctggactcaggggccctggatgggacgcaccgggtgaa

SEQ ID NO: 541 - DNA, 43 bp, Artificial Sequence
  DNA primer
cctggctggccgcgacctgagccgcctgccccaactggtcgga

SEQ ID NO: 542 - DNA, 37 bp, Artificial Sequence
  DNA primer
caggttctccaagccagcacccatgttcctggatgac

SEQ ID NO: 543 - DNA, 42 bp, Artificial Sequence
  DNA primer
gacccccggggaccccgccatgttgccgttgctatgaacaag

SEQ ID NO: 544 - DNA, 39 bp, Artificial Sequence
  DNA primer
ggcatctacgtcatccaccaggtgagcgtgggggcagac

SEQ ID NO: 545 - DNA, 39 bp, Artificial Sequence
  DNA primer
tctgcccacttcgaccccaaagtagaaaacaacccagac

SEQ ID NO: 546 - DNA, 39 bp, Artificial Sequence
  DNA primer
acctgccatgaaaccacacttgaagcaatggagacaacg

SEQ ID NO: 547 - DNA, 41 bp, Artificial Sequence
  DNA primer
ccggaggacctgctgttcttctacgagcacctcaggaaggg

SEQ ID NO: 548 - DNA, 40 bp, Artificial Sequence
  DNA primer
ctcagcggggcctcgctaccaatacttgattaaccacaag

SEQ ID NO: 549 - DNA, 60 bp, Artificial Sequence
  DNA primer
tgacttatcaccccaaccaggtagtagaaggctgttgttcagatatggctgttactttta

SEQ ID NO: 550 - protein, 219 aa, Artificial Sequence
  Sequence of human mucin or mucin domains of glycoprotein
PTLGDEGDTDLYDYYPEEDTEGDKVRATRTVVKFPTKAHTTPWGLFYSWSTASLDSQMPSSLHPTQESTKEQTTFPPRWTPNFTLHMESITFSKTPKSTTEPTPSPTTSEPVPEPAPNMTTLEPTPSPTTPEPTSEPAPSPTTPEPTSEPAPSPTTPEPTSEPAPSPTTPEPTPIPTIATSPTILVSATSLITPKSTFLTTTKPVSLLESTKKTIPELD

SEQ ID NO: 551 - protein, 160 aa, Artificial Sequence
  Sequence of human mucin or mucin domains of glycoprotein
APDTRPAPGSTAPPAHGVTSAPDTRPAPGSTAPPAHGVTSAPDTRSAPGSTAPAAHGVTSAPDTRSVPGSTAPQAHGVTSAPDTRPAPGSTAPPAHGVTSAPDTRPVPGSTAPPAHGVTSAPDTRPAPGSTAPPAHGVTSAPDTRPAPGSTAPQAHGVTS

SEQ ID NO: 552 - protein, 160 aa, Artificial Sequence
  Sequence of human mucin or mucin domains of glycoprotein
PPTPSATTQAPPSSSAPPETTAAPPTPPATTPAPPSSSAPPETTAAPPTPSATTPAPLSSSAPPETTAVPPTPSATTLDPSSASAPPETTAAPPTPSATTPAPPSSPAPQETTAAPITTPNSSPTTLAPDTSETSAAPTHQTTTSVTTQTTTTKQPTSAP

SEQ ID NO: 553 - protein, 148 aa, Artificial Sequence
  Sequence of human mucin or mucin domains of glycoprotein
SPPPTSTTTLPPTTTPSPPTTTTTTPPPTTTPSPPITTTTTPPPTTTPSPPISTTTTPPPTTTPSPPTTTPSPPTTTPSPPTTTTTTPPPTTTPSPPTTTPITPPASTTTLPPTTTPSPPTTTTTTPPPTTTPSPPTTTPITPPTSTT

SEQ ID NO: 554 - protein, 149 aa, Artificial Sequence
  Sequence of human mucin or mucin domains of glycoprotein
TQTPTTTPITTTTTVTPTPTPTGTQTPTPTPITTTTTVTPTPTPTGTQTPTSTPITTTTTVTPTPTPTGTQTPTMTPITTTTTVTPTPTPTGTQTPTTTPISTTTTVTPTPTPTGTQTPTSTPITTTTTVTPTPTPTGTQTPTTTPITT

SEQ ID NO: 555 - protein, 146 aa, Artificial Sequence
  Sequence of human mucin or mucin domains of glycoprotein
EISSHSTPSFSSSTIYSTVSTSTTAISSLPPTSGTMVTSTTMTPSSLSTDIPFTTPTTITHHSVGSTGFLTTATDLTSTFTVSSSSAMSTSVIPSSPSIQNTETSSLVSMTSATTPNVRPTFVSTLSTPTSSLLTTFPATYSFSSS

SEQ ID NO: 556 - protein, 144 aa, Artificial Sequence
  Sequence of human mucin or mucin domains of glycoprotein
MTSATTPNVRPTFVSTLSTPTSSLLTTFPATYSFSSSMSASSAGTTHTESISSPPASTSTLHTTAESTLAPTTTTSFTTSTTMEPPSTTAATTGTGQTTFTSSTATFPETTTPTPTTDMSTESLTTAMTSPPITSSVTSTNTVT

SEQ ID NO: 557 - protein, 151 aa, Artificial Sequence
  Sequence of human mucin or mucin domains of glycoprotein
TTPTPTTDMSTESLTTAMTSPPITSSVTSTNTVTSMTTTTSPPTTTNSFTSLTSMPLSSTPVPSTEVVTSGTINTIPPSILVTTLPTPNASSMTTSETTYPNSPTGPGTNSTTEITYPTTMTETSSTATSLPPTSPLVSTAKTAKTPTTNL

SEQ ID NO: 558 - protein, 135 aa, Artificial Sequence
  Sequence of human mucin or mucin domains of glycoprotein
TTTETTSHSTPGFTSSITTTETTSHSTPSFTSSITTTETTSHDTPSFTSSITTSETPSHSTPSSTSLITTTKTTSHSTPSFTSSITTTETTSHSAHSFTSSITTTETTSHNTRSFTSSITTTETNSHSTTSFTSS

SEQ ID NO: 559 - protein, 143 aa, Artificial Sequence
  Sequence of human mucin or mucin domains of glycoprotein
LPVTSPSSASTGHATPLLVTDTSSASTGHATPLPVTDASSVSTDHATSLPVTIPSAASTGHTTPLPVTDTSSASTGQATSLLVTDTSSVSTGDTTPLPVTSTSSASTGHVTPLHVTSPSSASTGHATPLPVTSLSSASTGDTM

SEQ ID NO: 560 - protein, 143 aa, Artificial Sequence
  Sequence of human mucin or mucin domains of glycoprotein
TTSAPTTSTTSAPTTSTISAPTTSTTSATTTSTTSAPTPRRTSAPTTSTISASTTSTTSATTTSTTSATTTSTISAPTTSTTLSPTTSTTSTTITSTTSAPISSTTSTPQTSTTSAPTTSTTSGPGTTSSPVPTTSTTSAPTT

SEQ ID NO: 561 - protein, 122 aa, Artificial Sequence
  Sequence of human mucin or mucin domains of glycoprotein
TSATSSRLPTPFTTHSPPTGTTPISSTGPVTATSFQTTTTYPTPSHPHTTLPTHVPSFSTSLVTPSTHTVIIPTHTQMATSASIHSMPTGTIPPPTTIKATGSTHTAPPMTPTTSGTSQSPS

SEQ ID NO: 562 - protein, 86 aa, Artificial Sequence
  Sequence of human mucin or mucin domains of glycoprotein
SIHSMPTGTIPPPTTIKATGSTHTAPPMTPTTSGTSQSPSSFSTAKTSTSLPYHTSSTHHPEVTPTSTTNITPKHTSTGTRTPVAH

SEQ ID NO: 563 - protein, 89 aa, Artificial Sequence
  Sequence of human mucin or mucin domains of glycoprotein
AMTMTSVGHQSMTPGEKALTPVGHQSVTTGQKTLTSVGYQSVTPGEKTLTPVGHQSVTPVSHQSVSPGGTTMTPVHFQTETLRQNTVAP

SEQ ID NO: 564 - protein, 142 aa, Artificial Sequence
  Sequence of human mucin or mucin domains of glycoprotein
TTETATSGPTVAAADTTETNFPETASTTANTPSFPTATSPAPPIISTHSSSTIPTPAPPIISTHSSSTIPIPTAADSESTTNVNSLATSDIITASSPNDGLITMVPSETQSNNEMSPTTEDNQSSGPPTGTALLETSTLNST

SEQ ID NO: 565 - protein, 149 aa, Artificial Sequence
  Sequence of human mucin or mucin domains of glycoprotein
LSTTPVDTSTPVTNSTEARSSPTTSEGTSMPTSTPSEGSTPFTSMPVSTMPVVTSEASTLSATPVDTSTPVTTSTEATSSPTTAEGTSIPTSTLSEGTTPLTSIPVSHTLVANSEVSTLSTTPVDSNTPFTTSTEASSPPPTAEGTSMP

SEQ ID NO: 566 - protein, 149 aa, Artificial Sequence
  Sequence of human mucin or mucin domains of glycoprotein
VTRTTRSSAGLTGKTGLSAGVTGKTGLSAEVTGTTRLSAGVTGTTGPSPGVTGTTGTPAGVTGTTELSAGVTGKTGLSSEVTETTGLSYGVKRTIGLSAGSTGTSGQSAGVAGTTTLSAEVTGTTRPSAGVTGTTGLSAEVTEITGISA

SEQ ID NO: 567 - protein, 151 aa, Artificial Sequence
  Sequence of human mucin or mucin domains of glycoprotein
ESSASSDSPHPVITPSRASESSASSDGPHPVITPSRASESSASSDGPHPVITPSRASESSASSDGPHPVITPSRASESSASSDGPHPVITPSRASESSASSDGPHPVITPSRASESSASSDGPHPVITPSRASESSASSDGPHPVITPSRA

SEQ ID NO: 568 - protein, 149 aa, Artificial Sequence
  Sequence of human mucin or mucin domains of glycoprotein
SGASTATNSDSSTTSSGASTATNSDSSTTSSEASTATNSESSTTSSGASTATNSESSTVSSRASTATNSESSTTSSGASTATNSESRTTSNGAGTATNSESSTTSSGASTATNSESSTPSSGAGTATNSESSTTSSGAGTATNSESSTV

SEQ ID NO: 569 - protein, 148 aa, Artificial Sequence
  Sequence of human mucin or mucin domains of glycoprotein
GTTTASMAGSETTVSTAGSETTTVSITGTETTMVSAMGSETTTNSTTSSETTVTSTAGSETTTVSTVGSETTTAYTADSETTAASTTGSEMTTVFTAGSETITPSTAGSETTTVSTAGSETTTVSTTGSETTTASTAHSETTAASTMG

SEQ ID NO: 570 - protein, 300 aa, Artificial Sequence
  Sequence of human mucin or mucin domains of glycoprotein
TSSGDTDYDDYDDIPDVPATRTEVKFSTNTKVHTTHWSLLAAAPSTSQDSQMISLPPTHKPTKKQSTFIHTQSPGFTTLPETMESNPTFYSLKLNTVLIPSPTTLEPTSTQATPEPNIQPMLTTSTLTTPEHSTTPVPTTTILTTPEHSTIPVPTTAILTTPKPSTIPVPTTATLTTLEPSTTPVPTTATLTTPEPSTTLVPTTATLTTPEHSTTPVPTTATLTTPEHSTTPVPTTATLTTPEPSTTLTNLVSTISPVLTTTLTTPESTPIETILEQFFTTELTLLPTLESTTTIIPEQN

SEQ ID NO: 571 - DNA, 20 bp, Artificial Sequence
  DNA primer
tcgttggcctgattctcccc

SEQ ID NO: 572 - DNA, 20 bp, Artificial Sequence
  DNA primer
gccagaggtttttgctcagg

SEQ ID NO: 573 - DNA, 19 bp, Artificial Sequence
  DNA primer
caggacaacgttcacacgg

(19) United States
(12) Patent Application Publication
(10) Pub. No.: US 2019/0330601 A1
(43) Pub. Date: Oct. 31, 2019

(54) A CELL-BASED ARRAY PLATFORM

(71) Applicant: UNIVERSITY OF COPENHAGEN, Copenhagen K (DK)

(72) Inventors: Eric BENNETT, Lyngby (DK); Yoshiki NARIMATSU, Hellerup (DK); Catharina STEENTOFT, Copenhagen O (DK); Zhang YANG, Gentofte (DK); Ulla MANDEL, Copenhagen O (DK); Henrik CLAUSEN, Copenhagen O (DK)

(21) Appl. No.: 16/300,196

(22) PCT Filed: May 11, 2017
(86) PCT No.: PCT/EP2017/061385

§ 371 Date: Nov. 9, 2018

(30) Foreign Application Priority Data
May 13, 2016 (EP) 16169643.0

(51) Int. Cl. C12N 9/10; C12P 21/00; G01N 33/68; C12N 5/071; C12N 15/85

(52) U.S. Cl. CPC C12N 9/1048; C12P 21/005; G01N 33/6842; C12N 15/907; C12N 15/85; G01N 2333/4728; C12N 2015/8518; C12N 5/0602

(57) ABSTRACT

The present invention relates to a method for display of a plurality of mammalian glycans on cells or proteins for probing biological interactions and identifying glycan structures involved. A plurality of mammalian cells is genetically engineered in a combinatorial approach to differentially express the human glycome. Genetic engineering of the cell produces a plurality isogenic cells with different repertoires of glycosyltransferases and display of glycans that is used to interpret biological interactions. The plurality of engineered cells display glycans with and without the context of specific proteins exogeneously expressed, and is useful for detection and optimization of biological interactions for example binding of lectins, antibodies, viruses and bacteria and glycoproteins.

### CROSS REFERENCE TO RELATED APPLICATIONS

This application is a U.S. national phase application of International PCT Patent Application No. PCT/EP2017/061385, which was filed on May 11, 2017, which claims priority to European Application No. 16169643.0, filed May 13, 2016, each of which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is GLYD_001_01US_ST25.txt The text file is 130 KB, was created on July 11, 2019, and is being submitted electronically via EFS-Web.

### FIELD OF THE INVENTION

The present invention relates to a plurality of mammalian cells with different capacities for posttranslational modifications that are useful for displaying and probing biological interactions involving glycans in an arrayable format. The pluralities of mammalian cells are genetically engineered in a combinatorial approach to express different repertoires of glycosyltransferases and interpretable capacities for glycosylation. The plurality of mammalian cells can comprise one or more exogenously added genes encoding polypeptides of interest, wherein the polypeptide of interest is expressed and display different posttranslational modifications in a combinatorial way and dependent on the engineering of the cells. The plurality of engineered cells display glycans with and without the context of specific proteins exogeneously expressed, and is useful for detection of biological interactions for example binding of lectins, antibodies, viruses and bacteria.

The present invention also relates to methods for generating mammalian cells displaying different glycans, glycoproteins and compositions comprising the glycoproteins, as well as genome engineering, cell-based assays, and their uses.

### BACKGROUND OF THE INVENTION

The glycome of mammalian cells includes all glycans on glycoproteins, glycolipids, proteoglycans and glycosylphosphatidylinositol (GPI) anchored proteins, and comprise a highly diverse set of different glycan structures (Rillahan 2011). The glycome is generated post-translationally through a non-template driven process directed by over 200 glycosyltransferase genes and an equally large number of accessory genes encoding enzymes, transporters, adapters and other proteins required for sugar nucleotide synthesis and transport as well as organization of the glycosylation process in the ER and Golgi complex (Hansen 2015). Differential expression of enzymes and their distinct specificities dictate the unique spectrum of structures produced by a given cell. The glycome of mammalian cells, tissues and organisms play pivotal biological roles in normal and disease states, and many of these roles are directed by protein-carbohydrate and carbohydrate-carbohydrate interactions (Paulson 2006). Many pathogens have glycan-binding-proteins (GBPs) that recognize host glycan structures as receptors for attachment enabling colonization and toxin entry (Sharon 2004, Ilver 2003). Eukaryotic organisms have developed GBPs that recognize pathogen glycans as part of the innate immune system. A large number of mammalian GBPs have also evolved to recognize endogenous host glycans, and GBP receptor interactions mediate a variety of functions in the organism including cell-cell adhesion, trafficking and cell signaling (Taylor 2014). The functions of many mammalian GBPs have been clarified, but there are still many with unknown roles.

The binding specificity of mammalian and microbial GBPs towards glycans and glycoconjugates has been extensively studied. Microbial GBPs mediate the attachment of microbes and microbial toxins to host cells via cell-surface glycan ligands. The membrane envelopes of for example influenza viruses (and others viruses such as Sendai, Newcastle disease and measles) are studded with hemagglutinins, and these viral GBPs bind to sialic acid-containing glycan ligands to initiate endocytosis (Stevens 2006). Some non-enveloped viruses in the reovirus (rotavirus) families also bind cell-surface sialic acids on host cells through a shallow pocket on the surface of the capsid (Yu 2014). Many bacteria produce adhesins that use glycans for attachment to host cells.

Progress has been hampered in part by the diversity and complexity of the glycome and technical difficulties in probing interactions. Identification of the fine structural details of ligands for GBPs is confounded by the fact that glycoproteins and glycoconjugates most often carry many different glycan structures as a result of for example heterogeneity and sites of glycosylation, and further that GBPs may selectively recognize glycans in context of the protein or glycoconjugate. Moreover, GBP receptor interactions with ligands may be controlled by particular presentations of the glycoconjugate in cellular systems, such as for example microdomains in cell membranes. The affinity of GBPs for their glycan ligands is typically low (Kd of micromolar to millimolar), and multivalent interactions are required to achieve a biological effect.

Studies of the binding specificities of GBPs were greatly advanced by the development of glycan-arrays displaying hundreds of homogeneous oligosaccharides produced chemically or chemoenzymatically or isolated from natural sources (Blixt 2004). Most recent versions of glycan microarrays use array printing technologies developed for printing cDNA microarrays on glass slides (Paulson 2006). The method of attachment of the glycan to the solid support and attachment may either be noncovalent or covalent. Several glycan array formats are based on noncovalent association of glycans or modified glycans with appropriately prepared surfaces. The surface to which the glycans are attached is critical for the subsequent interrogation with labeled GBP, as low background binding is essential for specific binding to be detected. Regardless of format, the utility of glycan arrays depends on the types and diversity of glycan structures it contains and limitations governed by the surface and mechanisms of coupling to this surface. The ideal array would contain the entire glycome of an organism on a single array. However, current arrays are limited to displaying libraries of natural and synthetic glycans that can be practically and technically assembled. While glycan arrays have advanced our understanding of the binding specificities of GBPs from a large variety of sources (Palma 2014, Geissner 2014, Arthur 2014), the current glycan arrays display oligosaccharide structures without the context of proteins and lipids (glycoconjugates) and the cell membrane. Moreover, the current glycan arrays only display a limited subset of the mammalian glycome mainly due to difficulties in synthesis or isolation of appropriate structures.

There is thus a need for improved methods for characterization of the binding properties of GBPs and biological effects in cells and on particular glycoconjugates including glycoproteins, glycolipids and proteoglycans. Such improved methods are bound to drive discovery of novel drugs targeting the interaction between GBP's and the glycan.

### SUMMARY OF THE INVENTION

The glycan array platforms available in the art are based on synthesized and/or isolated oligosaccharide glycans immobilized on slides or membranes, and these arrays are limited by: i) availability and cost of producing the different glycans; and ii) the unnatural display format of the glycans without context of the glycoconjugate and/or cell surface. In contrast the present invention overcomes these limitations by the display of glycans on natural proteins and cell surfaces, and furthermore presents an unlimited and cost effective source of arrayed glycans. This is accomplished by engineering mammalian cells to produce different glycoforms on natural cell surface or on expressed target protein, allowing expansion of library in complexity and volume by standard engineering and cell culture technologies.

The present invention relates to a plurality of isogenic mammalian cells, wherein one or more glycogenes have been inactivated and/or introduced to alter the glycosylation capacity and display of glycans on the cell surface and in secretome of said cells.

An object of the present invention relates to use of the plurality of isogenic mammalian cells with different glycosylation capacities for display of glycans, and use of the plurality of mammalian cells in binding assays for probing interactions with glycans in the context of native glycoconjugates and the cell membrane.

Another object of the present invention relates to use of the plurality of isogenic mammalian cells with different glycosylation capacities for display of glycans, and use of the plurality of mammalian cells to isolate released glycoconjugates for display of glycoforms and probing binding interactions.

The present invention relates to a plurality of isogenic mammalian cells, wherein one or more glycogenes have been inactivated and/or introduced to alter the glycosylation capacity, and in which a plurality of mammalian proteins are expressed to display the encoded proteins with different glycoforms on the cell surface of said cells.

An object of the present invention relates to use of mammalian cells with different glycosylation capacities for display of glycans, and in which a plurality of mammalian proteins are expressed in different glycoforms to probe binding interactions and other biological effects including pharmaceutical effects.

Another object of the present invention relates to use of mammalian cells with different glycosylation capacities for display of glycans, and in which a plurality of glycovariants of a mammalian protein are released to probe binding interactions.

An object of the present invention relates to a plurality of isogenic mammalian cells comprising one or more glycosyltransferase genes that have been inactivated, and that have different stable glycosylation capacities.

### LEGENDS TO THE FIGURE

FIG. 1A shows overview of the glycan display strategy. Glycans will be displayed and probed in a stepwise approach. Step 1 addresses the type of glycoconjugate (Panel 1B), Step 2 probes type of glycan involved (Panel 1C), Step 3 addresses the glycostructure including branching and elongation (Panel 1D), Step 4 determines type of capping (Panel 1E) and finally Step 5 elucidates role of glycan modifications not driven by glycosyltransferases (Panel 1F). The genes have been grouped according to the step in which they are involved, and gene group number reflects step number.

FIG. 2 shows the glycan display strategy for each Initiation step (See FIGS. 1A, 1B). Glycans will be displayed and probed according to type of glycosylation (initiation). FIG. 2A addresses N-glycans, FIG. 2B addresses O-Glc, O-Fuc and O-GlcNAc, FIG. 2C addresses O-Mannosylation, FIG. 2D addresses O-GalNAc, FIG. 2E addresses glycolipids, and FIG. 2F addresses Glucosaminoglycans (GAGs).

FIG. 3 shows analysis of novel Mabs 4B7 and 6C5 specificity by immunofluorescence cytology. Antibodies 4B7 and 6C5 react specifically with MUC1 carrying Tn-O-glycans as illustrated by binding in MDA-MB-231 cell line engineered to display only Tn-O-glycans (F, G) and no binding to the corresponding wt cells (A, B). Similar staining pattern is seen with established antibodies to Tn-MUC1 (C, H) and Tn (D, I) whereas Mab 3C9 to ST stain wt MDA-MB-231 cells (E) and not the engineered cells (J).

FIG. 4 shows Immunohistochemical staining of breast tissues with novel MAb 6C5 and established Mab 5E5 (Tn-MUC1). Both Mabs stain breast tumor cells (A, C) and not normal cells (B, D).

FIG. 5 shows CAS9—(panel 5A) and U6gRNAEPB (panel 5B) vector maps. The vectors are used for expressing CAS9 protein and gRNA's respectively for targeted knock out experiments.

FIG. 6A shows map of EPB71 vector which is used is used for ZFN driven targeted integration into AAVS1 site in HEK/Human cells.

FIG. 6B shows schematic representation of the human PPP1R12C locus, known as the AAVS1 safe harbour integration locus. InvAAVS1; inverted AAVS1 ZFN binding sites for

AAVS1 ZFN ObLiGaRe mediated donor target integration at the AAVS1 safe harbour site. SH #1; CHO-K1 safe harbour site #1 landing pad and binding site for CHO SH #1 ZFNs. CMV-IE; CMV immediate early promoter. GOI; gene of interest ORF. BgH; bovine growth hormone poly-(A) and 3′URT. Ins; insulator sequences.

FIG. 6C shows map of EPB69 donor vector which is used for ZFN driven targeted integration into the SH1 site.

FIG. 7 shows cartoon of cell surface display constructs. Truncated CD34 (left) and MUC1 (right) C-terminal fragments are used as scaffold to display glycosylated polypeptide on cell surface. FIG. 7B shows maps of the corresponding DNA constructs.

FIG. 8 shows IHC of HEK293 wt cell lines and HEK293 SimpleCell lines transiently expressing MUC1, MUC7 or GP1Ba. The MUC1, MUC7 and GP1Ba DNA fragments were inserted into MUC1 truncated reporter scaffold (See FIG. 7) before transfection. Mock cells did not receive any DNA. For IHC the 5E5 and 5E10 antibodies were used. The 5E5 antibody only reacted with MUC1 and MUC7 transfected SimpleCells (SC), whereas none of the corresponding wt cells were stained. The 5E10 antibody stained both HEK293 wt and SimpleCells (SC) transfected with MUC1 whereas it did not react with cells from MUC7 or GP1Ba transfections. Neither GP1Ba or Mock transfected cells were stained by any of the two antibodies.

FIG. 9A shows staining of HEK293 MGAT5 ko and HEK293 wt cells with Phytohemagglutinin-L lectin (L-PHA) labeling. After knock-out of MGAT5 the HEK cells are not stained with L-PHA, demonstrating that the binding interaction with the HEK293 cells were likely through β6-branch of tetraantennary N-glycans from a N-glycoprotein.

FIG. 9B-D shows lectin binding to glycoengineered cell lines with knock out of indicated genes. Cells were labeled with biotinylated lectins followed by Streptavidin with Alexa 488 before analyzing on a FACSCalibur. The y-axis represent mean flourescense intensities. The lectins used are indicated on each Figure. The following Glycodesigns were analysed: WT cells (black bars in all figures), cells with knock out of B4GALNT3/4 (white bar in FIG. 9B), cells with knock out of B4GALT1/2/3/4 (white bars in FIG. 9B), cells with knock out of MGAT5 (white bar in FIG. 9D).

FIG. 10 shows the QCgRNA amplicon principle where a tri-primer PCR set up, using QCGFOR, QCGRNA-Primer, QCGREV primers (See FIG. 10A and actual primer sequences hereunder) and EPB104 U6 promoter plasmid (FIG. 10B) as template, allows for generation of amplicons containing U6promoter and primer encoded gRNA and trcRNA elements (QCgRNA amplicon). These amplicons are co-transfected with Cas9—for CRISPR/Cas9 targeting.

FIG. 11 shows immunofluorescence cytology with antibodies directed to O-GalNAc glycans and demonstrates that binding with HEK293 cells are likely through an O-glycoprotein. HEK293 cells without C1GALT activity (Simple Cells) were not stained by 3C9 antibody (C) whereas wt HEK293 gave strong staining (A). Mab 5F4 on the other hand only stained HEK293 cells with knock out of C1GALT (D) and not wt HEK293 (B).

FIG. 12A shows immunofluorescence cytology with antibodies directed to O-GalNAc glycans and demonstrates that HEK293 cells with COSMC knock out (indirect inactivation of C1GALT1 activity) are stained by the Tn specific antibody 5F4 whereas the STn specific antibody 3F1 does not stain these cells. When combining COSMC knock out with knock in of ST6GALNAC1 the 3F1 (STn) staining is positive whereas 5F4 (Tn) staining is lost demonstrating modified capping when manipulating group 4 capping enzymes (Table 5).

FIG. 12B-F shows lectin binding to glycoengineered cell lines. Cells were labeled with biotinylated lectins followed by Streptavidin with Alexa 488 before analyzing on a FACSCalibur. Data are mean flourescense intensities. The lectins are indicated on each figure. The following Glycodesigns were analysed: WT cells (black bars in all figures), cells with knock out of ST3GAL1/2/3/4/5/5 and ST6GAL1/2 (white bars in FIG. 12B), cells with knock out of ST3GAL1/2/3/4/5/6 (white bars in FIG. 12C), cells with knock out of ST6GAL1/2 (white bars in FIG. 12D), cells with knock out of ST3GAL3/4/6 and ST6GAL1/2 (white bars in FIG. 12E), and cells with knock out of ST3GAL3/4/5 (white bars in FIG. 12F.

FIG. 13A/B/C shows how editing glycogenes in cells influences glycoforms on secreted proteins. GLA enzyme was expressed in 11 glycoengineered cell lines (d1-d11) and the secreted GLA protein was purified and digested with Chymotrypsin before glycopeptide analysis by MS. For each of the three N-glycans of GLA at positions N139, N192, N215, a graphic depiction of the two major glycoforms identified is shown. The glycodesigns (knock out and knock in) for the engineered cell lines, are included to the left and the glycoforms from GLA expressed in wt cell line is included at the top.

FIG. 14A/B shows glycan display of IgG1. IgG1 was expressed in cells with the genetic designs indicated at the left with knock out (KO) and knock in (KI) of glycogenes. Recombinant expressed IgG1 was purified from cell culture supernatants using Protein G sepharose and glycans released by PNGaseF were labeled with APTS before analyzing by capillary Electrophoresis on a 3500XL Genetic Analyser instrument. Knock out of FUT8 is shown with only a few combinations and all shown designs were unaffected.

FIG. 15 shows loss of O-glycoosylaton of EPO expressed in engineered cells. Glycopeptide analysis was performed on EPO produced in two cell lines; wt cells and HEK293 cells with triple knock out of GALNT1/T2/T3 (ΔT1/T2/T3). The O-glycan at S126 was analysed by tryptic digest and followed by label free LC/MS quantification of the O-glycopeptide EAISPPDAASAAPLR-131 The abundances of glycosylated peptides were normalized to the relative abundances of the naked peptides in each individual LC/MS chromatograph.

FIG. 16 shows a comprehensive strategy to generate mAbs towards aberrant O-glycoproteins. SimpleCells displaying homogeneous Tn and/or STn O-glycans are generated by targeted KO of COSMC or C1GALT1. The O-glycan site occupancy is controlled by the repertoire of GALNAC-Ts in the selected cell line, and can also be engineered by targeted KO or KI of different enzymes in the GALNAC-T family. The SimpleCells provide an unlimited source of immunogens with homogenous cancer associated O-glycosylation either in the format of different cell extracts or recombinant expressed and purified glycoproteins. One application illustrated in this invention was to use lectin affinity purified glycoproteins from breast and ovarian cancer SimpleCell lines (MDA-MB-231 and OVCAR-3) or microvesicles isolated from the culture media of a pancreatic cancer SimpleCell line (T3M4) to immunize mice. The created antibody libraries are screened on the original cancer cell line as well as the engineered SimpleCell line by immunocytochemistry and clones showing preferred reactivity to the latter is selected for further characterization including western blotting, immunohistochemistry, ELISA and mass spectrometry.

FIG. 17 shows the generation of the mAb 6C5. (a) A cell lysate from MDA-MB-231 SC was purified by lectin affinity chromatography using VVA. The enriched Tn-glycoproteins were used as an immunogen and Abs were generated by mouse hybridoma technology. 41 out of 480 hybridoma wells tested, produced Abs reacting with MDA-MB-231 SC as validated on trypsinated, acetone fixed cells. Seven of the 41 Abs exhibited preferred reactive towards SC compared to WT, while the clone designated 6C5 showed no reactivity to the WT cells. MAb 6C5 was cloned and characterized by ICC staining on MDA-MB-231 SC and WT grown on cover slide using anti-Tn (mAb 1E3) as control. (b) Additional characterization was performed on a panel of acetone fixed SC including Colo-205, IMR-32, MCF7 and HepG2 with and without neuraminidase. (c) Immunoprecipitation (IP) was performed with 6C5 on MDA-MB-231 SC lysates and analyzed on western blot. Staining with either 6C5, the secondary anti-Ig-HRP alone or an anti-Tn control (VVA) showed that mAb 6C5 recognizes a <50 kDa glycoprotein.

FIG. 18 illustrates immunohistochemistry with Mab 6C5 in tissue microarrays of breast cancer tissues (a-d), ovarian cancer (e) stomach cancer (f) and the corresponding tumor adjacent normal tissue (g-i). Mab 6C5 showed cell surface immunofluorescence in four types of breast cancers i.e. carcinoma simplex (a), infiltrating duct carcinoma (b), scirrhous carcinoma (c), atypical medullary carcinoma (d) as well as serous papillary cystadenocarcinoma from ovary (e) and adenocarcinoma from stomach (f). Cancer adjacent normal breast (g) and ovary tissue (h) did not react with mAb 6C5, whereas cancer adjacent normal appearing stomach tissue presented with strong intracellular granular staining in most mucous producing cells (left pointing arrow) and a few of those also gave a more homogenous pattern throughout the cell (right pointing arrow) (i).

FIG. 19A/B/C shows that 6C5 specifically recognizes a GALNAC-T7 dependent epitope on FXYD5. FXYD5 was knocked out in the HEK293 SC background and cells were stained by ICC (FIG. 19A-a) as well as FACS (FIG. 19A-b) with an anti-FXYD5 mAb (NCC-MC53) as well as 6C5 and an anti-Tn control (VVA). Cell lysates of HEK293 WT, SC, SC GALNT1/T2/T3 triple KO, SC GALNT7 KO and SC FXYD5 KO was analyzed by western blot (FIG. 19B-c), and the anti-FXYD5 mAb and 6C5 mAb was used for IP with either SC or SC GALNT7 KO cell lysate as input (FIG. 19B-d) confirming that while 6C5 staining disappears upon GALNT7 KO, FXYD5 expression is unchanged. To validate the finding a full length FXYD5 construct was expressed in the SC FXYD5 KO cells and IP was performed on cell lysate from either SC or SC FXYD5 KO+FXYD5-recombinant and detected with anti-FXYD5 mAb, 6C5 or anti-Tn (VVA) (FIG. 19C-e). A 30 mer peptide covering aa 81-110 of FXYD5 (TDGPLVTDPETHKSTKAAHPTDDTTTLSER) was purchased and in vitro glycosylated with either GalNAcT1 or GalNAcT1 and T7 in combination. Peptides and glycopeptides were tested for 6C5 and anti-Tn reactivity (VVA) by ELISA including a MUC1 20 mer 3Tn glycopeptide (AHGVTSAPDTRPAPGSTAPP) as well as a 30 mer OTS8 5Tn glyco-peptide (KAPLVPTQRERGTKPPLEELSTSATSDHDH) as controls (FIG. 19C-f).

FIG. 20 shows loss of LacDiNac structure on GLA enzyme expressed in engineered cells. GLA enzyme was expressed in WT cells and cells with double knock out of B4GALNT3/4 (LDN KO). Secreted GLA protein was purified and digested with Chymotrypsin before glycopeptide analysis by MS. A) Extracted ion chromatogram (XIC) of the precursor ions at m/z 1210.1586, z=3+ assigned to ₁₃₅ADVGNKTCAGFPGSF₁₄₉ glycopeptide bearing NeuAcHex₄HexNAc₅ N-glyco structure. Based on the MSMS analysis two N-glycoforms were proposed, either with neutral or sialyted LacDiNac terminal epitope. B) XIC of the fragment ions diagnostic for SiaLacDiNAc, LacDiNAc and NeuAcHexNAc are presented. A part of MSMS spectrum (m/z range 250-800) is presented as insert.

### DETAILED DISCLOSURE OF THE INVENTION

As described above the present invention relates to a plurality of isogenic mammalian cells, wherein one or more glycogenes have been inactivated and/or introduced to alter the glycosylation capacity and display of glycans on the cell surface of said cells.

In some embodiments of the present invention the plurality of mammalian cells comprises two or more glycosyltransferase genes that have been inactivated.

Group 2 (Type of Glycoconjugate)

In some embodiments of the present invention the plurality of mammalian cells comprises or consists of mammalian cells with individual and combinatorial knock out of the glycogenes listed as group 2 genes suitable for determining the types of glycoconjugates involved in observed interactions. Determining changes in interactions with a plurality of mammalian cells with knock out of MGAT1 (N-Glycans) and/or COSMC (O-GalNac) and/or B4GALT7 (Glycosaminoglycans, GAG) and/or B4GALT5/6 (Glycosphingolipids) and/or POMGNT1 (O-Man) is used to identify if said interaction occurs through the type of glycoconjugate indicated in parenthesis, such that loss or reduction in measured interactions with mammalian cells with knock out of one or more of the named gene(s) confer that the corresponding type(s) of glycoconjugate(s) is responsible for the interaction as indicated.

Group 1 (Initiation)

All Subtypes of O-GalNAc Linked Mucin-Type O-Glycans:

In some embodiments of the present invention the plurality of mammalian cells comprises or consists of mammalian cells with individual and combinatorial knock out of the GALNT glycogenes (listed in Table 5 under group 1 genes for N-glycans). Determining changes in interactions with a plurality of mammalian cells with knock out of GALNT1 and/or GALNT2 and/or GALNT3 and/or GALNT4 and/or GALNT6 and/or GALNT7 and/or GALNT10 and/or GALNT11 and/or GALNT12 and/or GALNT13 and/or GALNT14 and/or GALNT16 and/or GALNT18 is used to identify if said interaction occurs through subsets of O-GalNAc glycoproteins controlled by one or more of the 20 GALNTs, respectively, such that loss or reduction in measured interactions with mammalian cells with knock out of one or more of the named gene(s) confer that the O-glycoprotein(s) responsible for the interaction requires glycosylation by the corresponding GALNT(s).

O-Xylose,

In some other embodiments of the present invention the plurality of mammalian cells comprises or consists of mammalian cells with individual and combinatorial knock out of the XYLT1 and/or XYLT2 glycogenes for determining if O-xylose type glycoproteins are involved in observed interactions. Determining changes in interactions with a plurality of mammalian cells with knock out of XylT1/XylT2 is used to identify if said interaction occurs through O-Xylose glycoproteins, such that loss or reduction in measured interactions with mammalian cells with knock out of one or more of the named gene(s) confer that the O-glycoprotein(s) responsible for the interaction requires glycosylation by the XYLT(s).

All Subtypes O-Fucose,

In some other embodiments of the present invention the plurality of mammalian cells comprises or consists of mammalian cells with individual and combinatorial knock out of the O-glycan glycogenes (listed in Table 5 under group 1 genes for O-Glycans), suitable for determining subsets of O-Fucosylated type glycoproteins involved in observed interactions. Determining changes in interactions with a plurality of mammalian cells with knock out of POFUT1 and/or POFUT2 is used to identify if said interaction occurs through fucosylated O-glycan type of glycoconjugate, such that loss or reduction in measured interactions with mammalian cells with knock out of one or more of the named gene(s) confer that the O-glycoprotein(s) responsible for the interaction requires glycosylation involving the corresponding POFUT activity.

POMT's (O-Mannose)

In some other embodiments of the present invention the plurality of mammalian cells comprises or consists of mammalian cells with individual and combinatorial knock out of the O-glycan glycogenes (listed in Table 5 under group 1 genes for O-Glycans), suitable for determining subsets of O-Mannose type glycoproteins involved in observed interactions. Determining changes in interactions with a plurality of mammalian cells with knock out of POMT1 and/or POMT2 and/or TMTC1 and/or TMTC2 and/or TMTC3 and/or TMTC4 is used to identify O-mannose type of glycoconjugate, such that loss or reduction in measured interactions with mammalian cells with knock out of the gene(s) confer that the O-Mannosylated glycoconjugate responsible for the interaction requires glycosylation involving initiation by POMT1 or POMT2 or TMTC1 or TMTC2 or TMTC3 or TMTC4.

TMTC's (O-Mannose)

In some other embodiments of the present invention the plurality of mammalian cells comprises or consists of mammalian cells with individual and combinatorial knock out of the O-glycan glycogenes (listed in Table 5 under group 1 genes for O-Glycans), suitable for determining subsets of O-Mannose type glycoproteins involved in observed interactions. Determining changes in interactions with a plurality of mammalian cells with knock out of TMTC1 and/or TMTC2 and/or TMTC3 and/or TMTC4 is used to identify an O-mannose type of glycoconjugate, such that loss or reduction in measured interactions with mammalian cells with knock out of the gene(s) confer that the O-Mannosylated glycoconjugate responsible for the interaction requires glycosylation involving initiation by one of the TMTC1-4 genes.

POGLUT1 Only One Gene

In some other embodiments of the present invention the plurality of mammalian cells comprises or consists of mammalian cells with individual and combinatorial knock out of the O-glycan glycogenes (listed in Table 5 under group 1 genes for O-Glycans), suitable for determining subsets of O-glycan type glycoproteins involved in observed interactions. Determining changes in interactions with a plurality of mammalian cells with knock out of POGLUT1 is used to identify if said interaction occurs through the O-glucose type of glycoconjugate, such that loss or reduction in measured interactions with mammalian cells with knock out of POGLUT1 confer that the O-glycoprotein(s) responsible for the interaction requires O-Glucose modification.

All Subtypes of N-Linked Glycoproteins:

In some other embodiments of the present invention the plurality of mammalian cells comprises or consists of mammalian cells with individual and combinatorial knock out of the STT3A/B (initiation) and ALG3/6/8/9/12 (oligomannose synthesis) glycogenes (listed in Table 5 under the group 1 genes for N-Glycans) suitable for determining subsets of N-linked type glycoproteins involved in observed interactions. Determining changes in interactions with a plurality of mammalian cells with knock out of STT3A and/or STT3B and/or ALG3 and/or ALG6 and/or ALG8 and/or ALG9 and/or ALG12 is used to identify if said interaction occurs through subsets of N-linked glycoproteins controlled by one of the two STT3 catalytic units or one of the five ALG enzymes, such that loss or reduction in measured interactions with mammalian cells with knock out of the named gene confer that the N-glycoprotein(s) responsible for the interaction requires glycosylation conferred by the corresponding STT3A or STT3B or one of the ALG's, including ALG3, ALG6, ALG8, ALG9, ALG10 and ALG12.

All Subtypes of C-Mannosylated Glycoproteins:

In some other embodiments of the present invention the plurality of mammalian cells comprises or consists of mammalian cells with individual and combinatorial knock out of the DPY19L1, DPY19L2, DPY19L3 and DPY19L4 glycogenes (listed in Table 5 under group 1 genes for C-mannosylation) suitable for determining subsets of C-mannosylation type glycoproteins involved in observed interactions. Determining changes in interactions with a plurality of mammalian cells with knock out of DPY19L1 and/or DPY19L2 and/or DPY19L3 and/or DPY19L4 is used to identify if said interaction occurs through subsets of C-mannosylated glycoproteins controlled by one or more of the four DPY19L genes, such that loss or reduction in measured interactions with mammalian cells with knock out of one or more of the named gene(s) confer that the C-mannosylated protein(s) responsible for the interaction as indicated.

Group 3

N-Glycan Branching

In some other embodiments of the present invention the plurality of mammalian cells comprises or consists of mammalian cells with individual and combinatorial knock out of MGAT2 or MGAT3 (for mono antennary), MGAT3, MGAT4A, MGAT4B or MGAT5 (for bi antennary), MGAT3, MGAT4A, MGAT4B or MGAT3/5 (for tri-antennary) and knock out of MGAT3 combined with knock-in of MAGAT5 and MGAT4A and/or MGAT4B (for tetra-antennary) glycogenes (listed in Table 5, group 3 genes) suitable for determining N-linked branched glycans involved in observed interactions. Determining changes in interactions with a plurality of mammalian cells with knock out and/or knock in of MGAT2 and/or MGAT3 and/or MGAT4A and/or MGAT4B and/or MGAT5 is used to identify if said interaction occurs through N-linked antennae controlled by one or more of these enzymes, such that loss or reduction in measured interactions with mammalian cells with knock out of one or more of the named gene(s) confer that the N-linked antennae(s) is responsible for the interaction as indicated.

N-Glycan Oligomannose Trimming

In other embodiments of the present invention the plurality of mammalian cells comprises or consists of mammalian cells with individual and combinatorial knock out of MAN1A1, MAN1A2, MAN1B1, MAN1C1, MAN2A1, MAN2A2 or MOGS or GANAB (oligomannosidase trimming) glycogenes (listed in Table 5, group 3 genes) suitable for determining N-linked branched glycans involved in observed interactions. Determining changes in interactions with a plurality of mammalian cells with knock out and/or knock in of MAN1A1 and/or MAN1A2 and/or MAN1B1 and/or MAN1C1 and/or MAN2A1 and/or MAN2A2 and/or MOGS and/or GANAB is used to identify if said interaction occurs through N-linked antennae controlled by one or more of these enzymes such that loss or reduction in measured interactions with mammalian cells with knock out of one or more of the named gene(s) confer that the oligomannose trimming is responsible for the interaction as indicated.

O-Glycan Branching

In some other embodiments of the present invention the plurality of mammalian cells comprises or consists of mammalian cells with individual and combinatorial knock out of GCNT1, GCNT2, GCNT3, GCNT4, GCNT6, GCNT7, B3GNT6 or B3GNT2 glycogenes (listed in Table 5, group 3 genes) suitable for determining O-linked branching in Core2 Core3 and Core4 structures, involved in observed interactions. Determining changes in interactions with a plurality of mammalian cells with knock out of GCNT1 and/or GCNT2 and/or GCNT3 and/or GCNT4 and/or GCNT6 and/or GCNT7 and/or B3GNT6 and/or B3GNT6 is used to identify if said interaction occurs through O-linked branched structures by one or a plurality of the branching enzymes, such that loss or reduction in measured interactions with mammalian cells with knock out of one or more of the named gene(s) confer that the O-linked branched structure is responsible for the interaction as indicated.

N and O-Elongation (LacNAc 1-3/1-4/LacdiNAc), O-GalNAc Core 1-4, PolyLAc/Branching

In yet other embodiments of the present invention the plurality of mammalian cells comprises or consists of mammalian cells with individual and combinatorial knock out of B3GNT2/3/4/6/7/8/9, B4GALT1, B4GALT2, B4GALT3 or B4GALT4 (type2) and/or B4GALT1/2/3/4/5 (type1) and/or B4GALNACT3/4 and/or GCNT2 glycogenes (listed in Table 5, group 3 genes) suitable for determining N, and O-linked elongated LacNac (polylacNAc when repeated), LacdiNAc and branched structures respectively, involved in observed interactions. Determining changes in interactions with a plurality of mammalian cells with knock out of B3GNT2/3/4/6/7/8/9 and/or B4GALT1/2/3/4 and/or B3GALT1/2/3/4/5 and/or B4GALNACT3/4 and/or GCNT2 is used to identify if said interaction occurs through N or O-linked elongated structures by one or a plurality of the elongation enzymes, such that loss or reduction in measured interactions with mammalian cells with knock out of one or more of the named gene(s) confer that the N or O-linked branched structure is responsible for the interaction as indicated.

Glycosaminoglycans and Glycolipids Branching/Elongation

In some embodiments of the present invention the plurality of mammalian cells comprises or consists of mammalian cells with individual and combinatorial knock out of the glycogenes listed as group 2 genes suitable for determining the types of glycoconjugates involved in observed interactions. Determining changes in interactions with a plurality of mammalian cells with knock out of EXTL2/3 (Heparan Sulphate) and/or CSGALNACT1/2 (chondroitin/chondoitin-sulfate) and/or A4GALT (glycolipids globo) and/or B3GNT5 (glycolipids lacto) and/or B4AGLNT1 (glycolipids ganglio) is used to identify if said interaction occurs through the type of glycoconjugate indicated in parenthesis, such that loss or reduction in measured interactions with mammalian cells with knock out of one or more of the named gene(s) confer that the corresponding type(s) of glycoconjugate(s) is responsible for the interaction as indicated.

Group 4

NeuAc's/Polysialylation Capping

In yet other embodiments of the present invention the plurality of mammalian cells comprises or consists of mammalian cells with individual and combinatorial knock out of genes involved in N and O-glycan and glycolipid capping (sialylation); ST3GAL1/2/3/4/5/6 (α2,3NeuAc capping/sialylation) and/or ST6GAL1/2 (α2,6NeuAc capping/sialylation) and/or ST8SIA1/2/3/4/5/6 (capping by poly-sialylation) and/or ST6GALNAC1/2/3/4/5/6 (α2,6NeuAc capping/sialylation) (glycogenes listed in Table 5, group 4 genes) suitable for determining the capped (sialylated or fucosylated) glycan structure involved in observed interactions. Determining changes in interactions with a plurality of mammalian cells with knock out of ST3GAL1/2/3/4/5/6 and/or ST6GAL1/2 and/or ST8SIA1/2/3/4/5/6 and/or ST6GALNAC1/2/3/4/5/6 is used to identify if said interaction occurs through the type of capping indicated in parenthesis, such that loss or reduction in measured interactions with mammalian cells with knock out of one or more of the named groups of genes confer that the type of capping is responsible for the interaction as indicated.

Fucosylation Capping

In yet other embodiments of the present invention the plurality of mammalian cells comprises or consists of mammalian cells with knock out of genes involved in O-glycan capping by fucosylation; FUT1/2 (α1,2 fucosylation) and/or FUT3/4/5/6/7/9/10/11 (α1,3/4 fucosylation) (glycogenes are listed in Table 5, group 4 genes) suitable for determining type of O-fucosylation involved in observed interactions. Determining changes in interactions with a plurality of mammalian cells with knock out of FUT1/2 and/or FUT3/4/5/6/7/9/10/11 is used to identify if said interaction occurs through the type of fucosylation indicated in parenthesis, such that loss or reduction in measured interactions with mammalian cells with knock out of one or more of the named gene groups confer that the corresponding type of fucosylation is responsible for the interaction as indicated.

Group 5

Sulfation, Acetylation and Other Modifications

In yet other embodiments of the present invention the plurality of mammalian cells comprises or consists of mammalian cells with one or more knock out of CHST1/2/3/4/5/6/7/8/9/10 (SO₄²⁻ sulfation) and/or CASD1 (AcO⁻ acetylation) glycogenes (listed in Table 5, group 5 genes) suitable for determining modification of glycan structures, involved in observed interactions. Determining changes in interactions with a plurality of mammalian cells with knock out of CHST1/2/3/4/5/6/7/8/9/10 and/or CASD1 is used to identify if said interaction occurs through type of glyco-modification indicated in parenthesis, such that loss or reduction in measured interactions with mammalian cells with knock out of one or more of the named gene(s) confer that the corresponding type of modification is responsible for the interaction as indicated.

HEK293 Missing Genes Knock In

In yet other embodiments of the present invention the plurality of mammalian cells comprises or consists of HEK293 cells with knock in of one or more of the following human glycogenes that are not expressed or expressed at low levels in HEK293 cells (Table 6): A3GALT2, A4GNT, ABO, ALG1L2, B3GALNT1, B3GALT2, B3GNT6, B4GALNT2, FUT5, FUT7, FUT9, GALNT15, GALNT5, GALNT9, GALNTL5, GALNTL6, GALNT19/WBSCR17, GCNT3, GCNT4, GCNT7, GLT1D1, GLT6D1, HAS1, MGAT4C, MGAT4D, ST6GAL2, ST6GALNAC1, ST8SIA1, ST8SIA3, ST8SIA4, CHST2, GAL3ST3, HS3ST1, HS3ST4, HS3ST5, NDST3.

In some embodiments of the present invention comprises mammalian cells displaying N-glycans with only α2,3NeuAc capping by knock out of ST6GAL1 and/or ST6GAL2

In some other embodiments of the present invention comprises mammalian cells displaying N-glycans with only α2,6NeuAc capping by knock out of one or more of ST3GAL1, ST3GAL2, ST3GAL3, ST3GAL4, ST3GAL5 and ST3GAL6.

In some embodiments of the present invention the plurality of mammalian cells comprises mammalian cells displaying N-glycans without NeuAc capping by knock out of ST3GAL3/4/6 and/or ST6GAL1/2.

Another object of the present invention relates to a plurality of mammalian cells comprising one or more glycosyltransferase genes that have been introduced stably by site-specific gene or non-site-specific knock in and with different glycosylation capacities.

Another object of the present invention relates to a plurality of mammalian cells comprising one or more glycosyltransferase genes that have been introduced transiently and with different glycosylation capacities.

In some embodiments of the present invention the plurality of mammalian cells comprises two or more glycosyltransferases that have been introduced stably by site-specific or non-site-specific gene knock in.

An aspect of the present invention relates to a plurality of mammalian cells comprising one or more glycosyltransferase genes introduced stably by site-specific or non-site-specific gene knock in, and furthermore comprising one or more endogenous glycosyltransferase genes that have been inactivated by knock out, and with different glycosylation capacities.

In some embodiments of the present invention the cell is a human cell.

In some embodiments of the present invention the mammalian cell expresses at least 95% of the human glycogenes identified in Table 1 or heterologous homologs hereof, or at least 50%, such as 55%, such as 60%, such as 65%, such as 70%, such as 75%, such as 80%, such as 85%, such as 90% of human glycogenes identified in Table 1 or heterologous homologs hereof.

In some other embodiments of the present invention the mammalian cell is derived from human kidney.

In further embodiments of the present invention the mammalian cell is selected from the group consisting of NS0, SP2/0, YB2/0, HEK293, HUVEC, HKB, PER-C6, NS0, or derivatives of any of these cells.

In some embodiments of the present invention is the mammalian cell a HEK293 cell.

In some other embodiments of the present invention the plurality of mammalian cells furthermore encodes one or more exogenous proteins of interest, such as a glycoprotein of interest.

In further embodiments of the present invention are the glycosyltransferases that are inactivated belonging to the CAZy families as listed in Table 1.

In yet other embodiments of the present invention are the glycosyltransferases that are inactivated belonging to same subfamily of isoenzymes in a CAZy family.

In yet other embodiments of the present invention is the glycosylation non-galactosylated (for example by knock-out of B4GALT1-6).

In some embodiments of the present invention the glycosylation comprises biantennary N-glycans (for example by knock-out of ST6GAL1/2).

In some other embodiments of the present invention does the glycosylation not comprise poly-LacNAc (for example by knock-out of B3GNT2/3/4/7/8/9).

In some other embodiments of the present invention does the glycosylation not comprise LacDiNAc (for example by knock-out of B4GALNT3/4).

In yet other embodiments of the present invention comprises mammalian cells displaying N-glycans without fucose by knock-out of FUT8.

Glycosylation in mammalian cells is a non-template driven process involving more than 200 glycosyltransferases and other enzymes and transporters, and the repertoire of these genes expressed in a given cell is the major determinant of the glycome produced.

GTf Genes

Mammalian cells have a large number of glycosyltransferase genes and over 200 distinct genes have been identified and their catalytic properties and functions in glycosylation processes partially determined (Ohtsubo 2006; Lairson 2008; Bennett 2012; Schachter 2014). These genes are classified in homologous gene families with related structural folds in the CAZy database (www.cazy.org). The encoded glycosyltransferases catalyse different steps in the biosynthesis of glycosphingolipids, glycoproteins, GPI-anchors, and proteoglycans (together termed glycoconjugates), as well as oligosaccharides found in mammalian cells. Enormous diversity exists in the structures of glycans on these molecules, and biosynthetic pathways for different types of glycans have been worked out (Kornfeld 1985; Tarp 2008; Bennett 2012; Schachter 2014), although our understanding of which glycosyltransferase enzyme(s) that catalyze a particular linkage in the biosynthesis of the diverse set of glycoconjugates produced in a mammalian cell is not complete. Glycosylation in cells is a non-template driven process that relies on a number of factors many of which are unknown for producing the many different glycoconjugates and glycan structures with a high degree of fidelity and differential expression and regulation in cells. These factors may include: expression of the glycosyltransferase proteins; the subcellular topology and retention in the ER-Golgi secretory pathway, the synthesis, transport into, and availability of sugar nucleotide donors in the secretory pathway; availability of acceptor substrates; competing glycosyltransferases; divergence and/or masking of glycosylation pathways that affect availability of acceptor substrates and/or result in different structures; and the general growth conditions and nutritional state of cells.

GTf Isoenzymes

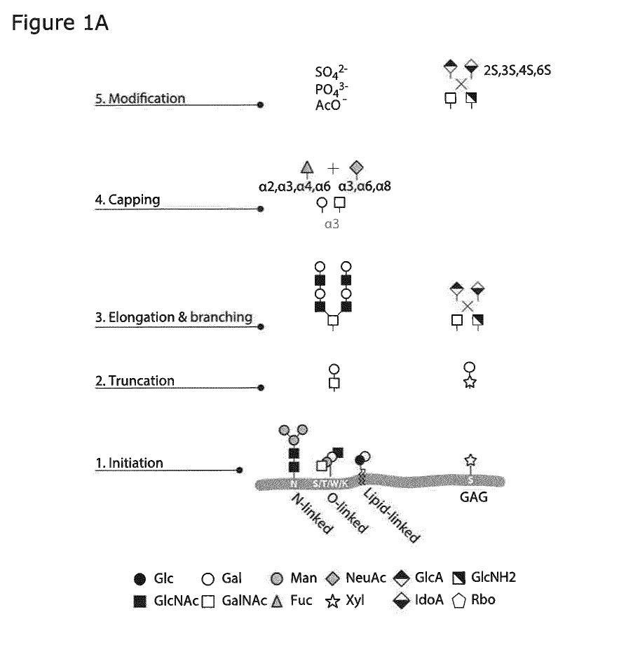

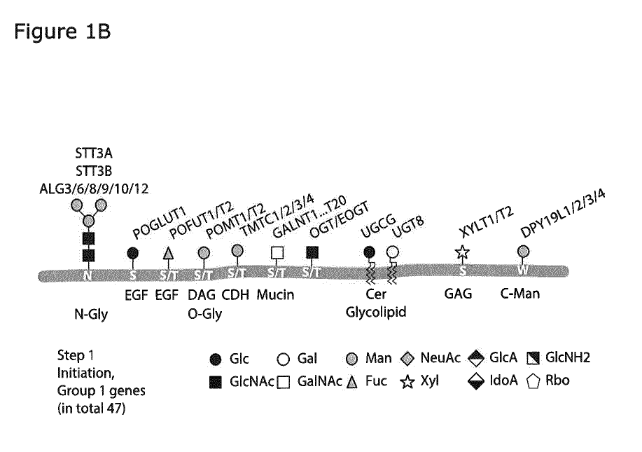

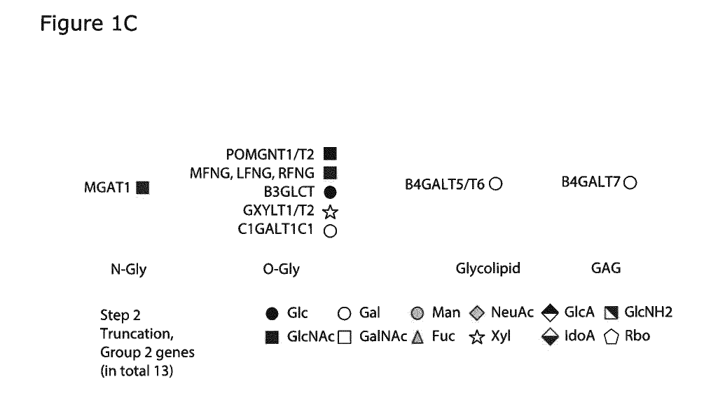

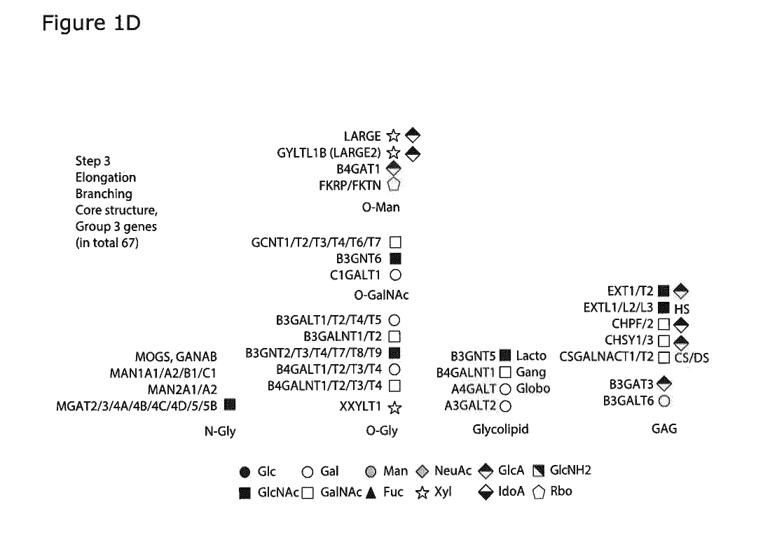

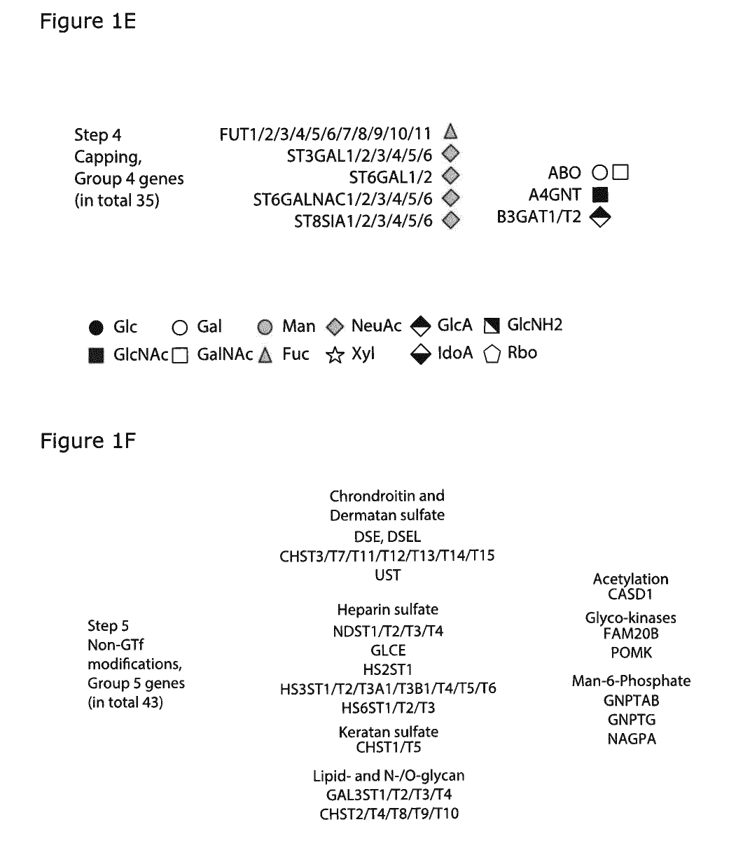

A number of the glycosyltransferase genes have high degree of sequence similarity and these have been classified into subfamilies encoding closely related putative isoenzymes, which have been shown to or predicted to serve related or similar functions in biosynthesis of glycans in cells (Tsuji 1996; Amado 1999; Narimatsu 2006; Bennett 2012). Examples of such subfamilies include the polypeptide GalNAc-transferases (GalNT1 to 20), α2,3sialyltransferases (ST3GAL1-6), α2,6sialyltransferases (ST6GAL1 and 2), α2,6sialyltransferases (ST6GALNAC1 to 6), α2,8sialyltransferases (ST8SIA1 to 6), β4galactosyltransferases (B4GALT1 to 7), 33galactosyltransferases (B3GALT1 to 6), β3GlcNAc-transferases (B3GNT2 to 9), β6GlcNAc-transferases (GCNT1-7 including GCNT2A, 2B and 2C (also known as C2GnT1-7 and IGnT2A to C), β4GalNAc-transferases (B4GALNT1 to 4), 33glucuronyltransferase (B3GAT1 to 3), α3/4-fucosyltransferases (FUT1 to 11), O-fucosyltransferases (POFUT1 and 2), O-glucosyltransferases (POGLUT 1 and 2), β4GlcNAc-transferases (MGAT4A to C), 36GlcNAc-transferases (MGAT5 and 5B), and hyaluronan synthases (HAS1 to 3) (Hansen 2014) (See Table 1 and FIG. 1 for overview of genes and proteins including nomenclature used).

GTf Subfamily Functions

These subfamilies of isoenzymes are generally poorly characterized and the functions of individual isoenzymes unclear. In most cases the function of isoforms are predicted from in vitro enzyme analysis with artificial substrates and these predictions have often turned out to be wrong or partially incorrect (Marcos 2004). Isoenzymes may have different or partially overlapping functions or may be able to provide partial or complete backup in biosynthesis of glycan structures in cells in the absence of one or more related glycosyltransferases. It is therefore not possible to reliably predict how deficiency of a particular gene in these subfamilies will affect the glycosylation pathways and glycan structures produced on the different glycoconjugates in a cell.

Engineering GTfs in Cells

Only little information exists as to the effects of knock out of glycosyltransferase genes in mammalian cell lines. For human cell lines only a few spontaneous mutants of glycosyltransferase genes have been identified. For example the colon cancer cell line LSC derived from LS174T has a mutation in the COSMC chaperone that leads to misfolded and non-functional core1 synthase C1GalT (Ju 2008). The COSMC gene is also mutated in the human lymphoblastoid Jurkat cell line (Ju 2008; Steentoft 2011). A series of CHO cell lines (Lec cell lines) with deficiency in one glycosyltransferase gene was originally generated by random mutagenesis follow by lectin selection, and the isolated lectin-resistant mutant clones were later shown to have defined mutations in the glycosyltransferase genes Mgat1 (Lec1), Mgat5 (Lec4), and B4galt1 (Lec20) (Patnaik 2006).

Knock Out of Glycosylation Genes in Cell Lines

The limited information of effects of knock out of glycosyltransferase genes in cell lines is partly due to past difficulties with making knock outs in cell lines before the recent advent of precise gene editing technologies (Steentoft 2014). Thus, until recently essentially only one glycosyltransferase gene, FUT8, had been knocked out in a directed approach using two rounds of homologous recombination including massive clone screening efforts. The conventional gene disruption by homologous recombination is typically a very laborious process as evidenced by this knock out of Fut8 in CHO, as over 100,000 clonal cell lines were screened to identify a few growing Fut8−/− clones (Yamane-Ohnuki 2004) (U.S. Pat. No. 7,214,775). With the advent of the Zinc finger nuclease (ZFN) gene targeting strategy it became less laborious to disrupt genes, which was first demonstrated by knock out of the Fut8 gene in a CHO cell line, where additional two other genes unrelated to glycosylation were also effectively targeted (Malphettes 2010). More recently, TALENs and the CRISPR/Cas9 editing strategies have emerged, and the latter editing strategy was used to knock out the Fut8 gene (Ronda 2014).

It is thus clear that targeted genetic engineering is now a tool the skilled person may use but editing of the glycosylation genes in mammalian cells and animals are prone to substantial uncertainty, and thus identifying the optimal engineering targets for display of a given glycan structure will require extensive experimental efforts. Therefore a random type approach involving testing of a multiplicity of different glycogene and glycoform variations may be beneficial.

Overexpression of Glycosylation Genes in Cell Lines

It is noteworthy that transient or stable overexpression of a glycosyltransferase gene in a cell most often result in only partial changes in the glycosylation pathways in which the encoded enzyme is involved. A number of studies have attempted to overexpress e.g. the core2 C2GnT1 enzyme in CHO to produce core2 branched O-glycans, the ST6GAL1 sialyltransferase to produce α2,6linked sialic acid capping on N-glycoproteins (El Mai 2013), and the ST6GALNAC1 sialyltransferase to produce α2,6linked sialic acid on O-glycoproteins forming the cancer-associated glycan STn (Sewell 2006). However, in all these studies heterogeneous and often unstable glycosylation characteristics in transfected cell lines have been obtained. This is presumably partly due to competing endogenous glycosyltransferase activities whether acting with the same substrates or diverging pathway substrates. Other factors may also explain the heterogeneous glycosylation characteristics.

Display of Protein Glycoforms by Recombinant Expression

Human cells produce a variety of complex glycan structures not found in other mammalian cells. HEK293 has been the preferred human cell line for expression of recombinant proteins (Walsh 2014), and this cell line produce complex type N and O-glycans with both α2,3 and α2,6 sialic acid capping of N-glycans, extensive fucosylation, and LacDiNAc structures (Bohm 2015).

Genetic engineering of human cells have recently been introduced to alter the glycosylation of recombinant expressed proteins. Human cell lines including HEK293 with inactivation of the COSMC gene have been produced, and these cell lines produce O-glycans with truncated GalNAcα1-O-Thr structure with variable degree of sialic acid capping (Stentoft 2013).

Sugar chains of glycoproteins are roughly divided into two types, namely a sugar chain which binds to asparagine (N-glycoside-linked sugar chain) and a sugar chain which binds to other amino acid such as serine, threonine (O-glycoside-linked sugar chain), based on the binding form to the protein moiety (FIG. 1).

For the present invention it is to be understood that the sugar chain terminus which links to the protein or lipid moiety is called a reducing end, and the opposite side is called a non-reducing end. It is known that the N-glycoside-linked sugar chain includes a high mannose type in which mannose alone binds to the non-reducing end of the core structure; a complex type in which the non-reducing end side of the core structure has at least one parallel branches of galactose-N-acetylglucosamine (hereinafter referred to as “Gal-GlcNAc”) and the non-reducing end side of Gal-GlcNAc has a structure of sialic acid, bisecting N-acetylglucosamine or the like; a hybrid type in which the non-reducing end side of the core structure has branches of both of the high mannose type and complex type.

The glycome of a cell is the sum of all glycan structures produced in that cell. Individual glycan structures may have important biological functions, and example serve as ligands for carbohydrate-binding proteins such as lectins, toxins and adhesins found in bacteria and viruses. The glycome of a mammalian and human cell is vast with 1,000s of different glycan structures on different types of glycolipids, glycoproteins, proteoglycans (glycoconjugates) and also free oligosaccharides. There exist a number of ways to isolate and characterize these glycoconjuagtes, but it is often very difficult to obtain homogeneous structures with respect to the glycan part and to prepare comprehensive isolation of all structures found in the glycome. Advances in organic synthesis and chemoenzymatic strategies of glycans have facilitated access to pure glycan structures, which has led to generation of glycan libraries representing the glycome, although so far not comprehensive. These advances has enabled development of glycan array technologies where individual glycans are attached to microarray slides, and used for display of parts of the glycome for interrogation of biological interactions with glycans (Rillahan 2011, Blixt 2004, Padler-Karavani 2012). However, the current glycan arrays only display limited subsets of the glycome of cells and they display glycans without the context of proteins, proteoglycans and lipids as well as the cell membrane.

There is a need for development of new methods for the display of mammalian and preferably human glycomes that are comprehensive and with the context of glycoconjugates and the cell membrane.

Moreover there is a need to develop a plurality of mammalian cells that display the mammalian and preferably the human glycomes in a comprehensive way, and such that individual glycan structures can be probed and assessed by interpretation as contributing to a particular biological interaction measured by use of the cell or shed/secreted proteins or components from the cell.

This present invention discloses a gene engineering method involving knock out and knock in of over 250 mammalian and human glycosyltransferase and related glycogenes to develop a plurality of mammalian, such as human cells displaying differential parts of the cell glycome, and use of such plurality of engineered mammalian cells to display and probe the glycome in the context of the glycoconjugate structure and/or cell membrane. Moreover the invention provides a method for step-by-step assessing and interpreting the biosynthetic pathway(s) and glycan structure(s) involved in biological interactions probed by use of the plurality of mammalian cells displaying the glycome.

In one aspect, ZFN targeting designs for inactivation of glycosyltransferase genes are provided.

In another aspect, TALEN targeting designs for inactivation of glycosyltransferase genes are provided.

In yet another aspect, CRISPR/Cas9 based targeting for inactivation of glycosyltransferase genes is provided.

In certain embodiments, the invention provides mammalian cells with inactivation of two or more glycosyltransferase genes encoding isoenzymes with partially overlapping glycosylation functions in the same glycosylation pathway, and for which inactivation of two or more of these genes is required for loss of said glycosylation functions in the cell.

In certain embodiments, the invention provides mammalian cells with inactivation of two or more glycosyltransferase genes encoding isoenzymes with partially overlapping glycosylation functions in the same glycosylation pathway and biosynthetic step, and for which inactivation of two or more of these genes is required for loss of said glycosylation functions in the cell.

In certain embodiments, the invention provides mammalian cells with inactivation of two or more glycosyltransferase genes encoding isoenzymes with no overlapping glycosylation functions in the same glycosylation pathway, and for which inactivation of two or more of these genes is required for loss of said glycosylation functions in the cell.

In some other embodiments, the invention provides mammalian cells with inactivation of two or more glycosyltransferase genes encoding enzymes with unrelated glycosylation functions in the same glycosylation pathway, and for which inactivation of two or more genes is required for abolishing said glycosylation functions in the cell.

In some other embodiments, the invention provides mammalian cells with inactivation of two or more glycosyltransferase genes encoding enzymes with unrelated glycosylation functions in different glycosylation pathways, and for which inactivation of two or more genes is required for desirable glycosylation functions in the cell.

In some other embodiments, the present invention provides a defined set of 266 glycosyltransferase and related glycogenes with which to combinatorially construct the plurality of mammalian cells capable of displaying the glycome in a plurality of mammalian cells with capability to address the glycome on said cells and/or products from these in biological assays, and to interpret glycans involved in biological assays with respect to the glycosyltransferase and related genes controlling biosynthesis of such glycan(s) and the structure(s).

In some other embodiments, the present invention provides a limited set of up to 47 glycosyltransferase and related glycogenes with which to combinatorially construct the plurality of mammalian cells capable of displaying the glycoconjugate type (the initiation step 1, FIG. 1B and Table 5, Group 1) in an addressable and interpretable way.

In yet other embodiments, this invention provides a combinatorial design for inactivation(s) and/or introduction of a limited set of up to 13 glycosyltransferase and related glycogenes required for displaying the truncated human glycome on all types of plurality of mammalian cells capable of displaying the glycoconjugate type (the truncation step 2, FIG. 1C and Table 5. Group 2) in an addressable and interpretable way.

In yet other embodiments, this invention provides a combinatorial design for inactivation(s) and/or introduction of a limited set of up to 67 glycosyltransferase and related glycogenes required for display of elongated and branched core structures for the human glycome on all types of glycoconjugates in a plurality of mammalian cells (the elongation, brancing core structures, step 3, FIG. 1D and Table 5, Group 3) in an addressable and interpretable way.

In yet other embodiments, this invention provides a combinatorial design for inactivation(s) and/or introduction of a limited set of up to 35 glycosyltransferase and related glycogenes required for display of glycan capping of the human glycome on all types of glycoconjugates in a plurality of mammalian cells (the capping, step 4, FIG. 1E and Table 5, Group 4) in an addressable and interpretable way.

In yet other embodiments, this invention provides a combinatorial design for inactivation(s) and/or introduction of a limited set of up to 44 glycosyltransferase and related glycogenes required for display of non-GTf modifications including sulfonation, acetylation and phosphorylation of the human glycome on all types of glycoconjugates in a plurality of plurality of mammalian cells (the non-GTf modifications, step 5, FIG. 1F and Table 5, Group 5) in an addressable and interpretable way.

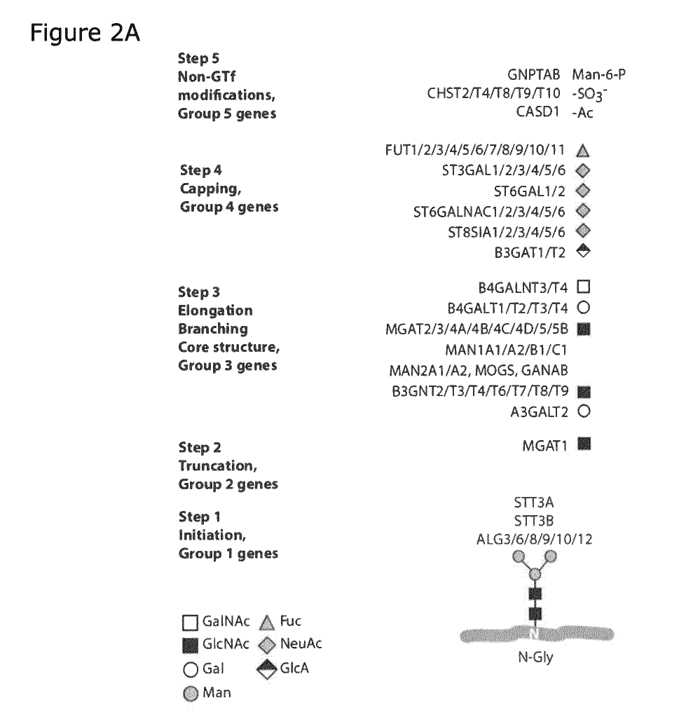

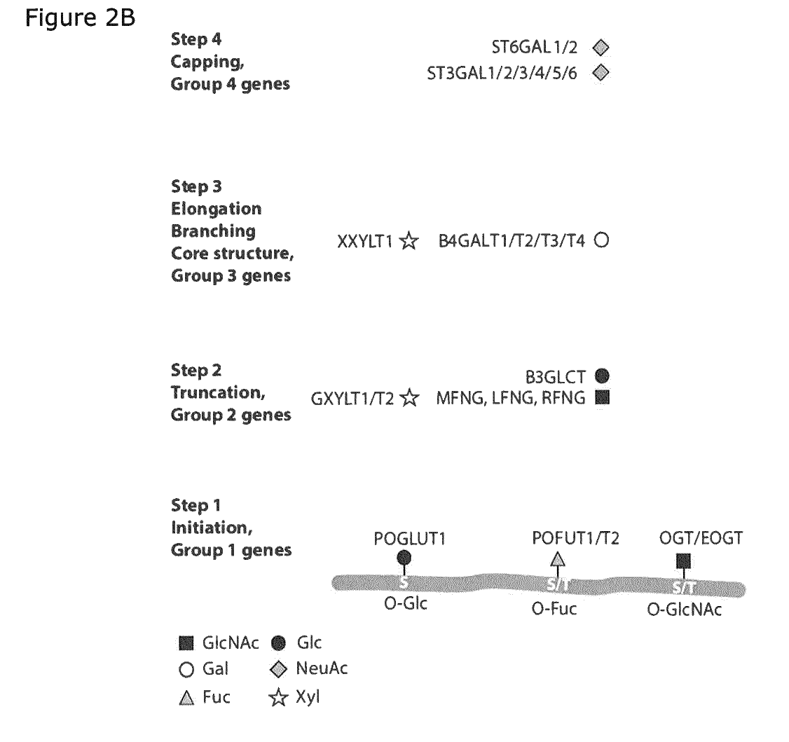

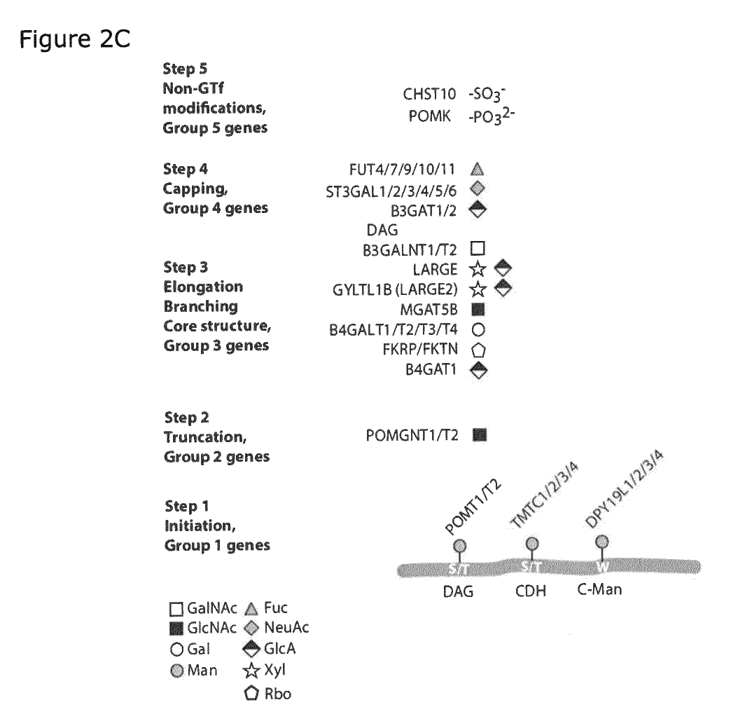

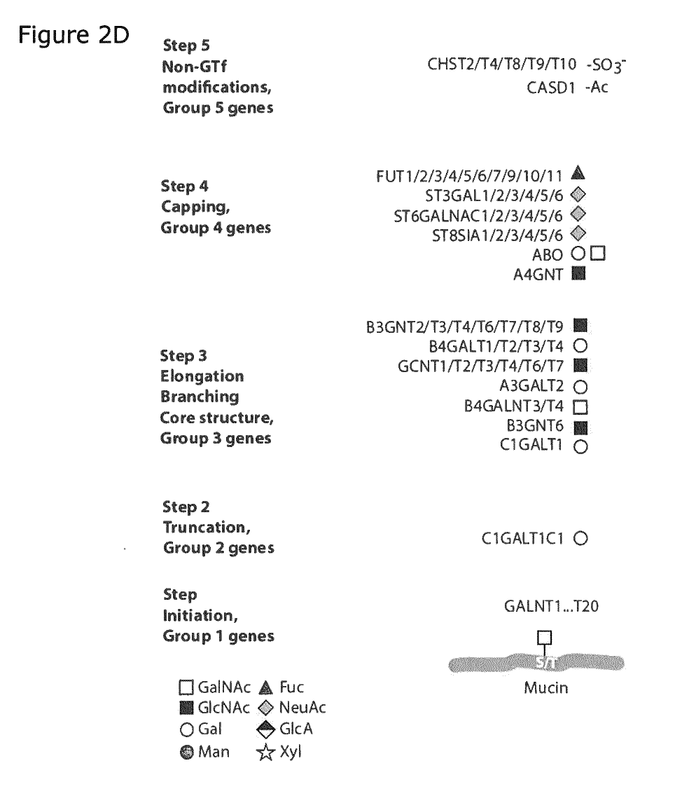

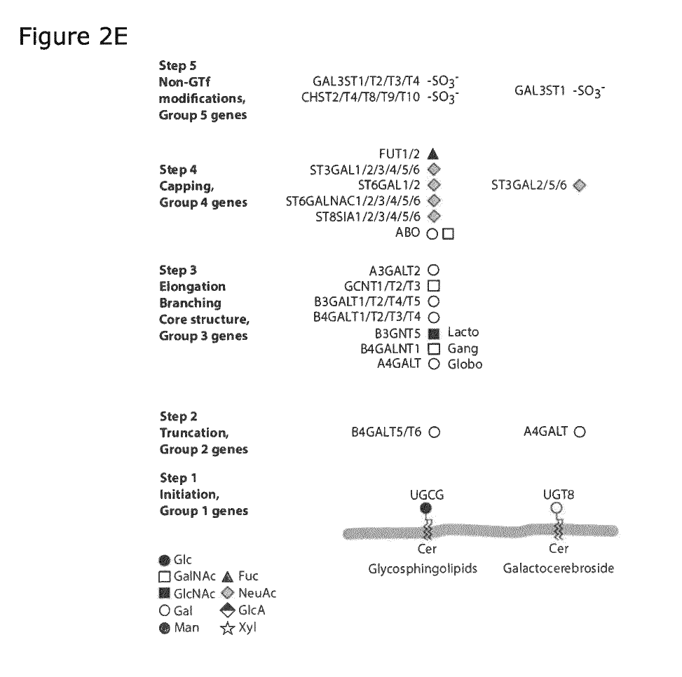

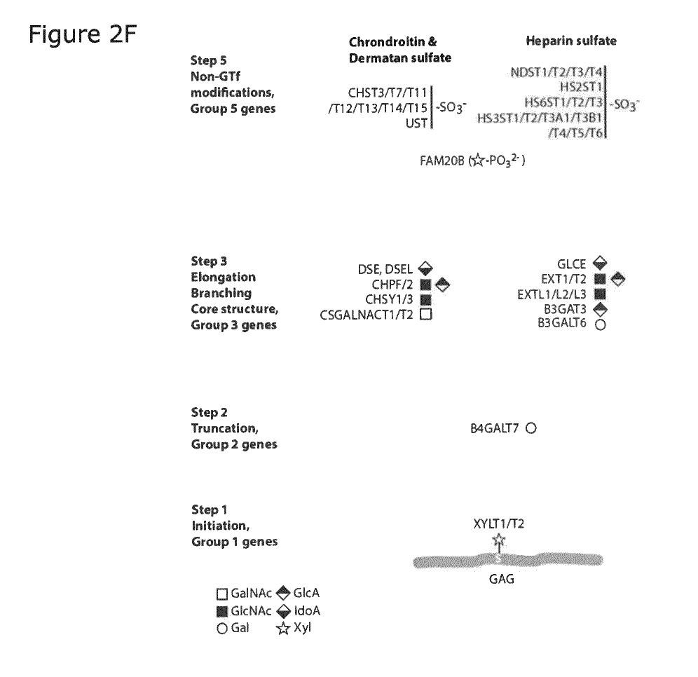

In certain embodiments, the invention provides inactivation and/or introduction of two or more genes required for display of various N-glycans in a plurality of mammalian cells. Genes involved in N-glycosylation include but are not limited to genes listed in FIG. 2A. May include genes not expressed in HEK293 (Group 6 genes, Table 6).

In yet other embodiments, this invention provides a combinatorial design for inactivation(s) and/or introduction of a limited set of glycosyltransferase genes and related glycogenes required for display of various O-Glc/O-Fut glycans in a plurality of mammalian cells (the O-Glc/O-fut glycan pathways, Step 1-4, FIG. 2B) in an addressable and interpretable way.

In certain embodiments, the invention provides inactivation and/or introduction of two or more genes required for display of various O-Gluc/O-Fut glycans in a plurality of mammalian cells. Genes involved in O-Gluc/O-Fut glycosylation include but are not limited to genes listed in FIG. 2B. May include genes not expressed in HEK293 (Group 6 genes, Table 6).

In yet other embodiments, this invention provides a combinatorial design for inactivation(s) and/or introduction of a limited set of glycosyltransferase genes and related glycogenes required for display of various O-Man glycans in a plurality of mammalian cells (the O-Man glycan pathways, Step 1-5, FIG. 2C) in an addressable and interpretable way.

In certain embodiments, the invention provides inactivation and/or introduction of two or more genes required for display of various O-Man glycans in a plurality of mammalian cells. Genes involved in O-Man glycosylation include but are not limited to genes listed in FIG. 2A. May include genes not expressed in HEK293 (Group 6 genes, Table 6).

In yet other embodiments, this invention provides a combinatorial design for inactivation(s) and/or introduction of a limited set of glycosyltransferase genes and related glycogenes required for display of various O-GalNac glycans in a plurality of mammalian cells (the O-GalNac glycan pathways, Step 1-5, FIG. 2D) in an addressable and interpretable way.

In certain embodiments, the invention provides inactivation and/or introduction of two or more genes required for display of various O-GalNac glycans in a plurality of mammalian cells. Genes involved in O-GalNac glycosylation include but are not limited to genes listed in FIG. 2D. May include genes not expressed in HEK293 (Group 6 genes, Table 6).

In yet other embodiments, this invention provides a combinatorial design for inactivation(s) and/or introduction of a limited set of glycosyltransferase genes and related glycogenes required for display of various Glycolipids in a plurality of mammalian cells (the Glycolipid pathways, Step 1-5, FIG. 2E) in an addressable and interpretable way.

In certain embodiments, the invention provides inactivation and/or introduction of two or more genes required for display of various Glycolipids glycans in a plurality of mammalian cells. Genes involved in Glycolipid synthesis include but are not limited to genes listed in FIG. 2E. May include genes not expressed in HEK293 (Group 6 genes, Table 6).

In yet other embodiments, this invention provides a combinatorial design for inactivation(s) and/or introduction of a limited set of glycosyltransferase genes and related glycogenes required for display of various Glycosaminoglycans in a plurality of mammalian cells (the Glycolipid pathways, Step 1-5, FIG. 2F) in an addressable and interpretable way.

In certain embodiments, the invention provides inactivation and/or introduction of two or more genes required for display of various Glycosaminoglycans in a plurality of mammalian cells. Genes involved in Glycosaminoglycan synthesis include but are not limited to genes listed in FIG. 2F. May include genes not expressed in HEK293 (Group 6 genes, Table 6).

The present inventors have used different nuclease-mediated (ZFN, TALEN, CRISPR/Cas9) knock out and knock in in human HEK293 cells to explore the potential for display of the glycome in an addressable and interpretable way.

One object of the present invention is to provide a plurality of isogenic mammalian cells that display one or more of the following posttranslational modification patterns on proteins, lipids, and proteoglycans on the cell surface:

Another related object of the present invention is to provide a plurality of isogenic mammalian cells that display one or more of the following posttranslational modification patterns on proteins, lipids, and/or proteoglycans secreted and/or released from said cells:

a) eliminated β4-branched tetraantennary N-glycans (for example by knock-out of MGAT4A and MGAT4B),

b) eliminated β6-branched tetraantennary structures (for example by knock-out of MGAT5),

c) elimination of L-PHA lectin labeling (for example by knock-out of MGAT5),

d) homogenous biantennary N-glycans (for example by knock-out of MGAT4A, MGAT4B and MGAT5),

e) abolished galactosylation on N-glycans (for example by knock-out of B4GALT1 and B4GALT3),

f) elimination of poly-LacNAc (for example by knock-out of B3GNT2),

g) heterogeneous tetraantennary N-glycans without trace of sialylation (for example by knock-out of ST3GAL3/4/6, ST6GAL1 and ST6GAL2),

h) biantennary N-glycans without sialylation (for example by knock-out of MGAT4A/4B/5, ST3GAL3/4/6, ST6GAL1 and ST6GAL2),

i) lack of sialic acid (for example by knock-out of one or more of ST3GAL3, ST3GAL4, ST3GAL6, ST6GAL1 and ST6GAL2),

j) uncapped LacNAc termini (for example by knock-out of B4GALT1/2/3/4/5/6),

k) homogenous biantennary N-glycans capped by α2,6NeuAc (for example by knock-out of MGAT4A/4B/5 and one or more of ST3GAL1, ST3GAL2, ST3GAL3, ST3GAL4, ST3GAL5 and ST3GAL6),

l) homogenous α2,6NeuAc capping (for example by knock-out of one or more of ST3GAL1, ST3GAL2, ST3GAL3, ST3GAL4, ST3GAL5 and ST3GAL6), or

m) homogenous biantennary N-glycans capped by α2,3NeuAc (for example by knock-out of ST6GAL1 and ST6GAL2).

n) Elimination of LacDiNAc (for example by knock-out of B4GALNT3/4).

One, two, three, four, five, six, seven, eight, nine, ten, eleven, twelve, or more of these effects can be combined to generate specific posttranslational modification patterns.

The genes involved in this highly complex glycosylation machinery have been examined by the present inventors by various combinations of inactivation of C1GALT1, ALG3/9, A4GALT, B3GNT2/4/5/8, B4GALNT3/4, B4GALT1/2/3/4/5/6/7, DPY19L2/3/4, FUT4/8, GALNT1/2/3/6/7/10/11/12/13/14/16/18, GCNT1, GLT1D1, GLT8D1/2, GTDC1, MGAT1/2/3/4A/4B, POMGNT1, POMT1/2, ST3GAL1/2/3/4/5/6, ST6GAL1/2, ST6GALNAC1/2/3/4, ST8SIA2/4/5, STT3A/3B, TMTC1/2/3/4, UGT8, FUT8, B3GNT2, GNPTAB, GNPTG, ST6GALNACT1, COSMC, MGAT5 and introduction of ST6GALNACT1, B4GALT1/T2/T3/T4, B3GNT6, GCNT1, ST3GAL4, ST6GAL1, ST6GALNAC1, TMTC3, FUT8, ST3GAL4/6, ST6GAL1/2, MGAT3/4A/5, B3GNT2, GNPTAB, GNPTG, GALNT1/3, CHST1/3/15/, GAL3ST2/4 and effects have been identified.

In one aspect of the present invention MGAT4A and MGAT4B is knocked out in the cell to eliminate β4-branched tetraantennary N-glycans.

In another aspect of the present invention MGAT5 is knocked out in the cell to eliminate β6-branched tetraantennary structures.

In yet another aspect of the present invention MGAT5 is knocked out in the cell leading to loss of L-PHA lectin labelling.

In a further aspect of the present invention MGAT4A, MGAT4B and MGAT5 are knocked out in the cell leading to homogenous biantennary N-glycans.

In another aspect of the present invention B4GALT1 and B4GALT3 is knocked out in the cell leading to abolished galactosylation on N-glycans.

In a further aspect of the present invention B3GNT2 is knocked out in the cell leading to elimination of poly-LacNAc.

In a further aspect of the present invention B4GALNT3 and B4GALNT4 is knocked out in the cell leading to elimination of LacDiNAc (GalNAcβ1-4GlcNAcβ), as knock out of individual genes did not completely eliminate LacDiNAc detection.

In another aspect of the present invention ST3GAL4 and ST4GAL6 are knocked out in the cell leading to homogeneous α2,6 sialylation of N-glycans.

In another aspect of the present invention ST3GAL4, ST4GAL6, and ST6GAL1 are knocked out in the cell leading to homogeneous lack of sialylation of N-glycans.

In another aspect of the present invention ST6GAL1 is knocked out in the cell leading to homogeneous α2,3 sialylation of N-glycans.

In another aspect of the present invention GNPTAB is knocked out in the cell leading to elimination of mannose-6-phosphate (M6P) tagging of N-glycans and increase in sialylated N-glycans of lysosomal enzyme proteins.

In another aspect of the present invention ALG9 is knocked out in the cell leading to reduction in M6P tagging of N-glycans and increase in sialylated N-glycans of lysosomal enzyme proteins.

In another aspect of the present invention ALG12 is knocked out in the cell leading to truncated high-mannose and hybrid type N-glycans with M6P tagging of N-glycans and increase in sialylated N-glycans of lysosomal enzyme proteins.

In another aspect of the present invention ALG8 is knocked out in the cell leading to increase in hybrid type N-glycans with M6P tagging and LacNAc with sialic acids of N-glycans of lysosomal enzyme proteins.

In another aspect of the present invention NAGPA is knocked out in the cell leading to increase in GlcNAc residues on M6P tagged N-glycans of lysosomal enzyme proteins.

In another aspect of the present invention ALG3 is knocked out in the cell leading to increase in truncated high-mannose and hybrid type N-glycans with M6P tagging of N-glycans of lysosomal enzyme proteins.

In another aspect of the present invention MGAT1 is knocked out in the cell leading to elimination of complex type N-glycans and increase in truncated high-mannose N-glycans and unchanged M6P tagging of N-glycans of lysosomal enzyme proteins.

In another aspect of the present invention MOGS is knocked out in the cell leading to high mannose type N-glycans with Glc residues and reduced M6P tagging of N-glycans of lysosomal enzyme proteins.

In another aspect of the present invention MGAT1 and GNPTAB are knocked out in the cell leading to elimination of complex type N-glycans and increase in truncated high-mannose N-glycans without M6P tagging of N-glycans of lysosomal enzyme proteins.

In another aspect of the present invention MGAT2 and GNPTAB are knocked out in the cell leading to marked increase in monoantennary N-glycans with sialic acids and without M6P tagging of N-glycans of lysosomal enzyme proteins.

In another aspect of the present invention GNPTAB and ST6GAL1 are knocked out and ST3GAL4 is knocked in in the cell leading to complex type N-glycans with α2,3-linked sialic acids and without M6P tagging of N-glycans of lysosomal enzyme proteins.

In another aspect of the present invention GNPTAB, ST3GAL4 and ST3GAL6 are knocked out and ST6GAL1 is knocked in in the cell leading to complex type N-glycans with α2,6-linked sialic acids and without M6P tagging of N-glycans of lysosomal enzyme proteins.

In another aspect of the present invention B4GALT1 and/or FUT8 is knocked out in the cell leading to more homogeneous GOF type N-glycans on IgG.

In another aspect of the present invention MGAT3 is knocked out in the cell leading to elimination of bisecting N-glycans on IgG and lysosomal proteins.

In another aspect of the present invention B4GALT1 is knocked in in the cell leading to homogeneous G2F type N-glycans on IgG.

In another aspect of the present invention B4GALT1 is knocked in and FUT8 knocked out in the cell leading to homogeneous G2 type N-glycans on IgG.

In another aspect of the present invention B4GALT1 and ST6GAL1 are knocked in and MGAT3 is knocked out in the cell leading to more homogeneous G2S1F type N-glycans with one sialylation on IgG.

In another aspect of the present invention B4GALT1 and ST6GAL1 are knocked in and MGAT3 and FUT8 are knocked out in the cell leading to more homogeneous G2S1 type N-glycans with one sialylation on IgG.

In another aspect of the present invention MGAT2 is knocked out in the cell leading to homogeneous monoantennary N-glycans on IgG.

In another aspect of the present invention MGAT2, MGAT3, ST6GAL1, ST3GAL4 and ST3GAL6 are knocked out and B4GALT1 is knocked in in the cell leading to homogeneous monoantennary G1F type N-glycans on IgG.

In another aspect of the present invention MGAT2, MGAT3, ST6GAL1, ST3GAL4, ST3GAL6 and FUT8 are knocked out and B4GALT1 is knocked in in the cell leading to homogeneous monoantennary G1 type N-glycans on IgG.

In another aspect of the present invention MGAT2, MGAT3 and ST6GAL1 are knocked out and B4GALT1 and ST3GAL4 are knocked in in the cell leading to homogeneous monoantennary type N-glycans with α2,3-linked sialic acids on IgG.

In another aspect of the present invention MGAT2, MGAT3, ST6GAL1 and FUT8 are knocked out and B4GALT1 and ST3GAL4 are knocked in in the cell leading to homogeneous monoantennary type N-glycans with α2,3-linked sialic acids and without fucose on IgG.

In another aspect of the present invention MGAT2, MGAT3, ST3GAL4, and ST3GAL6 are knocked out and B4GALT1 and ST6GAL1 are knocked in in the cell leading to homogeneous monoantennary type N-glycans with α2,6-linked sialic acids on IgG.

In another aspect of the present invention MGAT2, MGAT3, ST3GAL4, ST3GAL6, and FUT8 are knocked out and B4GALT1 and ST6GAL1 are knocked in in the cell leading to homogeneous monoantennary type N-glycans with α2,6-linked sialic acids and without fucose on IgG.

O-GalNAc Glycosylation:

In one aspect of the present invention GALNT1 and/or GALNT2 and/or GALNT3 and/or GALNT4 and/or GALNT7 and/or GALNT10 and/or GALNT11 and/or GALNT13 are knocked out in the cell to eliminate part of or all O-glycan attachments to proteins.

In one aspect of the present invention GALNT1 and/or GALNT2 and/or GALNT3 are knocked out in the cell to eliminate O-glycosylation of erythropoietin.

In another aspect of the present invention COSMC and/or C1GALT1 are knocked out in the cell leading to homogeneous truncated O-glycans with GalNAcα1-O-Ser/Thr structures.

In another aspect of the present invention COSMC and/or C1GALT1 are knocked out and ST6GALNT1 is knocked in in the cell leading to homogeneous truncated O-glycans with NeuAcα2-6GalNAcα1-O-Ser/Thr structures.

O-Man Glycosylation:

In one aspect of the present invention POMGNT1 is knocked out in the cell leading to homogeneous truncated O-glycans with Manα1-O-Ser/Thr structures.

In another aspect of the present invention POMT1 and/or POMT2 is knocked out in the cell leading to elimination of O-Man glycans on a subset of proteins including α-dystroglycan.

In another aspect of the present invention TMTC1 and/or TMTC2 and/or TMTC3 and/or TMTC4 is knocked out in the cell leading to elimination of O-Man glycans on a subset of proteins including cadherins and protocadherins.

Glycosphingolipids:

In one aspect of the present invention B4GALT5 and/or B4GALNT6 are knocked out in the cell leading to homogeneous truncated glycolipids with Glc-Cer structures.

Gene Editing Strategies:

In one aspect of the present invention is one or more of the above mentioned genes knocked out using zinc finger nucleases ZFN. ZFNs can be used for inactivation of any genes disclosed herein. ZFNs comprise a zinc finger protein (ZFP) and a nuclease (cleavage) domain.

In one aspect of the present invention is one or more of the above mentioned genes knocked out using TALENs. TALENs can be used for inactivation of any genes disclosed herein.

In one aspect of the present invention is one or more of the above mentioned genes knocked out using CRISPR/Cas9. CRISPR/Cas9 can be used for inactivation of any genes disclosed herein.

In yet other embodiments, this invention provides mammalian cells with different well-defined N-glycosylation capacities that enable recombinant production of glycoprotein therapeutics with N-glycans comprised of either biantennary, triantennary, or tetraantennary N-glycans with or without poly-LacNAc, with or without α2,6NeuAc capping, and with or without α2,3NeuAc capping.

In yet other embodiments, this invention provides mammalian cells with different well-defined N-glycosylation capacities that enable recombinant production of Lysosomal glycoprotein therapeutics with N-glycans with and without M6P tagging, with and without α2,6NeuAc capping, with or without α2,3NeuAc capping, with and without high mannose, with and without Glc residues, and with and without GlcNAc-1-phosphate residues.

In yet another aspect also provided is an isolated cell comprising any of the proteins and/or polynucleotides as described herein. In certain embodiments, one or more glycosyltransferase genes are inactivated (partially or fully) in the cell. Any of the cells described herein may include additional genes that have been inactivated, for example, using zinc finger nucleases, TALENs and/or CRISPR/Cas9 designed to bind to a target site in the selected gene. In certain embodiments, provided herein are cells or mammalian cells in which two or more glycosyltransferase genes have been inactivated, and cells or mammalian cells in which one or more glycosyltransferase and related glycogenes have been inactivated and one or more glycosyltransferase genes introduced.

In some embodiments, this invention provides a cell with inactivation of the second step in the O-GalNAc glycosylation pathway, and that produces truncated O-GalNAc O-glycans without sialic acid capping.

In some embodiments, this invention provides a cell with inactivation of the second step in the O-Xyl glycosylation pathway, and that produces truncated O-Xyl O-glycans without proteoglycan chains.

In some embodiments, this invention provides a cell with inactivation of the second step in the O-Man glycosylation pathway, and that produces truncated O-Man O-glycans without sialic acid capping.

In some embodiments, this invention provides a cell with inactivation of the second step in the O-Man, O-Xyl and O-GalNAc glycosylation pathways, and that produces truncated O-Man, O-Xyl, and O-GalNAc O-glycans.

In some embodiments, this invention provides a cell with inactivation of the second step in the glycosphingolipid glycosylation pathway, and that produces truncated glycosphingolipid glycans.

In some embodiments, this invention provides a cell with inactivation ad/or modification of the M6P tagging process of N-glycans, and that produces lysosomal enzyme proteins with no or lower or higher levels of M6P tagged N-glycans.

Thus, in another aspect, provided herein are methods for inactivating one or more cellular glycosyltransferase and related genes (e.g., MGAT1, MGAT2, MGAT3, MGAT4A, MGAT4B, MGAT4C, MGAT5, MGAT5B, B4GALT1, B4GALT2, B4GALT3, B4GALT4, B3GNT2, B3GNT8, ST3GAL3, ST3GAL4, ST3GAL6, FUT8, GNPTAB, ALG9, ALG12, ALG8, NAGPA, ALG3; MOGS, and ST6GAL1 genes (as listed in Table 1) in a cell, by use of methods comprising genome perturbation, gene-editing and/or gene disruption capability such as nucleic acid vector systems related to Clustered Regularly Interspaced Short Palindromic Repeats (CRISPR) and components thereof, nucleic acid vector systems encoding fusion proteins comprising zinc finger DNA-binding domains (ZF) and at least one cleavage domain or at least one cleavage half-domain (ZFN) and/or nucleic acid vector systems encoding a first transcription activator-like (TAL) effector endonuclease monomer and a nucleic acid encoding a second cleavage domain or at least one cleavage half-domain (TALEN). Introduction into a cell of either of the above mentioned nucleic acid cleaving agents (CRISPR, TALEN, ZFN) are capable of specifically cleaving a glycosyltransferase gene target site as a result of cellular introduction of: (Rillahan 2011) a nucleic acid encoding pair of either ZF or TAL glycosyltransferase gene target binding proteins each fused to Fok1 endonuclease, wherein at least one of said ZF or TAL polypeptides is capable of specifically binding to a nucleotide sequence located upstream from said target cleavage site, and the other ZF or TAL protein is capable of specifically binding to a nucleotide sequence located downstream from the target cleavage site, whereby each of the zinc finger proteins are independently bound to and surround the nucleic acid target followed by target nucleic acid disruption by double stranded breakage mediated by the fused endonuclease cleaving moieties, (Hansen 2015) a nucleotide sequence encoding a CRISPR-Cas system guide RNA that hybridizes glycosyltransferase gene target sequence, and b) a second nucleotide sequence encoding a Type-II Cas9 protein, wherein components (a) and (b) are located on same or different vectors of the system, wherein the guide RNA is comprised of a chimeric RNA and includes a guide sequence and a trans-activating cr (tracr) sequence, whereby the guide RNA targets the glycosyltransferase gene target sequence and the Cas9 protein cleaves the glycosyltransferase gene target site.

In yet other embodiments, this invention provides mammalian cells with inactivation of one or more glycosyltransferase genes and with stable introduction of one or more glycosyltransferases to enhance fidelity of desirable glycosylation features and/or introduce improved glycosylation features and/or novel glycosylation features.

In certain embodiments mammalian cells with inactivation of one or more sialyltransferases, and/or galactosyltransferases, and/or glucosyltransferases, and/or GlcNAc-transferases, and/or GalNAc-transferases, and/or xylosyl-transferase, and/or glucuronosyltransferases, mannosyltransferases, and/or fucosyltransferases, and in which one or more glycosyltransferases have been stably introduced are provided.

There are a number of methods available for introduction of exogenous genes such as glycosyltransferase genes in mammalian cells and selecting stable clonal mammalian cells that harbor and express the gene of interest. Typically the gene of interest is co-transfected with a selection marker gene that favors mammalian cells expressing the selection marker under certain defined media culture conditions. The media could contain an inhibitor of the selection marker protein or the media composition could stress cell metabolism and thus require increased expression of the selection marker. The selection marker gene may be present on same plasmid as gene of interest or on another plasmid.

In some embodiments, introduction of one or more exogenous glycosyltransferase(s) is performed by plasmid transfection with a plasmid encoding constitutive promotor driven expression of both the glycosyltransferase gene and a selectable antibiotic marker, where the selectable marker could also represent an essential gene not present in the host cell such as GS system (Sigma/Lonza), and/or separate plasmids encoding the constitutive promotor driven glycosyltransferase gene or the selectable marker. For example, plasmids encoding ST6GalNAc-I and Zeocin have been transfected into cells and stable ST6Gal-I expressing lines have been selected based on zeocin resistance

In some other embodiments, introduction of one or more exogenous glycosyltransferase(s) is performed by site-directed nuclease-mediated insertion.

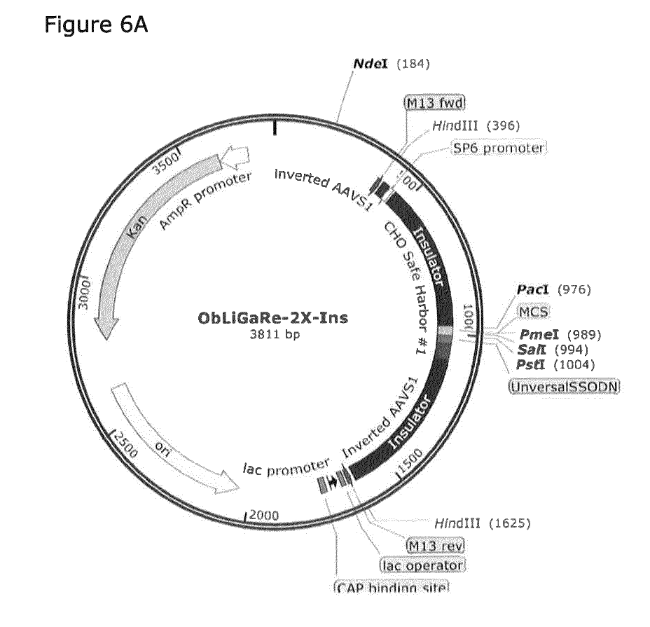

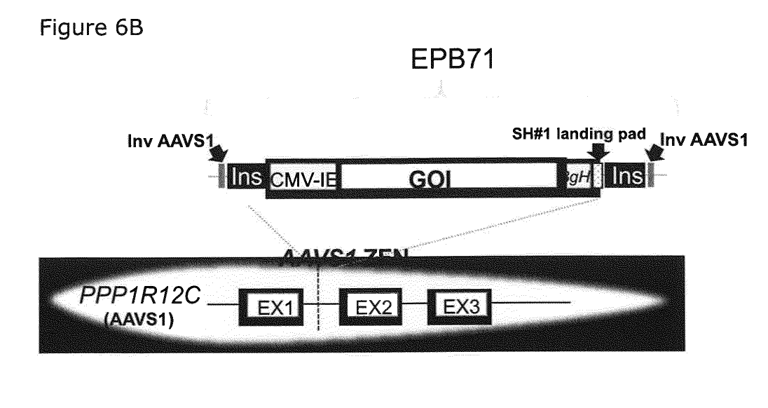

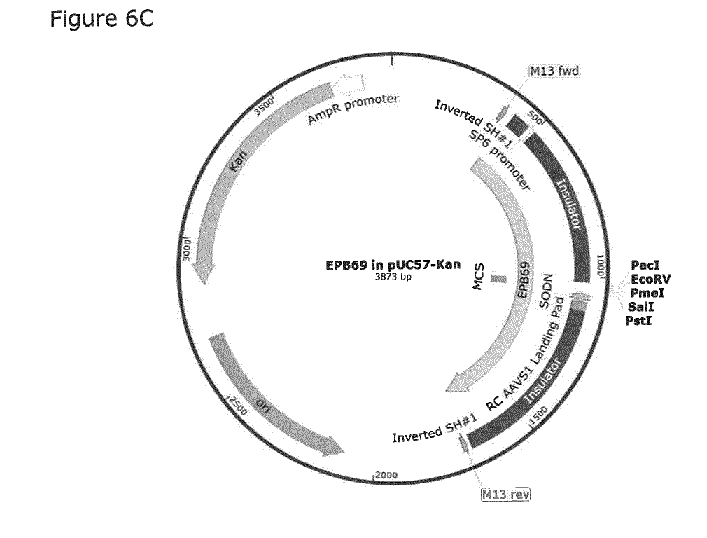

In some embodiments, a method for stably expressing at least one product of an exogenous nucleic acid sequence in a cell by introduction of double stranded breaks at the PPP1R12C or Safe Harbor #1 genomic locus using ZFN nucleic acid cleaving agents and an exogenous nucleic acid sequence that by a homology dependent manner or via compatible flanking ZFN cutting overhangs is inserted into the cleavage site and expressed. Safe Harbor sites are sites in the genome that upon manipulation do not lead to any obvious cellular or phenotypic consequences. In addition to the aforementioned sites, several other sites have been identified such as Safe Harbor #2, CCR5 and Rosa26. Besides ZFN technology, TALEN and CRISPR tools can also provide for integrating exogenous sequences into mammalian cells or genomes in a precise manner. In doing so it should be evaluated; i) to what extend epigenetic silencing and ii) what the desired expression level of the gene of interest should be. In the examples enclosed herein, site-specific integration of human glycosyltransferases e.g. ST6GALNT1 using the ObLiGaRe insertion strategy is based on a CMV expression driven insulator flanked vector design (Example 4, FIG. 6).

In yet another aspect, the disclosure provides a method of producing a recombinant protein of interest in a host cell, the method comprising the steps of: (a) providing a host cell comprising two or more endogenous glycosyltransferase genes; (b) inactivating the endogenous glycosyltransferase genes of the host cell by any of the methods described herein; and (c) introducing an expression vector comprising a transgene, the transgene comprising a sequence encoding a protein of interest, into the host cell, thereby producing the recombinant protein for display with a plurality of glycoforms. In certain embodiments, the protein of interest comprises e.g. MUC1 or an antibody, e.g., a monoclonal antibody.

In yet another aspect, the disclosure provides a method of producing a recombinant protein of interest in a cell, the method comprising the steps of: (a) providing a cell comprising one or more endogenous glycosyltransferase gene; (b) inactivating the endogenous glycosyltransferase gene(s) of the host cell; (c) introducing one or more glycosyltransferase gene(s) in the cell by any of the methods described herein; and (d) introducing an expression vector comprising a transgene, the transgene comprising a sequence encoding a protein of interest, into the cell, thereby producing the recombinant protein protein for display with a plurality of glycoforms. In certain embodiments, the protein of interest comprises e.g.

erythropoietin or an antibody, e.g., a monoclonal antibody.

Another aspect of the disclosure encompasses a method for producing a recombinant protein with a plurality of more homogeneous and/or novel and/or functionally beneficial glycosylation. The method comprises expressing the protein in a mammalian cell line deficient in two or more glycosyltransferase genes and/or deficient in one or more glycosyltransferase genes combined with one or more gained glycosyltransferase genes. In one specific embodiment, the cell line is a Human embryonic kidney (HEK293) cell line. In some embodiments, the cell line comprises inactivated chromosomal sequences encoding any endogenous glycosyltransferases. In some embodiments, the inactivated chromosomal sequences encoding any endogenous glycosyltransferases is monoallelic and the cell line produces a reduced amount of said glycosyltransferases. In some other embodiments, the inactivated chromosomal sequences encoding encoding any endogenous glycosyltransferases are biallelic, and the cell line produces no measurable said glycosyltransferases. In some other embodiments, the recombinant protein has more homogeneous and/or novel and/or functionally beneficial glycosylation. In some embodiments, the plurality of recombinant proteins with different glycoforms has at least one property that is improved relative to a similar recombinant protein produced by a comparable cell line not deficient in said endogenous glycosyltransferases, for example, biological binding, immunogenicity, increased bioavailability, increased efficacy, increased stability, increased solubility, improved half-life, improved clearance, improved pharmacokinetics, and combinations thereof. The recombinant protein can be any protein, including a therapeutic protein. Exemplary proteins include those selected from but not limited to a mucin, cell membrane protein, an antibody, an antibody fragment, a growth factor, a cytokine, a hormone, a lysosomal enzyme, a clotting factor, and functional fragment or variants thereof.

The disclosure may also be used to identify target genes for modification and use this knowledge to glycoengineer an existing mammalian cell line previously transfected with DNA coding for the protein of interest.

In any of the cells and methods described herein, the cell or cell line can be a HEK293 (e.g., HEK293-F, HEK293-H, HEK293-T, HEK293-6E), COS, VERO, MDCK, WI38, V79, B14AF28-G3, BHK, HaK, NS0, SP2/0-Ag14, HeLa, and PERC6.

Mammalian cells as described herein can also be used to display and glycooptimize N-glycoproteins including without intend for limitation for example α1-antitrypsin, gonadotropins, lysosomal targeted enzyme proteins (e.g. Glycocerebrosidase, alpha-Galactosidase, alpha-glucosidase, sulfatases, glucuronidase, iduronidase). Known human glycosyltransferase genes are assembled in homologous gene families in the CAZy database and these families are further assigned to different glycosylation pathways in Hansen et al. (Hansen, Lind-Thomsen et al. 2014). Table 1 lists all human glycosyltransferase genes in CAZy GT families with NCBI Gene IDs and assignment of confirmed or putative functions in biosynthesis of different mammalian glycoconjugates (N-glycans, O-GalNAc, O-GlcNAc, O-Glc, O-Gal, O-Fuc, O-Xyl, O-Man, C-Man, Glycosphingolipids, Hyaluronan, and GPI anchors). FIG. 1, panels 1B,1C,1D and 1E further graphically depicts confirmed and putative roles of the human CAZy GT families in biosynthesis of different glycoconjugates.

TABLE 1
Human GTf (in total 214)

| CAZy | Gene | Gene | |
|---|---|---|---|
| family⁽¹⁾ | ID⁽²⁾ | Symbol⁽³⁾ | Description⁽⁴⁾ |
| GTnc | 127550 | A3GALT2* | alpha 1,3-galactosyltransferase 2 (inactive) |
| GT32 | 53947 | A4GALT | α1,4-galactosyltransferase |
| GT32 | 51146 | A4GNT | α1,4-N-acetylglucosaminyltransferase |
| GT6 | 28 | ABO | ABO blood group |
| GT33 | 56052 | ALG1* | chitobiosyldiphosphodolichol β-mannosyltransferase |
| GT59 | 84920 | ALG10* | α1,2-glucosyltransferase |
| GT59 | 144245 | ALG10B* | α1,2-glucosyltransferase |
| GT4 | 440138 | ALG11* | α1,2-mannosyltransferase |
| GT22 | 79087 | ALG12* | α1,6-mannosyltransferase |
| GT1 | 79868 | ALG13* | UDP-N-acetylglucosaminyltransferase subunit |
| GT1 | 199857 | ALG14* | UDP-N-acetylglucosaminyltransferase subunit |
| GT33 | 200810 | ALG1L* | chitobiosyldiphosphodolichol β-mannosyltransferase- |
| | | | like |
| GT33 | 644974 | ALG1L2* | chitobiosyldiphosphodolichol β-mannosyltransferase- |
| | | | like 2 |
| GT4 | 85365 | ALG2* | α1,3/1,6-mannosyltransferase |
| GT58 | 10195 | ALG3* | α1,3- mannosyltransferase |
| GT2 | 29880 | ALG5* | dolichyl-phosphate b-glucosyltransferase |
| GT57 | 29929 | ALG6* | α1,3-glucosyltransferase |
| GT57 | 79053 | ALG8* | α1,3-glucosyltransferase |
| GT22 | 79796 | ALG9* | α1,2-mannosyltransferase |
| GT31 | 8706 | B3GALNT1 | b1,3-N-acetylgalactosaminyltransferase 1 |
| GT31 | 148789 | B3GALNT2 | b1,3-N-acetylgalactosaminyltransferase 2 |
| GT31 | 8708 | B3GALT1 | UDP-Gal:bGlcNAc β 1,3-galactosyltransferase, |
| | | | polypeptide 1 |
| GT31 | 8707 | B3GALT2 | UDP-Gal:bGlcNAc b1,3-galactosyltransferase, |
| | | | polypeptide 2 |
| GT31 | 8705 | B3GALT4 | UDP-Gal:bGlcNAc-b1,3-galactosyltransferase, |
| | | | polypeptide 4 |
| GT31 | 10317 | B3GALT5 | UDP-Gal:bGlcNAc-b1,3-galactosyltransferase, |
| | | | polypeptide 5 |
| GT31 | 126792 | B3GALT6 | UDP-Gal:bGal-b1,3-galactosyltransferase |
| | | | polypeptide 6 |
| GT43 | 27087 | B3GAT1 | b1,3-glucuronyltransferase 1 |
| GT43 | 135152 | B3GAT2 | b1,3-glucuronyltransferase 2 |
| GT43 | 26229 | B3GAT3 | b1,3-glucuronyltransferase 3 |
| GT31 | 145173 | B3GLCT | Beta-1,3-glucosyltransferase |
| GT31 | 10678 | B3GNT2 | UDP-GlcNAc:bGal b1,3-N- |
| | | | acetylglucosaminyltransferase 2 |
| GT31 | 10331 | B3GNT3 | UDP-GlcNAc:bGal b1,3-N- |
| | | | acetylglucosaminyltransferase 3 |
| GT31 | 79369 | B3GNT4 | UDP-GlcNAc:bGal b1,3-N- |
| | | | acetylglucosaminyltransferase 4 |
| GT31 | 84002 | B3GNT5 | UDP-GlcNAc:bGal b1,3-N- |
| | | | acetylglucosaminyltransferase 5 |
| GT31 | 192134 | B3GNT6 | UDP-GlcNAc:bGal b1,3-N- |
| | | | acetylglucosaminyltransferase 6 |
| GT31 | 93010 | B3GNT7 | UDP-GlcNAc:bGal b1,3-N- |
| | | | acetylglucosaminyltransferase 7 |
| GT31 | 374907 | B3GNT8 | UDP-GlcNAc:bGal b1,3-N- |
| | | | acetylglucosaminyltransferase 8 |
| GT31 | 84752 | B3GNT9 | UDP-GlcNAc:bGal b1,3-N- |
| | | | acetylglucosaminyltransferase 9 |
| GT2 | 146712 | B3GNTL1 | UDP-GlcNAc:bGal b1,3-N- |
| | | | acetylglucosaminyltransferase-like 1 |
| GT12 | 2583 | B4GALNT1 | b1,4-N-acetyl-galactosaminyl transferase 1 |
| GT12 | 124872 | B4GALNT2 | b1,4-N-acetyl-galactosaminyl transferase 2 |
| GT7 | 283358 | B4GALNT3 | b1,4-N-acetyl-galactosaminyl transferase 3 |
| GT7 | 338707 | B4GALNT4 | b1,4-N-acetyl-galactosaminyl transferase 4 |
| GT7 | 2683 | B4GALT1 | UDP-Gal:bGlcNAc b1,4- galactosyltransferase, |
| | | | polypeptide 1 |
| GT7 | 8704 | B4GALT2 | UDP-Gal:bGlcNAc b1,4- galactosyltransferase, |
| | | | polypeptide 2 |
| GT7 | 8703 | B4GALT3 | UDP-Gal:bGlcNAc b1,4- galactosyltransferase, |
| | | | polypeptide 3 |
| GT7 | 8702 | B4GALT4 | UDP-Gal:bGlcNAc b1,4- galactosyltransferase, |
| | | | polypeptide 4 |
| GT7 | 9334 | B4GALT5 | UDP-Gal:bGlcNAc b1,4- galactosyltransferase, |
| | | | polypeptide 5 |
| GT7 | 9331 | B4GALT6 | UDP-Gal:bGlcNAc b1,4- galactosyltransferase, |
| | | | polypeptide 6 |
| GT7 | 11285 | B4GALT7 | xylosylprotein b1,4-galactosyltransferase, |
| | | | polypeptide 7 |
| GT49 | 11041 | B4GAT1 | UDP-GlcNAc:bGal bl,3-N- |
| | | | acetylglucosaminyltransferase 1 |
| GT31 | 56913 | C1GALT1 | core 1 synthase, galactosyltransferase 1 |
| GT31 | 29071 | C1GALT1C1 | C1GALT1-specific chaperone 1 |
| GT25 | 51148 | CERCAM | cerebral endothelial cell adhesion molecule |
| | | | (inactive) |
| GT7/31 | 79586 | CHPF | chondroitin polymerizing factor |
| GT7/31 | 54480 | CHPF2 | chondroitin polymerizing factor 2 |
| GT7/31 | 22856 | CHSY1 | chondroitin sulfate synthase 1 |
| GT7/31 | 337876 | CHSY3 | chondroitin sulfate synthase 3 |
| GT25 | 79709 | COLGALT1 | collagen b(1-O)galactosyltransferase 1 |
| GT25 | 23127 | COLGALT2 | collagen b(1-O)galactosyltransferase 2 |
| GT7 | 55790 | CSGALNACT1 | chondroitin sulfate N- |
| | | | acetylgalactosaminyltransferase 1 |
| GT7 | 55454 | CSGALNACT2 | chondroitin sulfate N- |
| | | | acetylgalactosaminyltransferase 2 |
| GT2 | 8813 | DPM1* | dolichyl-phosphate mannosyltransferase polypeptide |
| | | | 1 |
| GTnc | 23333 | DPY19L1 | dpy-19-like 1 (C. elegans) |
| GTnc | 283417 | DPY19L2 | dpy-19-like 2 (C. elegans) |
| GTnc | 147991 | DPY19L3 | dpy-19-like 3 (C. elegans) |
| GTnc | 286148 | DPY19L4 | dpy-19-like 4 (C. elegans) |
| GT61 | 285203 | EOGT | EGF domain-specific O-linked N-acetylglucosamine |
| | | | transferase |
| GT47/64 | 2131 | EXT1 | exostosin glycosyltransferase 1 |
| GT47/64 | 2132 | EXT2 | exostosin glycosyltransferase 2 |
| GT47/64 | 2134 | EXTL1 | exostosin-like glycosyltransferase 1 |
| GT64 | 2135 | EXTL2 | exostosin-like glycosyltransferase 2 |
| GT47/64 | 2137 | EXTL3 | exostosin-like glycosyltransferase 3 |
| GTnc | 79147 | FKRP* | fukutin related protein |
| GTnc | 2218 | FKTN* | fukutin |
| GT11 | 2523 | FUT1 | fucosyltransferase 1, H blood group |
| GT10 | 84750 | FUT10 | fucosyltransferase 10, α1,3 fucosyltransferase |
| GT10 | 170384 | FUT11 | fucosyltransferase 11, α1,3 fucosyltransferase |
| GT11 | 2524 | FUT2 | fucosyltransferase 2 secretor status include |
| GT10 | 2525 | FUT3 | fucosyltransferase 3, Lewis blood group |
| GT10 | 2526 | FUT4 | fucosyltransferase 4, α1,3 fucosyltransferase, |
| | | | myeloid-specific |
| GT10 | 2527 | FUT5 | fucosyltransferase 5, α1,3 fucosyltransferase |
| GT10 | 2528 | FUT6 | fucosyltransferase 6, α1,3 fucosyltransferase |
| GT10 | 2529 | FUT7 | fucosyltransferase 7, α1,3 fucosyltransferase |
| GT23 | 2530 | FUT8 | fucosyltransferase 8, α1,6 fucosyltransferase |
| GT10 | 10690 | FUT9 | fucosyltransferase 9, α1,3 fucosyltransferase |
| GT27 | 2589 | GALNT1 | polypeptide N-acetylgalactosaminyltransferase 1 |
| GT27 | 55568 | GALNT10 | polypeptide N-acetylgalactosaminyltransferase 10 |
| GT27 | 63917 | GALNT11 | polypeptide N-acetylgalactosaminyltransferase 11 |
| GT27 | 79695 | GALNT12 | polypeptide N-acetylgalactosaminyltransferase 12 |
| GT27 | 114805 | GALNT13 | polypeptide N-acetylgalactosaminyltransferase 13 |
| GT27 | 79623 | GALNT14 | polypeptide N-acetylgalactosaminyltransferase 14 |
| GT27 | 117248 | GALNT15 | polypeptide N-acetylgalactosaminyltransferase 15 |
| GT27 | 57452 | GALNT16 | polypeptide N-acetylgalactosaminyltransferase 16 |
| GT27 | 374378 | GALNT18 | polypeptide N-acetylgalactosaminyltransferase 18 |
| GT27 | 2590 | GALNT2 | polypeptide N-acetylgalactosaminyltransferase 2 |
| GT27 | 2591 | GALNT3 | polypeptide N-acetylgalactosaminyltransferase 3 |
| GT27 | 8693 | GALNT4 | polypeptide N-acetylgalactosaminyltransferase 4 |
| GT27 | 11227 | GALNT5 | polypeptide N-acetylgalactosaminyltransferase 5 |
| GT27 | 11226 | GALNT6 | polypeptide N-acetylgalactosaminyltransferase 6 |
| GT27 | 51809 | GALNT7 | polypeptide N-acetylgalactosaminyltransferase 7 |
| GT27 | 26290 | GALNT8 | polypeptide N-acetylgalactosaminyltransferase 8 |
| GT27 | 50614 | GALNT9 | polypeptide N-acetylgalactosaminyltransferase 9 |
| GT27 | 168391 | GALNTL5 | polypeptide N-acetylgalactosaminyltransferase-like 5 |
| GT27 | 442117 | GALNTL6 | polypeptide N-acetylgalactosaminyltransferase-like 6 |
| GT27 | 64409 | WBSCR17 | Williams-Beuren syndrome chromosome region 17 |
| GT6 | 26301 | GBGT1* | globoside α1,3-N-acetylgalactosaminyltransferase 1 |
| | | | (Forssman) |
| GT14 | 2650 | GCNT1 | glucosaminyl (N-acetyl) transferase 1, core 2 |
| GT14 | 2651 | GCNT2 | glucosaminyl (N-acetyl) transferase 2, I-branching |
| | | | enzyme |
| GT14 | 9245 | GCNT3 | glucosaminyl (N-acetyl) transferase 3, mucin type |
| GT14 | 51301 | GCNT4 | glucosaminyl (N-acetyl) transferase 4, core 2 |
| GT14 | 644378 | GCNT6 | glucosaminyl (N-acetyl) transferase 6 |
| GT14 | 140687 | GCNT7 | glucosaminyl (N-acetyl) transferase family member 7 |
| GT4 | 144423 | GLT1D1* | glycosyltransferase 1 domain containing 1 |
| GT6 | 360203 | GLT6D1* | glycosyltransferase 6 domain containing 1 |
| GT8 | 55830 | GLT8D1* | glycosyltransferase 8 domain containing 1 |
| GT8 | 83468 | GLT8D2* | glycosyltransferase 8 domain containing 2 |
| GT4 | 79712 | GTDC1* | glycosyltransferase-like domain containing 1 |
| GT8 | 283464 | GXYLT1 | glucoside xylosyltransferase 1 |
| GT8 | 727936 | GXYLT2 | glucoside xylosyltransferase 2 |
| GT8 | 2992 | GYG1* | glycogenin 1 |
| GT8 | 8908 | GYG2* | glycogenin 2 |
| GT8/49 | 120071 | GYLTL1B | glycosyltransferase-like 1B, LARGE2 |
| GT3 | 2997 | GYS1* | glycogen synthase 1 (muscle) |
| GT3 | 2998 | GYS2* | glycogen synthase 2 (liver) |
| GT2 | 3036 | HAS1* | hyaluronan synthase 1 |
| GT2 | 3037 | HAS2* | hyaluronan synthase 2 |
| GT2 | 3038 | HAS3* | hyaluronan synthase 3 |
| GT90 | 79070 | KDELC1* | KDEL motif-containing protein 1 |
| GTnc | 143888 | KDELC2* | KDEL motif-containing protein 2 |
| GT8/49 | 9215 | LARGE | β1,3-xylosyltransferase |
| GT31 | 3955 | LFNG | O-fucosylpeptide 3-βN- |
| | | | acetylglucosaminyltransferase |
| GT31 | 4242 | MFNG | O-fucosylpeptide 3-βN- |
| | | | acetylglucosaminyltransferase |
| GT13 | 4245 | MGAT1 | mannosyl α1,3glycoprotein β1,2N- |
| | | | acetylglucosaminyltransferase |
| GT16 | 4247 | MGAT2 | mannosyl α1,6glycoprotein β2N- |
| | | | acetylglucosaminyltransferase |
| GT17 | 4248 | MGAT3 | mannosyl β1,4glycoprotein β1,4N- |
| | | | acetylglucosaminyltransferase |
| GT54 | 11320 | MGAT4A | mannosyl α1,3glycoprotein β1,4N- |
| | | | acetylglucosaminyltransferase |
| GT54 | 11282 | MGAT4B | mannosyl α1,3glycoprotein β1,4N- |
| | | | acetylglucosaminyltransferase |
| GT54 | 25834 | MGAT4C | mannosyl α1,3glycoprotein β1,4N- |
| | | | acetylglucosaminyltransferase |
| GTnc | 152586 | MGAT4D | mannosyl α1,3glycoprotein β1,4N- |
| | | | acetylglucosaminyltransferase-like |
| GT18 | 4249 | MGAT5 | mannosyl α1,6glycoprotein β1,6N- |
| | | | acetylglucosaminyltransferase |
| GT18 | 146664 | MGAT5B | mannosyl α1,6glycoprotein β-1,6-N-acetyl- |
| | | | glucosaminyltransferase, isozyme B |
| GT41 | 8473 | OGT | O-linked N-acetylglucosamine (GlcNAc) transferase |
| GT4 | 5277 | PIGA | phosphatidylinositol glycan anchor biosynthesis, |
| | | | class A |
| GT22 | 9488 | PIGB | phosphatidylinositol glycan anchor biosynthesis, |
| | | | class B |
| GT50 | 93183 | PIGM | phosphatidylinositol glycan anchor biosynthesis, |
| | | | class M |
| GT76 | 55650 | PIGV | phosphatidylinositol glycan anchor biosynthesis, |
| | | | class V |
| GT22 | 80235 | PIGZ | phosphatidylinositol glycan anchor biosynthesis, |
| | | | class Z |
| GTnc | 8985 | PLOD3 | procollagen-lysine, 2-oxoglutarate 5-dioxygenase 3 |
| GT65 | 23509 | POFUT1 | O-fucosyltransferase 1 |
| GT68 | 23275 | POFUT2 | O-fucosyltransferase 2 |
| GT90 | 56983 | POGLUT1 | O-glucosyltransferase 1 |
| GT13 | 55624 | POMGNT1 | O-linked mannose N-acetylglucosaminyltransferase |
| | | | 1, β1, 2 |
| GT61 | 84892 | POMGNT2 | O-linked mannose N-acetylglucosaminyltransferase |
| | | | 2, β1, 4 |
| GT39 | 10585 | POMT1 | O-mannosyltransferase 1 |
| GT39 | 29954 | POMT2 | O-mannosyltransferase 2 |
| GT35 | 5834 | PYGB* | phosphorylase, glycogen; brain |
| GT35 | 5836 | PYGL* | phosphorylase, glycogen, liver |
| GT35 | 5837 | PYGM* | phosphorylase, glycogen, muscle |
| GT31 | 5986 | RFNG | O-fucosylpeptide 3βN-acetylglucosaminyltransferase |
| GT29 | 6482 | ST3GAL1 | β-galactoside α-2,3-sialyltransferase 1 |
| GT29 | 6483 | ST3GAL2 | β-galactoside α-2,3-sialyltransferase 2 |
| GT29 | 6487 | ST3GAL3 | β-galactoside α-2,3-sialyltransferase 3 |
| GT29 | 6484 | ST3GAL4 | β-galactoside α-2,3-sialyltransferase 4 |
| GT29 | 8869 | ST3GAL5 | β-galactoside α-2,3-sialyltransferase 5 |
| GT29 | 10402 | ST3GAL6 | β-galactoside α-2,3-sialyltransferase 6 |
| GT29 | 6480 | ST6GAL1 | β-galactosamide α-2,6-sialyltranferase 1 |
| GT29 | 84620 | ST6GAL2 | β-galactosamide α-2,6-sialyltranferase 2 |
| GT29 | 55808 | ST6GALNAC1 | α-N-acetyl-neuraminyl-2,3-β-galactosyl-1,3-N- |
| | | | acetylgalactosaminide α-2,6-sialyltransferase 1 |
| GT29 | 10610 | ST6GALNAC2 | α-N-acetyl-neuraminyl-2,3-β-galactosyl-1,3)-N- |
| | | | acetylgalactosaminide α-2,6-sialyltransferase 2 |
| GT29 | 256435 | ST6GALNAC3 | α-N-acetyl-neuraminyl-2,3-β-galactosyl-1,3)-N- |
| | | | acetylgalactosaminide α-2,6-sialyltransferase 3 |
| GT29 | 27090 | ST6GALNAC4 | α-N-acetyl-neuraminyl-2,3-β-galactosyl-1,3-N- |
| | | | acetylgalactosaminide α-2,6-sialyltransferase 4 |
| GT29 | 81849 | ST6GALNAC5 | α-N-acetyl-neuraminyl-2,3-β-galactosyl-1,3-N- |
| | | | acetylgalactosaminide α-2,6-sialyltransferase 5 |
| GT29 | 30815 | ST6GALNAC6 | α-N-acetyl-neuraminyl-2,3-β-galactosyl-1,3-N- |
| | | | acetylgalactosaminide α-2,6-sialyltransferase 6 |
| GT29 | 6489 | ST8SIA1 | α-N-acetyl-neuraminide α-2,8-sialyltransferase 1 |
| GT29 | 8128 | ST8SIA2 | α-N-acetyl-neuraminide α-2,8-sialyltransferase 2 |
| GT29 | 51046 | ST8SIA3 | α-N-acetyl-neuraminide α-2,8-sialyltransferase 3 |
| GT29 | 7903 | ST8SIA4 | α-N-acetyl-neuraminide α-2,8-sialyltransferase 4 |
| GT29 | 29906 | ST8SIA5 | α-N-acetyl-neuraminide α-2,8-sialyltransferase 5 |
| GT29 | 338596 | ST8SIA6 | α-N-acetyl-neuraminide α-2,8-sialyltransferase 6 |
| GT66 | 3703 | STT3A | subunit of the oligosaccharyltransferase complex |
| | | | (catalytic) |
| GT66 | 201595 | STT3B | subunit of the oligosaccharyltransferase complex |
| | | | (catalytic) |
| GTnc | 10329 | TMEM5* | transmembrane protein 5 |
| GTnc | 83857 | TMTC1 | transmembrane and tetratricopeptide repeat |
| | | | containing 1 |
| GTnc | 160335 | TMTC2 | transmembrane and tetratricopeptide repeat |
| | | | containing 2 |
| GTnc | 160418 | TMTC3 | transmembrane and tetratricopeptide repeat |
| | | | containing 3 |
| GTnc | 84899 | TMTC4 | transmembrane and tetratricopeptide repeat |
| | | | containing 4 |
| GT21 | 7357 | UGCG | UDP-glucose ceramide glucosyltransferase |
| GT24 | 56886 | UGGT1* | UDP-glucose glycoprotein glucosyltransferase 1 |
| GT24 | 55757 | UGGT2* | UDP-glucose glycoprotein glucosyltransferase 2 |
| GT1 | 54658 | UGT1A1* | UDP glucuronosyltransferase 1 family, polypeptide |
| | | | Al |
| GT1 | 54575 | UGT1A10* | UDP glucuronosyltransferase 1 family, polypeptide |
| | | | A10 |
| GT1 | 54659 | UGT1A3* | UDP glucuronosyltransferase 1 family, polypeptide |
| | | | A3 |
| GT1 | 54657 | UGT1A4* | UDP glucuronosyltransferase 1 family, polypeptide |
| | | | A4 |
| GT1 | 54579 | UGT1A5* | UDP glucuronosyltransferase 1 family, polypeptide |
| | | | A5 |
| GT1 | 54578 | UGT1A6* | UDP glucuronosyltransferase 1 family, polypeptide |
| | | | A6 |
| GT1 | 54577 | UGT1A7* | UDP glucuronosyltransferase 1 family, polypeptide |
| | | | A7 |
| GT1 | 54576 | UGT1A8* | UDP glucuronosyltransferase 1 family, polypeptide |
| | | | A8 |
| GT1 | 54600 | UGT1A9* | UDP glucuronosyltransferase 1 family, polypeptide |
| | | | A9 |
| GT1 | 10941 | UGT2A1* | UDP glucuronosyltransferase 2 family, polypeptide |
| | | | A1 |
| GT1 | 79799 | UGT2A3* | UDP glucuronosyltransferase 2 family, polypeptide |
| | | | A3 |
| GT1 | 7365 | UGT2B10* | UDP glucuronosyltransferase 2 family, polypeptide |
| | | | B10 |
| GT1 | 10720 | UGT2B11* | UDP glucuronosyltransferase 2 family, polypeptide |
| | | | B11 |
| GT1 | 7366 | UGT2B15* | UDP glucuronosyltransferase 2 family, polypeptide |
| | | | B15 |
| GT1 | 7367 | UGT2B17* | UDP glucuronosyltransferase 2 family, polypeptide |
| | | | B17 |
| GT1 | 54490 | UGT2B28* | UDP glucuronosyltransferase 2 family, polypeptide |
| | | | B28 |
| GT1 | 7363 | UGT2B4* | UDP glucuronosyltransferase 2 family, polypeptide |
| | | | B4 |
| GT1 | 7364 | UGT2B7* | UDP glucuronosyltransferase 2 family, polypeptide |
| | | | B7 |
| GT1 | 133688 | UGT3A1* | UDP glycosyltransferase 3 family, polypeptide A1 |
| GT1 | 167127 | UGT3A2* | UDP glycosyltransferase 3 family, polypeptide A2 |
| GT1 | 7368 | UGT8 | UDP glycosyltransferase 8 |
| GT8 | 152002 | XXYLT1 | xyloside xylosyltransferase 1 |
| GT14 | 64131 | XYLT1 | xylosyltransferase I |
| GT14 | 64132 | XYLT2 | xylosyltransferase II |

⁽¹⁾GT classification system (Lombard et al. (2013). Nucl Acid Res 42: D1P: D490-D495
⁽²⁾Gene ID, GenBank
⁽³⁾Approved HGNC gene symbol
⁽⁴⁾Official full name, UniProt
*GTfs not included in the deconstruction scheme in FIG. 1 and Table 5

TABLE 2 lists are known human non-glycosyltransferase genes that modify glycans including sulfotransferases with NCBI Gene IDs and assignment of confirmed or putative functions in biosynthesis of different mammalian glycoconjugates.

TABLE 2
Human Non-GTfs (in total 62)

| PTM | Gene | Gene | |
|---|---|---|---|
| Family⁽¹⁾ | ID⁽²⁾ | Symbol⁽³⁾ | Description⁽⁴⁾ |
| Acetylation | 64921 | CASD1 | CAS1 domain-containing protein 1 |
| Sulfo-T | 8534 | CHST1 | Carbohydrate sulfotransferase 1 |
| Sulfo-T | 9486 | CHST10 | Carbohydrate sulfotransferase 10 |
| Sulfo-T | 50515 | CHST11 | Carbohydrate sulfotransferase 11 |
| Sulfo-T | 55501 | CHST12 | Carbohydrate sulfotransferase 12 |
| Sulfo-T | 166012 | CHST13 | Carbohydrate sulfotransferase 13 |
| Sulfo-T | 113189 | CHST14 | Carbohydrate sulfotransferase 14 |
| Sulfo-T | 51363 | CHST15 | Carbohydrate sulfotransferase 15 |
| Sulfo-T | 9435 | CHST2 | Carbohydrate sulfotransferase 2 |
| Sulfo-T | 9469 | CHST3 | Carbohydrate sulfotransferase 3 |
| Sulfo-T | 10164 | CHST4 | Carbohydrate sulfotransferase 4 |
| Sulfo-T | 23563 | CHST5 | Carbohydrate sulfotransferase 5 |
| Sulfo-T | 4166 | CHST6 | Carbohydrate sulfotransferase 6 |
| Sulfo-T | 56548 | CHST7 | Carbohydrate sulfotransferase 7 |
| Sulfo-T | 64377 | CHST8 | Carbohydrate sulfotransferase 8 |
| Sulfo-T | 83539 | CHST9 | Carbohydrate sulfotransferase 9 |
| OST | 1603 | DAD1 | Dolichyl-diphosphooligosaccharide--protein |
| | | | glycosyltransferase subunit DAD1 |
| OST | 1650 | DDOST | dolichyl-diphosphooligosaccharide-protein |
| | | | glycosyltransferase subunit (non-catalytic) |
| Donor | 22845 | DOLK* | dolichol kinase |
| Donor | 1798 | DPAGT1* | dolichyl-phosphate (UDP-N-acetylglucosamine) N- |
| | | | acetylglucosaminephosphotransferase 1 |
| Donor | 8818 | DPM2* | Dolichol phosphate-mannose biosynthesis regulatory |
| | | | protein |
| Donor | 54344 | DPM3* | dolichyl-phosphate mannosyltransferase polypeptide 3 |
| Epimerase | 29940 | DSE | Dermatan-sulfate epimerase |
| Epimerase | 92126 | DSEL | dermatan-sulfate epimerase-like protein precursor |
| Kinase | 9917 | FAM20B | Glycosaminoglycan xylosylkinase |
| Sulfo-T | 9514 | GAL3ST1 | galactose-3-O-sulfotransferase 1 |
| Sulfo-T | 64090 | GAL3ST2 | galactose-3-O-sulfotransferase 2 |
| Sulfo-T | 89792 | GAL3ST3 | galactose-3-O-sulfotransferase 3 |
| Sulfo-T | 79690 | GAL3ST4 | Galactose-3-O-sulfotransferase 4 |
| Epimerase | 26035 | GLCE | D-glucuronyl C5-epimerase |
| Man-6-P | 79158 | GNPTAB | N-acetylglucosamine-1-phosphate transferase, alpha |
| | | | and beta subunits |
| Man-6-P | 84572 | GNPTG | N-acetylglucosamine-1-phosphate transferase, gamma |
| | | | subunit |
| Degradation | 10855 | HPSE | heparanase |
| Sulfo-T | 9653 | HS2ST1 | heparan sulfate 2-O-sulfotransferase 1 |
| Sulfo-T | 9957 | HS3ST1 | heparan sulfate D-glucosaminyl3-O-Sulfo T2 |
| Sulfo-T | 9956 | HS3ST2 | heparan sulfate D-glucosaminyl3-O-Sulfo T2 |
| Sulfo-T | 9955 | HS3ST3A1 | heparan sulfate D-glucosaminyl3-O-Sulfo T3A1 |
| Sulfo-T | 9953 | HS3ST3B1 | heparan sulfate D-glucosaminyl3-O-Sulfo T3B1 |
| Sulfo-T | 9951 | HS3ST4 | heparan sulfate D-glucosaminyl3-O-Sulfo T4 |
| Sulfo-T | 222537 | HS3ST5 | heparan sulfate D-glucosaminyl3-O-Sulfo T5 |
| Sulfo-T | 64711 | HS3ST6 | heparan sulfate D-glucosaminyl3-O-Sulfo T6 |
| Sulfo-T | 9394 | HS6ST1 | heparan sulfate 6-O-sulfotransferase 1 |
| Sulfo-T | 90161 | HS6ST2 | heparan sulfate 6-O-sulfotransferase 2 |
| Sulfo-T | 266722 | HS6ST3 | heparan sulfate 6-O-sulfotransferase 3 |
| Sulfo-T | 3340 | NDST1 | heparan N-deacetylase/N-sulfotransferase-1 |
| Sulfo-T | 8509 | NDST2 | heparan N-deacetylase/N-sulfotransferase-2 |
| Sulfo-T | 9348 | NDST3 | heparan N-deacetylase/N-sulfotransferase-3 |
| Sulfo-T | 64579 | NDST4 | heparan N-deacetylase/N-sulfotransferase-4 |
| Man-6-P | 51172 | NAGPA | N-acetylglucosamine-1-phosphodiester alpha-N- |
| | | | acetylglucosaminidase |
| OST | 100128731 | OST4 | dolichyl-diphosphooligosaccharide--protein |
| | | | glycosyltransferase subunit 4 |
| GPI- | 5279 | PIGC* | phosphatidylinositol glycan anchor biosynthesis, class C |
| anchor | | | |
| GPI- | 5283 | PIGH* | phosphatidylinositol glycan anchor biosynthesis, class H |
| anchor | | | |
| GPI- | 51227 | PIGP* | phosphatidylinositol glycan anchor biosynthesis, class P |
| anchor | | | |
| GPI- | 9091 | PIGQ* | phosphatidylinositol glycan anchor biosynthesis, class Q |
| anchor | | | |
| Kinase | 84197 | POMK | protein-O-mannose kinase |
| OST | 91869 | RFT1 | RFT1 homolog |
| OST | 6184 | RPN1 | ribophorin I |
| OST | 6185 | RPN2 | ribophorin II |
| Degradation | 23213 | SULF1 | sulfatase 1 |
| Degradation | 55959 | SULF2 | sulfatase 2 |
| OST | 7991 | TUSC3 | Tumor suppressor candidate 3 |
| Sulfo-T | 10090 | UST | uronyl 2-Sulfo Transferase |

⁽¹⁾Genes are grouped after PTM (post translational modification) function (OST, oligosaccharyltransferase; GPI, Glycosylphosphatidylinositol anchor; Sulfo-T, sulfotransferase)
⁽²⁾Gene ID, GenBank
⁽³⁾Approved HGNC gene symbol
⁽⁴⁾Official full name. UniProt
*Non-GTfs not included in the deconstruction scheme in FIG. 1 and Table 5

The present inventors previously explored the glycosylation capacity of CHO cells using a genetic knock out screen focused on the N-glycosylation pathway, and demonstrated that the N-glycome of cells can be modified by a combinatorial knock out approach. The present inventors also demonstrated that site-directed knock in of one glycosyltransferase resulted in efficient glycosylation, but it is still difficult to design and built more complex glycosylation traits in homogeneous form by knock in of glycosyltransferase genes primarily because the relative expression levels of these enzymes participate in determining the glycosylation capacities and stable knock in of multiple genes is still a challenge.

By listing reported RNA sequencing data of a CHO-K1 line (Xu 2011) with RNA sequencing data from the human kidney HEK293 cells (Human Protein Atlas) it is obvious that CHO cells express a limited number of the annotated glycosyltransferases and other glycogenes (Table 3). Accordingly HEK293 and other human cells in general have a substantially broader glycosyltransferase gene repertoire compared to CHO. HEK293 cells express a large number of glycogenes not expressed in CHO cells, which provides HEK293 with considerable more complex glycosylation capacities compared to CHO. Such capacities of significance for the present invention include for example extensive fucosylation (FUTs), capping by α2,6sialic acids (ST6GAL1) and LacdiNAc (B4GALNT3/4), N-glycan branching (MGAT4s), O-GalNAc glycan density and branching (GALNTs and GCNTs), O-Man glycosylation and branching (POMTs and MGAT5B), glycolipids with globo, ganglio and lactoseries structures (A4GALT, B4GALNT1, B3GNT5), and more extensive sulfation of proteoglycans and glycoproteins.

TABLE 3
GTf genes, available RNA_seq data for HEK293 and CHO-K1

| | | | CHO-K1 |
|---|---|---|---|
| CAZy | Gene | HEK293 | (RNA mapping |
| family⁽¹⁾ | symbol⁽²⁾ | (fpkm)⁽³⁾ | Depth)⁽⁴⁾ |
| GTnc | A3GALT2 | 0.0 | nd |
| GT32 | A4GALT | 3.5 | 0.0 |
| GT32 | A4GNT | 0.0 | 0.0 |
| GT6 | ABO | 0.0 | nd |
| GT33 | ALG1 | 22.9 | 41.2 |
| GT59 | ALG10 | 7.9 | 0.0 |
| GT59 | ALG10B | 6.4 | 0.0 |
| GT4 | ALG11 | 8.8 | 20.2 |
| GT22 | ALG12 | 16.1 | 68.7 |
| GT1 | ALG13 | 35.9 | na |
| GT1 | ALG14 | 5.7 | 54.6 |
| GT33 | ALG1L | 2.4 | nd |
| GT33 | ALG1L2 | 0.0 | nd |
| GT4 | ALG2 | 16.2 | 51.7 |
| GT58 | ALG3 | 59.4 | 37.6 |
| GT2 | ALG5 | 58.1 | 70.8 |
| GT57 | ALG6 | 12.8 | 22.2 |
| GT57 | ALG8 | 44.6 | 22.2 |
| GT22 | ALG9 | 13.6 | 98.0 |
| GT31 | B3GALNT1 | 0.0 | 37.0 |
| GT31 | B3GALNT2 | 19.9 | 0.0 |
| GT31 | B3GALT1 | 0.2 | 0.0 |
| GT31 | B3GALT2 | 0.0 | 0.0 |
| GT31 | B3GALT4 | 0.6 | 16.1 |
| GT31 | B3GALT5 | 0.1 | 0.0 |
| GT31 | B3GALT6 | 19.9 | 42.2 |
| GT43 | B3GAT1 | 0.5 | 0.0 |
| GT43 | B3GAT2 | 0.8 | 0.0 |
| GT43 | B3GAT3 | 30.0 | 30.6 |
| GT31 | B3GLCT | 8.8 | nd |
| GT31 | B3GNT2 | 8.2 | 188.6 |
| GT31 | B3GNT3 | 0.1 | 0.0 |
| GT31 | B3GNT4 | 1.4 | 0.0 |
| GT31 | B3GNT5 | 12.1 | 0.0 |
| GT31 | B3GNT6 | 0.0 | nd |
| GT31 | B3GNT7 | 0.1 | 0.0 |
| GT31 | B3GNT8 | 0.2 | 0.0 |
| GT31 | B3GNT9 | 2.1 | 0.0 |
| GT2 | B3GNTL1 | 5.5 | nd |
| GT12 | B4GALNT1 | 2.1 | 0.0 |
| GT12 | B4GALNT2 | 0.0 | 0.0 |
| GT7 | B4GALNT3 | 12.7 | 0.0 |
| GT7 | B4GALNT4 | 18.9 | 0.0 |
| GT7 | B4GALT1 | 10.4 | 36.0 |
| GT7 | B4GALT2 | 59.9 | 40.7 |
| GT7 | B4GALT3 | 38.7 | 74.0 |
| GT7 | B4GALT4 | 6.3 | 28.4 |
| GT7 | B4GALT5 | 9.4 | 71.3 |
| GT7 | B4GALT6 | 5.6 | 71.3 |
| GT7 | B4GALT7 | 17.5 | 169.3 |
| GT49 | B4GAT1 | 39.7 | 0.0 |
| GT31 | C1GALT1 | 6.6 | 25.5 |
| GT31 | C1GALT1C1 | 31.3 | 120.2 |
| GT25 | CERCAM | 16.5 | nd |
| GT7/31 | CHPF | 14.1 | 332.3 |
| GT7/31 | CHPF2 | 12.8 | 129.7 |
| GT7/31 | CHSY1 | 14.6 | 0.0 |
| GT7/31 | CHSY3 | 1.2 | 0.0 |
| GT25 | COLGALT1 | 70.6 | nd |
| GT25 | COLGALT2 | 2.5 | nd |
| GT7 | CSGALNACT1 | 0.4 | 0.0 |
| GT7 | CSGALNACT2 | 7.3 | 0.0 |
| GT2 | DPM1 | 41.6 | 77.6 |
| GTnc | DPY19L1 | 15.3 | nd |
| GTnc | DPY19L2 | 1.6 | nd |
| GTnc | DPY19L3 | 8.9 | nd |
| GTnc | DPY19L4 | 15.1 | nd |
| GT61 | EOGT | 5.4 | nd |
| GT47/64 | EXT1 | 35.3 | 83.2 |
| GT47/64 | EXT2 | 40.6 | 136.1 |
| GT47/64 | EXTL1 | 0.1 | 2.1 |
| GT64 | EXTL2 | 9.3 | 179.4 |
| GT47/64 | EXTL3 | 17.9 | 78.7 |
| GTnc | FKRP | 15.9 | nd |
| GTnc | FKTN | 7.0 | nd |
| GT11 | FUT1 | 0.5 | 0.0 |
| GT10 | FUT10 | 7.4 | 0.0 |
| GT10 | FUT11 | 13.8 | 0.0 |
| GT11 | FUT2 | 0.2 | 0.0 |
| GT10 | FUT3 | 0.2 | 0.0 |
| GT10 | FUT4 | 2.0 | 0.0 |
| GT10 | FUT5 | 0.0 | 0.0 |
| GT10 | FUT6 | 0.5 | 0.0 |
| GT10 | FUT7 | 0.0 | 0.0 |
| GT23 | FUT8 | 10.7 | 165.8 |
| GT10 | FUT9 | 0.0 | 0.0 |
| GT27 | GALNT1 | 19.0 | 0.0 |
| GT27 | GALNT10 | 7.5 | nd |
| GT27 | GALNT11 | 18.3 | 63.0 |
| GT27 | GALNT12 | 2.3 | 0.0 |
| GT27 | GALNT13 | 3.6 | 0.0 |
| GT27 | GALNT14 | 2.6 | 0.0 |
| GT27 | GALNT15 | 0.0 | 0.0 |
| GT27 | GALNT16 | 6.2 | 0.0 |
| GT27 | GALNT18 | 11.5 | 0.0 |
| GT27 | GALNT2 | 40.0 | 324.3 |
| GT27 | GALNT3 | 11.5 | 0.0 |
| GT27 | GALNT4 | 2.1 | nd |
| GT27 | GALNT5 | 0.0 | 0.0 |
| GT27 | GALNT6 | 3.7 | 0.0 |
| GT27 | GALNT7 | 22.3 | 43.9 |
| GT27 | GALNT8 | 1.0 | 0.0 |
| GT27 | GALNT9 | 0.0 | 0.0 |
| GT27 | GALNTL5 | 0.0 | 0.0 |
| GT27 | GALNTL6 | 0.0 | nd |
| GT27 | GALNT19/WBSCR17 | 0.0 | 63.0 |
| GT6 | GBGT1 | 0.5 | 11.4 |
| GT14 | GCNT1 | 4.0 | 0.0 |
| GT14 | GCNT2 | 6.0 | 0.0 |
| GT14 | GCNT3 | 0.0 | 0.0 |
| GT14 | GCNT4 | 0.0 | 0.0 |
| GT14 | GCNT6 | 0.1 | nd |
| GT14 | GCNT7 | 0.0 | nd |
| GT4 | GLT1D1 | 0.0 | nd |
| GT6 | GLT6D1 | 0.0 | nd |
| GT8 | GLT8D1 | 35.9 | nd |
| GT8 | GLT8D2 | 8.6 | nd |
| GT4 | GTDC1 | 7.7 | nd |
| GT8 | GXYLT1 | 17.2 | nd |
| GT8 | GXYLT2 | 1.0 | nd |
| GT8 | GYG1 | 25.9 | nd |
| GT8 | GYG2 | 5.9 | nd |
| GT8/49 | GYLTL1B | 7.7 | 0.0 |
| GT3 | GYS1 | 36.8 | nd |
| GT3 | GYS2 | 0.1 | nd |
| GT2 | HAS1 | 0.0 | 0.0 |
| GT2 | HAS2 | 0.5 | 0.0 |
| GT2 | HAS3 | 1.2 | 2.1 |
| GT90 | KDELC1 | 15.7 | nd |
| GTnc | KDELC2 | 24.6 | nd |
| GT8/49 | LARGE | 11.6 | 22.0 |
| GT31 | LFNG | 0.3 | 24.0 |
| GT31 | MFNG | 1.7 | 0.0 |
| GT13 | MGAT1 | 47.9 | 60.6 |
| GT16 | MGAT2 | 18.6 | 138.2 |
| GT17 | MGAT3 | 0.3 | 0.0 |
| GT54 | MGAT4A | 7.8 | 0.0 |
| GT54 | MGAT4B | 52.4 | 0.0 |
| GT54 | MGAT4C | 0.0 | nd |
| GTnc | MGAT4D | 0.0 | nd |
| GT18 | MGAT5 | 13.3 | 19.8 |
| GT18 | MGAT5B | 0.8 | 0.0 |
| GT41 | OGT | 60.4 | 39.3 |
| GT4 | PIGA | 7.8 | 8.6 |
| GT22 | PIGB | 7.1 | 75.6 |
| GT50 | PIGM | 7.5 | 54.0 |
| GT76 | PIGV | 4.6 | nd |
| GT22 | PIGZ | 0.5 | nd |
| GTnc | PLOD3 | 19.7 | nd |
| GT65 | POFUT1 | 18.2 | 126.2 |
| GT68 | POFUT2 | 12.7 | 15.3 |
| GT90 | POGLUT1 | 11.4 | nd |
| GT13 | POMGNT1 | 33.8 | 101.5 |
| GT61 | POMGNT2 | 28.0 | nd |
| GT39 | POMT1 | 26.8 | 0.0 |
| GT39 | POMT2 | 16.9 | 0.0 |
| GT35 | PYGB | 19.4 | nd |
| GT35 | PYGL | 42.6 | nd |
| GT35 | PYGM | 0.9 | nd |
| GT31 | RFNG | 33.2 | 157.7 |
| GT29 | ST3GAL1 | 3.6 | 195.5 |
| GT29 | ST3GAL2 | 8.2 | 28.3 |
| GT29 | ST3GAL3 | 3.4 | 75.0 |
| GT29 | ST3GAL4 | 10.0 | 33.1 |
| GT29 | ST3GAL5 | 7.1 | 43.7 |
| GT29 | ST3GAL6 | 4.2 | 29.1 |
| GT29 | ST6GAL1 | 6.5 | 0.0 |
| GT29 | ST6GAL2 | 0.0 | 0.0 |
| GT29 | ST6GALNAC1 | 0.0 | 0.0 |
| GT29 | ST6GALNAC2 | 0.8 | 0.0 |
| GT29 | ST6GALNAC3 | 2.5 | 0.0 |
| GT29 | ST6GALNAC4 | 5.9 | 66.7 |
| GT29 | ST6GALNAC5 | 1.1 | 0.0 |
| GT29 | ST6GALNAC6 | 10.9 | 24.7 |
| GT29 | ST8SIA1 | 0.0 | 0.0 |
| GT29 | ST8SIA2 | 0.4 | 0.0 |
| GT29 | ST8SIA3 | 0.0 | 0.0 |
| GT29 | ST8SIA4 | 0.0 | 0.0 |
| GT29 | ST8SIA5 | 0.3 | 0.0 |
| GT29 | ST8SIA6 | 0.1 | 0.0 |
| GT66 | STT3A | 93.7 | nd |
| GT66 | STT3B | 58.7 | nd |
| GTnc | TMEM5 | 17.6 | nd |
| GTnc | TMTC1 | 4.4 | na |
| GTnc | TMTC2 | 5.0 | na |
| GTnc | TMTC3 | 9.3 | na |
| GTnc | TMTC4 | 8.4 | na |
| GT21 | UGCG | 8.6 | 0.0 |
| GT24 | UGGT1 | 15.4 | nd |
| GT24 | UGGT2 | 6.3 | 95.0 |
| GT1 | UGT1A1 | 0.0 | 95.7 |
| GT1 | UGT1A10 | 0.0 | nd |
| GT1 | UGT1A3 | 0.0 | nd |
| GT1 | UGT1A4 | 0.0 | nd |
| GT1 | UGT1A5 | 0.0 | nd |
| GT1 | UGT1A6 | 0.0 | nd |
| GT1 | UGT1A7 | 0.0 | nd |
| GT1 | UGT1A8 | 0.0 | nd |
| GT1 | UGT1A9 | 0.0 | nd |
| GT1 | UGT2A1 | 0.0 | 0.0 |
| GT1 | UGT2A3 | 0.0 | nd |
| GT1 | UGT2B10 | 0.0 | 0.0 |
| GT1 | UGT2B11 | 0.0 | nd |
| GT1 | UGT2B15 | 0.0 | nd |
| GT1 | UGT2B17 | 0.0 | 0.0 |
| GT1 | UGT2B28 | 0.0 | 0.0 |
| GT1 | UGT2B4 | 0.0 | 0.0 |
| GT1 | UGT2B7 | 0.0 | nd |
| GT1 | UGT3A1 | 0.0 | nd |
| GT1 | UGT3A2 | 2.5 | nd |
| GT1 | UGT8 | 7.9 | 0.0 |
| GT8 | XXYLT1 | 23.6 | nd |
| GT14 | XYLT1 | 0.8 | 0.0 |
| GT14 | XYLT2 | 13.2 | 5.9 |

⁽¹⁾GT classification system (Lombard et al. 2013, Nucl Acid Res 42: D1P: D490-D495),
⁽²⁾Approved HGNC gene symbol
⁽³⁾Gene expression levels in HEK293 is expressed as fpkm (fragments Per Kilobase of transcript per Million) adapted from Human Protein Atlas (http://www.proteinatlas.org/)
⁽⁴⁾Gene expression in the CHO-K1 cell line is based on WGS sequencing depth adapted from Xu et al. 2011, Nature Biotech 29:735-742, ‘nd’ means not detectable and ‘na’ means not analysed.

TABLE 4
Non-GTfs, available RNA_seq data
HEK293 and CHO-K1 (in total 62)

| | | | CHO K1 |
|---|---|---|---|
| CAZy | Common | HEK293 | (RNA Seq |
| classification | gene name | (FPKM) | Mapping Depth) |
| Acetylation | CASD1 | 7.6 | nd |
| Sulfo-T | CHST1 | 17.9 | 0.0 |
| Sulfo-T | CHST10 | 16.5 | 0.0 |
| Sulfo-T | CHST11 | 4.9 | 23.4 |
| Sulfo-T | CHST12 | 12.8 | 246.5 |
| Sulfo-T | CHST13 | 0.5 | na |
| Sulfo-T | CHST14 | 11.1 | 96.6 |
| Sulfo-T | CHST15 | 4.9 | 0.0 |
| Sulfo-T | CHST2 | 0.0 | 36.2 |
| Sulfo-T | CHST3 | 4.0 | 0.0 |
| Sulfo-T | CHST4 | 0.4 | 0.0 |
| Sulfo-T | CHST5 | 0.1 | 0.0 |
| Sulfo-T | CHST6 | 0.2 | 0.0 |
| Sulfo-T | CHST7 | 5.0 | na |
| Sulfo-T | CHST8 | 1.2 | 0.0 |
| Sulfo-T | CHST9 | 2.1 | 0.0 |
| OST | DAD1 | 173.8 | 1678.8 |
| OST | DDOST | 187.8 | 288.3 |
| Donor | DOLK | 13.5 | nd |
| Donor | DPAGT1 | 30.3 | 59.0 |
| Donor | DPM2 | 42.9 | nd |
| Donor | DPM3 | 150.5 | 115.6 |
| Epimerase | DSE | 13.7 | nd |
| Epimerase | DSEL | 0.1 | 0.0 |
| Kinase | FAM20B | 14.7 | nd |
| Sulfo-T | GAL3ST1 | 0.8 | 0.0 |
| Sulfo-T | GAL3ST2 | 0.2 | 0.0 |
| Sulfo-T | GAL3ST3 | 0.0 | 0.0 |
| Sulfo-T | GAL3ST4 | 0.8 | 0.0 |
| Epimerase | GLCE | 18.2 | 90.8 |
| Man-6-P | GNPTAB | 12.8 | nd |
| Man-6-P | GNPTG | 20.9 | nd |
| Degradation | HPSE | 2.2 | 36.7 |
| Sulfo-T | HS2ST1 | 19.3 | 29.0 |
| Sulfo-T | HS3ST1 | 0.0 | 0.0 |
| Sulfo-T | HS3ST2 | 0.1 | 0.0 |
| Sulfo-T | HS3ST3A1 | 11.9 | 0.0 |
| Sulfo-T | HS3ST3B1 | 4.8 | 0.0 |
| Sulfo-T | HS3ST4 | 0.0 | 0.0 |
| Sulfo-T | HS3ST5 | 0.0 | 0.0 |
| Sulfo-T | HS3ST6 | 0.1 | 0.0 |
| Sulfo-T | HS6ST1 | 14.0 | 96.8 |
| Sulfo-T | HS6ST2 | 36.7 | 0.0 |
| Sulfo-T | HS6ST3 | 0.5 | 0.0 |
| Sulfo-T | NDST1 | 21.8 | 0.0 |
| Sulfo-T | NDST2 | 15.8 | 65.8 |
| Sulfo-T | NDST3 | 0.0 | 65.8 |
| Sulfo-T | NDST4 | 0.3 | 65.8 |
| Man-6-P | NAGPA | 10.8 | nd |
| Donor | OST4 | 185.9 | nd |
| GPI | PIGC | 26.2 | 38.3 |
| GPI | PIGH | 23.1 | 6.7 |
| GPI | PIGP | 26.4 | 225.9 |
| GPI | PIGQ | 21.7 | 25.9 |
| Kinase | POMK | 1.2 | nd |
| Donor | RFT1 | 17.4 | nd |
| OST | RPN1 | 113.8 | 584.4 |
| OST | RPN2 | 129.2 | 1174.7 |
| Degradation | SULF1 | 2.5 | 0.0 |
| Degradation | SULF2 | 14.5 | 106.8 |
| Donor | TUSC3 | 69.7 | nd |
| Sulfo-T | UST | 13.6 | 74.4 |

⁽¹⁾Genes are grouped after PTM (post translational modification) function (OST, oligosaccharyltransferase; GPI, Glycosylphosphatidylinositol anchor; Sulfo-T, sulfotransferase)
⁽²⁾Approved HGNC gene symbol
⁽³⁾Gene expression levels in HEK203 is expressed as fpkm (fragments Per Kilobase of transcript per Million) adapted from Human Protein Atlas (http://www.proteinatlas.org/)
⁽⁴⁾Gene expression in the CHO-K1 cell line is based on WGS sequencing depth adapted from Xu et al. 2011, Nature Biotech 29:735-742, ‘nd’ means not detectable and ‘na’ means not analysed.

To explore the feasibility of engineering the glycosylation capacity in a human cell and display different glycomes, the present invention employed a nuclease-mediated (ZFNs, TALENs, CRISPR/Cas9) KO screen in a human embryonic kidney HEK293 cell line. The present invention designed a KO screen of genes encoding glycosyltransferases with potential to control early steps in glycosylation of lipids, proteins, and proteoglycans expressed in HEK293 (FIG. 1). The screen was designed to sequentially probe glycogenes in a systematic way targeting select groups of genes.

The present invention probed the effects of the knock out screen and display of a cancer-associated glycoform by immunostaining of engineered HEK293 cells with a panel of monoclonal antibodies to different truncated O-glycoforms including Tn, STn, and T.

The present invention probed the effects of the knock out screen and display of a cancer-associated glycoform by immunostaining of engineered HEK293 cells expressing a cell-membrane chimeric reporter construct containing a human protein sequence derived from human MUC1.

The present invention relates to reporter constructs useful for displaying specific human protein sequences with different glycoforms. The present invention used a protein sequence derived from the tandem repeat of human MUC1 mucin for display of cancer-associated glycoforms of MUC1. The reporter construct was designed to generate a chimeric type 1 transmembrane protein based on sinal peptide sequences derived from platelet GB1bα (amino acid 1-41) or MUC1 (amino acid 1-51) fused to enhanced cyan fluorescent protein (ECFP) linked to a interchangeable polypeptide region fused to the membrane anchoring domain of CD34 (amino acids 129-279) or MUC1 (ENST00000611571.4 amino acid 1039-1196).

The present invention probed the effect of displaying different glycoforms of MUC1 in the reporter construct on HEK293 cells with the monoclonal antibody 5E5 detecting a cancer-associated Tn glycoforms on MUC1 (Tarp 2007).

The present invention used ZFNs, TALENs and CRISPR/Cas9 to target and knock out glycosyltransferase genes in a combinatorial approach involved in lipid, protein and proteoglycans (FIG. 1). The display strategy is designed to probe involvement of glycans in a step-by-step consecutive approach addressing: i) type of glycoconjugate(s) involved by targeting the first and initiation step in formation of each glycoconjugate (FIG. 1, Step 1, panel 1B); ii) type of glycan(s) involved by targeting the second step in formation of each type of oligosaccharide structure on glycoconjugates (FIG. 1, Step 2, panel 1C); iii) structure of glycan(s) involved by targeting the elongation and branching steps in each type of oligosaccharide structure on glycoconjugates (FIG. 1, Step 3, panel 1D); and iv) capping of glycan(s) involved by targeting the capping steps in formation of each type of oligosaccharide structure on glycoconjugates (FIG. 1, Step 4, panel 1E). The genes targeted in each step are shown in (FIG. 1) and listed in Tables 1, 2 and 5.

Steps 2-4 are different depending on type of glycoconjugate. Type of glycoconjugate is identified by displaying glycans using a multiplicity of mammalian cells with mutations in the genes included in Step 1, see FIG. 1B.

For each glycoconjugate and for each of steps 2, 3, 4 and 5 targeted glycan arrays may be displayed on a multiplicity of mammalian cells with mutations in glycogenes shown in FIG. 2, panel 2A (N-glycosylation), panel 2B (O-Gluc, O-Fut), panel 2C (O-Man), panel 2D (O-GalNac), panel 2E (Glycolipids) and Panel 2F (Glucosaminoglycans).

Introduction of New Glycosylation Capacity in HEK293 Cells.

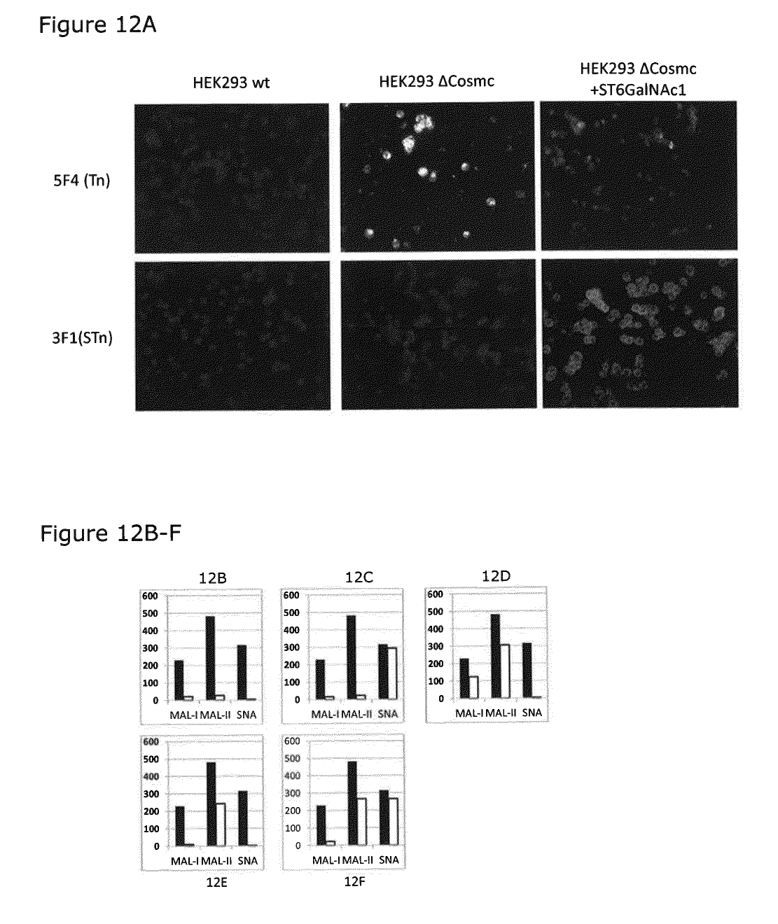

The present invention further provides strategies to display glycoforms not normally found in HEK293 cells. The invention provides a strategy to develop mammalian cells with defined and/or more homogenous glycosylation capacities that serves as template for de novo engineering of desirable glycosylation capacities by introduction of one or more glycosyltransferases using site-directed gene integration and/or classical random integration after transfection of cDNA and/or genomic constructs. The strategy involves inactivating glycosyltransferase genes to obtain a homogenous glycosylation capacity in a particular desirable type of glycosylation, for example using the design matrix developed herein for all types of glycosylation of glycoconjugates, and for example but not limited to inactivation of the C1GALT1 and/or COSMC genes to truncate O-glycans in a HEK293 cell and obtaining a cell without sialic acid capping of O-GalNAc glycans. In such a cell the de novo introduction of one or more new glycosylation capacities that utilize the more homogenous truncated glycan product obtained by one or more glycosyltransferase gene inactivation events, will provide for non-competitive glycosylation and more homogeneous glycosylation by the de novo introduced glycosyltransferases. For example but not limited to introduction of a α2,6 sialyltransferase such as ST6GALNAC1 into a mammalian cell with inactivated C1GALT1 and/or COSMC genes (FIG. 12). The general principle of the strategy is to simplify glycosylation of a particular pathway, e.g. O-glycosylation, to a point where reasonable homogeneous glycan structures are being produced in the mammalian cell in which one or more glycosyltransferase gene inactivation events has been introduced in a deconstruction process as provided in the present invention for N-glycosylation. Taking such mammalian cell with deconstructed and simplified glycosylation capacity, and introduce de novo desirable glycosylation capacities that build on the glycan structures produced by the deconstruction.

Knock Out Targeting Strategy.

It is clear to the person skilled in the art that inactivation of a glycosyltransferase gene can have a multitude of outcomes and effects on the transcript and/or protein product translated from this. Targeted inactivation experiments performed herein involved PCR and sequencing of the introduced alterations in the genes as well as RNAseq analysis of clones to determine whether a transcript was formed and if potential novel splice variations have possibly introduced new protein structures. Moreover, methods for determining presence of protein from such transcripts are available and include mass spectrometry and SDS-PAGE Western blot analysis with relevant antibodies detecting the most N-terminal region of the protein products.

The targeting constructs were generally designed to target the first 1/3 of the open reading frame (ORF) of the coding regions but other regions were also targeted. For most clones, out of frame mutations (Indels) that introduced premature stop, non-sense codons or incorrect splicing were selected. This would be expected to produce truncated proteins if any protein at all and without the catalytic domain and hence enzymatic activity. The majority of KO clones exhibited out of frame insertions and/or deletions (indels), and most targeted genes were present with two alleles, while some were present with 1 or 3 alleles, respectively. In a few cases larger deletions were found and these disrupted one or more exons and exon/intron boundaries also resulting in truncated proteins.

Most ER-Golgi glycosyltransferases share the common type 2 transmembrane structure with a short cytosolic tail that may direct retrograde trafficking and residence time, a non-cleaved signal peptide containing a hydrophobic transmembrane α-helix domain for retention in the ER-Golgi membrane, a variable length stem or stalk region believed to displace the catalytic domain into the lumen of the ER-Golgi, and a C-terminal catalytic domain required for enzymatic function (Colley 1997). Only polypeptide GalNAc-transferases has an additional C-terminal lectin domain (Bennett 2012). The genomic organization of glycosyltransferase genes varies substantially with some genes having a single coding exon and others more than 10-15 coding exons, although few glycosyltransferase genes produce different splice variants encoding different protein products.

Inactivation of glycosyltransferase genes in some of the first coding regions may thus have a multitude of effects on transcript and protein products if these are made: i) one or more transcripts may be unstable and rapidly degraded resulting in little or no transcript and/or protein; ii) one or more transcripts may be stable but not or only poorly translated resulting in little or no protein synthesis; iii) one or more transcripts may be stable and translated resulting in protein synthesis; iv) one or more transcripts may result in protein synthesis but protein products are degraded due to e.g. truncations and/or misfolding; v) one or more transcripts may result in protein synthesis and stable protein products that are truncated and enzymatically inactive; and vi) one or more transcripts may result in protein synthesis and stable protein products that are truncated but have enzymatic activity.

It is evident for the skilled in the art that gene inactivation that lead to protein products with enzymatic activity is undesirable, and these event are easily screened for by the methods used in the present invention, e.g. but not limited to lectin/antibody labeling and glycoprofiling of proteins expressed in mutant cells.

However, it is desirable to eliminate potential truncated protein products that may be expressed from mutated transcripts as these may have undesirable effects on glycosylation capacity. Thus, truncated protein products from type 2 glycosyltransferase genes containing part of or entire part of the cytosolic, and/or the transmembrane retention signal, and/or the stem region, and/or part of the catalytic domain may exert a number of effects on glycosylation in cells. For example, the cytosolic tail may compete for COP-I retrograde trafficking of Golgi resident proteins (Eckert, Reckmann et al. 2014), the transmembrane domain may compete for localization in ER-Golgi and potential normal associations and/or aggregations of proteins, and the stem region as well as part of an inactive catalytic domain may have similar roles or part of roles in normal associations and/or aggregations of proteins in ER-Golgi. Such functions and other unknown ones may affect specific glycosylation pathways that the enzymes are involved in, specific functions of isoenzymes, or more generally glycosylation capacities of a cell. While these functions and effects are unknown and unpredictable today, it is an inherent part of the present invention that selection of mammalian cell clones with inactivated glycosyltransferase genes includes selection of editing events that do not produce truncated protein products.

An object of the present invention relates to a cell comprising one or more glycosyltransferase genes that have been inactivated, and that displays new and/or more homogeneous glycans.

In some embodiments of the present invention the cell comprises two or more glycosyltransferase genes that have been inactivated.

Another object of the present invention relates to a cell comprising one or more glycosyltransferase genes that have been introduced stably by site-specific gene or non-site-specific knock in and that display new and/or more homogeneous glycans.

In some embodiments of the present invention the cell comprises two or more glycosyltransferase that have been introduced stably by site-specific or non-site-specific gene knock in.

An aspect of the present invention relates to a cell comprising one or more glycosyltransferase genes introduced stably by site-specific or non-site-specific gene knock in, and furthermore comprising one or more endogenous glycosyltransferase genes that have been inactivated by knock out, and that displays new and/or more homogeneous glycans.

A further aspect of the present invention relates to a cell comprising two or more glycosyltransferase genes encoding isoenzymes with partial overlapping glycosylation functions in the same biosynthetic pathway and/or same biosynthetic step inactivated, and for which inactivation of two or more of these genes is required for display of new and/or more homogeneous glycans.

In some embodiments of the present invention the cell comprises one or more glycosyltransferase genes inactivated to block and truncate one or more glycosylation pathways.

A further aspect of the present invention relates to a cell comprising two or more glycosyltransferase genes inactivated to block and truncate one or more glycosylation pathways.

In some embodiments of the present invention the cell comprises targeted inactivation of one or more glycosyltransferase genes for which no transcripts are detectable.

A further aspect of the present invention relates to a cell comprising targeted inactivation of one or more glycosyltransferase genes for which no protein products are detectable.

Another aspect of the present invention relates to a cell comprising targeted inactivation of one or more glycosyltransferase genes for which no protein products with intact cytosolic and/or transmembrane region is detectable.

In some other embodiments of the present invention is the glycosyltransferase any one or more of the genes listed in Tables 1 and 2.

In some embodiments of the present invention are the glycosyltransferases that are inactivated working in the same glycosylation pathway.

In some other embodiments of the present invention are the glycosyltransferases that are inactivated working in the same glycosylation step.

In yet other embodiments of the present invention are the glycosyltransferases that are inactivated working in consecutive biosynthetic steps.

In some embodiments of the present invention are the glycosyltransferases that are inactivated retained in the same subcellular topology.

In some other embodiments of the present invention are the glycosyltransferases that are inactivated having similar amino acid sequence.

In further embodiments of the present invention are the glycosyltransferases that are inactivated belonging to the CAZy family.

In yet other embodiments of the present invention are the glycosyltransferases that are inactivated belonging to same subfamily of isoenzymes in a CAZy family.

In some embodiments of the present invention are the glycosyltransferases that are inactivated having similar structural retention signals (transmembrane sequence and length).

In some other embodiments of the present invention are the glycosyltransferase genes functioning in the same glycosylation pathway inactivated, and wherein they are not involved in the same glycosylation step.

In further embodiments of the present invention are the glycosyltransferase genes functioning in the same glycosylation pathway inactivated, and wherein they are involved in the same glycosylation step.

In some embodiments of the present invention the cell or cell line is a mammalian cell or cell line, or an insect cell or cell line.

In some other embodiments of the present invention the cell is derived from human kidney.

In further embodiments of the present invention the cell is selected from the group consisting of HEK293, NS0, SP2/0, YB2/0, HUVEC, HKB, PER-C6, NS0, or derivatives of any of these cells.

In some embodiments of the present invention is the cell a HEK293 cell.

In some other embodiments of the present invention the cell furthermore encodes an exogenous protein of interest.

In some embodiments of the exogenous protein of interest is an antibody, an antibody fragment, or a polypeptide, such as an IgG antibody.

In some embodiments of the invention the exogenous protein of interest is a lysosomal enzyme.

In some embodiments of the invention the exogenous protein of interest is a lysosomal enzyme, which lysosomal enzyme is expressed to comprise one or more posttranslational modifications independently selected from:

a) with α2,3NeuAc capping,

b) without α2,3NeuAc capping,

c) with α2,6NeuAc capping,

d) without α2,6NeuAc capping,

e) without LacDiNac structure,

f) high Mannose6phosphate,

g) low Mannose6phosphate, and

h) without bisecting glycoforms.

In some embodiments of the invention the exogenous protein of interest is a lysosomal enzyme, and the one or more endogenous glycogene inactivated and/or exogenous glycogene introduced independently in individual cells of said plurality of mammalian cells is selected from the list of GNPTAB, GNPTG, NAGPA, ALG3/6/8/9/10/12s, Mannosidases (MAN1A1, MAN1A2, MAN1B1, MAN1C1, MAN2A1, MAN2A2), MOGS, GANAB plus MGAT1/2 and Sialyl transferases.

In some embodiments of the invention the exogenous protein of interest is a lysosomal enzyme, and the one or more endogenous glycogene inactivated is GNPTAB, such as in order to increase sialic acids.

In some embodiments of the invention the exogenous protein of interest is a lysosomal enzyme, wherein said lysosomal enzyme has obtained increased mannose-6-phosphate (M6P) tagging of N-glycans and/or has obtained changed site occupancy of M6P, such as by knocking out a gene selected from ALG3, ALG8, NAGPA.

In some embodiments of the invention the exogenous protein of interest is a lysosomal enzyme, wherein said lysosomal enzyme has obtained increased high mannose structures, such as by knocking out a gene selected from MGAT1 and/or GNPTAB and/or MOGS.

In yet other embodiments of the present invention is the protein of interest a human protein.

In further embodiments of the present invention is the human protein a mucin or a fragment of a human mucin.

In some embodiments of the present invention is the glycosylation made more homogenous.

In some other embodiments of the present invention is the glycosylation non-sialylated.

In some other embodiments of the present invention is the glycosylation α2,3sialylated.

In some other embodiments of the present invention is the glycosylation α2,6sialylated.

In yet other embodiments of the present invention is the glycosylation non-galactosylated.

In some embodiments of the present invention comprises the glycosylation high mannose N-glycans.

In some other embodiments of the present invention does the glycosylation not comprise poly-LacNAc.

In yet other embodiments of the present invention is the glycosylation any combination without fucose.

One aspect of the present invention relates to a glycoprotein according to the present invention, which is a homogeneous glycoconjugate produced from a glycoprotein having a simplified glycan profile.

An object of the present invention is to provide a cell capable of expressing a gene encoding a polypeptide of interest, wherein the polypeptide of interest is expressed comprising one or more posttranslational modification patterns.

In some embodiments of the present invention is the posttranslational modification pattern a glycosylation.

The optimal glycoform displayed on cells may be identified by the following process:

(i) producing a plurality of isogenic cells with different glycosylation capacities by inactivating and/or introducing one or more glycosyltransferase genes in said mammalian cells and having at least one novel glycosylation capacity, and (ii) determination of the interaction of said cells displaying different glycans with a biomolecule for example a protein such as a lectin or antibody or carbohydrate-binding protein and/or a microorganism for example a virus or bacteria or fungi or parasite or components thereof in a binding assay in comparison with a reference in same binding assay; and (iii) determination of the cell(s) displaying glycan structure(s) (glycoforms) with the higher/highest binding activity and determination of the cell(s) glycogene genotype fingerprint which is correlated with the higher/highest binding activity level of said cell(s).

The above described process allows the identification of the optimal glycan structure for display of binding interaction. Those skilled in the art by using the genotype fingerprint identified in (iii) may generate an efficient engineered cell line with the optimal genotype for display of the glycan and glycoconjugate on said cell.

The above described process allows the identification of the optimal glycan structure for binding interaction. For those skilled in the art by using the genotype fingerprint identified in (iii) may generate an efficient engineered cell line with the optimal genotype for production of the glycoconjugate with said glycan.

One aspect of the present invention relates to a method for displaying on cell surface and/or secreting a glycoprotein having modified glycan profile wherein the cell producing the glycoprotein has more than one modification of one or more glycosyltransferase genes.

In some embodiments of the present invention has the cells been modified by glycosyltransferase gene knock-out and/or knock-in of an exogeneous DNA sequence coding for a glycosyltransferase.

In some embodiments of the present invention one or more endogenous gene selected from the group consisting of GALNT1/T2/T3/T4/T5/T6/T7/T8/T9/T10/T11/T12/T13/T14/T15/T16/T17/T18/T19/T20, POMT1/T2, TMTC1/TMTC2/TMTC3/TMTC4, XYLT1/2, POGLUT1, POFUT1/2, EOGT, UGT8, DPY19L1/2/3/4, SST3A, SST3B, ALG3/6/8/9/10/12 have been knocked out in a plurality of mammalian cells (FIG. 1, Step 1, group 1 genes).

In some embodiments of the present invention all endogenous gene selected from the group consisting of GALNT1/T2/T3/T4/T5/T6/T7/T8/T9/T10/T11/T12/T13/T14/T15/T16/T17/T18/T19/T20, POMT1/T2, TMTC1/TMTC2/TMTC3/TMTC4, XYLT1/2, POGLUT1, POFUT1/2, EOGT, UGT8, DPY19L1/2/3/4, SST3A, SST3B, ALG3/6/8/9/10/12 have been knocked out in a plurality of mammalian cells except for one specific gene selected from the same list (FIG. 1B, Step 1, group 1 genes).

In some embodiments of the present invention one or more exogenous gene selected from the group consisting of GALNT1/T2/T3/T4/T5/T6/T7/T8/T9/T10/T11/T12/T13/T14/T15/T16/T17/T18/T19/T20, POMT1/T2, TMTC1/TMTC2/TMTC3/TMTC4, XYLT1/2, POGLUT1, POFUT1/2, EOGT, UGT8, DPY19L1/2/3/4, have been knocked in in a plurality of mammalian cells (FIG. 1B, Step 1, group 1 genes).

In some embodiments of the present invention one or more endogenous gene selected from the group consisting of C1GALT1/COSMC, POMGNT1, POMGNT2, B4GALT7, MGAT1, GXYLT1/2, LFNG/MFNG/RFNG, B4GALT5/6, CSGALNACT1/T2, and EXTL2/L3 have been knocked out in a plurality of mammalian cells (FIG. 1C, Step 2, group 2 genes).

In some embodiments of the present invention one or more exogenous gene selected from the group consisting of C1GALT1/COSMC, POMGNT1, POMGNT2, B4GALT7, MGAT1, GXYLT1/2, LFNG/MFNG/RFNG, B4GALT5/6, CSGALNACT1/T2, and EXTL2/L3 have been knocked in in a plurality of mammalian cells (FIG. 1C, Step 2, group 2 genes).

In some embodiments of the present invention one or more endogenous gene selected from the group consisting of B4GALT1/T2/T3/T4/T5/T6/T7, B3GNT2/T3/T4/T5/T7/T8/T9, GCNT1/T2/T3/T4, MGAT2/3/4A/4B/5/5B, MAN1A1/2, MAN1B1, MAN1C1, MAN2A1/2, MOGS, GANAB, POMK/LARGE, A4GALT/B3GALNT1/B3GNT5/GBGT1, CS/HS/KS polymerase genes and CHPF/CHF2/CHSY1/CHSY3/EXT1/T2 have been knocked out in a plurality of mammalian cells (FIG. 1D, Step 3, group 3 genes).

In some embodiments of the present invention one or more exogenous gene selected from the group consisting of B4GALT1/T2/T3/T4/T5/T6/T7, B3GNT2/T3/T4/T5/T7/T8/T9, GCNT1/T2/T3/T4, MGAT2/3/4A/4B/5/5B, POMK/LARGE, A4GALT/B3GALNT1/B3GNT5/GBGT1, CS/HS/KS polymerase genes and CHPF/CHF2/CHSY1/CHSY3/EXT1/T2 have been knocked in in a plurality of mammalian cells (FIG. 1D, Step 3, group 3 genes).

In some embodiments of the present invention one or more endogenous gene selected from the group consisting of ST3GAL1/2/3/4/5/6, ST6GAL1/2, ST6GALNAC1/2/3/4/5/6, ST8SIA1/2/3/4/5/6, FUT1/2/3/4/5/6/7/8/9/10/11, and A4GNT/ABO have been knocked out in a plurality of mammalian cells (FIG. 1E, Step 4, group 4 genes).

In some embodiments of the present invention one or more exogenous gene selected from the group consisting of ST3GAL1/2/3/4/5/6, ST6GAL1/2, ST6GALNAC1/2/3/4/5/6, ST8SIA1/2/3/4/5/6, FUT1/2/3/4/5/6/7/8/9/10/11, and A4GNT/ABO have been knocked in in a plurality of mammalian cells (FIG. 1E, Step 4, group 4 genes).

In some embodiments of the present invention one or more endogenous gene selected from the group consisting of DSEL, CHST1/T2/T3/T4/T5/T6/T7T/8/T9/T10/T11/T12/T13/T15/T15, UST, GLCE, HS2ST1, HS3T1/T2/T3A1/T3B1/T4/T5/T6 and/or HS6ST1/T2/T3, NDST1/T2/T3/T4, and GAL3ST1/T2/T3/T4 and GNPTAB, GNPTG, NAGPA have been knocked out in a plurality of mammalian cells (FIG. 1F, Step 5, See table 5, group 5 genes).

In some embodiment of the present invention the plurality of mammalian cells comprises one or more cells with knock out of all genes in a group (or any one of the listings mentioned herein) except one gene in the same group or listing. It is to be understood that this may be used to isolate and investigate a single glycosylation capacity.

In some embodiment of the present invention the plurality of mammalian cells comprises one or more cells with knock out of just one gene in a group (or any one of the listings mentioned herein). It is to be understood that this may be used to isolate and investigate the relevance of this particular gene for the glycosylation capacity.

In some embodiments of the present invention one or more exogenous gene selected from the group consisting of DSEL, CHST1/T2/T3/T4/T5/T6/T7T/8/T9/T10/T11/T12/T13/T15/T15, UST, GLCE, HS2ST1, HS3T1/T2/T3A1/T3B1/T4/T5/T6 and/or HS6ST1/T2/T3, NDST1/T2/T3/T4, and GAL3ST1/T2/T3/T4 and GNPTAB, GNPTG, NAGPA have been knocked in in a plurality of mammalian cells (FIG. 1F, Step 5, See table 5, group 5 genes).

One aspect of the present invention relates to a method for producing a glycoprotein having a plurality of glycan profiles, the method comprising expressing such protein in a plurality of mammalian cells with inactivation of one or more glycosyltransferases, and/or knock in of one or more glycosyltransferases, or a combination hereof in a cell, and isolating said proteins from a plurality of mammalian cells.

In another embodiment the present invention relates to a method for producing a glycoprotein having a plurality of glycan profiles, and from this plurality of glycovariant protein identify those with improved (drug) properties. The selection may comprise analyzing the glycovariant proteins for activity in comparison with a reference glycoprotein in (a) suitable bioassay(s); and selection of the glycoform with the higher/highest/optimal activity.

In another aspect of the present invention is one or more of the above mentioned genes knocked out using transcription activator-like effector nucleases (TALENs).

TALENs are artificial restriction enzymes generated by fusing a TAL effector DNA binding domain to a DNA cleavage domain.

In yet another aspect of the present invention is one or more of the above mentioned genes knocked out using CRISPRs (clustered regularly interspaced short palindromic repeats).

CRISPRs are DNA loci containing short repetitions of base sequences. Each repetition is followed by short segments of “spacer DNA” from previous exposures to a virus.

CRISPRs are often associated with cas genes that code for proteins related to CRISPRs. The CRISPR/Cas system is a prokaryotic immune system that confers resistance to foreign genetic elements such as plasmids and phages and provides a form of acquired immunity.

CRISPR spacers recognize and cut these exogenous genetic elements in a manner analogous to RNAi in eukaryotic organisms.

The CRISPR/Cas system is used for gene editing (adding, disrupting or changing the sequence of specific genes) and gene regulation in species throughout the tree of life. By delivering the Cas9 protein and appropriate guide RNAs into a cell, the organism's genome can be cut at any desired location.

The cell of the present invention may by a cell that does not comprise the gene of interest to be expressed or the cell may comprise the gene of interest to be expressed. The cell that does not comprise the gene of interest to be expressed is usually called “a naked cell”.

The host cell of the present invention may be any host, so long as it can display different glycans in a plurality of isogenic subclones. Examples include a yeast cell, an animal cell, a mammalian cell, an insect cell, a plant cell and the like.

In some embodiments of the present invention is the cell selected from the group consisting of HEK293, NS0, SP2/0, YB2/0, YB2/3HL.P2.G11.16Ag.20, NS0, SP2/0-Ag14, BHK cell derived from a human kidney, a syrian hamster kidney tissue, antibody-producing hybridoma cell, human leukemia cell line (Namalwa cell), an embryonic stem cell, and fertilized egg cell.

In a preferred embodiment of the present invention is the cell a HEK293 cell.

The cell can be an isolated cell or in cell culture or a cell line.

In one aspect of the present invention is the cell or components of the cell an antigen or vaccine component.

In another aspect of the present invention is the cell or components of the cell displaying glycans associated with diseases and useful for stimulating antibodies with selective or exclusive reactivity with glycans and/or glycoforms of proteins associated with diseases.

The cells of the present invention may there for be used to treat immune diseases, cancer, viral or bacterial infections or other diseases or disorders mentioned above.

The protein of interest can be various types of proteins, and in particular proteins that are glycosylated when expressed recombinant in the cell.

In a preferred embodiment the protein is an integral membrane bound protein when expressed recombinant in the cell.

In another preferred embodiment the protein is a secreted protein when expressed recombinant in a cell.

In one aspect of the present invention is the protein an antigen or vaccine component.

The glycoproteins of the present invention may there for be use to treat immune diseases, cancer, viral or bacterial infections or other diseases or disorders mentioned above.

It should be noted that embodiments and features described in the context of one of the aspects of the present invention also apply to the other aspects of the invention.

Definitions

“Isogenic” refers to cells having the same or essentially the same genotype (same genes) except for specific genes specified to be inactivated and/or introduced in some individual cell. Accordingly a population of HEK cells may be isogenic, but may also have some variations in terms of individual genes being knocked in or knocked out in some cells of the isogenic cell population.

“a plurality” in relation to a plurality of cells refers to more than one cell, wherein a cell of said plurality is different from the other cells of said plurality. There may also be two or more such as a population of identical cells within this plurality of cells.

“A plurality of isogenic mammalian cells” may be used interchangeably with “a plurality of mammalian cells”. Unless otherwise specified this means the same.

“Cell array” refers to a plurality of unique cells, for example a plurality of mammalian cells having the exact same genotype. Accordingly, it is to be understood that a cell array will contain a population of one type of unique individual cells, a second population of a second type of unique individual cells, a third population of a third type of unique individual cells and so forth up to a limit in size of the cell array.

The term “glycome display library” as used herein refers to a cell array designed to display different glycomes of the cells used in a library, wherein each different unique cell within the library is trackable back so that the specific genetic manipulation with glycogenes within a particular cell within the library is known at any time. It is to be understood that the cells used in the display library is kept and maintained in a suitable cell bank, and with a unique identification so that any inactivated glycogenes and/or introduced glycogenes within a particular cell is known at any time this particular cell is used and so that this particular unique cell may be used for other purposes.

General Glycobiology

Basic glycobiology principles and definitions are described in Varki et al. Essentials of Glycobiology, 2nd edition, 2009.

“CAZy” refers to ‘Carbohydrate-Active enZYmes Database’ which describes the families of structurally-related catalytic and carbohydrate-binding modules (or functional domains) of enzymes that degrade, modify, or create glycosidic bonds. CAZy reference: Lombard V, Golaconda Ramulu H, Drula E, Coutinho P M, Henrissat B (2014) The Carbohydrate-active enzymes database (CAZy) in 2013. Nucleic Acids Res 42:D490-D495.

“CAZy Families” are subdivision of enzymes that catalyze breakdown, biosynthesis and/or modification of glycoconjugates.

“CAZy Subfamilies” are subgroups found within a family that share a more recent ancestor and, that are usually more uniform in molecular function.

“N-glycosylation” refers to the attachment of the sugar molecule oligosaccharide known as glycan to a nitrogen atom residue of a protein

“O-glycosylation” refers to the attachment of a sugar molecule to an oxygen atom in an amino acid residue in a protein.

“Galactosylation” means enzymatic addition of a galactose residue to lipids, carbohydrates or proteins.

“Sialylation” is the enzymatic addition of a neuraminic acid residue.

“Neuraminic acid” (or NeuAc) is a 9-carbon monosaccharide, a derivative of a ketononose.

“Monoantennary” N-linked glycan is a engineered N-glycan consist of the N-glycan core (Manα1-6(Manα1-3)Manβ1-4GlcNAcβ1-4GlcNAcβ1-Asn-X-Ser/Thr) that elongated with a single GlcNAc residue linked to C-2 and of the mannose α1-3. The single GlcNAc residue can be further elongated for example with with Gal or Gal and NeuAc residues.

“Biantennary” N-linked glycan is the simplest of the complex N-linked glycans consist of the N-glycan core (Manα1-6(Manα1-3)Manβ1-4GlcNAcβ1-4GlcNAcβ1-Asn-X-Ser/Thr) elongated with two GlcNAc residues linked to C-2 and of the mannose α1-3 and the mannose α1-6. This core structure can then be elongated or modified by various glycan structures.

“Triantennary” N-linked glycans are formed when an additional GlcNAc residue is added to either the C-4 of the core mannose α1-3 or the C-6 of the core mannose α1-6 of the bi-antennary core structure. This structure can then be elongated or modified by various glycan structures.

“Tetratantennary” N-linked glycans are formed when two additional GlcNAc residues are added to either the C-4 of the core mannose α1-3 or the C-6 of the core mannose α1-6 of the bi-antennary core structure. This core structure can then be elongated or modified by various glycan structures.

“Poly-LacNAc” poly-N-acetyllactosamine ([Galβ1-4GlcNAc]n; n≥2.)

“Glycoprofiling” means characterization of glycan structures resident on a biological molecule or cell.

“Glycosylation pathway” refers to assembly of monosaccharides into a group of related complex carbohydrate structures by the stepwise action of enzymes, known as glycosyltransferases. Glycosylation pathways in mammalian cells are classified as N-linked protein glycosylation, different O-linked protein glycosylation (O-GalNAc, O-GlcNAc, O-Fuc, O-Glc, O-Xyl, O-Gal), different series of glycosphingolipids, and GPI-anchors.

“Biosynthetic Step” means the addition of a monosaccharide to a glycan structure.

“Glycosyltransferases” are enzymes that catalyze the formation of the glycosidic linkage to form a glycoside. These enzymes utilize ‘activated’ sugar phosphates as glycosyl donors, and catalyze glycosyl group transfer to a nucleophilic group, usually an alcohol. The product of glycosyl transfer may be an O-, N-, S-, or C-glycoside; the glycoside may be part of a monosaccharide, oligosaccharide, or polysaccharide.

“Glycogenes” includes glycosyltransferases and related glycogenes, wherein related glycogenes comprise any other enzyme acting on glycans to modify their structure. This included but is not limited to sulfotransferases, epimerases and deacetylases. In some embodiments the related glycogene is selected from sulfotransferases, epimerases and deacetylases.

“Glycosylation capacity” means the ability to produce an amount of a specific glycan structure by a given cell or a given glycosylation process.

“Glycoconjugate” is a macromolecule that contains monosaccharides covalently linked to proteins or lipids

“Simple(r) glycan structure” is a glycan structure containing fewer mono-saccharides and/or having lower mass and/or having fewer antennae.

“Human like glycosylation” means having glycan structures resembling those of human cells. Examples including more sialic acids with α2,6 linkage (more α2,6 sialyltransferase enzyme) and/or less sialic acids with α2,3 linkage and/or more N-acetylneuraminic acid (Neu5Ac) and/or less N-glycolylneuraminic acid (Neu5Gc).

“Display” refers to presentation of a plurality of glycan structures on a cell or on one or more glycoconjugates for analysis of binding interactions or other assays probing biological functions.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

“Deconstruction” means obtaining cells producing a simpler glycan structures by single or stacked knock out of glycosyltransferases. Deconstruction of a glycosylation pathway means knock out of glycosyltransferases involved in each step in biosynthesis and identification of glycosyltransferases controlling each biosynthetic step.

“Modified glycan profile” refers to change in number, type or position of oligosaccharides in glycans on a given glycoprotein.

More “homogeneous glycosylation” means that the proportion of identical glycan structures observed by glycoprofiling a given protein expressed in one cell is larger than the proportion of identical glycan structures observed by glycoprofiling the same protein expressed in another cell.

General DNA and Molecular Biology Tools.

Any of various techniques used for separating and recombining segments of DNA or genes, commonly by use of a restriction enzyme to cut a DNA fragment from donor DNA and inserting it into a plasmid or viral DNA. Using these techniques, DNA coding for a protein of interest is recombined/cloned (using PCR and/or restriction enzymes and DNA ligases or ligation independent methods such as USER cloning) into a plasmid (known as an expression vector), which can subsequently be introduced into a cell by transfection using a variety of transfection methods such as calcium phosphate transfection, electroporation, microinjection and liposome transfection. Overview and supplementary information and methods for constructing synthetic DNA sequences, insertion into plasmid vectors and subsequent transfection into cells can be found in Ausubel et al, 2003 and/or Sambrook & Russell, 2001.

“Gene” refers to a DNA region (including exons and introns) encoding a gene product, as well as all DNA regions which regulate the production of the gene product, whether or not such regulatory sequences are adjacent to coding and/or transcribed sequences or situated far away from the gene which function they regulate. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

“Targeted Gene Modifications”, “Gene Editing” or “Genome Editing”

Gene editing or genome editing refer to a process by which a specific chromosomal sequence is changed. The edited chromosomal sequence may comprise an insertion of at least one nucleotide, a deletion of at least one nucleotide, and/or a substitution of at least one nucleotide. Generally, genome editing inserts, replaces or removes nucleic acids from a genome using artificially engineered nucleases such as Zinc finger nucleases (ZFNs), Transcription Activator-Like Effector Nucleases (TALENs), the CRISPR/Cas system, and engineered meganuclease re-engineered homing endonucleases. Genome editing principles are described in Steentoft 2014 and gene editing methods are described in references therein and also broadly used and thus known to person skilled in the art.

“Endogenous” sequence/gene/protein refers to a chromosomal sequence or gene or protein that is native to the cell or originating from within the cell or organism analyzed

“Exogenous” sequence or gene refers to a chromosomal sequence that is not native to the cell, or a chromosomal sequence whose native chromosomal location is in a different location in a chromosome or originating from outside the cell or organism analyzed

“Inactivated chromosomal sequence” refer to genome sequence that has been edited resulting in loss of function of a given gene product. The gene is said to be knocked out.

“Heterologous” refers to an entity that is not native to the cell or species of interest.

Multiple knock-outs. GALNT1/GALNT2/GALNT3 or in general gene names separated by/(slash) may refer to multiple knock-out meaning all genes are knocked out in same cell. Listing of more genes names may be abbreviated using numbers, for example GALNT1/2/3 means GALNT1/GALNT2/GALNT3. Alternatively, and accordingly in different embodiments of the present invention general gene names in a list separated by / (slash) may refer to one or more, such as two or more, three or more, four or more, etc, or all gene knock-outs from this list.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide.

The terms “polypeptide” and “protein” are used interchangeably to refer to a polymer of amino acid residues. These terms may also refer to glycosylated variants of the “polypeptide” or “protein”, also termed “glycoprotein”. “polypeptide”, “protein” and “glycoprotein” is used interchangeably throughout this disclosure.

The term “recombination” refers to a process of exchange of genetic information between two polynucleotides. For the purposes of this disclosure, “homologous recombination” refers to the specialized form of such exchange that takes place, for example, during repair of double-strand breaks in cells. This process requires sequence similarity between the two polynucleotides, uses a “donor” or “exchange” molecule to template repair of a “target” molecule (i.e., the one that experienced the double-strand break), and is variously known as “non-crossover gene conversion” or “short tract gene conversion,” because it leads to the transfer of genetic information from the donor to the target. Without being bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized homologous recombination often results in an alteration of the sequence of the target molecule such that part or all of the sequence of the donor polynucleotide is incorporated into the target polynucleotide.

As used herein, the terms “target site” or “target sequence” refer to a nucleic acid sequence that defines a portion of a chromosomal sequence to be edited and to which a targeting endonuclease is engineered to recognize, bind, and cleave.

“Targeted integration” is the method by which exogenous nucleic acid elements are specifically integrated into defined loci of the cellular genome. Target specific double stranded breaks are introduced in the genome by genome editing nucleases that allow for integration of exogenously delivered donor nucleic acid element into the double stranded break site. Thereby the exogenously delivered donor nuclei acid element is stably integrated into the defined locus of the cellular genome.

Sequence identity Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found on the GenBank website. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

All patent and non-patent references cited in the present application, are hereby incorporated by reference in their entirety.

The invention will now be described in further details in the following non-limiting examples.

### EXAMPLES

### Example 1

The purpose of the following examples are given as an illustration of various embodiments of the invention and are thus not meant to limit the present invention in any way. Along with the present examples the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Glycoengineering of Mammalian HEK293 Cells

The human HEK293 cell is the preferred cell line for transient expression of human proteins with high transfection efficiencies and at high protein production levels. Moreover, the natural glycosylation capacity of HEK293 cells is complex and includes all major types of glycoconjugates and types of protein and lipid glycosylation. Moreover, elaborations of each type of glycosylation are more diverse than many other cell types and include for example for O-GalNAc glycosylation core 2 structures and for N-linked glycosylation LacDiNAc capping structures. The present inventors used combinatorial precise gene edited knock out and knock in of human glycogenes to develop a plurality of HEK293 isogenic mammalian cells to serve as a library of cells with different capacities for glycosylation of lipids, glycoproteins and proteoglycans to provide a display platform of the human glycome in the context of endogenous HEK293 glycoconjugates. The present inventors also expressed full coding regions of human genes or a reporter construct containing parts of human genes in these cell panels to display individual human proteins or fragments thereof in a wide range of glycoforms directed by the glycosylation capacity of individuals HEK293 cells. The plurality of HEK293 cells with different glycosylation capacities with and without exogenous expressed reporter proteins was shown to enable detection of selective binding to a specific glycoform of a protein and further enable structural deconvolution of the glycan/glycopeptide epitope involved in the binding event.

Glycan arrays have represented state-of-the-art for probing GBP interactions with the glycome for the past decades. Glycan arrays, however, depend on a match between the types of glycan structures it displays and the specificity of the GBP being analyzed. The ideal array would contain the entire glycome of an organism on a single chip, so that any GBP could be assessed. In practice, however, current arrays are limited to displaying libraries of natural and synthetic glycans that can be practically assembled. Also synthesis of branched complex-type glycans of glycoproteins and glycolipids to cover the diversity of the mammalian glycome is not yet practical. Moreover, the current glycan arrays fail to present glycans in the context of glycoconjugates as well as the cell membrane. Preparation of O-GalNAc glycopeptide libraries have demonstrated the importance of presenting at least short O-glycans in the context of peptides for recognition on arrays by some antibodies with cancer-associated antibodies (Tarp 2007, Blixt 2010). Printed glycan arrays have dramatically advanced studies of biological interactions involving glycans, but there are inherent limitations in synthesis and availability of glycans and their presentation on printed arrays without the context of the full glycoconjugate structure and the surface of a cell. While a large number of lectins, antibodies and carbohydrate-binding proteins from diverse sources including animal, viral, and microbial sources have been characterized to bind specific glycan structures by probing glycan arrays, it is clear in some (many) cases that the minimum glycan epitope defined does not reflect the adhesion properties found from other studies. This suggests that the natural physiologic binding for these involve additional features of the binding epitope that could e.g. be more elaborated glycan structures as well as protein(s) and even cell surface context.

The inventors of the present invention have recently demonstrated that it is possible to stably engineer the N-glycosylation capacity of a cell line by knock out and knock in of distinct glycosyltransferase genes (Yang 2015), and that such engineered cells may be used to produce recombinant glycoprotein therapeutics with more homogeneous and/or improved glycosylation and biological properties. In the present invention, the present inventors employed a nuclease-mediated knock out screen in human HEK293 cells to explore the wider potential for engineering the glycosylation capacity in a human cells, and further to explore how this affected display of glycans on the cell and on shed glycoproteins.

The present inventors designed a step-by-step knock out strategy with sequential loss and/or gain of glycosylation capacities to display different glycomes. The step-wise strategy involves combinatorial targeting of the glycosyltransferase genes controlling: i) the initiation and glycoconjugate determination step (FIG. 1B, group 1 genes); ii) the second step in biosynthesis for each type of glycoconjugate (FIG. 1C, group 2 genes); iii) the elongation and branching steps partly shared for glycoconjugates (FIG. 1D, group 3 genes); and iv) the capping step that terminates the biosynthesis of glycans (FIG. 1E, group 4 genes). Finally, the present inventors also targeted enzyme glycogenes modifying glycan structures in step 5 (FIG. 1F, group 5 genes). The group of genes targeted in each step are shown in FIG. 1 and listed in Tables 1, 2, 5.

TABLE 5
Group 1-5 genes and designated functions (in total 206*)
Group 1. Initiation (47 genes)

| | |
|---|---|
| N-Gly (Asn) | ALG3/6/8/9/10/12, STT3A, STT3B |
| O-Gly (Ser/Thr) | POGLUT1 (O-Glc) |
| | POFUT1/T2 (O-Fuc) |
| | POMT1/T2, TMTC1/2/3/4 (O-Man) |
| | GALNT1-T20 (O-GalNAc) |
| | OGT, EOGT (O-GlcNAc) |
| Glycolipids (Ceramide) | UGCG (Ganglio-, Lacto-, Globo-) |
| | UGT8 (Galactocerebrosides) |
| GAG (Ser) | XYLT1/T2 (O-Xyl) |
| C-mannosyl (Trp) | DPY19L1/2/3/4 (C-Man) |

Group 2. Truncation (13 genes)

| | |
|---|---|
| N-Gly | MGAT1 |
| O-Gly | POMGNT1/T2 (O-Man) |
| | MFNG, LFNG, RFNG (O-Fuc) |
| | B3GLCT (O-Fuc) |
| | GXYLT1/T2 (O-Glc) |
| | C1GALT1C1 (O-GalNAc) |
| Glycolipids | B4GALT5/T6 (Glycosphingolipids) |
| GAG | B4GALT7 |

Group 3. Elongation, branching, core structures (67 genes)

| | |
|---|---|
| N-Gly | MGAT2/3/4A/4B/4C/4D/5/5B, MAN1A1/1A2/1B1/1C1, |
| | MAN2A1/A2/, MOGS, GANAB |
| N-Gly, O-Gly | C1GALT1, B3GNT6 (O-GalNAc) |
| Glycolipids | GCNT1/T2/T3/T4/T6/T7 (O-GalNAc) |
| | B3GALT1/T2/T4/T5 (O-Gly, N-Gly) |
| | B3GALNT1/T2 (O-Gly) |
| | B3GNT2/T3/T4/T7/T8/T9 (O-Gly) |
| | B4GALT1/T2/T3/T4 (O-Gly, N-Gly) |
| | B4GALNT3/T4 (O-Gly, N-Gly) |
| | B3GNTL1 |
| | B4GAT1 |
| | LARGE, LARGE2, FKRP, FKTN (O-Man) |
| | A4GALT (Globo) |
| | B4GALNT1 (Ganglio) |
| | B3GNT5 (Lacto, Galacto) |
| | XXYLT1 (O-Glc) |
| | A3GALT2 |
| GAG | B3GALT6, B3GAT3 |
| | EXT1/T2 (Heparan Sulphate) |
| | EXTL1/L2/L3 (Heparan Sulphate) |
| | CHPF/2, CHSY1/3 (Heparan Sulphate) |
| | CSGALNACT1/T2 (Chondroitin/Dermatan sulfate) |

Group 4. Capping (35 genes)

| | |
|---|---|
| N-Gly, O-Gly | FUT1/2/3/4/5/6/7/8/9/10/11 |
| Glycolipids | ST3GAL1/2/3/4/5/6 |
| | ST6GAL1/2 |
| | ST6GALNAC1/2/3/4/5/6 |
| | ST8SIA1/2/3/4/5/6 |
| | B3GAT1/T2 |
| | ABO |
| | A4GNT |

Group 5. Modifications (44 genes)

| | |
|---|---|
| N-Gly, O-glycan, | CHST2/T4/T5/T8/T9/T10 |
| Glycolipid (sulfatases) | GAL3ST1/T2/T3/T4 |
| GAG- | CHST3/T7/T11/T12/T13/T14/T15 |
| Chrondrotin/Dermatan/ | UST |
| Heparan sulfate | NDST1/T2/T3/T4 |
| (sulfatases) | HS2ST1, HS3ST1/T2/T3A1/T3B1/T4/T5/T6, HS6ST1/T2/T3 |
| | GLCE, DSE, DSEL |
| Keratan sulfate | CHST1/T6 |
| Acetylases | CASD1 |
| Glyco-kinases | FAM20B, POMK |
| Man-6-P | GNPTAB, GNPTG, NAGPA |

The present inventors utilized methods for enriching KO clones by FACS and high throughput screening by an amplicon labelling strategy (IDAA) (Duda 2014, Yang 2015). The majority of KO clones exhibited insertions and/or deletions (indels) in the range of +20 bps, and most targeted genes were present with two alleles, while some were present with 1 or 3-4 alleles. The present inventors targeted the identified human glycosyltransferase and related glycogenes (Table 1) in five different steps as outlined in FIG. 1A.

1. Targeting the initiation and glycoconjugate determination (step 1, group 1 genes). The present inventors identified a group of glycosyltransferase genes (group 1, FIG. 1B, Table 5) that when knocked out in HEK293 individually or in combination result in loss of display of one or more types of glycoconjugates or in some cases where multiple isoenzymes initiate protein glycosylation subsets of glycoconjugates. A plurality of mammalian cells with single and combinatorial knock out of group 1 genes are useful for display and screening of which type(s) of glycosylation and glycoconjugate(s) that are important for a given biological interaction with glycans. Group 1 genes are assigned to different types of glycosylation and glycoconjugates (FIG. 1B, Table 5), and partial or complete lack of or deficiency of one or more of these genes result in complete or partial loss of specific indicated types of glycosylation of lipids, proteins and proteoglycans. Testing biological interactions with the plurality of mammalian cells displaying differences in active group 1 genes, and observing changes in such interactions in one or more of the plurality of mammalian cells is used to identify group 1 genes affecting interactions and interpret the glycoconjugate(s) that are important for the interaction as outlined in FIG. 1B.

2. Targeting the second step in biosynthesis (step 2). The present inventors identified a group of glycosyltransferase genes (group 2, FIG. 1C, Table 5) that when knocked out in HEK293 individually or in combination result in loss of display of elongated glycans on one or more types of glycoconjugates. The genes relevant for each type of glycoconjugate are indicated in FIG. 2 panels A-F. A plurality of mammalian cells with single and combinatorial knock out of group 2 genes are useful for display and screening of which type(s) of glycosylation and glycoconjugate(s) that are important for a given biological interaction with glycans. Group 2 genes are assigned to different types of glycosylation pathways and glycoconjugates (FIG. 1C, Table 5), and partial or complete lack of or deficiency of one or more of these genes result in complete or partial loss of specific indicated types of glycosylation of lipids, glycoproteins and proteoglycans. Testing biological interactions with the plurality of mammalian cells displaying differences in active group 2 genes, and observing changes in such interactions in one or more of the plurality of mammalian cells is used to identify group 2 genes affecting interactions and interpret the type of glycosylation pathway(s) and glycoconjugate(s) that are important for the interaction as outlined in FIG. 1C.

3. Targeting the elongation and branching steps (step 3). The present inventors identified a group of glycosyltransferase genes (group 3, FIG. 1D, Table 5) that when knocked out in HEK293 individually or in combination result in loss of display of elongated and/or branched glycans on one or more types of glycoconjugates. The genes relevant for each type of glycoconjugate are indicated in FIG. 2 panels A-F. A plurality of mammalian cells with single and combinatorial knock out of group 3 genes are useful for display and screening of which type(s) of glycosylation pathway and more detailed structure of the glycoconjugate(s) that are important for a given biological interaction with glycans. Group 3 genes are assigned to different types of glycosylation pathways and glycoconjugates (FIG. 1D, Table 5), and partial or complete lack of or deficiency of one or more of these genes result in complete or partial loss of specific indicated types of glycosylation of lipids, proteins and/or proteoglycans. Testing biological interactions with the plurality of mammalian cells displaying differences in active group 3 genes, and observing changes in such interactions in one or more of the plurality of mammalian cells is used to identify group 3 genes affecting interactions and interpret the type of glycosylation pathway(s) and glycoconjugate(s) that are important for the interaction as outlined in FIG. 1D.

4. Targeting the capping step (step 4). The present inventors identified a group of glycosyltransferase genes (group 4, FIG. 1E, Table 5) that when knocked out in HEK293 individually or in combination result in loss of display of end-capping of glycans on one or more types of glycoconjugates. The genes relevant for each type of glycoconjugate are indicated in FIG. 2 panels A-F. A plurality of mammalian cells with single and combinatorial knock out of group 4 genes are useful for display and screening of which type(s) of glycosylation and terminal end-capping of glycan structures on glycoconjugate(s) that are important for a given biological interaction with glycans. Group 4 genes are assigned to different types of glycosylation pathways and glycoconjugates (FIG. 1E, Table 5), and partial or complete lack of or deficiency of one or more of these genes result in complete or partial loss of specific indicated types of glycosylation of lipids, proteins and/or proteoglycans. Testing biological interactions with the plurality of mammalian cells displaying differences in active group 4 genes, and observing changes in such interactions in one or more of the plurality of mammalian cells is used to identify group 4 genes affecting interactions and interpret the type of glycosylation pathway(s) and glycoconjugate(s) that are important for the interaction as outlined in FIG. 1E.

5. Targeting the glycan modifying step (step 5). The present inventors identified a group of genes encoding enzymes modifying glycans (group 5, FIG. 1F, Table 5) that when knocked out in HEK293 individually or in combination result in loss of display of modifications of glycans on one or more types of glycoconjugates. The genes relevant for each type of glycoconjugate are indicated in FIG. 2 panels A-F. A plurality of mammalian cells with single and combinatorial knock out of group 5 genes are useful for display and screening of which type(s) of glycosylation and modifications of glycan structures on glycoconjugate(s) that are important for a given biological interaction with glycans. Group 5 genes are assigned to different types of glycosylation pathways and glycoconjugates (FIG. 1F, Table 5), and partial or complete lack of or deficiency of one or more of these genes result in complete or partial loss of specific indicated types of modifications of glycans on lipids, proteins and/or proteoglycans. Testing biological interactions with the plurality of mammalian cells displaying differences in active group 5 genes, and observing changes in such interactions in one or more of the plurality of mammalian cells is used to identify group 4 genes affecting interactions and interpret the type of glycosylation pathway(s) and glycoconjugate(s) that are important for the interaction as outlined in FIG. 1F.

Applying above stepwise glycogene knock-out approach allows probing of glycan interaction using plurality of mammalian cells displaying defined arrays of glycans, which is useful and efficient way for probing the glycosylation capacities naturally present in the HEK293 host cell.

Targeted Insertion of New Glycosylation Capacities.

The present inventors also introduced new glycosylation capacities in HEK293 to enable display of more glycan structures. The present inventors used site-directed insertion to stably integrate and express one or more human glycosyltransferase genes. The present inventors identified a group of glycosyltransferase genes not expressed in HEK293 (group 6, Table 6) that when introduced into HEK293 individually or in combination enhance the glycosylation and glycan modifying capability of HEK293 cells, and result in display of new glycan structures or modifications of glycans in different types of glycosylation pathways and on different types of glycoconjugates. A plurality of mammalian cells with one or more genes from group 6 stably introduced and with or without one or more knock outs of any of the glycosyltransferase defined in groups 1-5 genes are useful for display and screening of which type(s) of glycosylation and modifications of glycan structures on glycoconjugate(s) that are important for a given biological interaction with glycans. Group 5 genes are assigned to different types of glycosylation pathways and glycoconjugates (FIG. 1F, Table 5), and partial or complete lack of or deficiency of one or more of these genes result in complete or partial loss of specific indicated types of modifications of glycans on lipids, proteins and/or proteoglycans. Testing biological interactions with the plurality of mammalian cells displaying differences in active group 5 genes, and observing changes in such interactions in one or more of the plurality of mammalian cells is used to identify group 4 genes affecting interactions and interpret the type of glycosylation pathway(s) and glycoconjugate(s) that are important for the interaction as outlined in FIG. 1.

A combinatorial list of all individual and stacked glycosyltransferase gene inactivation events required for display of different possible parts of the HEK293 human glycome may be generated from FIG. 2 panels A-E

TABLE 6
GTf and non-GTf genes where transcripts not
are detected in HEK293 (55 genes in total)

| CAZy | Gene | HEK293 |
|---|---|---|
| family⁽¹) | symbol⁽²⁾ | (fpkm)⁽³⁾ |
| GTnc | A3GALT2 | 0 |
| GT32 | A4GNT | 0 |
| GT6 | ABO | 0 |
| GT33 | ALG1L2 | 0 |
| GT31 | B3GALNT1 | 0 |
| GT31 | B3GALT2 | 0 |
| GT31 | B3GNT6 | 0 |
| GT12 | B4GALNT2 | 0 |
| GT10 | FUT5 | 0 |
| GT10 | FUT7 | 0 |
| GT10 | FUT9 | 0 |
| GT27 | GALNT15 | 0 |
| GT27 | GALNT5 | 0 |
| GT27 | GALNT9 | 0 |
| GT27 | GALNTL5 | 0 |
| GT27 | GALNTL6 | 0 |
| GT27 | GALNT19/WBSCR17 | 0 |
| GT14 | GCNT3 | 0 |
| GT14 | GCNT4 | 0 |
| GT14 | GCNT7 | 0 |
| GT4 | GLT1D1 | 0 |
| GT6 | GLT6D1 | 0 |
| GT2 | HAS1 | 0 |
| GT54 | MGAT4C | 0 |
| GTnc | MGAT4D | 0 |
| GT29 | ST6GAL2 | 0 |
| GT29 | ST6GALNAC1 | 0 |
| GT29 | ST8SIA1 | 0 |
| GT29 | ST8SIA3 | 0 |
| GT29 | ST8SIA4 | 0 |
| GT1 | UGT1A1 | 0 |
| GT1 | UGT1A10 | 0 |
| GT1 | UGT1A3 | 0 |
| GT1 | UGT1A4 | 0 |
| GT1 | UGT1A5 | 0 |
| GT1 | UGT1A6 | 0 |
| GT1 | UGT1A7 | 0 |
| GT1 | UGT1A8 | 0 |
| GT1 | UGT1A9 | 0 |
| GT1 | UGT2A1 | 0 |
| GT1 | UGT2A3 | 0 |
| GT1 | UGT2B10 | 0 |
| GT1 | UGT2B11 | 0 |
| GT1 | UGT2B15 | 0 |
| GT1 | UGT2B17 | 0 |
| GT1 | UGT2B28 | 0 |
| GT1 | UGT2B4 | 0 |
| GT1 | UGT2B7 | 0 |
| GT1 | UGT3A1 | 0 |
| Sulfo-T | CHST2 | 0 |
| Sulfo-T | GAL3ST3 | 0 |
| Sulfo-T | HS3ST1 | 0 |
| Sulfo-T | HS3ST4 | 0 |
| Sulfo-T | HS3ST5 | 0 |
| Sulfo-T | NDST3 | 0 |

⁽¹⁾GT classification system (Lombard et al. 2013, Nucl Acid Res 42: D1P: D490-D495),
⁽²⁾Approved HGNC gene symbol
⁽³⁾Gene expression levels in HEK293 is expressed as fpkm (fragments Per Kilobase of transcript per Million) adapted from Human Protein Atlas (http://www.proteinatlas.org/)

In summary, the gene editing strategy identifies the key glycogenes controlling decisive biosynthetic steps in glycosylation of glycolipid, glycoprotein and proteoglycans in HEK293 (FIG. 1), and demonstrates remarkable plasticity in tolerance for glycoengineering and ability to display distinct subsets of the human glycome. The present inventors provide design strategies for generation of HEK293 cells with and without display of all known types of glycoconjugates, with display of glycoconjugates with only truncated glycans, with display of glycoconjugates with different degree of elongation and branching of glycans, with display of glycans with and without capping, with display of novel glycans not normally displayed.

The design matrix based on 214 human glycosyltransferase genes and 62 glycan modifying enzymes was used to design combinations of knock out and knock in in human cells to generate a combinatorial library of isogenic cells displaying different glycans and glycan modifications, and with capacity for secretion and shedding of recombinant glycoproteins with different glycans.

The following methods were applied for nuclease-based targeting of glycogenes in HEK293 cells. All gene targeting was performed in HEK293 cells. All media, supplements and other reagents used were obtained from Sigma-Aldrich unless otherwise specified. HEK293 cells were cultured in DMEM supplemented with 4 mM L-glutamine and 10% FBS.

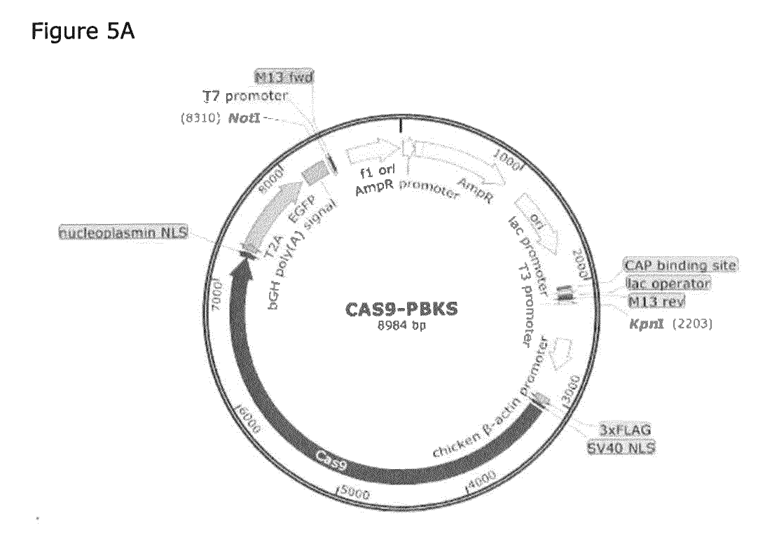

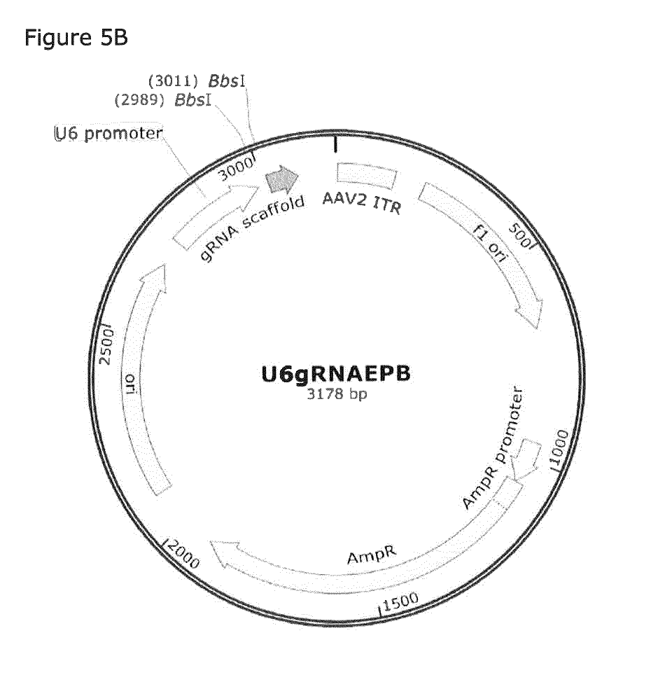

For ZFN and TALEN experiments cells were seeded at 0.5×10⁶ cells/mL in T25 flask (NUNC, Denmark) one day prior to transfection. 2×10⁶ cells and 2 μg endotoxin free plasmid DNA of each ZFN (Sigma, USA) or TALEN (ThermoScientifics/GeneArt, USA) were used for transfection. ZFNs were tagged with GFP and Crimson by a 2A linker as previously described (Duda 2014). Transfections were conducted by electroporation using Amaxa kit V and program U24 with Amaxa Nucleofector 2B (Lonza, Switzerland). Electroporated cells were subsequently placed in 3 mL growth media in a 6-well plate. For ZFN and TALEN experiments the intermediate/medium GFP/Crimson positive cell pool were enriched by FACS 72 h post nucleofection. Cells were single cell sorted again one week later to obtain single clones in round bottom 96 well plates. KO clones were identified by insertion deletion analysis (IDAA) as recently described (Yang 2015B), as well as when possible by immunocytology with appropriate lectins and monoclonal antibodies. Selected clones were further verified by TOPO cloning and Sanger sequencing for in detail characterization of mutations introduced. The strategy enabled fast screening and selection of KO clones with frameshift mutations, and on average the present inventors selected 2-5 clones from each targeting event. For CRISPR/Cas9 targeting 0.1×10⁶ cells were seeded in 24-wells the day prior to transfection, followed by PEI transfection (0.01% Polyethylenimin in 150 mM NaCl, pH=7) using 50 ul PEI, 25 ul 150 mM NaCl including 1 ug-Cas9 (Plasmid map in FIG. 5A) (encoding GFP-2A-fused Cas9) and50 ng QCgRNA amplicon or 1 ug U6-gRNA (Plasmid map in FIG. 5B) plasmid. Cells were incubated at 37 C. Two days post transfection the cell pool was enriched by FACS and the medium GFP positive cells were single cell sorted to obtain single clones in round bottom 96 well plates. KO clones were screened and identified as described above.

### Example 2

Determining the Glycosyltransferase Repertoire Expressed in a Mammalian HEK293 Cell Line.

For transcriptome analysis HEK293 cells were seeded at 0.25×10⁶cells/ml in 6 well plate and harvested at exponential phase 48 h post inoculation for total RNA extraction with RNeasy mini kit (Qiagen). RNA integrity and quality were checked by 2100-Bioanalyser (Agilent Technologies). Library construction and next generation sequencing was performed using Illumina HiSeq 2000 System (Illumina, USA) under standard conditions as recommended by the RNASeq service provider. The aligned data was used to calculate the distribution of reads on human reference genes and coverage analysis was performed.

The reported RNAseq analysis of the mammalian CHO-K1 (Xu 2011) was included for comparison since the cells are widely used for biopharm production of glycoproteins and accordingly the glycosylation capacity of CHO is well characterized (Yang 2015B, Xu 2011). Most orthologous human and CHO glycogenes could be assigned, but some genes were not identifiable and annotated in the CHO genome. Importantly, a large subset of glycogenes were found to be expressed in HEK293 and not in CHO (genes with RNA Mapping Depth of 0.0 for CHO in Table 3), while only a few genes were expressed in CHO and not in HEK293.

The transcriptome analyses of HEK293 compared with reported CHO data (Xu 2011) confirms that HEK293 displays a more complex and more human glycome with examples of the following notable features not found in CHO: Extensive fucosylation (FUTs), 2,6 sialic acid capping (ST6GAL1), LacDiNAc core (B4GALNT3/T4), core2 0-glycans (GCNT1), N-glycan branching (MGAT4s), O-GalNAc glycan density (GALNTs), O-Man glycosylation and branching (POMTs and MGAT5B), glycolipids with globo, ganglio and lactoseries structures (A4GALT, B4GALNT1, B3GNT5), and more extensive sulfation of proteoglycans and glycoproteins.

The transcriptome analyses of HEK293 cells also identifies a number of human glycosyltransferase and other glycogenes not expressed including for example several GALNTs and GCNTs (Table 6)

### Example 3

Gene Inactivation of Glycosyltransferase and Glycan Modifying Enzyme Genes in a Mammalian HEK293 Cell.

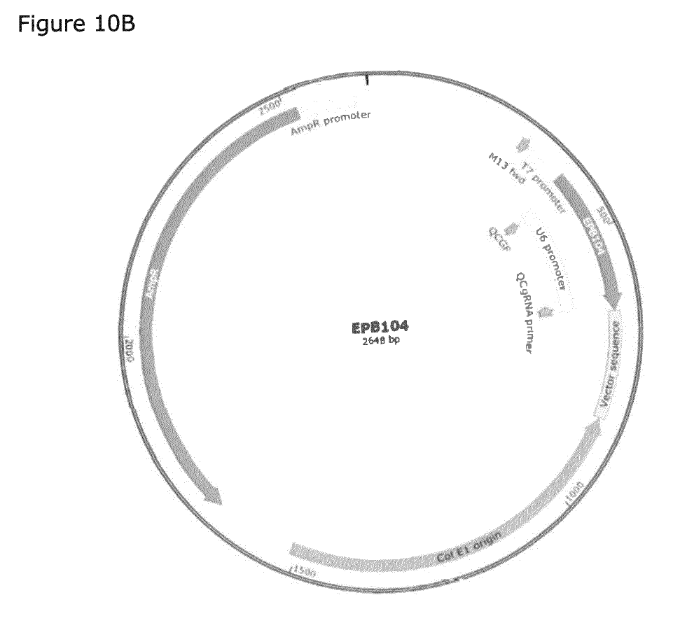

All the glycosyltransferase gene targeted inactivations were performed in HEK293 and cells were grown as described in Example 1. For ZFN and TALEN targeting cells were seeded at 0.5×10⁶ cells/mL in T25 flask (NUNC, Denmark) one day prior to transfection. 2×10⁶ cells and 2 pg endotoxin free plasmid DNA of each ZFN (Sigma, USA) were used for transfection. ZFN's were tagged with GFP and Crimson by a 2A linker (Duda 2015). Transfections were conducted by electroporation using Amaxa kit V and program U24 with Amaxa Nucleofector 2B (Lonza, Switzerland). Electroporated cells were subsequently plated in 3 mL growth media in a 6-well plate. Cells were moved to 30° C. for a 24 h cold shock. 72 h post nucleofection the intermediate positive cell pool for both GFP and Crimson were enriched by FACS. The present inventors utilized recent developed methods for enriching KO clones by FACS (GFP/Crimson tagged ZFNs) (Duda 2015). Cells were single cell sorted again one week later to obtain single clones in round bottom 96 well plates. CRISPR/Cas9 PEI targeting was performed by PEI transfection of 0.1×10⁶ preceded cells in 24wells, using 50 ng QCgRNA amplicon (FIG. 10A) or 1 ug U6gRNA plasmid (FIG. 5B) and 1 ug Cas9 plasmid (encoding GFP-2A-fused Cas9) (FIG. 5A).

QCgRNA amplicon were made with a tri-primer PCR set up, using QCGFOR, QCGRNA-Primer, QCGREV primers (See FIG. 10A) with the following primer sequences:

QCgF/gX/QCgR primers used for validating are the following:

QCGfor:
5′-ctcgatatcgaattcGAGGGCCTATTTCCCATGATTCC-3′
QCGrev:
5′-cgaattaacggtaccAAAAAAAGCACCGACTCGGTGCCACTTTTTCA
AGTTGATAACGGACTAGCCTTATTTTAACTTGCTATTTCTAGCTC-3′
gX:
5′-TTTAACTTGCTATTTCTAGCTCTAAAACnnnnnnnnnnnnnnnnnnn
nGGTGTTTCGTCCTTTCCACAAGAT-3

Two days post transfection the intermediate GFP positive cell pool was enriched by FACS. Cells were single cell sorted again one week later to obtain single clones in round bottom 96 well plates For TALENs the post transfected cell pool was single cell sorted without FACS. KO clones were identified by high throughput amplicon labeling strategy screening (IDAA), and when possible also by immunocytology with appropriate lectins and monoclonal antibodies. Selected clones were further verified by TOPO cloning of PCR products (Invitrogen, US) and in detail Sanger sequencing. The strategy enabled fast screening and selection of KO clones with appropriate inactivation mutations as outlined herein, and on average the present inventors selected 2-5 clones from each targeting event.

The majority of KO clones exhibited out of frame insertions and/or deletions (indels), and most targeted genes were present with two alleles, while some were present with 1 or 3 or 4 alleles.

The optimized DNA fragments used for genetic engineering of the human cells are included in Table 7 and 8. Table 7 provides a list of validated gRNAs for 279 human glycosyltransferase and glycan modifying enzymes listed in Tables 1 and 2, plus additional 14 glycan relevant genes. Table 8 provides a list of validated ZFN and Talen target sequences against selected human glycosyltransferases and glycan modifying enzymes listed in Tables 1 and 2.

TABLE 7
HEK293 Glyco-gene gRNA list for CRISPR/Cas9
engineering
For each gene 3 or 4 gRNA sequences were designed.
After evaluation by QuickChange based gRNA
amplicon validation procedure the optimal gRNA
was selected and such validated constructs are
shown as single gRNA sequence for that particular
gene. When validation has not been completed the
3-4 gRNA sequence candidates are included.

| | Gene name | |
|---|---|---|
| | (HGNC) | gRNA sequence |
| 1 | GALNT1 | TCCCACTGTACACTCACAAT |
| 2 | GALNT2 | GTGAAACGTGATCACCACGC |
| 3 | GALNT3 | TATGGAAGTAACCATAACCG |
| 4 | GALNT4 | AACAGTGGCCTATATCTTCG |
| 5 | GALNT5 | GATGACTTCGATTACTGGAC |
| 6 | GALNT6 | GAAGAGCAAGTGGACCCCCC |
| 7 | GALNT7 | ATGCCCAACCGAGGCGGCAA |
| 8 | GALNT8 | GTAGTCTCGCGTGTCGGGGA |
| 9 | GALNT9 | CGCACTGTCGCGGATCCGAG |
| 10 | GALNT10 | CTCTCTCAGCATCGGTCATG |
| 11 | GALNT11 | TATGCTTATCAGTGACCGCT |
| 12 | GALNT12 | TTCTTCTAGCAGGATATCCG |
| 13 | GALNT13 | TTAATACGTGCCCGTCTTCG |
| 14 | GALNT14 | CTTACAGGACTACACGCGGG |
| 15 | GALNT15 | GCTGGCTGAGGTCGTCCACG |
| 16 | GALNT16 | GTAATGGCGGGTGTCCCGGA |
| 17 | GALNT17 | AGTTCACATTGACCTCGCAG |
| 18 | GALNT18 | TGAGATAGAAGAGTACCCGC |
| 19 | GALNT19 | AGGTTGGCCACCTCGGCGCG |
| 20 | GALNT20 | ATACTCTGTTCACCTCACAG |
| 21 | C1GALT1 | ACAACACTTTGTTACAACGC |
| | | CCAAGTAGCTTTGACGTGTT |
| | | CAACACTTTGTTACAACGCT |
| 22 | COSMC | GTAGGTGATGATGCTCATGG |
| 23 | POMT1 | GAGCTCCAACACTATCTGGT |
| 24 | POMT2 | CTTCGAGGCGGTCGGCTGGT |
| 25 | POMGNT1 | GAGGGACACATGGGCCTTCG |
| 26 | GOLPH3 | GCAACGCCGCCGACAAGGAG |
| | | AGGGCGACTCCAAGGAAACG |
| | | GAGCAGGACGACGACGACAA |
| | | GCTGGGCCTCAAGGACCGCG |
| 27 | GOLPH3L | TTATTTCAGTGCGACGGGCC |
| | | TCTTGCTTATTTCAGTGCGA |
| | | CTTCTTCCATAAGAGTAAGG |
| | | GGATATCCGCCTTACTCTTA |
| 28 | LGALS1 | GGTGGCCTGTGGCAATCGGC |
| | | CGATGGTGTTGGCGTCGCCG |
| | | GGAGAGTGCCTTCGAGTGCG |
| 29 | ATG7 | CTTGAAAGACTCGAGTGTGT |
| | | TAGCTGGGCAGCAACGGGCT |
| | | CTTCCAAGGTCAAAGGACGA |
| 30 | VAMP7 | CTCTTGAACCGTAAGTAGTC |
| | | ACTTGAGAACTCGCTATTCA |
| | | CACACCAAGCATGTTTGGCA |
| 31 | LGALS7 | CTCATCATCGCGTCAGACGA |
| | | GGTGCTGAGAATTCGCGGCT |
| | | CTGCCCGAGGGCATCCGCCC |
| 32 | FAM20C | GTGTTCCTACTACTGCTCCA |
| 33 | B4GALNT3 | CATAGATTCGCACAACCCTG |
| 34 | B4GALNT4 | CAGTGAGACCGACGGCCGGG |
| 35 | LDLR | GTCTGTCACCTGCAAATCCG |
| 36 | PCSK9 | AGTGACCACCGGGAAATCGA |
| 37 | | |
| 38 | STT3A | ATGTTGTTCTTAGCATAAGA |
| 39 | STT3B | TTGGGTGTATCACTAGCTGC |
| 40 | ST6GAL1 | TGTATCCTCAAGCAGCACCC |
| 41 | ST6GAL2 | AGCTGGGTACAGGCTCAGCG |
| 42 | ST6GALNAC1 | ACGGTGTCAGAGAAGCACCA |
| 43 | ST6GALNAC2 | GAGCCCCCGCCAGCCATACG |
| 44 | ST6GALNAC3 | GTATCCATAGTGAGTTCGAA |
| 45 | ST6GALNAC4 | TGTGTGTGAGACGACACGCA |
| 46 | ST6GALNAC5 | CTAGTGTACAGCAGCCTCGG |
| 47 | ST6GALNAC6 | TCTTCCATTACGGCTCCCTG |
| 48 | MUC1 | GCATTCTTCTCAGTAGAGCT |
| 49 | ST3GAL1 | TCCAAGTCGATGGTCTTGAA |
| 50 | ST3GAL2 | GTGGCTGTCAAACCAGTCGG |
| 51 | ST3GAL3 | GATTCTAGCCCACTTGCGAA |
| 52 | ST3GAL4 | TTACCCGCTTCTTATCACTC |
| 53 | ST3GAL5 | ATTTGAGCACAGGTATAGCG |
| 54 | ST3GAL6 | TGAGAATCACTGTCGTATTA |
| 55 | B4GALT7 | TGACCTGCTCCCTCTCAACG |
| 56 | CHST11 | GATAAAGGATCCCAAGCAAG |
| 57 | CHST12 | CGTGTGCAAGTAGAAGTGCG |
| 58 | B3GAT1 | GCTAGGATGTCCCGTCTCTT |
| 59 | B3GAT2 | AAAGAAGCGGGTGAAAAGCG |
| 60 | B3GAT3 | TCGGAGTTCCGCTTGCAGCT |
| 61 | B4GALT1 | GGAGTCTCCACACCGCTGCA |
| 62 | B4GALT2 | AGTAGAGGATGACGGCCACG |
| 63 | B4GALT3 | TCCCTGATCTCGGCCAAATA |
| 64 | B4GALT4 | ACCCACGAAGTAGTTACTGG |
| 65 | B4GALT5 | TTCGGAGTGCTTATGCCAAG |
| 66 | B4GALT6 | CTCTTTATGGTACAAGCTCG |
| 67 | B3GNT1 | CATCGCCACCAGCATGAGCG |
| 68 | B3GNT2 | GTTCCAGTATGCCTCGGGAG |
| 69 | B3GNT3 | GCAGCCACCGGCGATCCCCG |
| 70 | B3GNT4 | GTATCCTTGGAACAGCCTGA |
| 71 | B3GNT5 | CTCACAATGTGATTATCGAT |
| | | CTCTTAAGCACACCTCAGCG |
| | | CCAATACTTGATTAACCACA |
| 72 | B3GNT6 | GCGGGGACCTTGGCGCCTGG |
| 73 | B3GNT7 | GCTCCTGCAGAAACTGACCA |
| 74 | B3GNT8 | GGAGTGTGAGCAGTGCTGAC |
| 75 | B3GNT9 | GCTTATTGCTGTCAAGTCGG |
| 76 | MGAT1 | CCCTCAGTCAGCGCTCTCGA |
| 77 | MGAT2 | GTTCGGCGTCCAGCAACGGT |
| 78 | MGAT3 | TCCTCGGCCGCCTTGCTGGG |
| 79 | MGAT4A | CTTTGTCTTGGTATACTACA |
| 80 | MGAT4B | GGAGAGCCTCAAGCGCTCCA |
| 81 | MGAT4C | TGTGGCAGCTAGGTAGCGAT |
| 82 | MGAT4D | GGTATTTCCACTGTTAACAG |
| 83 | MGAT5 | GCTGTCATGACTCCAGCGTA |
| 84 | MGAT5B | CCACCAAGGTACCTTCTGTG |
| 85 | FUT1 | CAGGGTGATGCGGAATACCG |
| 86 | FUT2 | AGTGCTAGCCTCAACATCAA |
| 87 | FUT3 | TGTCCGTAGCAGGATCAGGA |
| 88 | FUT4 | CCTCCACGCCTGCGGACGCG |
| 89 | FUT5 | GGCAGTGGAACCTGTCACCG |
| 90 | FUT6 | GGACCCATTAGGGTACACAG |
| 91 | FUT7 | TAGCGGGTGCAGGTGTCGCT |
| 92 | FUT8 | ACCTTGCTGTTTTATATAGG |
| 93 | FUT9 | GTGAACGGTCCGTTGTGAGA |
| 94 | FUT10 | GGGACCACCAGAGCATAATG |
| 95 | FUT11 | CGCGCAGCTCTGGGACGCCG |
| 96 | POFUT1 | AAAGCTGCTAAACCGTACCT |
| 97 | POFUT2 | GGAAGGCTTCAACCTGCGCA |
| 98 | LFNG | GATGAAGCGGTCATACTCCA |
| 99 | MFNG | GCGCAAGCGGTGGAAAGCCC |
| 100 | RFNG | GCCACCCTGGACCCTCTCGG |
| 101 | POGLUT1 | GAGGATCTAACTCCTTTCCG |
| 102 | GXYLT1 | AATTGTTTTAGCTTGACAAC |
| | | AACAATCTCTGCGAAGCACA |
| | | TTCGCAGAGATTGTTCTTGC |
| 103 | GXYLT2 | ACCCGAGCTCTGGATCCACC |
| 104 | XXYLT1 | GCAGTGAGCGCAGCGCGACG |
| 105 | B3GALT1 | TCAGCCACCTAACAGTTGCC |
| 106 | B3GALT2 | CCTGTGACATACACTTTCCG |
| 107 | B3GALT4 | GGAAGCTTGCAGTGGTCCCG |
| 108 | B3GALT5 | TTTCCCCCACGTCTGCCGGA |
| 109 | B3GALT6 | CTTCGAGTTCGTGCTCAAGG |
| | | CGGACGACGACTCCTTCGCG |
| | | AGAAGCCCCAGTAGAGGCGG |
| 110 | B3GALTL | GTTTAGCCAGCTGATGAAGG |
| | | CATCAGCTGGCTAAACAAGA |
| | | CAAACGTACTGCGGTAACAA |
| 111 | B3GALNT1 | CGTTCTATCACATTGTAGTG |
| 112 | B3GALNT2 | GAAACGTGATAAGAAGCACC |
| 113 | B4GALNT1 | ACCGGGATGTGTGCGTAGCG |
| 114 | B4GALNT2 | CCGTGGACTGGGTACCCAAA |
| 115 | GCNT1 | TAGTCGTCAGGTGTCCACCG |
| 116 | GCNT2 | ATAGCAGGTAGCTTCATCAA |
| 117 | GCNT3 | ACTGTTCAGGGGTCACCCGA |
| 118 | GCNT4 | GCAGCCATAGGGTTAAAAAC |
| 119 | GCNT6 | GGTCAAATAGATGACGTAGG |
| 120 | GCNT7 | ACTGCTCCAGGATTTCTCGG |
| 121 | ST8SIA1 | CGTTGGGCAGCCGGTAGACG |
| 122 | ST8SIA2 | TGCCATCGTGGGCAACTCGG |
| 123 | ST8SIA3 | GATGAGCGATAAAATCAGCA |
| 124 | ST8SIA4 | AGATGCGCTCCATTAGGAAG |
| 125 | ST8SIA5 | ATACAGGATCTGTTGCAGCA |
| 126 | ST8SIA6 | GGAGCGTCCTCAGCGCTGCG |
| 127 | XYLT1 | ACAACAGCAACTTCGCACCC |
| 128 | XYLT2 | GACAGTTCAGCAGGGCGACG |
| 129 | CSGALNACT1 | GGTACCCCTCCTTCCCCGTG |
| 130 | CSGALNACT2 | GTATTATCAAGCCCTCCTAC |
| 131 | CHPF | GGAACACCACACGCTCCAGC |
| 132 | CHPF2 | CAGTGAAGTAGAGTAACCGA |
| 133 | CHSY1 | GTACATCAAAGGAGACCGTC |
| 134 | CHSY3 | TCTCGGCTAAAGATCATGCC |
| 135 | EXT1 | GTAGAACCTGGAGCCCTCGA |
| 136 | EXT2 | TCGGCTGGCAGCCTAACAAC |
| 137 | EXTL1 | GTAGAAGCGAGAGCCCTCAA |
| 138 | EXTL2 | CAGCTACCAGTAATAATACG |
| 139 | EXTL3 | GGTGGGGAACGAGCTGTGCG |
| 140 | GBGT1 | GTTGGCGCCCATCGTCTCCG |
| 141 | OGT | GCAACCTATTCTTCTCTAAC |
| 142 | EOGT | GTTTGCAGCTATGTCGACAT |
| 143 | POMGNT2 | ACTGAGGATCGACTACCCGA |
| 144 | LARGE | CCTGGAGGTGCGCATGCGCG |
| 145 | FKRP | GCACCAGGACGGTGACACGG |
| 146 | FKTN | GAGTCTATCCCGTCTAGCCG |
| 147 | KDELC1 | CTGAATATAGAAATAGCGGG |
| 148 | KDELC2 | GATTTGACTACGACTTTGAA |
| 149 | GLT1D1 | GCTCTTCATCTCTATAGGGG |
| 150 | GTDC1 | TCAGAGTGTGATACACACTG |
| 151 | GLT8D1 | GCTCTCCGACATGCAGTAGA |
| 152 | GLT8D2 | GCTATTGATGGCAGCCATAG |
| 153 | DAG1 | ACATTTCAGTGAGCGCTACA |
| 154 | CDH1 | ATAGGCTGTCCTTTGTCGAC |
| 155 | CDH2 | TTACACTGTACCGCAGTGAA |
| | | CCGACAGCTGACCCGAGATG |
| | | GCTATCTGCTCGCGATCCAG |
| 156 | LARGE2 | CTTCATTAGCCCATAGAGGC |
| | | CCGTGTCCAGGACAATGACG |
| | | CCAGAGCTCCGAGATGTCAG |
| 157 | MMP23E5 | GGCCTGATGCACTCACAACA |
| 158 | MMP23E7 | AGGAACGTGACCTTCCGCTG |
| 159 | TMTC1 | GACCTGCCAGTCATAGCACA |
| 160 | TMTC2 | GCCTGAACCATGCCATTGGA |
| 161 | TMTC3 | ACTGCTGGACAGTTTCTCCG |
| 162 | TMTC4 | GCTGCGTGCAAAACACACAA |
| 163 | SDF2 | AGCCGCAAGTAACGACACCC |
| 164 | SDF2L | GCAATCGGTGACCGGCGTAG |
| 165 | A3GALT2 | CTAAGAGGCCAAGTGTAAGT |
| | | CGGCAGATCCTACTTACACT |
| | | GCTTTTACCTGAATTTAGGG |
| 166 | A4GALT | TCCGGGGCGCCCCAAGGCAG |
| | | GTCTGCACCCTGTTCATCAT |
| | | GCACGTTGTGGGAGAGCCCA |
| 167 | A4GNT | ACTTCAGGGTGAACTGGTAG |
| | | CTACGGAACAGGAGACCAAA |
| | | AATCTTGGCAGCAGACTCTA |
| 168 | ABO | AATGTGCCCTCCCAGACAAT |
| 169 | NGLY1 | ACTAGACTCTTGCCTGTCAG |
| 170 | UGCG | TTAGGATCTACCCCTTTCAG |
| 171 | UGGT1 | CTACTATCATGCAATATTGG |
| 172 | UGGT2 | TTCGCAGCTCGGCTCCGGGA |
| | | GGCAGTCACCGACTTGGACG |
| | | CGGGGTCTCGGGCCACTTCG |
| 173 | UGT8 | TGGTACTGTTAAAGATCCCT |
| | | GGGCATGGTTGCCAACCATC |
| | | TGTGATAGCTCATCTTTTAG |
| 174 | HAS1 | CATGGTCGACATGTTCCGCG |
| 175 | HAS2 | TGAAAAGGCTAACCTACCCT |
| 176 | HAS3 | GGTGGTGGATGGCAACCGCC |
| 177 | GYS1 | GACGAAGGCGAAGGTGACAG |
| 178 | GYS2 | ACTGTAAGAAGAATGCTTCG |
| | | AGTTCTTCGACTTCCCACTG |
| | | GGAGCAGTACAAACCTTTAT |
| 179 | GYG1 | GACCAGGGCACCTTTGGCGT |
| 180 | GYG2 | GAGAGTCCAACAGTGAAGCT |
| 181 | GLT6D1 | GGGAAGGGACTTTCGACAGG |
| 182 | CERCAM | GTCTGCCCCTTCAGCCCGCA |
| 183 | COLGALT1 | GGTGGCTACGGACCACAACA |
| | | ATAACACGTCAACTGTGCTG |
| | | GAAGAGTTTGTACCATTCCG |
| 184 | COLGALT2 | ACTACGTTCAGATTCGAGAA |
| | | CCACCTTCCTAATTGACCTC |
| | | GTAGAAAGTCAGCTTGTCCG |
| 185 | B3GNTL1 | CAGTCCACAACGCTGAACCG |
| 186 | ENPP1 | GCAGTTTCCAAGCTCAACAC |
| 187 | GBE1 | GGCTGTGTACCACATCTAAG |
| 188 | PIGA | ACACTCTCTCGGGTTAGCCC |
| | | CCGTACCCATAATATATGCA |
| | | TTTTCGATTTCCATAAGCAT |
| 189 | PIGB | AAGTGCGGAATGGAGCCGGG |
| | | TTCGCAGCTTTATCTTGCCG |
| | | TACCTTGTACTTCAACACCC |
| 190 | PIGC | TCAACCTGTGACTAACACCA |
| | | GGAGCTCTTCCAGGAATCGC |
| | | AGTCCCTAAAAGCCAATGGG |
| 191 | PIGH | GGCAGGACGGGGAGTAGTAG |
| | | GCAGAATTCCCGGCAGGACG |
| | | ACTTGCCATTACCTCGCAGA |
| 192 | PIGM | GAAGACGCCATAGAAAACCA |
| | | CCCCTCCGTGACGAAGCGCG |
| | | ACTTTCCAAAGAGCTCGCTG |
| 193 | PIGP | TACAGTACTTTACCTCGTGT |
| | | CAGGAATAAAGGCCCACACG |
| | | AACTTACTTTTGAGGCCAAT |
| 194 | PIGQ | CACCTGGTGCCACTGCCGGC |
| | | CGGCACGTTCTGGAGCTGCG |
| | | CATCTTCTATGACCAGCGCC |
| 195 | PIGV | AGTTGGTCCACAAAGCCTGA |
| | | AGGCTTTGTGGACCAACTCG |
| | | AACTGTTGAGACCCTTACGG |
| 196 | PIGZ | GCGTAGCATCTGTAGCAGCT |
| | | GCAACAACTGGATCTGAAGA |
| | | GCACATAGCCCGTCTGCGGA |
| 197 | PYGB | GTTGAAGCTCTTCCGCACCT |
| | | AGGTGCGGAAGAGCTTCAAC |
| | | AGCGCGAAGAAGTAGTCGCG |
| 198 | PYGL | TGACGGACCAGGAGAAGCGG |
| | | TCTCCACGCCCACGATGCCG |
| | | CGCGAAGTAGTAGTCGCGGG |
| 199 | PYGM | CGAGTGTGAAATGCAGGTGC |
| | | AGCAAAGTAGTAGTCTCGTG |
| | | ACGAGACTACTACTTTGCTC |
| 200 | TMEM5 | CATTGAGCAGTCACATCGCT |
| | | CCAGCGATGTGACTGCTCAA |
| | | AGAGAAGGAAAGTCAATCGT |
| 201 | DPY19L1 | AATGACGGTCATTTTCAAAG |
| | | TCTCCCTTTCCAATGTTGAG |
| | | CTCACCTCTCAACATTGGAA |
| 202 | DPY19L2 | AGCCAGTCTAAGGGGCGGCG |
| | | CAGACTTTGGATCCTCCCCG |
| | | GCAGCTCCGGGAAAAGGTGC |
| 203 | DPY19L3 | GAGCTGTGACATAGATCGCC |
| | | TGTACCACTGAGTAGCCAGC |
| | | GCTGGCTACTCAGTGGTACA |
| 204 | DPY19L4 | TGTCTTGCAGCGGTTACTAG |
| | | ACTTATCAGCATACCATGAA |
| | | CATACCATGAACGGAAATTC |
| 205 | SCFD1 | CCATTTAAACAGGGTTAATT |
| | | GGAGTGGAAAACTCTCCAGC |
| | | GAAGTCTTATGATTTAACTC |
| 206 | SCFD2 | GATGTCCCGTAGGATCTCCA |
| | | AGCTAATCATGTCCCAGCGG |
| | | CAACATGAACTACACGGCCG |
| 207 | ALG1 | GCAATGCTAGGCAGACCTGG |
| | | TGACGAGCTTGCTTCCACAA |
| | | GCAGAACGAGGGGATGGTTG |
| 208 | ALG2 | TGTTCAGGCTGGCTAGACGG |
| | | TTTTCTTAAACGACTATACA |
| | | GGCCCCAATTGACTGGATAG |
| 209 | ALG3 | TTGTACTATGCCACCAGCCG |
| | | CCGAGGCACTGACATCCGCA |
| | | GATCTATCACCAGACCTGCA |
| 210 | ALG5 | AGCAGTTGTAAATGCAACGA |
| | | TCTTCATGTCGATGGAGTGC |
| | | GTAGGTGAGTCCCATATGCT |
| 211 | ALG6 | GGCTGCCATTCTTTACAGAA |
| | | AGAGTCTTCTTAGAACCTGC |
| | | AGACTCTTCCCGGTTGATCG |
| 212 | ALG8 | GTGCGGTGCCGCAGCAATGG |
| | | GCGGCGCTCACAATTGCCAC |
| | | TGCGAGTCCCTTACTATGTG |
| 213 | ALG9 | GTGTACATACAGAAGCTACT |
| | | CGTTGATAGCCATGACTGGA |
| | | GGCCATTCAGTGCAGCTCTT |
| 214 | ALG10 | TGATCACTCCAATTGACACC |
| | | GGCTTGTACCTGGTGTCAAT |
| | | CTGCCATTTGGATCTTTGGA |
| 215 | ALG10B | GCAGGAATGGCGCAGCTAGA |
| | | GCTAGAGGGTTACTGTTTCT |
| | | TGTAGGGCTCTCGCAGCGCC |
| 216 | ALG11 | GTTCCACATACAATGAGCCC |
| | | GCAGCAGTCTGATTCCCCAA |
| | | CCATACTGCAATGCTGGTGG |
| 217 | ALG12 | AGTTCCCCGGAGTCGTCCCC |
| | | CTGCGATCACCACTGGCCCG |
| | | GCGAAAGCACGTAAACCGCG |
| 218 | ALG13 | ACCCTGTACAAAATGCATAA |
| | | AATGGCCCGAAAGAGGCAAG |
| | | GCTCCGAAATGGCCCGAAAG |
| 219 | ALG14 | GTGCGTTCTCGTTCTAGCTG |
| | | GAAGCACTACCCATATTCGC |
| | | AAGATACTGAGAGACTCCCG |
| 220 | DPM1 | GTAGTCCTCGCAGGTCTCGG |
| | | TTCTCGCGCTCGTTGTAGGT |
| | | ACCAGCAGCCACACGATGAG |
| 221 | DPM2 | CGAGGCCGAGTCCCACCACC |
| | | TGATCAGGCTAACGGCGACG |
| | | CTTCACCTACTACACCGCCT |
| 222 | DPM3 | GCCCTGACCACGGGAGCCTT |
| | | GCGGACACCAGCAAGTAGGC |
| | | GCCTACTTGCTGGTGTCCGC |
| 223 | DPAGT1 | AGTTGGTGAAATAGACCATG |
| | | GAAGGGCTTGGGCACCACAA |
| | | GATGCAGGCCAAGTATCGGG |
| 224 | RFT1 | CCCCACTGAGACATGCTCTG |
| | | GGTCTGGCTCCAGTCTCGCT |
| | | CGTTAGCCACAGCAGGTTGA |
| 225 | RPN1 | AGAGGCAAGCTTCACACGCA |
| | | GTAGCTCTCCACATTTCGAG |
| | | AGTAGGTCCTCAGAGCGCGT |
| 226 | RPN2 | CTATGATTGTCAGGGCCAAC |
| | | GACAATCATAGCCAGCACCT |
| | | CTACCTCACCAAGCATGACG |
| 227 | DDOST | GCGCAAACCGCGCCAAGCAA |
| | | TCCAGCAGCACTAAGGTGCG |
| | | AATGAGTCTCCCGCACGTTG |
| 228 | DAD1 | CGGTAGTGTCTGTCATTTCG |
| | | GTAACCGAACTGCAGCGCCC |
| | | GTTCGGTTACTGTCTCCTCG |
| 229 | TUSC3 | CACGCCGTAGGCAAGCGGGG |
| | | AGGAAGGGAAAGCTCCCGGT |
| | | CTGCTCTGCATCCAGCTCGG |
| 230 | OST4 | TCTTCTCCGCAGGATGATCA |
| | | GCCCAGCATGTTGGCGAAGA |
| | | GCCATCTTCGCCAACATGCT |
| 231 | DOLK | GCCAGCACCGATCCACTCAG |
| | | CCACGAGTATCGGTCCCATA |
| | | CCGATACTCGTGGTGCGCCG |
| 232 | CHST1 | CGTGGTAGAGGGGCTCAAAC |
| | | TCTTGCCCTGGGTGAAGCGG |
| | | GCCTAGCATGACCCGCCGGT |
| 233 | CHST2 | AAGGAGTGCGGAGGTCCAAA |
| | | TGGTGTACGTGTTCACCACG |
| | | GCTATTCAACCAGAATCCCG |
| 234 | CHST3 | GTTGGCATCTGCTAGAGCTT |
| | | GGAGCCGCCCAGACCGGCCG |
| | | ATGAGCAGCACGTGGCGCCG |
| 235 | CHST4 | CCTTCAAGCAGAGCACCGCC |
| | | GCTCATGTCGCACAAGAAGA |
| | | AAGAGGCTGGACTGTCTCCG |
| 236 | CHST5 | ACTGTCTTGCTGGAGAACCG |
| | | TCTTCATCATCTCCCGGCCA |
| | | CATGCCACGCGGGCTCCATC |
| 237 | CHST6 | CGTGCCACGCGGGCTCCATT |
| | | CACAGCCATGTGCAGCGTTG |
| | | CCTGTCCGACCTCTTCCAGT |
| 238 | CHST7 | GTCCCCTCCGTATTGGACGG |
| | | CGGTTCCCAAGCAACCTCAG |
| | | GCTGGTTAAAGAGTTCGCCC |
| 239 | CHST8 | CCCTGCGACCTGGAACAATG |
| | | TGCAGCTCCGAACAGCAGGA |
| | | GCTGGGGGGCGAGCTCCGTA |
| 240 | CHST9 | TCAATTCTGAGAGATCTACT |
| | | GACCAGTCATTCACAAGGAG |
| | | AAGCTTTAAGTAAGTCCACA |
| 241 | CHST10 | AGGCGCTCCATGTAGACCAG |
| | | ACTCATCAGAAACGTCTGCA |
| | | TATTCGGTCCAGGACAAACT |
| 242 | CHST13 | CTGCCGGTGCACCTTCGCCA |
| | | CCTGTAGCCGCCACTCACGC |
| | | TCTTGCGCGGAGATGGCGCG |
| 243 | CHST14 | CTGCTGCTCATGATCGAGCG |
| | | GCTGTGCCCTCGCGGCCGGG |
| | | CTGTCCGCACACCGCCCGCA |
| 244 | CHST15 | TGCATACAGCTGTTACCCGA |
| | | TTATTATCAGTCCAAAAACG |
| | | GGTTTTTGCGCTTCAAAAAG |
| 245 | GAL3ST1 | GATCCGGGCCAACGGCTCGG |
| | | AAGAACACGATGTTGCGCCG |
| | | GCGGAACAGGATGTTGAGCA |
| 246 | GAL3ST2 | AACATGATGTTGGTGACCGG |
| | | CGAGACCCACAACCTGTCCG |
| | | TAGCGCGCCAGGAAGAGCCA |
| 247 | GAL3ST3 | GTCATGTGCTTGGGGCGCGG |
| | | TGCCGAGCGCCACAACCTGA |
| | | ACTTCGTGCACCCGGCCACG |
| 248 | GAL3ST4 | TTGGGTAGCCAAACTGGTAG |
| | | GTAAAAGGCTACCGCCCACA |
| | | TGAACCTCATGTGGTGACAG |
| 249 | HS2ST1 | AATTGAGCAGCGACATACAA |
| | | TCTAAAGTGGCATCTTGCCG |
| | | GCAAGATGCCACTTTAGATG |
| 250 | HS3ST1 | AGGAGCTTCTGCGGAAAGCG |
| | | ATCATCATCGGCGTGCGCAA |
| | | GAAGTGGACCTCGTTCTCCG |
| 251 | HS3ST2 | CAGCGTGATCTGGCTCTCGA |
| | | GACATGTTGAAGATGCGTCG |
| | | CGTGTAATCAGAGATGGCAC |
| 252 | HS3ST3A1 | CGTCCTGGCCGGAGGCCCGA |
| | | GCTGGCGGTGTGGCCGGCGG |
| | | GCCTCCTCGCCGTCGTCGCG |
| 253 | HS3ST3B1 | GCGAGCTTCCTCCTCACCGG |
| | | ATAGAGCCAGACGCAGAGCA |
| | | ATGTTCCTGTACTCGTGCGC |
| 254 | HS3ST4 | CTTCTTCTCCCCATAGTCGG |
| | | GGATCGCCTCCAGCAGCGCG |
| | | CGTGCACCCGGACGTGCGGG |
| 255 | HS3ST5 | CACCCAGTCGACCTTCAATG |
| | | CGCGCCCTGCAGTTTAAGCG |
| | | CCTGCTGCACGAGTTCCGGA |
| 256 | HS3ST6 | TCGCGTCACGAAGTAGCTGG |
| | | GACATGGCGTGGATGCGGCG |
| | | GGACACGAAGCTGATCGTGG |
| 257 | HS6ST1 | CGCTCAGGTAGCGGGACACG |
| | | ATGCAACGACGTCTTCCACG |
| | | GGAGCTGCCGCCCTGCTACG |
| 258 | HS6ST2 | GGAGGACGATCACGGCAAAT |
| | | GGTGCCGGACCCGTACCGCT |
| | | CCGCGGGTGAAATTGTAGCG |
| 259 | HS6ST3 | CCTCCACATCCAGAAGACGG |
| | | CCTGGTGAAGAACATCCGGC |
| | | TCTCCTTCTTGCCAGGCCGG |
| 260 | NDST1 | ACGCTCACTGACAAGGGCCG |
| | | CGTCCAGGTTGACATACTTG |
| | | GTACTGTGTGGCCTACGGCG |
| 261 | NDST2 | ACATTGACTGATAATACCCA |
| | | ACTGCTAGACCGGTACTGCG |
| | | CTACTGAGCGCCCAGCTCAA |
| 262 | NDST3 | TCTGCTTACTACCTGTACAG |
| | | GTTGATATGGTAGGTGTTGG |
| | | CTACAAATACTAGGACTGTG |
| 263 | NDST4 | GTAGACCCCTGAGTGATGTG |
| | | ACATCACTCAGGGGTCTACC |
| | | ACATTCAGCTGTATGCAGCT |
| 264 | UST | TGTTGTACACCACCTGGCTT |
| | | CTACCCTGTTGTACACCACC |
| | | AGAATCTTGTCGGAGAAGCA |
| 265 | DSEL | TGGCTAGTAGAGAATGCACC |
| | | GGAAATGTACGAGTATTCCA |
| | | GTATTCCAAGGTCCGCTCAT |
| 266 | GLCE | AAGCCACTACTCGAACGCCG |
| | | TAACAACTATATGAACCACG |
| | | TTGAAGCCCCCAACAACAGG |
| 267 | FAM20B | TACATCAGCTGCCAACCGGG |
| | | AATGATGACTGGCTTGCGGG |
| | | CACTGGGCTGCAATCTCCCA |
| 268 | MOGS | CCTGAGCTTAGGAGTCCCCG |
| 269 | GANAB | TGGCTGATCCACCAATAGCC |
| 270 | MAN1A1 | GAACTTCTCCGTCAGGCGGA |
| | | TGGGGGCACGAAGTCCACCG |
| | | TGCCCTTTTCTCGCGGATGG |
| 271 | MAN1A2 | GTCAACATTCGATTTATTGG |
| 272 | MAN1B1 | GCGGTGATCGAGCCTGAGCA |
| 273 | MAN1C1 | GCTATAAGCGTTATGCAATG |
| 274 | MAN2A1 | GGTCAGCTTGAAATTGTGAC |
| 275 | MAN2A2 | GGAGCTGCCGTTTGACAACG |
| 276 | MANEA | ATGTCATCATGGCAAAGTTT |
| 277 | GNPTAB | TAAACAACGTCAATCGGCAT |
| 278 | GNPTG | GCTGCTCACCCAAACGCGTT |
| 279 | NAGA | TAGCAAGTCGTCGTCGCGGG |

### Example 4

Gene Insertion of Glycosyltransferase and Glycan Modifying Enzyme Genes in a Mammalian HEK293 Cell.

Target specific integration (knock in/KI) was directed towards PP1R12C known as the AAVS1 Safe Harbor locus (CompoZ™ Targeted Integration Kit—AAVS1, SigmaAldrich) (See Table 8). A modified ObLiGaRe strategy (Maresca 2013) was used where two inverted ZFN binding sites flank the donor plasmid gene of interest to be knocked in (FIG. 6B). Firstly a shuttle vector designated EPB71-AAVS1-2X-Ins was synthesized (Plasmid map see FIG. 6A). EPB71-AAVS1-2X-Ins was designed in such a way, that any cDNA or DNA sequence encoding the full open reading frame of a glycosyltransferase or chimeric protein possessing a Golgi targeting and retention sequence fused with a catalytic enzyme said glycosyltransferase domain can be inserted into a multiple cloning site where transcription is initiated and driven by CMV IE promotor and terminated by a bGH terminator. In order to minimize epigenetic silencing, two insulator elements flanking the transcription unit were included. In addition a Safe Harbor #1 “landing pad” (SH #1 landing pad) was included just upstream of the 3′ inverted ZFN binding site FIG. 6A.

As an example, a full length ST6GAL1 open reading frame was inserted directionally into EPB71-AAVS1-2X-Ins generating EPB71-AAVS1-2X-Ins -ST6GAL1. Transfection and sorting of HEK293 cell clones was performed as described previously (Duda 2015). Clones were initially screened by positive SNA lectin staining and selected clones further analyzed by 5′ and 3′ junction PCR to confirm correct targeted integration event into AAVS1 site in HEK293. The allelic copy number of integration was determined by WT allelic PCR. Subsequently, full length human MGAT4A open reading frame was inserted directionally into 2nd AAVS1 allele of the HEK293 ST6GAL1 KI clone, followed by inserting full human MGAT5 to “SH landing pad” using same strategy as described above for ST6GAL1. The “landing pad” encodes the Safe Harbor #1 (SH1) sequence derived from the CHO genome, that has successfully been utilized by us (Duda 2015) and others for ZFN mediated target integration in CHO cells and thus represents a unique site when integrated in human cells devoid of this sequence. This allows for subsequent donor target integration into the SH1 site using CompoZ™ Targeted Integration Kit—CHO-SH #1, SigmaAldrich and a donor vector designated EPB69 (Plasmid map see FIG. 6C). The EPB69 donor vector allows for insertion of any gene of interest as described above for EPB71 flanked by inverted SH #1 ZFN binding sites instead of AAVS1 binding sites. EPB69 possesses a landing pad encoding AAVS1 ZFN binding site and since the endogenous AAVS1 binding sites are destroyed by target integration of the first KI construct, the EPB69 contained landing pad can be used for target integration of a third EPB71 KI contruct. In this way stacking of multiple KI constructs can be achieved. We also developed an alternative method for targeted delivery of genes based on CRISPR/Cas9 targeting using an approach designated Blunt end KI. As an example AAVS1 locus was targeted using gRNA AAVS1-1; 5′-ggggccactagggacaggattgg-3′. EPB71-AAVS1-2X-Ins -ST6GAL1 was used as template for donor amplication of CMVIE-ST6GalI-BgH ORF (ST6GalI amplicon) by HI-proof reading polymerase. Blunt end KI was achieved by transfecting cells with ST6Gall amplicon (1 ug), AAVS1-1 gRNA plasmid (2 ug) and Cas9 plasmid (2 ug) for 2 million cells. Target integration was confirmed by Junction-PCR flanking both donor and AAVS1 locus. This approach allows for blunt end KI or ObLiGaRe mediated donor integration into any of the non active HEK293 transcription units listed in Table 6.

TABLE 8
HEK293 Glyco-gene ZFN/Talen list
ZFN and Talen target gene sequences. For all these genes ZFN/Talen
constructs were generated and tested efficient for knock-out.

| Gene name | |
|---|---|
| (HGNC) | Target sequence |
| | HEK293 ZFN |
| AAVS1 | ACCCCACAGTGGggccacTAGGGACAGGAT |
| B3GNT2 | TCCCGAGGCATACTGGAAccgagaGCAAGAGAAGCTGAA |
| B4GALT5 | TGGAAGCCTTCTGATTGCatgccTCGGTGGAAGGTAGGGTG |
| B4GALT6 | CTGTCTGTACTTCATCTATgtggcCCCAGGCATCGGTAAGCA |
| B4GALT7 | GTCGGCGGCATCCTGCTGctctcCAAGCAGCACTACCGG |
| C1GALT1 | TACCTGTTTCCACTACttttttAGGGTCCTGGTTGCT |
| CDX2 | AACTTCGTCAGCCCCccgcagTACCCGGACTACGGCGGTT |
| COSMC | CCCCAACCAGGTAGTAgaaggctGTTGTTCAGATATGGCTGTT |
| GALNT1 | CTGGATGCCCATTGTgagtgtACAGTGGGATGGCTG |
| GALNT10 | GACCTTACCCCATGAccgatGCTGAGAGAGTGGATCAG |
| GALNT11 | GACCGCTTGGGCTACcacagaGATGTGCCAGACACAAGG |
| GALNT12 | CTTGAGACATCCCCGGATAtcctgCTAGAAGAAGTGATC |
| GALNT13 | AGCCTTTGCTGGCAAgaataAAGGAAGACAGGTAAGAA |
| GALNT14 | ACCTTCACCTACATCGAGtctgccTCGGAGCTCAGAGGGGGTG |
| GALNT2 | GTCGGCCCTACTCAGGACcgtggtCAGGTGAGGCCAGGAGAT |
| GALNT3 | TTCAACAAACCTTCTccttatGGAAGTAACCATAAC |
| GALNT4 | TTCATGCCTCCGCAGGAGccggccGTGCCAGGGAGCTGGGGTC |
| GALNT5 | CCAGTAATCGAAGTCATCaatgaTAAGGATATGAGGTA |
| GALNT6 | GGCCACCACAGGACCccaatgCCCCTGGGGCAGATGGAA |
| GALNT7 | CCTGTGGTACCATGGCctcatGTTGAAGGAGTAGAAGTG |
| GALNT8 | CATCCCCGACACGCGAGACtacaggTGGGATGAAcCAGGCT |
| GALNT9 | AGCCCGCACTGTCgcggaTCCGAGAGGACCGGCGTC |
| GALNTL1 | CCGAGTGGCTGCCGCCCATgctgcaGCGGGTGAAGGAGGTGAG |
| GALNTL2 | AGCGTCATCCTCTGTttccatGATGAGGCCTGGTCC |
| GALNTL5 | CTGGTGTTCCTGGACagccacTGTGAGGTGAACAGAG |
| GALNTL6 | TCCGGACCCGTCTCCTGGgggcaTCTATGGCCAGAGGAGAAG |
| KL | CCCTCTTCCCTTTGCAGCcatcaaGCTGGATGGGGTGGATGTC |
| MAPK15 | TACCGCAGCCGCGTCTATcaggtGCTCCGGCTCTCGAC |
| MBTPS1 | AACATCGCCCGCTTTtcttcAAGGGGAATGACTACCTG |
| MGAT3 | GTCTTCCTGGACCACTTCccgcccGGCGGCCGGCAGGACGGC |
| MGAT4A | CTTTGTCTTGGTATACTACatggcaAAATGGGAAAGGTAAGGA |
| MGAT5 | ATCCTGGACCTCAGCAAAaggtacATCAAGGCACTGGCAGAA |
| MSLN | CTCGAGGACCCTGGCtggagaGACAGGGCAGGTAAGGTC |
| MUC1 | CTGCTCCTCACAGTGCTTAcaggtGAGGGGCACGAGGTGGGG |
| MUC16 | AACATCCTCTTTGATTCCtggattAAGGGACACCAGGACGTC |
| NEU4 | GGCTTCCCAGCCCCCgccccCAACAGGCCACGGGATGAC |
| PCSK5 | TACCACTTCTACCATAGCaggacGATTAAAAGGTCAGTT |
| PCSK9 | CTACTCCCCAGCCTCAGCtcccgAGGTAGGTGCTGGGG |
| POMGNT1 | AGCCAAGGCTCTgctgaGGAGCCTGGGCAGCCAGG |
| ST3GAL1 | ATGATCCTGGTGCCCTTCaagaccATCGACTTGGAGTGGGTG |
| ST6GALNACI | CACAAAGACGACCCAAGGaaatggGGGCCAGACCAGGAA |
| ST6GALNACII | AGCCAACACAAAGCCccgtaTGGCTGGCGGGGGC |
| STT3A | GACCTGCAGCTCCTCGTCttcatGTTTCCAGGTATGTG |
| STT3B | TGCTGCAAGCTTATGCtttcttGCAGTATCTGAGAGACCG |
| TUSC3 | TTCTCCTGCTGCTGCTGCtctgcATCCAGCTCGGGGGAGGA |
| WBSCR17 | GGACGCCGGAGACCCTTCtctcccCATCAGGTCTGTGGCT |
| ST6GAL1 | ATGATCCTGGTGCCCTTCaagaccATCGACTTGGAGTGGGTG |
| ST3GAL2 | CCCTACCTGGACTCAGGGGccctggATGGGACGCACCGGGTGAA |
| B4GALT1 | CCTGGCTGGCCGCGACCTGagccgcCTGCCCCAACTGGTCGGA |
| ST3GAL3 | CAGGTTCTCCAAGCCagcaccCATGTTCCTGGATGAC |
| B4GALT3 | GACCCCCGGGGACCCCGCcatgttGCCGTTGCTATGAACAAG |
| B4GALT4 | GGCATCTACGTCATCcaccaGGTGAGCGTGGGGGCAGAC |
| ST3GAL4 | TCTGCCCACTTCGACcccaaaGTAGAAAACAACCCAGAC |
| ST6GAL2 | ACCTGCCATGAAACCACACttgaaGCAATGGAGACAACG |
| B3GNTL1 | CCGGAGGACCTGCTGTTCttctaCGAGCACCTCAGGAAGGG |
| B3GNT5 | CTCAGCGGGGCCTCGCtaccaaTACTTGATTAACCACAAG |
| | HEK293 Talen |
| COSMC | TGACTTATCACCCCAACCAGGTAgtagaaggctgttGTTCAGATATGGCTGTTACTTTTA |

### Example 5

Display of the Human Glycome on Human HEK293 Cells.

A plurality of HEK293 cells engineered as described in above examples express subsets of the human glycome on glycoconjugates found on the cell surface and/or shed/secreted into the culture medium. Probing biological interactions with the displayed glycome on cells may be carried out by a multitude of established experimental methods including but not limited to different types of immunocytology and fluorescence-activated cell sorting (FACS), adsorption, enzyme-linked immunosorbent assays (ELISA), radioimmunoassays, and any other assay capable of determining an interaction of a biological molecule, antibody, lectin, adhesin and/or pathogen to a cell. The preferred method is amenable for high-throughput (HTP) analysis of a large array of isogenic cells and with ability to quantitate the degree of biological interaction with cells.

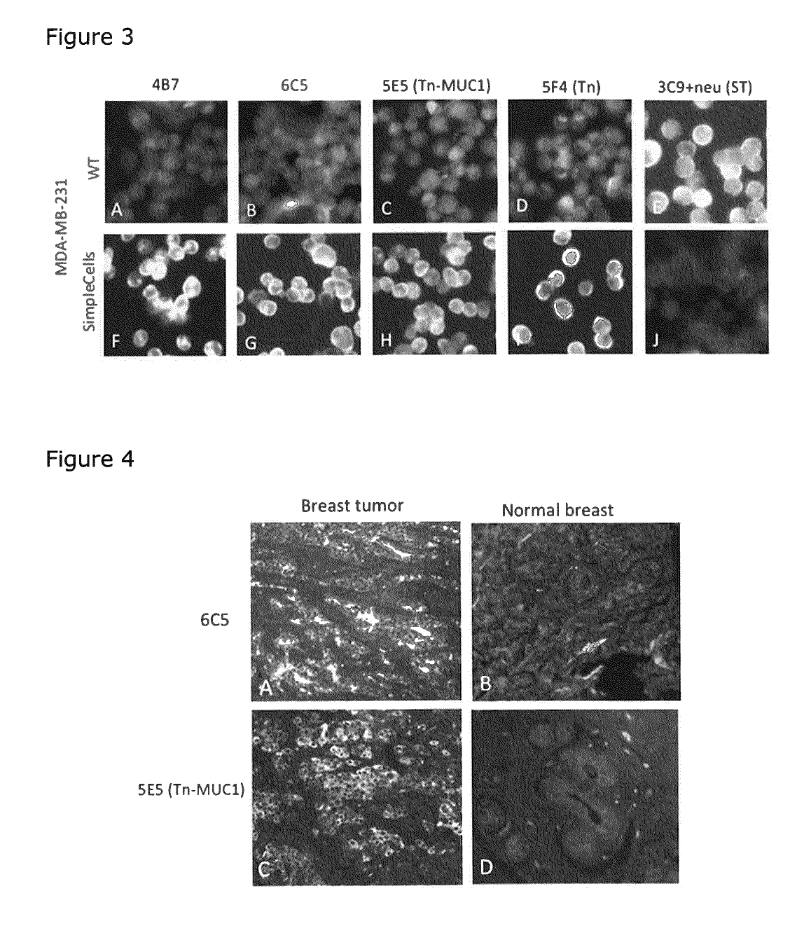

Here, the present inventors initially used immunocytology to demonstrate the feasibility to probe display of common glycan structures with glycan-binding antibodies, and to interpret defining structural and glycoconjugate features of the binding glycan from the differential binding patterns (FIGS. 3, 8, 9, 11 and 12). A plurality of HEK293 isogenic cells with combinatorial knock out and/or knock in of glycosyltransferase genes were reacted with a panel of glycan binding antibodies with known binding specificities. Examples from all steps in the combinatorial gene editing strategy outlined in EXAMPLE 1, FIG. 1A were included.

1. Targeting the initiation and glycoconjugate determination (step 1, group 1 genes). To demonstrate capability to use a cell-based HEK293 glycan display to decipher contribution of glycoconjugates to a biological interaction, a plurality of HEK293 cells with knock out of group 1 genes were used to display and probe with monoclonal antibody.

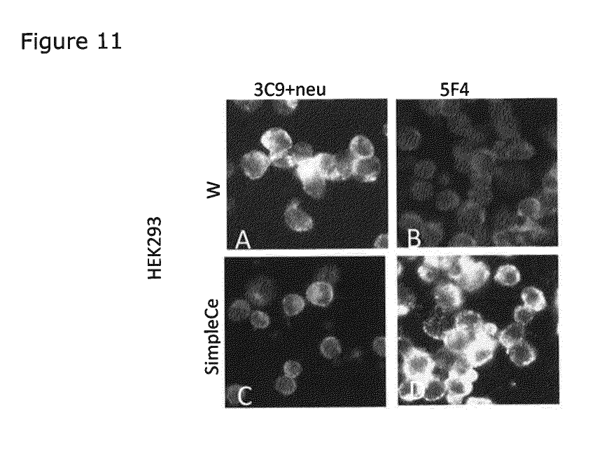

2. Targeting the second step in biosynthesis (step 2, group 2 genes). To demonstrate capability to use a cell-based HEK293 glycan display to decipher contribution of glycoconjugates and glycan structures to a biological interaction, a plurality of HEK293 cells with knock out of group 2 genes were probed with monoclonal antibodies (MAbs) 3C9 (anti-T) and 5F4 (anti-Tn) directed to O-GalNAc glycans found only on O-glycoproteins. As shown in FIG. 11 only HEK293 cells with knock out of C1GALT1 (and/or COSMC) were not stained by the 3C9 antibody, demonstrating that the binding interaction with the HEK293 cells were likely through an O-glycoprotein. Furthermore, As shown in FIG. 11 only HEK293 cells with knock out of C1GALT1 (and/or COSMC) were stained by the 5F4 antibody, demonstrating that the binding interaction with the HEK293 cells were likely through an O-glycoprotein. Both patterns of reactivity are in agreement with the determined binding specificities of these MAbs (Steentoft 2011).

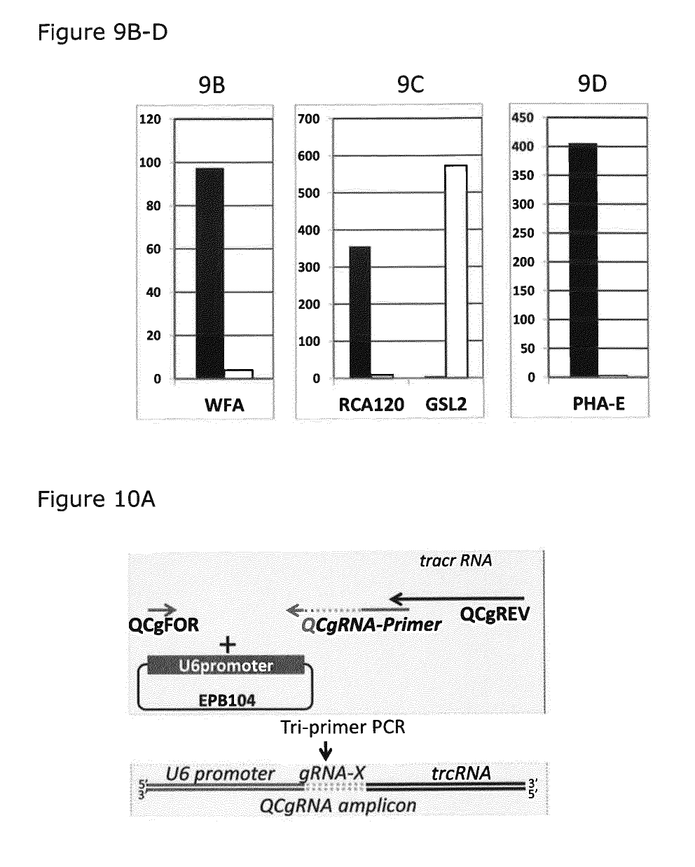

3. Targeting the elongation and branching steps (step 3, group 3 genes). To demonstrate capability to use a cell-based HEK293 glycan display to decipher contribution of elongation and/or branching of glycans on one or more types of glycoconjugates, a plurality of HEK293 cells with knock out of group 3 genes, including MGAT5, B4GALT1/2/3/4 and/or B4GALNT1/2. When probing MGAT5 ko cells with animal lectins PHA-L, which is preferentially reactive with tetraantennary N-glycans found on N-glycoproteins. As shown in FIG. 9A only HEK293 cells with knock out of MGAT5 were not stained by the PHA-L lectin, demonstrating that the binding interaction with the HEK293 cells were likely through an N-glycoprotein with loss of tetraantennary N-glycans. This pattern of reactivity is in agreement with previous studies. For addressing elongation of Lactose and LacDiNAc structures cells with knock-out of endogeneous B4GALT and B4GALNT capacities were used for display. More specifically the engineering targeted genes encoding B4GALT1/2/3/4 and/or B4GALNT1/2 which are involved in the synthesis/elongation of Lactose (Galβ1-4GlcNAc) and LacDiNAc (GalNAcβ1-4GlcNAc) structures. For obtaining glycostructures without LacDiNac, cells with knock out of B4GALNT3/4 were generated and analysed for lectin binding. The cells have lost binding to WFA lectin as shown in FIG. 9B demonstrating complete removal of LacDiNAc, whereas single gene knock outs only caused partial loss of LacDiNacs (not shown). For obtaining glycans with less elongated structures we did stacked knock out of B4GALT1/2/3/4 and analysed by lectin staining. As shown in FIG. 9C the cells lost binding to RCA120 lectin but gained binding to GSL2, demonstrating shortened glycostructures in which Galactose is lost (RCA120) concomitant with increased exposure of GlcNAc (GSL2). For obtaining reduced branching we engineered cells by knock out of MGAT5 and analysed by lecting binding. As shown in FIG. 9D the MGAT5 ko cells could not bind PHA-L lectin, demonstrating that branching was lost.

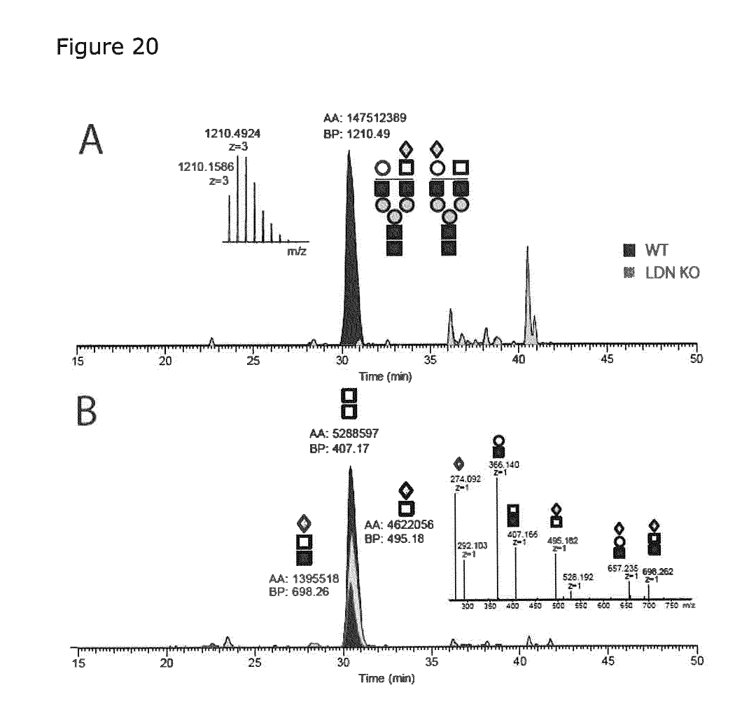

Using GLA enzyme as a secreted reporter protein we analysed LacDiNac by MSMS analysis. GLA enzyme was expressed in wt and engineered HEK cell lines and GLA glycopeptides were purified by ion exchange chromatography before digesting with chymotrypsin and analysis of glycopeptides by MSMS (procedures are described in more detail in Example 6A). The analysis showed that LacDiNac structures were completely eliminated when GLA was expressed in cells with double knock out of B4GALNT3/4 (FIG. 20A.

4. Targeting the capping step (step 4). To demonstrate capability to use a cell-based HEK293 glycan display to decipher contribution of capping of glycans on one or more types of glycoconjugates, a plurality of HEK293 cells with knock out of group 4 genes can be probed with animal lectins (MAL II and SNA) with preferential reactivity with α2,3 and α2,6 sialic acid capping, respectively, and MAbs (1B2) with preferential reactivity with unsubstituted poly-LacNAc chains found on N-glycoproteins and O-glycoproteins. Another example is knock-in of ST6GALNACT1 into HEK293 cells with COSMC knock out. The immunohistochemistry data shown in FIG. 12A demonstrate that Tn and STn capping can be modified by engineering group 4 genes.

By applying more extensive engineering designs glycans with exact tailored SA may be displayed. A multiplicity of HEK293 cells with single or multiple knock out of genes comprising ST3GAL1/2/3/4/5/6 and/or ST6GAL1/2 was investigated. By stacked knock out of ST3GAL1/2/3/4/5/6 and ST6GAL1/2 we could remove both and sialylations on N-glycans as well as on 0 glycans. This was demonstrated by lectin staining using MAL1, MAL2 and SNA lectins, which all lost binding to the engineered cells as shown in FIG. 12B. Cells displaying exclusively α2,6 sialylation and no α2,3 sialylation was obtained by stacked knock out of ST3GAL1/2/3/4/5/6 as shown in FIG. 12C where the specific lectins MAL1 and MAL2 did not stain the cells, whereas staining with SNA lectin, which is specific for sialylation, was unaffected. For obtaining exclusive sialylation and no sialylation cells with double knock out of ST6GAL1/2 were generated. As shown in FIG. 12D these cells had lost binding of SNA lectin but retained binding of MAL1 and MAL2 demonstration complete and selective loss of sialylation. For selective removal of sialylation on N-glycans cells with stacked knock out of ST3GAL3/4/6 and ST6GAL1/2 were generated. Cell staining shown in FIG. 12E show binding of MAL2 lectin, which is specific for glycans on O-glycostructures, whereas binding of the MAL1 and SNA lectins specific for sialylations on N-glycans was lost. This demonstrates that these cells only sialylate on O-glycans, whereas neither or sialylation was added onto N-glycans. For specific elimination of sialylation on N-glycans cells with knock out of ST3GAL3/4/6 were generated and analysed for lectin binding. As shown in FIG. 12F binding to MAL1 was lost whereas binding to MAL2 and SNA was retained demonstrating selective loss of sialylation without affecting sialylation on N-glycans or sialylation of O-glycans.

The result demonstrate that the cells had different sialylation capacities allowing cell surface display of a range of different sialylation glycoforms useful for probing protein-glycan interactions. The exact need for cell engineering to obtain specific capping events may be evaluated using more detailed engineering and display experiments, including knock out of subsets of indicated stacked ko designs and possibly ki of glycosyltransferases for optimal capping density and specificity. Such systematic display analysis may give glycovariants with improved drug features as described in Example 6.

The following Lectins were used (binding specificities indicated) to evaluate and illustrate effects of glycoengineering:

WFA (Wisteria floribunda) lectin recognize glycostructure terminating in GalNAc;

RCA (Ricinus communis Agglutinin) recognize glycostructures terminating in Galactose;

MAL1 (Maackia amurensis leukoagglutinin 1) recognize N-glycan structures terminating with α2,3 Sialic Acid;

MAL2 (Maackia amurensis leukoagglutinin 2) recognize O-glycan structures terminating with α2,3 Sialic Acid;

SNA (Sambucus nigra) lectin recognize glycan structures terminating with α2,6 Sialic Acid;

GSL2 (Griffonia simplicifolia) lectin recognize N-glycans terminating in GlcNAc

5. Targeting the glycan modifying step (step 5). To demonstrate capability to use a cell-based HEK293 glycan display to decipher contribution of modification of glycans on one or more types of glycoconjugates, a plurality of HEK293 cells with knock out of group 5 genes can be probed with suitable antibody or other assay.

6. To expand the glycan display of HEK293 cells to include glycosylation features not normally found in wildtype HEK293 cells and display the entire human glycome, the present inventors identified the group of glycosyltransferase genes not expressed in HEK293 (Table 6) that when introduced into HEK293 individually or in combination enhance the glycosylation and glycan modifying capability of HEK293 cells. To demonstrate that new glycosylation features are introduced, the present inventors used targeted insertion of a human sialyltransferase, ST6GALNACT1, and demonstrate that introduction of this induce display of the cancer-associated O-glycan STn only when combined with gene engineering to truncate O-glycans by knock out of C1GALT1 or COSMC (FIG. 12). Overexpression of ST6GALNACT1 in human cancer cell lines has been shown to induce heterogenous STn expression (Marcos 2011), but site-directed insertion of one or two copies of human ST6GALNACT1 driven by the CMV promotor does not override the normal O-glycosylation pathway in cells. Thus, the applied engineering strategy provides an improved display of homogeneous glycans required for use of the cell-based display technology and interrogation and identification of the glycan structure(s) involved in biological interactions.

HEK293 cells were fixed in ice-cold acetone for 5-8 min. Monoclonal antibodies were incubated overnight at 4° C. followed by incubation with FITC conjugated Rabbit anti-mouse Ig (F 0261, Dako, Denmark) for 40 min at RT. Slides were mounted with Vectashield (Vector labs, CA, USA) and examined in a Zeiss fluorescence microscope.

### Example 5b

A systematic approach for determining which glycomodification that influence a given activity, for example binding to a given virus, may comprise the following:
1. Determination of type of glycosylation is accomplished by generating a multiplicity of mammalian cells engineered in the 19 glycogenes involved in the truncation step, which is step 2 in FIG. 1 and genes are listed in FIG. 1 and Table 5). Two types of result will indicate that a certain type of glycosylation is responsible for the activity investigated, firstly truncation of the glycotype may abolish the activity or alternatively truncation of all other glycotypes does not affect the activity. After elucidating type of glycosylation the following steps may be run in parallel
2. After determination of type of glycosylation (from 1.) the relevant initiation type may be obtained by generating a multiplicity of mammalian cells with modification of genes involved in initiation, such initiation sub-arrays can be made for each type of glycosylation derived from 1. Thus the maximum number of initiation glycogenes to be investigated is 20, namely the 20 GALNT genes involved in O-GalNac type glycosylation (FIG. 2B). All genes involved in initiation of the various glycoforms are included in FIG. 2 panels A-F.
3. Role of elongation and branching for activity of the given type of glycosylation (from 1.) may be obtained by generating a multiplicity of mammalian cells with modification of genes involved in elongation and branching, the relevant sub-arrays can be made for each type of glycosylation. The maximum number of elongation and branching glycogenes to be investigated ranges 5-20 depending on type of glycosylation as evident from genes listed in FIG. 2 panels A-F.
4. For determining the role of capping of N- and O-glycans for activity a multiplicity of mammalian cells engineered in the 31 genes comprising the group 4 genes listed in Table 5 and in FIGS. 1-2. Loss of activity upon knock-out of one of these genes or combinations hereof suggest importance of the corresponding capping event for activity.
5. Role of non-GTf modifications is investigated by generating a multiplicity of mammalian cells with modification of the genes involved in non-GTf modifications for the particular type of glycosylation (Group 5 genes, Table 5). The maximum number of elongation and branching glycogenes to be investigated ranges 5-20 depending on type of glycosylation as evident from genes listed in FIG. 2 panels A-F.

### Example 6

Display of Different Glycoforms of Recombinant Expressed Human Proteins in HEK293 Cells.

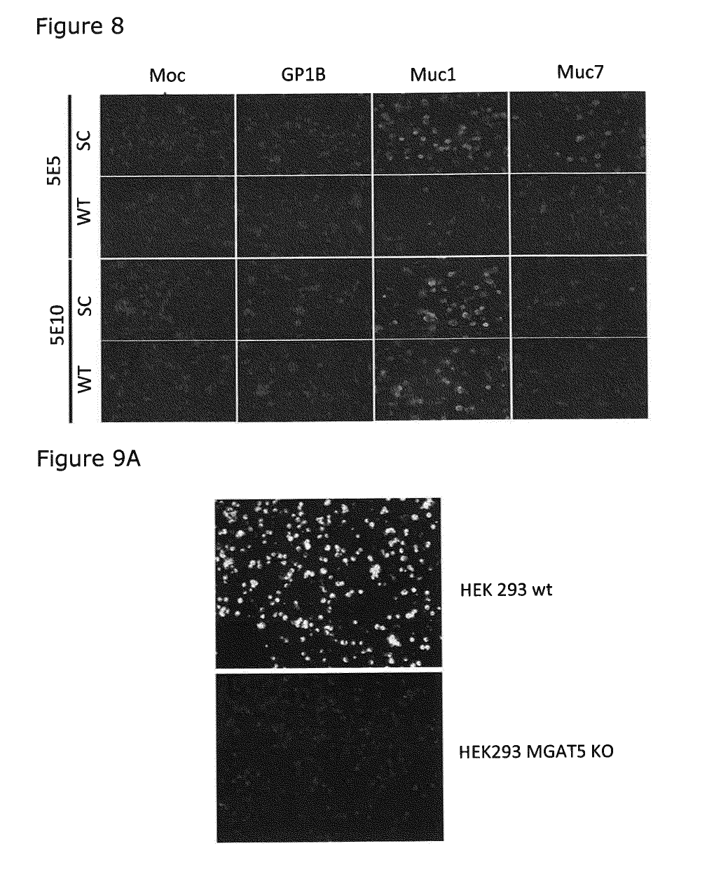

A plurality of HEK293 cells engineered as described in above examples have different glycosylation capacities, and are suitable for recombinant expression of human and other species proteins with different glycoforms to display such proteins on the cell surface and/or shed into the culture medium. An appropriate signal peptide is used to direct trafficking into ER and Golgi for glycosylation, and provided the gene encoding the protein of interest does not already have an efficient signal peptide. To demonstrate use of the cell-based for display of different glycoforms of a human protein not expressed in HEK293 cells, the present inventors used an expression construct encoding for the cell membrane bound mucin, MUC1. As shown in FIG. 8 MUC1 is displayed on the cell surface on a plurality of HEK293 cells and detectable by a MAb 5E10 reactive with all glycoforms of MUC1. In contrast, MAbs reactive with a subset of glycoforms such as MAb 5E5 that specifically reacts with the Tn glycoform of MUC1, are only reactive with HEK293 cells displaying MUC1 with the truncated Tn O-glycans. The binding reactivity of MAb 5E5 with the plurality of HEK293 cells with different gene engineering and glycosylation capacities, clearly indicates that the glycan on MUC1 interacting with the MAb is an O-glycan and of the Tn structure. This is in agreement with the previously determined binding specificity of this MAb (Tarp 2007).

To demonstrate that the cell-based array also can be used to develop protein arrays with proteins carrying glycans replicating the different glycosylation capacities of the plurality HEK293 cells, the present inventors analysed shed MUC1 found in the culture medium from the plurality of HEK293 cells expressing human MUC1. We used an ELISA capture assay with the MAb HMFG2 or 5E10 for capture and biotinylated MAb 5E5 for detection (Wandall 2010). The capture ELISA assay was performed using Nunc-Immuno MaxiSorp F96 plates (Nunc) coated with 1 ug/mL HMFG2 (MUC1) in carbonate-bicarbonate buffer (pH 9.6) overnight at 4° C. Plates were blocked with BSA/Triton X-100 buffer (1% BSA, 1% Triton X-100, 3 mM KCl, 0.5 M NaCl, and 8 mM phosphate buffer (pH 7.4)) for 1 h at RT and incubated with serially diluted spend culture medium and/or cell lysates from cell lines for 2 hrs at RT followed by washing. When neuraminidase treatment (0.1 U/mL Chlostridium Perfringes VI (Sigma) was performed for 1 hr at 37° C. a second blocking step was included for 15 min followed by washing. Culture supernatants of mouse MAbs IgG 5E5 and as controls for glycoforms IgM 3C9 (T), 5F4 (Tn) and 1B2 (poly-LacNAc) were applied for 1 hr followed by rabbit anti-mouse IgM HRP-conjugated antibody (Southern Biotech). Plates were developed with TMB+ one-step substrate system (Dako), reactions stopped with 0.5 M H2SO4, and read at 450 nm. As illustrated in FIG. 8 only MUC1 captured from medium of HEK293 cells with engineered capacity to produce Tn O-glycans was reactive. The same result was obtained using SDS-PAGE Western blot analysis.

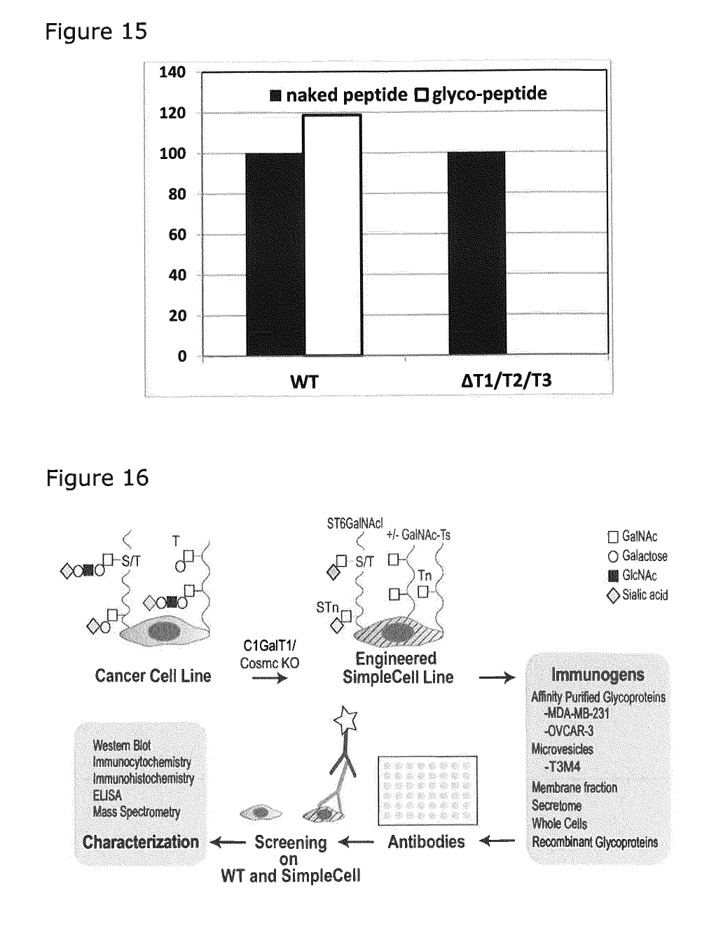

To further demonstrate that the cell-based array also can be used to analyse proteins carrying glycans replicating the different glycosylation capacities of the plurality of HEK293 cells, the present inventors analysed Erythropoietin (EPO) produced in HEK293 cell lines with knock out of GALNT1/2/3. As shown in FIG. 15 the EPO molecule completely lost O-glycans on the EPO glycopeptide EAISPPDAASAAPLR-131, which contains the O-glycan at S126, demonstrating that initiation of O-glycans on a secreted protein may be completely inhibited by defined glycoengineering. The cell based display procedure may be used to design optimal glycosylation capacities for modifying glycan initiation events at any site.

Using cell based display glycans may be displayed on the following types of proteins; Lysosomal enzymes including human iduronate 2-sulfatase (IDS), human arylsulfatase B (N-acetylgalactosamine-4-sulfatase) (ARSB), human lysosomal α-glucosidase (GAA), human alpha-galactosidase (GLA), human beta-glucuronidase (GUSB), human alpha-L-iduronidase (IDUA), human iduronate 2-sulfatase (IDS), human beta-hexosaminidase alpha (HEXA), human beta-hexosaminidase beta (HEXB), human lysosomal α-mannosidase (mannosidase alpha class 2B member 1) (MAN2B1), human glucosylceramidase (GBA), human lysosomal acid lipase/cholesteryl ester hydrolase (lipase A, lysosomal acid type) (LIPA), human aspartylglucosaminidase (N(4)-(beta-N-acetylglucosaminyl)-L-asparaginase) (AGA), and human galactosylceramidase (GALC).

Antibodies, IgG, IgG fragments, Ig fusion proteins, receptor-Fc fusion proteins, Bispecific IgG formats, Interleukin recepter fusion proteins

Anticoagulants/Coagulation Factors including coagulation factor II (2), coagulation factor V (F5), coagulation factor VII (F7), coagulation factor VIII (F8), coagulation factor IX (F9), coagulation factor X (F10), or coagulation factor XIII (F13), plasminogen activator tPA (PLAT), tPA fragments, erythropoietin (EPO)

Cytokines including Interferons alpha/beta/gamma (IFNA2, IFNA14, IFNB1, IFNG), Interleukin 2/11 (IL2, IL11), colony stimulating factor 2 (alias: granulocyte macrophage colony-stimulating factor) (CFS2), colony stimulating factor 3 (alias: granulocyte colony-stimulating factor) (CSF3) ( ), alpha-lantitrypsin (SERPINA1), plasma protease C1 inhibitor (alias: complement C1 esterase inhibitor) (SERPING1), anti-Thrombin (alias: Antithrombin-III) (SERPINC1), protein C (alias: autoprothrombin IIA) (PROC),human chorionic gonadotropin, alpha peptide (CGA), /Luteinizing hormone LH (LHB), Follicle-stimulating hormone FSH (FSHB), Thyroid-stimulating hormone TSH (TSHB), Glycodelin, progestagen-associated endometrial protein (PAEB), PDGF, Platelet-derived growth factor subunit A and B (PGDFA, PDGFB), TNFalpha, tumor necrosis factor (TNF), cytotoxic T-lymphocyte associated protein 4 (CTLA4) and fusion protein(s), VEGFR, vascular endothelial growth factor receptors 1/2 fusion protein, Bone morphogenetic protein 2, BMP-2 (BMP2), Dornase alpha or Pulmozyme (Human human deoxyribonuclease I, DNASE1)

### Example 6A

Lysosomal replacement enzymes represent an increasing group of therapeutic biologics essential for serious and rare congenital deficiencies. Many of these enzymes are effective in alleviating deleterious effects of these diseases in some organs but not in others including bone, heart, kidney and brain, and all enzymes are used in very high doses presenting a huge economic burden on society. Replacement enzymes are delivered intravenously and taken up by cells through different receptors that transport them to the lysosome, and N-glycan structures attached to these enzymes can hugely influence their organ targeting, speed of uptake and circulation. Accordingly there is a need for better glycan-display procedures for optimizing lysosomal replacement enzymes. Such procedure should display the enzymes with N-glycan structures which differ in parameters like degrees of sialic acid capping, type(s) of sialic acids (α2,3 vs α2,6 linkage), amount of M6P tagging, and exposed mannose. Ideally the display should also address different distribution of these features between the different glycosylation sites of the enzyme. Given the complexity of the glycoprocessing involved in synthesis, addition and site specificity of M6P on lysosomal enzymes a random approach like the cell based glycan array is optimal for investigating and optimizing the glycans on this class of enzymes.

To demonstrate that the cell-based array could be used to display lysosomal enzymes with different glycans in an interpretable fashion a display experiment using a model lysosomal enzyme was performed. Display of glycans on enzymes of pharmaceutical interest would facilitate development of glycovariant enzyme isoforms with improved drug features.

For optimizing glycans on lysosomal enzymes, a series of glycoengineered HEK293 cells were generated using CRISPR/Cas9 mediated gene modification to display glycans with most relevance for replacement lysosomal enzymes. The genes addressed were glycosyltransferases important for regulating N-glycan branching (MGAT1/2/4B/5, group 3 in Table 5), galactosylation (B4GALT1/3, group 3 in Table 5), terminal capping by sialylation (ST3GAL4/6/ST6GAL1, group 4 in Table 5) and core a6-fucosylation (FUT8, group 4 in Table 5). Furthermore we engineered the enzymes involved in N-glycan precursor trimming, including glucosidases (MOGS/GANAB) and mannosidases (MANEA/MAN1A1/1A2/1B1/1C1/2A1/2A2) (added) as well as enzymes involved in modifying the M6P tag (GNPTAB/GNPTG/NAGPA, group 5 in Table 5) and finally we addressed the N-glycan precursor synthesis pathway (ALG1/2/3/5/6/8/9/11/12/13/14, group 1 in Table 5). For knock-out and knock-in of genes the gRNAs shown in Table 7 were used for procedures described in Examples 3 and 4 respectively. The model enzyme chosen for the glycan display was alpha-galactosidase (GLA) which is the active pharmaceutical ingredient of two marketed replacement products for treating Fabry disease. The two products are Fabrazyme from Genzyme/Sanofi and Replagal from Shire. The GLA protein is a homodimer with 3 N-glycan sites on each subunit at Asn139, Asn192, and Asn215 (Lee 2003). For display of different glycans on the GLA enzyme it was expressed transiently in the glyco-engineered cell lines described above.

Expression constructs containing the entire coding sequence of human GLA was cloned into BamH1 site of the pTT5 expression vector (Durocher 2002). Engineered HEK293-6E cells were cultured in DMEM/high glucose medium supplemented with 10% FBS and 1% Glutamax. 60% confluent cells were seeded in T75 flasks the day prior to transfection. Plasmid was transfected into cells using PEI, by mixing 30 ul PEI (0.1% linear 25 k Polyethylenimin in 150 mM NaCl, pH 7.0) with 10 ug-GLA.pTT5 expression plasmid in 2 ml Opti-MEM Medium. One day after transfection, culture medium was changed to F17 Medium supplemented with 2% Glutamax and 1% TN1 (Tryptone N1). Culture supernatant was collected after incubating the cells at 37 C for another 2 to 3 days in F17 medium. Secreted GLA was purified from culture supernatant by ion exchange on a DEAE column. The culture supernatant was centrifuged at 3,000 g for 20 min and then further filtered for clarification through a 0.45 μm filter. After dilution with 3 volumes of 25 mM MES (pH 6.0) the resulting solution was loaded onto a DEAE sepharose fast-flow column pre-equilibrated with the same buffer. Elution was carried out by applying 0.2 M sodium chloride in 25 mM MES (pH 6.0) and the fractions containing the recombinant GLA were determined by enzyme activity assay and collected. Purity and rough titer of GLA was evaluated by Coomassie staining of SDS-PAGE gels.

For N-glycan profiling of purified GLA approximately 10 μg purified enzyme was reduced and alkylated followed by chymotrypsin digestion at a 1:25 chymotrypsin:protein ratio. Digests were loaded onto a stage tip containing 3 layers of C18 membrane. Glycopeptides were eluted with 50% MEOH in 0.1% formic acid, and then dried and re-solubilized in 0.1% formic acid. Samples were analyzed on an OrbiTrap Fusion MS (Thermo Fisher Scientific). Data processing was carried out using Proteome Discoverer 1.4 software (Thermo Fisher Scientific) with similar preprocessing and processing procedures. The exact masses of glycopeptide were subtracted from the corresponding precursor ion mass. Fragmentation spectra of candidate-matched glycopeptides associated with each protein were inspected to verify accuracy of sequence and site assignments.

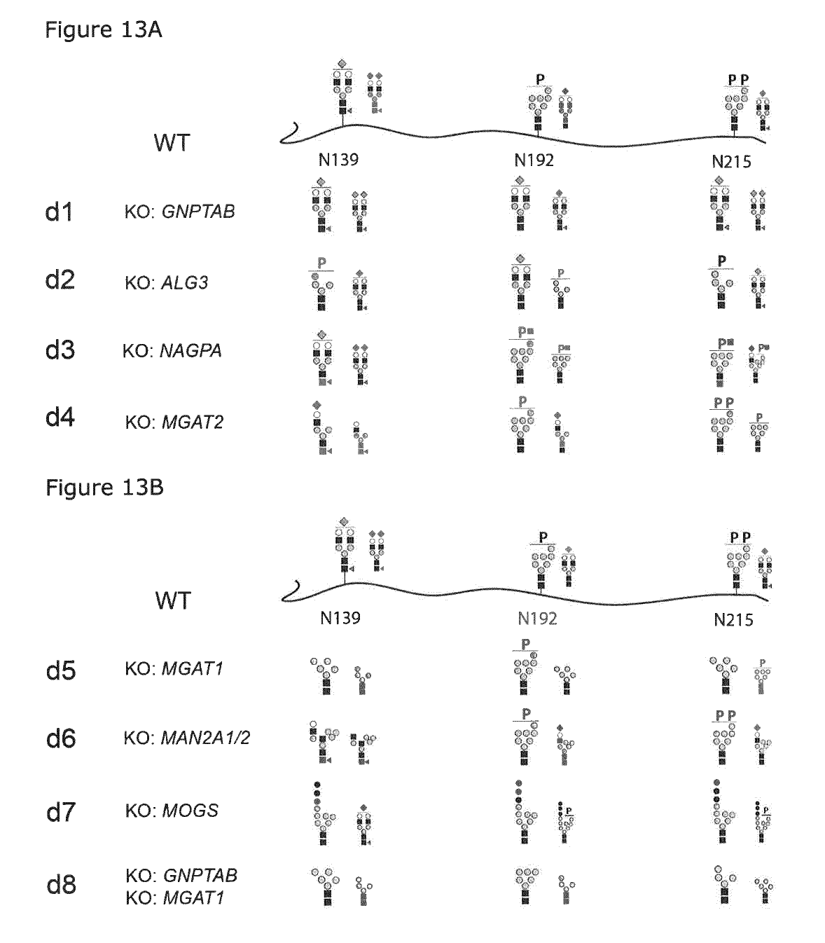

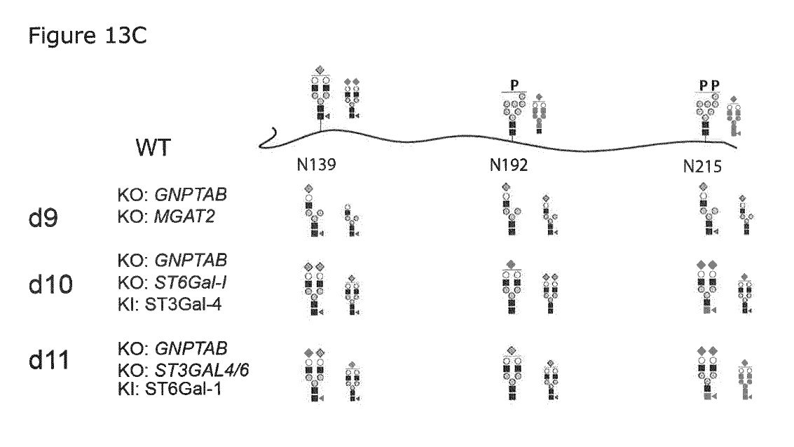

Sialic acid density and type of sialic acids, 2,3-linked or 2,6 linked, both influence circulating half-life of glycoproteins and for display of GLA enzyme with higher or changed sialic-acids we investigated cells with glycodesigns comprising various combinations of knockout or knockin of GNPTAB, ST3GAL4, ST3GAL6, ST6GAL1, MGAT1 and MGAT2. As shown in FIG. 13 we could dramatically change the sialic acids on the GLA enzyme. Knockout of GNPTAB resulted in enzyme without M6P glycans but dramatic increase in sialic acids (FIG. 13A-d1). Furthermore the high sialic acid content could be made homogeneous α2,3 linked SA by expressing GLA in cell lines with stacked knockout of GNPTAB and ST6GAL1 combined with knockin of ST3GAL4 (FIG. 13C-d10). For obtaining high content of homogeneous α2,6 linked sialylation on complex type N-glycans the GLA enzyme was expressed in cells with stacked knockout of GNPTAB, ST3GAL4 and ST3GAL6 combined with knockin of ST6GAL1 as shown in FIG. 13C-d11.

Simpler branching of N-glycans was obtained by double knock out of GNPTAB and MGAT2 that resulted in cells producing homogenous monoantennary structures with sialic acids and without M6P (FIG. 13C-d9).

M6P is critical for uptake of lysosomal enzyme drugs into target cells via M6P receptors. For displaying glycans with higher M6P content we generated cells with knock out of the ALG genes involved in synthesis of the N-glycan oligomannose structure. Using cell lines with single knockout of ALG3, ALG8 or ALG12 for displaying glycans on the GLA enzyme we achieved modification of both the overall M6P content and the site specific content as well as the general glycostructure. For example knockout of ALG3 resulted in increase in truncated high-mannose and hybrid structures with M6P tagging as shown in FIG. 13A-d2, where M6P become the dominant glycoform on both N-glycan sites 1 and 3. The occurrence of M6P onto the first site demonstrate that modifying glycogenes can drive M6P distribution on lysosomal enzyme in completely new ways.

Glycoproteins with exposed mannose residues will bind to Mannose receptors, which are predominantly found on macrophages and liver cells, resulting in specific targeting to these organs whereas circulation time in serum will be shortened. For modifying mannose structures we engineered cells by various knockout combinations involving the MGAT1, MOGS and GNPTAB genes. When expressing the GLA enzyme in MGAT1 knockout cells we obtained mannose and M6P structures only (FIG. 13B-d5) and the double knockout of MGAT1 and GNPTAB results in all three N-glycan sites having mannose structures without any M6P (FIG. 13B-d8)

Expression of the GLA enzyme in cells with knockout of the uncovering enzyme NAGPA resulted in increase of GlcNAc residues on the M6P tagged N-glycans (FIG. 13A-d3). When expressing the GLA enzyme in cells with knock out of the MOGS glycosidase we obtained high mannose type N-glycans with Glc residues and reduced M6P tagging of N-glycans (FIG. 13B-d7), and when the two mannosidase genes MAN2A1/2 were both knocked out in the cells the GLA enzyme showed hybrid N-glycans without changing M6P.

For the engineered cell lines the yield of GLA protein was similar to wt cells and galactosidase enzyme activity was not lost in any of the GLA preparations. In addition to the major trends shown for the glycoforms we observed minor glycan species with LacDiNAc or bisecting structures which may be avoided by additional combinatorial knock out of B4GALT3/4 (avoid LacDiNac) and/or knock out of MGAT3 (avoid Bisecting). Furthermore minor peaks of uncovered GlcNac structures on M6P were occasionally observed, probably due to somewhat low expression of NAGPA in our HEK293 cells. This may be optimized by knock in or overexpression of the NAGPA gene.

This example shows that the cell based display technology was readily applied to a secreted lysosomal enzyme. Site specific glycoanalysis of displayed GLA enzyme variants showed that using cell based display current investigators could induce dramatic changes of all glycan parameters critical for lysosomal delivery and circulation time. More specifically sialylation, M6P content, M6P distribution between sites, exposed Mannoses and branching of the glycostructures could be modified.

### Example 6b

Antibodies constitute the largest class of therapeutic biologics. Human IgG1s contain one conserved N-glycosylation site at N297. N-glycans on IgG1 are critical for their biological functions and glycoengineering has been applied to optimize ADCC functions. Thus, removal or lowering of the core fucose on the IgG glycans has proven effective for boosting ADCC effect (Yamane-Uhnuki 2004, Umana 1999). Lowering of fucose levels by way of gene engineering was originally obtained through a tour-de-force using two rounds of homologous recombination to eliminate both alleles of the fut8 gene in CHO (Yamane-Uhnuki 2004). The therapeutic IgG mogamulizumab currently in clinical use is produced in CHO cells with knock out of fut8. Reduction in the level of fucose on IgG has also been obtained by overexpression of MGAT3 (GnTIII) enzyme in CHO cells, which interferes with fut8 mediated fucosylation of N-glycans and results in production of IgG1 with minimal fucose glycoproteins with bisecting N-acetylglucosamine (GlcNAc) (Umana 1999), also for this strategy one antibody product, obinutuzumab (Gazyva), has now been marketed. More antibodies with low fucose content derived by these strategies or other approaches are currently in clinical trials for treating different cancers. However, there is a need for testing the role of distinct features of the N-glycans on IgG including branching status, exposed GlcNAc and galactose residues as well as sialic acid capping and the type of sialic acid linkage to evaluate biological effects.

To display different glycan structures on antibodies we expressed the human IgG1 antibody Trastuzumab (Herceptin) in glycoengineered cells by transient or stable expression as described in preceding Examples. Expression constructs containing the entire coding sequences of human IgG1, heavy and light chains were cloned into EcoR1/BamH1 site of the pTT5 transient expression vector (Durocher 2002). Capillary Electrophoresis laser-induced fluorescent detection (CE-LIF) was used for glycoprofiling. IgG was purified by protein G sepharose. HiTrap™ Protein G HP (GE Healthcare, US) was pre-equilibrated and washed in PBS and IgG was eluted with 0.1 M Glycine (pH 2.7). N-glycans from purified IgG were released by PNGase F (New England BioLabs), captured on MagnaBind Carboxyl Derivatized Beads (Thermo-Fischer) and labeled with 8-Aminopyrene-1,3,6-trisulfonate (APTS) (Sigma-Aldrich) before elution in water and run with formamide on a 3500XL Genetic Analyzer from Hitachi Applied Biosystems (Thermo Fischer).

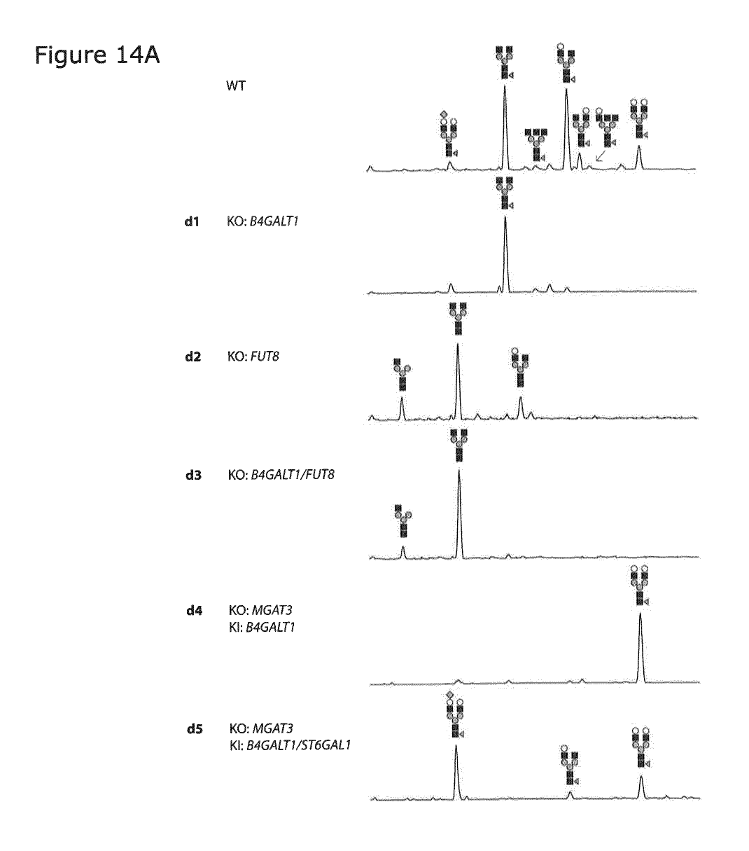

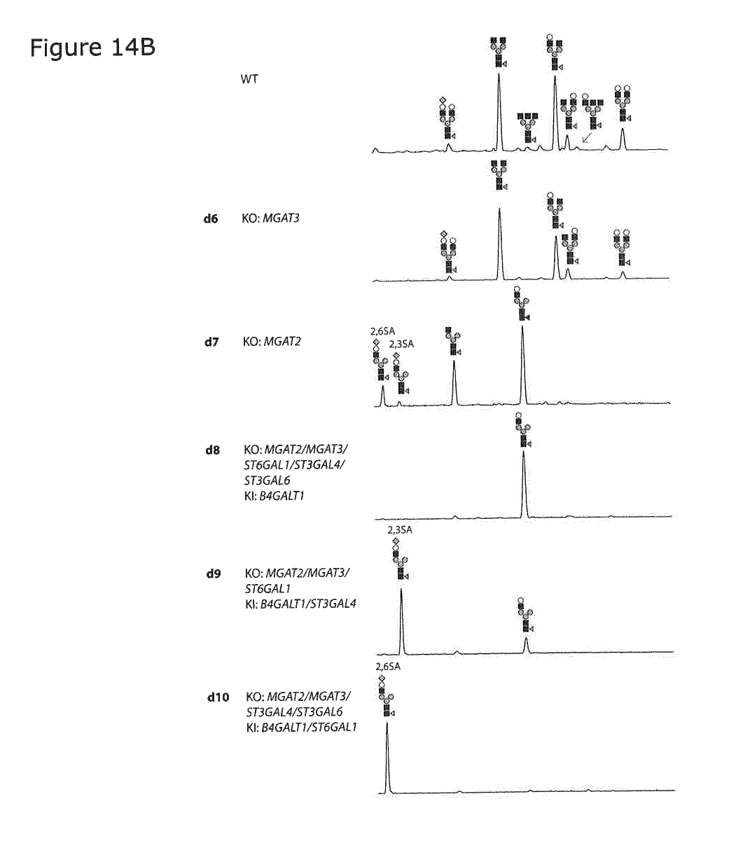

More homogeneous N-glycans with simpler glycoforms consisting of the pentasaccharide (Gn2Man3) with and without fucose (GOF/GO) were obtained with stable knock out of B4GALT1 and/or FUT8 genes, respectively (FIG. 14A, d1,d2,d3). Further knockout of MGAT3 increased homogeneity (FIG. 14B, d6).

Targeted KI of human B4GALT1 produced a highly homogeneous G2F glycoforms (FIG. 14A, d4) and in combination with knock out of FUT8 G2. Furthermore, surprisingly, additional KI of human ST6GAL1 produced IgG1 with highly homogeneous G2S1F (FIG. 14A, d5). Further knockout of FUT8 and MGAT3 increase homogeneity and eliminated fucose.

Knock out of MGAT2 as predicted resulted in heterogeneous monoantennary N-glycans on IgG1 (FIG. 14B, d7), and further knock out of MGAT3, ST6GAL1, ST3GAL4, and ST3GAL6 with and without knockin of B4GALT1 resulted in essentially homogeneous monoantennary G1 glycoform with and without fucose (FIG. 14B, d8). Moreover, further knock in of ST3GAL4 or ST6GAL1 resulted in G1S1 with α2,3 sialic acid capping or highly homogeneous G1S1 α2,6 sialic acid capping, respectively (FIG. 14B, d9,d10).

The genetic engineering of cells resulted in the display of highly diverse and in most cases highly homogeneous N-glycans on recombinant IgG1 suitable for testing and design of improved glycoforms of therapeutic antibodies.

### Example 6C

A systematic approach for determining which glycomodification that influence and improve activity of an lysosomal enzyme, may comprise the following:

Generating a multiplicity of mammalian cells with modification of genes resulting in glycoforms with modified (‘extreme’) N-glycans (high/low) with respect to the following parameters: M6P content, Sialic Acid content, Ratio between α2,3/α2,6 Sialic acids, and exposed Mannose content.

Express the enzyme in the glycoengineered cell lines and produce a multiplicity of glycovariants of the enzyme.

Screen the multiplicity of enzyme glycovariants for optimized drug effect in relevant in-vitro assay and/or animal model.

Identify which glycovariants (and glycodesigns) that have improved drug function.

Additional round(s) of glycoengineering and screening (steps 1-4) may be applied to secure optimal glycovariant candidate.

The optimization aims at identifying a glycovariant of the enzyme which ultimately give improved clinical performance with respect to one or more parameters including efficacy, dosing, potency, purity, less side-effects and better safety. The assays used for screening will monitor biomarkers/reporters for one or more of these parameters.

For an enzyme glycovariant with improved drug function a production cell line may be developed by transferring the glycodesign to any mammalian cell based production platform.

### Example 6D

A systematic approach for determining which glycomodification that influence and improve activity of a therapeutic IgG antibody, may comprise the following:

Generating a multiplicity of mammalian cells with modification of genes resulting in glycoforms with modified N-glycans with respect to branching, bisecting GlcNAc incorporation, galactosylation, capping by sialic acid and linkage, and fucosylation.

Express the IgG in the glycoengineered cell lines and produce a multiplicity of glycovariants of the antibody.

Screen the multiplicity of IgG glycovariants for optimized drug effect in relevant in-vitro assay and/or animal model.

Identify which glycovariants (and glycodesigns) that have improved drug function.

Additional round(s) of glycoengineering and screening (steps 1-4) may be applied to improve homogeneity and secure optimal glycovariant candidate.

The optimization aims at identifying a glycovariant of the IgG which ultimately give improved clinical performance with respect to one or more parameters including efficacy, dosing, potency, purity, less side-effects and better safety. The assays used for screening will monitor biomarkers/reporters for one or more of these parameters.

For an IgG glycovariant with improved drug function a production cell line may be developed by transferring the glycodesign to any mammalian cell based production platform.

### Example 7

Display of Different Glycoforms of Specific Domains of Human Proteins Using a Reporter Design in HEK293 Cells.

In this Example the present inventors sought to test if the cell-based array could be used to display specific domains of human proteins in a common reporter construct that targets the domain to the cell surface and displays this with different glycans in an interpretable fashion. Such a strategy would enable display of glycans on isolated domains of biological interest such as for example small domains of the large mucins with clustered O-glycans that are difficult to express as whole proteins due to their large size, folded domains in proteins like Notch with diverse types O-glycans, folded domains of large membrane proteins with N-glycans, and more. A list of human mucin tandem repeat domains is presented in Table 9.

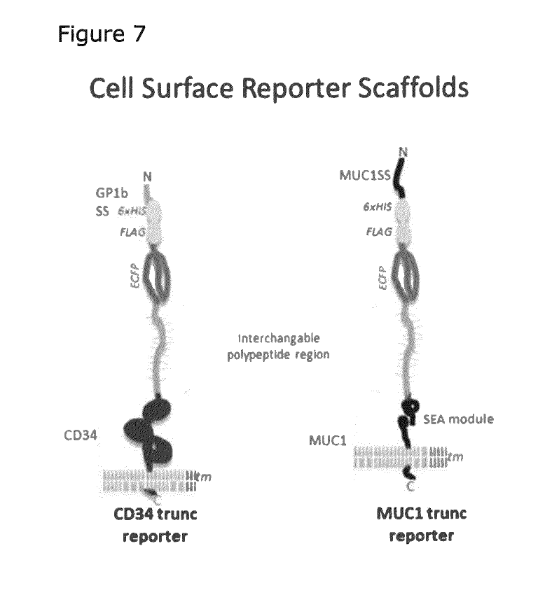

The present inventors designed and a reporter construct in the pcDNA3-neo (Invitrogen) vector synthesized by Genewiz, USA, encoding a chimeric type 1 transmembrane protein based on a signal peptide sequences derived from platelet GB1bα (amino acid 1-41) or MUC1 (amino acid 1-51) fused to enhanced cyan fluorescent protein (ECFP) linked to a interchangeable polypeptide region fused to the membrane anchoring domain of CD34 (amino acids 129-279) or MUC1 (ENST00000611571.4 amino acid 1039-1196), generating two designs of cell surface reporters (MUC1-CSR or CD34-CSR, FIG. 7). The reporter designs enable insertion of any polypeptide encoding DNA segment into the interchangeable polypeptide region (FIG. 7B). As an example, MUC1 tandem repeat fragment (amino acid 142-300; GenBank: AAA60019.1) was inserted into the interchangeable polypeptide region of both MUC1/CD34-CSR, generating MUC1-MUC1-CSR or MUC1-CD34-SCR (FIG. 7). 2 ug of either MUC1 reporter was transfected into a plurality of HEK293 cells with approximately 1.5×10⁶ cells in 200 ul BTX express using a BTX electroporator, a single 230V pulse followed by seeding into 3 ml medium in a 6well. 4 days post nucleofection G418 selection was applied to the cells for stable clone selection. Heterogeneous stable cell pools were selected and analyzed by immunocytology and/or FACS as described in previous Examples.

The following MAbs were used to detect display of MUC1 tandem repeats (5E10), the Tn-glycoform of MUC1 (5E5), the Tn glycan on any protein (5F4), and anti-FLAG MAb for detection and quantification of reporter expression. All HEK293 cell clones were reactive with anti-FLAG and the general MUC1 Mab (5E10), whereas only the HEK293 clones with COSMC knock out expressing Tn glycoforms were reactive with the Tn-MUC1 Mab (5E5) (FIG. 8). In general the reactivity was heterogeneous in the stable cell pools, but this was changed by single cell cloning where after the reactivity of the clones was homogeneous and correlated with ECFP fluorescence. Both MUC1-MUC1-CSR and MUC1-CD34-CSR reporters produced similar reactivity patterns with the MAbs tested, demonstrating similar cell surface display properties of the two reporter designs developed (FIG. 8).

### Example 7b

In this example the present inventors demonstrate the feasibility of the cell-based array for display of specific glycoforms of defined molecules expressed on the cell surface of glycoengineered cells. Such a strategy would enable display of defined glycans on defined domains of large molecules such as mucins with clustered O-glycans that are normally difficult to express molecules on the HEK293 cell surface. A full length MUC1 pcDNA3.1neo construct C-terminally fused to EYFP (Singh 2008) was transfected into HEK293wt or HEK293SC. 3 days post transfection, cells were trypsinized and dried on teflon coated slides followed by IHC (Mandel 1999) using 5E5 and 5E10 as primary antibodies as described above, followed by incubation with Alexa546 coupled rabbit anti-mouse secondary anti-bodies, as previously described. As shown in FIG. 8 the general MUC1 monoclonal 5E10 reacted with both transiently expressing MUC HEK293wt and SC cells. In contrast, 5E5 only reacted with transiently MUC1 expressing HEK293SC's and not HEK293wt cells, thus demonstrating the usefulness of the glycoengineered cells in display of natural full length cell surface molecules carrying defined glycan structures.

TABLE 9
Display of the Human Mucinome
Sequences of human mucins and mucin domains of glycoproteins used for design of
reporter constructs to display the characteristic tandem repeat regions of mucins
carrying high density of O-glycans. These sequences were introduced in the
reporter construct described in FIG. 7, and reporter constructs expressed in a
multiplicity of glycoengineered HEK293 cell lines.

| ORF | Construct | Encoded AA sequence |
|---|---|---|
| GP1BA | EPB105 | PTLGDEGDTDLYDYYPEEDTEGDKVRATRTVVKFPTKAHTTPWGLFYSW |
| | | STASLDSQMPSSLHPTQESTKEQTTFPPRWTPNFTLHMESITFSKTPKST |
| | | TEPTPSPTTSEPVPEPAPNMTTLEPTPSPTTPEPTSEPAPSPTTPEPTSEPA |
| | | PSPTTPEPTSEPAPSPTTPEPTPIPTIATSPTILVSATSLITPKSTFLTTTKPV |
| | | SLLESTKKTIPELD |
| MUC1 | EPB109a | APDTRPAPGSTAPPAHGVTSAPDTRPAPGSTAPPAHGVTSAPDTRSAPG |
| | | STAPAAHGVTSAPDTRSVPGSTAPQAHGVTSAPDTRPAPGSTAPPAHGV |
| | | TSAPDTRPVPGSTAPPAHGVTSAPDTRPAPGSTAPPAHGVTSAPDTRPA |
| | | PGSTAPQAHGVTS |
| MUC7 | EPB109b | PPTPSATTQAPPSSSAPPETTAAPPTPPATTPAPPSSSAPPETTAAPPTPSA |
| | | TTPAPLSSSAPPETTAVPPTPSATTLDPSSASAPPETTAAPPTPSATTPAPP |
| | | SSPAPQETTAAPITTPNSSPTTLAPDTSETSAAPTHQTTTSVTTQTTTTKQ |
| | | PTSAP |
| MUC2 | EPB110 | SPPPTSTTTLPPTTTPSPPTTTTTTPPPTTTPSPPITTTTTPPPTTTPSPPIST |
| (TR1) | | TTTPPPTTTPSPPTTTPSPPTTTPSPPTTTTTTPPPTTTPSPPTTTPITPPAST |
| | | TTLPPTTTPSPPTTTTTTPPPTTTPSPPTTTPITPPTSTT |
| MUC2 | EPB111 | TQTPTTTPITTTTTVTPTPTPTGTQTPTPTPITTTTTVTPTPTPTGTQTPTST |
| (TR2) | | PITTTTTVTPTPTPTGTQTPTMTPITTTTTVTPTPTPTGTQTPTTTPISTTTT |
| | | VTPTPTPTGTQTPTSTPITTTTTVTPTPTPTGTQTPTTTPITT |
| MUC3A | EPB112 | EISSHSTPSFSSSTIYSTVSTSTTAISSLPPTSGTMVTSTTMTPSSLSTDIP |
| (TR1) | | FTTPTTITHHSVGSTGFLTTATDLTSTFTVSSSSAMSTSVIPSSPSIQNTE |
| | | TSSLVSMTSATTPNVRPTFVSTLSTPTSSLLTTFPATYSFSSS |
| MUC3A | EPB113 | MTSATTPNVRPTFVSTLSTPTSSLLTTFPATYSFSSSMSASSAGTTHTESI |
| (TR2) | | SSPPASTSTLHTTAESTLAPTTTTSFTTSTTMEPPSTTAATTGTGQTTFTS |
| | | STATFPETTTPTPTTDMSTESLTTAMTSPPITSSVTSTNTVT |
| MUC3A | EPB114 | TTPTPTTDMSTESLTTAMTSPPITSSVTSTNTVTSMTTTTSPPTTTNSFTS |
| (TR3) | | LTSMPLSSTPVPSTEVVTSGTINTIPPSILVTTLPTPNASSMTTSETTYPNS |
| | | PTGPGTNSTTEITYPTTMTETSSTATSLPPTSPLVSTAKTAKTPTTNL |
| MUC3A | EPB115 | TTTETTSHSTPGFTSSITTTETTSHSTPSFTSSITTTETTSHDTPSFTSSIT |
| (TR4) | | TSETPSHSTPSSTSLITTTKTTSHSTPSFTSSITTTETTSHSAHSFTSSITT |
| | | TETTSHNTRSFTSSITTTETNSHSTTSFTSS |
| MUC4 | EPB116 | LPVTSPSSASTGHATPLLVTDTSSASTGHATPLPVTDASSVSTDHATSLP |
| (TR) | | VTIPSAASTGHTTPLPVTDTSSASTGQATSLLVTDTSSVSTGDTTPLPVT |
| | | STSSASTGHVTPLHVTSPSSASTGHATPLPVTSLSSASTGDTM |
| MUC5AC | EPB117 | TTSAPTTSTTSAPTTSTISAPTTSTTSATTTSTTSAPTPRRTSAPTTSTISA |
| | | STTSTTSATTTSTTSATTTSTISAPTTSTTLSPTTSTTSTTITSTTSAPISST |
| | | TSTPQTSTTSAPTTSTTSGPGTTSSPVPTTSTTSAPTT |
| MUC6 | EPB118 | TSATSSRLPTPFTTHSPPTGTTPISSTGPVTATSFQTTTTYPTPSHPHTTLP |
| (TR1) | | THVPSFSTSLVTPSTHTVIIPTHTQMATSASIHSMPTGTIPPPTTIKATGS |
| | | THTAPPMTPTTSGTSQSPS |
| MUC6 | EPB119 | SIHSMPTGTIPPPTTIKATGSTHTAPPMTPTTSGTSQSPSSFSTAKTSTSL |
| (TR3) | | PYHTSSTHHPEVTPTSTTNITPKHTSTGTRTPVAH |
| MUC9 | EPB120 | AMTMTSVGHQSMTPGEKALTPVGHQSVTTGQKTLTSVGYQSVTPGEKT |
| | | LTPVGHQSVTPVSHQSVSPGGTTMTPVHFQTETLRQNTVAP |
| MUC13 | EPB121 | TTETATSGPTVAAADTTETNFPETASTTANTPSFPTATSPAPPIISTHSSS |
| | | TIPTPAPPIISTHSSSTIPIPTAADSESTTNVNSLATSDIITASSPNDGLIT |
| | | MVPSETQSNNEMSPTTEDNQSSGPPTGTALLETSTLNST |
| MUC17 | EPB122 | LSTTPVDTSTPVTNSTEARSSPTTSEGTSMPTSTPSEGSTPFTSMPVSTM |
| | | PVVTSEASTLSATPVDTSTPVTTSTEATSSPTTAEGTSIPTSTLSEGTTPL |
| | | TSIPVSHTLVANSEVSTLSTTPVDSNTPFTTSTEASSPPPTAEGTSMP |
| MUC19 | EPB123 | VTRTTRSSAGLTGKTGLSAGVTGKTGLSAEVTGTTRLSAGVTGTTGPSP |
| | | GVTGTTGTPAGVTGTTELSAGVTGKTGLSSEVTETTGLSYGVKRTIGLSA |
| | | GSTGTSGQSAGVAGTTTLSAEVTGTTRPSAGVTGTTGLSAEVTEITGISA |
| MUC20 | EPB124 | ESSASSDSPHPVITPSRASESSASSDGPHPVITPSRASESSASSDGPHP |
| | | VITPSRASESSASSDGPHPVITPSRASESSASSDGPHPVITPSRASESSA |
| | | SSDGPHPVITPSRASESSASSDGPHPVITPSRASESSASSDGPHPVITPS |
| | | RA |
| MUC21 | EPB125 | SGASTATNSDSSTTSSGASTATNSDSSTTSSEASTATNSESSTTSSGAS |
| | | TATNSESSTVSSRASTATNSESSTTSSGASTATNSESRTTSNGAGTATN |
| | | SESSTTSSGASTATNSESSTPSSGAGTATNSESSTTSSGAGTATNSESS |
| | | TV |
| MUC22 | EPB126 | GTTTASMAGSETTVSTAGSETTTVSITGTETTMVSAMGSETTTNSTTSS |
| | | ETTVTSTAGSETTTVSTVGSETTTAYTADSETTAASTTGSEMTTVFTAGS |
| | | ETITPSTAGSETTTVSTAGSETTTVSTTGSETTTASTAHSETTAASTMG |
| mGp1ba | EPB127 | TSSGDTDYDDYDDIPDVPATRTEVKFSTNTKVHTTHWSLLAAAPSTSQD |
| | | SQMISLPPTHKPTKKQSTFIHTQSPGFTTLPETMESNPTFYSLKLNTVLIP |
| | | SPTTLEPTSTQATPEPNIQPMLTTSTLTTPEHSTTPVPTTTILTTPEHSTIPV |
| | | PTTAILTTPKPSTIPVPTTATLTTLEPSTTPVPTTATLTTPEPSTTLVPTTATL |
| | | TTPEHSTTPVPTTATLTTPEHSTTPVPTTATLTTPEPSTTLTNLVSTISPVLT |
| | | TTLTTPESTPIETILEQFFTTELTLLPTLESTTTIIPEQN |

### Example 8

Display of Homogeneous Cancer-Associated Glycomes and Use of Cells Displaying these for Generation and Discovery of Antibodies with Cancer-Associated or Cancer-Specific Reactivity.

Aberrant glycosylation is a hallmark of cancer and most types of human glycosylation known are affected in various ways. A large number of antibodies with reactivity to aberrant glycans expressed mainly or exclusively in cancer have been produced and characterized in the last 3-4 decades, and the vast majority of these react with immature and truncated glycans that are normal biosynthetic intermediates in the normal biosynthetic pathways of glycans. However, more recently it has emerged that antibodies may react with certain truncated glycans in the context of a specific protein or protein sequence and such MAbs have surprisingly high affinities and specific reactivity with cancer (Tarp 2008). The clearest example of this class of antibodies is the 5E5 MAb with specificity for Tn-MUC1 as demonstrated in the previous examples (Tarp 2007). This and similar antibodies to human Tn-glycoproteins have all been generated using rather homogeneous glycopeptide immunogens (Tarp 2008), and selection of such antibodies using whole cells or cell extracts have in general failed. The present inventors reasoned that the major obstacle for selection of such antibodies rested in two factors: i) the general heterogeneity found in glycosylation in cancer cells, where individual proteins are displayed literarily with hundreds of different glycans limits the possibility for stimulation of specific antibodies to such aberrant glycoprotein epitopes; and ii) the difficulty in screening and selection of antibodies reactive with aberrant glycoprotein epitopes without availability of homogeneous antigens for screening. The present inventors therefore developed and tested if the cell-based glycan display technology could be used to induce and select specific antibodies to aberrant glycoprotein epitopes. A general scheme for the comprehensive strategy is depicted in FIG. 16.

The present inventors first tested if they could generate a MAb with 5E5 characteristics by immunizing mice with a human cell displaying a homogenous O-GalNAc glycoproteome with Tn glycans as well as the MUC1 membrane protein. The present inventors used HEK293 cells engineered to only display Tn O-glycans, and transfected these with the full coding MUC1 pCDNA3 construct described in above Examples, as well as a human breast cancer cell line MDA-MB-231 engineered to only display Tn O-glycans that endogenously express MUC1.

The present inventors next tested immunization with Tn-glycoproteins extracted from cell lysates or culture medium of the engineered MDA-MB-231 by VVA lectin chromatography. As illustrated in FIG. 3 immunization with VVA enriched extracts of the MDA-MB-231 cell line engineered to display only Tn O-glycans led to generation of a novel MAb 4B7 that was shown to react specifically with MUC1 carrying Tn-glycans. Another novel MAb designated 6C5 that was selected based on a plurality of parental cells engineered to display aberrant glycans. 6C5 showed a high degree of cancer specificity as exemplified by tissue staining as shown in FIG. 4.

The present inventors next tested immunization with exosomal fractions and membrane factions collected by ultracentrifugation from the pancreatic cell line T3M4 engineered to only display STn/Tn O-glycans. Both exosome and membrane fraction generated strong polyclonal responses.

The present inventors next tested immunization with extracts from other cell lines engineered to only display STn/Tn O-glycans including the gastric cancer cell line AGS and the ovarian carcinoma cell line OVCAR-3.

Two major classes of immunogens can be generated from the engineered SC; i) different cell extracts including affinity purified glycoproteins, membrane extracts, microvesicles, secretomes and whole cells or ii) or recombinant expressed and purified glycoproteins (FIG. 16). In the current study we used affinity enriched cell lysates from breast (MDA-MB-231) and ovarian (OVCAR-3) cancer SimpleCell lines as well as microvesicles purified by sequential centrifugation steps of conditioned medium from a pancreatic cancer (T3M4) SimpleCell. The affinity enriched lysates were isolated by Triton-X100 extraction followed by lectin chromatography using the Tn-binding lectin Vicia Villosa (VVA) and elution with GalNAc. While MDA-MB-231 SC only express the Tn-glycoform, OVCAR-3 SC express a mixture of STn and Tn and was therefore neuraminidase treated prior to lectin binding. After mouse immunization and hybridoma fusion the obtained antibodies were screened by immunocytochemistry on trypsinated acetone fixed SC and the isogenic WT and antibodies with preferred SC reactivity was selected. Using microvesicles as an immunogen we obtained a significant number of hybridoma wells producing Abs with strong binding to the T3M4 SC (146 out of 480 wells) however only clone, 5D10, did not also react with T3M4 WT cells. When using lectin enriched lysates as the immunogen source we obtained less Ab producing hybridomas however multiple clones with preferred SC reactivity were isolated (8 out of 41 and 5 out of 15 SC positive wells for MDA-MB-231 SC and OVCAR-3 SC respectively). We selected one hybridoma well from each fusion for subcloning and thus generated mAb 6C5 (MDA-MB-231), 1D5 (OVCAR-3) and 5D10 (T3M4). All three mAbs were IgG1 and exhibited strong binding to the respective SC and no binding to the corresponding WT. We selected mAb 6C5 for further characterization including Western blotting, immunocytochemistry (ICC) on a panel of cancer SimpleCells, immunohistochemistry (IHC) on cancer and normal tissue followed by antigen identification as described below.

Characterization of mAb 6C5

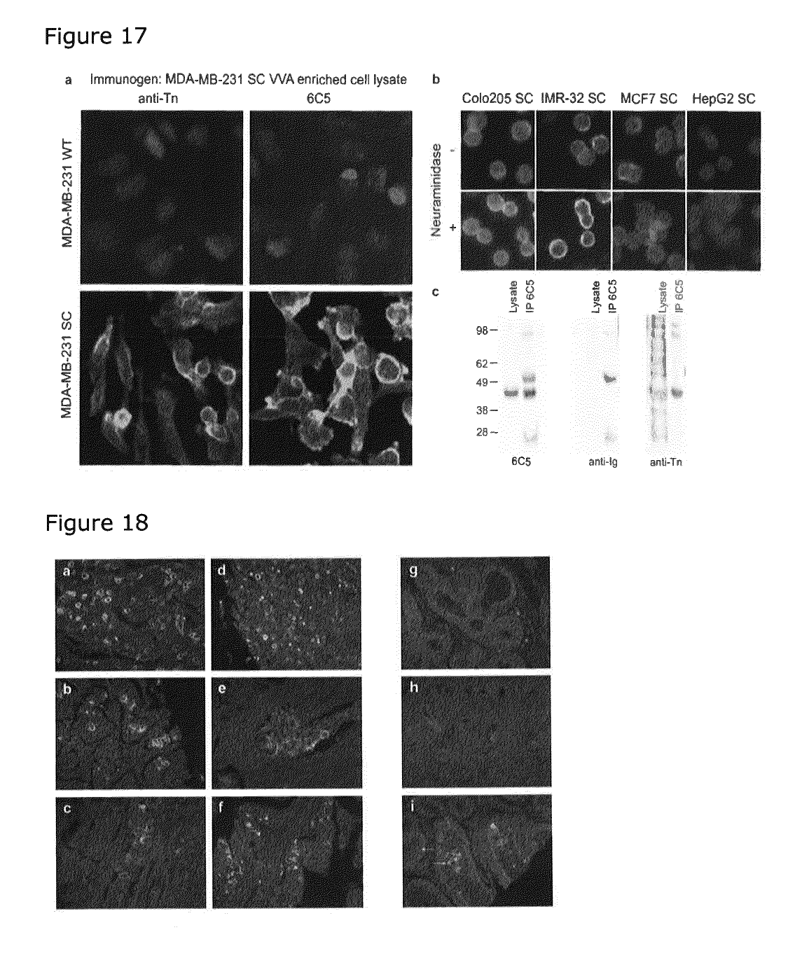

As an initial characterization we used mAb 6C5 to stain our panel of SCs from various tissue origin including HEK293, T3M4, HeLa, Colo-205, IMR-32, MCF7 and HepG2 with and without neuraminidase treatment to remove sialic acid. While most SC lines were stained strongly by mAb 6C5, MCF7 SC only displayed very faint staining and HepG2 SC were completely negative. This experiment confirmed that mAb 6C5 is not a Tn-hapten antibody, but on the contrary recognizes a Tn-glycoprotein antigen either differentially expressed or dependent of differentially expressed GALN-Ts. Neuraminidase seemed to enhance the staining of 6C5 indicating that the preferred glycoform is Tn which is consistent with the fact that MDA-MB-231 SC used for immunization does not express the STn glycoform48. Western blot of MDA-MB-231 SC cell lysates as well 6C5-immunopreciptated (IP) lysate showed that 6C5 recognizes a ˜50 kDa protein that is Tn-glycosylated as validated by VVA staining (FIG. 17). Moreover, mAb 6C5 also immunopreciptated (IP) the same 50 kDa band.

An immunohistological study using mAb 6C5 was performed on three tissue microarrays (TMAs) representing paraffin-embedded cores from four different types of breast cancer, three different types of ovarian cancer and adenocarcinomas of stomach as well as normal appearing tissue adjacent to cancer. The result is summarized in Table 10 and representative images displayed in FIG. 18.

Staining revealed that all three cancers were positive with mAb 6C5. Breast cancer had the highest number of positive cores (carcinoma simplex: 14/25, atypical medullary carcinoma: 6/13, infiltrating duct carcinoma: 6/13 and scirrhous carcinoma: 7/12). Ovarian cancer had less positive cores (serous papillary cyst adenomas: 10/47, mucinous carcinomas: 3/6, and endometrioid adenocarcinomas: 4/7). In stomach 7/22 adenocarcinomas were stained. The percentage of positive cells in all the tested tumors varied from less than 30% to more than 60% (Table 1).

TABLE 10
Summary of immunohistology with mAb 6C5.

| | | Negative | <30% | 30-60% | >60% |
|---|---|---|---|---|---|
| Tissues | Cases | #(%) | #(%) | #(%) | #(%) |
| Breast | | | | | |
| Cancer | 68 | 30(44%)¹ | 21(31%) | 10(15%) | 7(10%) |
| Normal | 8 | 8(100%)² | 0(0%) | 0(0%) | 0(0%) |
| Ovary | | | | | |
| Cancer | 59 | 39(66%)³ | 18(31%) | 1(2%) | 1(2%) |
| Normal | 10 | 10(100%) | 0(0%) | 0(0%) | 0(0%) |
| Stomach | | | | | |
| Cancer | 22 | 15(68%)⁴ | 3(14%) | 2(9%) | 2(9%) |
| Normal | 45 | 45(100%)⁵ | 0(0%) | 0(0%) | 0(0%) |

The tissue microarray cores were classified according to cell surface membrane staining. ¹1 core had granular intracellular staining. ²2 cores had granular intracellular staining. ³11 cores had granular intracellular staining. ⁴4 cores had granular intracellular staining. ⁵15 cores had strong Golgi-like staining of mucous producing cells. Four of those also had homogeneous staining throughout the cytoplasm.

The staining pattern observed with mAb 6C5 was mainly membraneous and cytoplasmic, although a subset of the three cancers only showed a weak punctuate granular intracellular staining (Table 10). In a few cancer cores mAb 6C5 labelled vascular endothelium and single dispersed cells possibly representing immune cells or detached cancer cells.

The tested TMAs also contained cores representing normal appearing tissues adjacent to cancer. Eight normal breast cores were examined, six of them were completely negative (FIG. 18) while two cores showed staining with mAb 6C5 although restricted to a very faint granular intracellular staining in few cells. Normal appearing ovarian tissues was completely negative (10/10). In normal appearing stomach strong intracellular Golgi-like staining was seen with mAb 6C5 in mucous producing cells (15/41) and in 4 of those cores a small fraction of mucous producing cells stained homogenously throughout the cytoplasm. No vascular endothelium staining was observed in any of the normal tissue cores

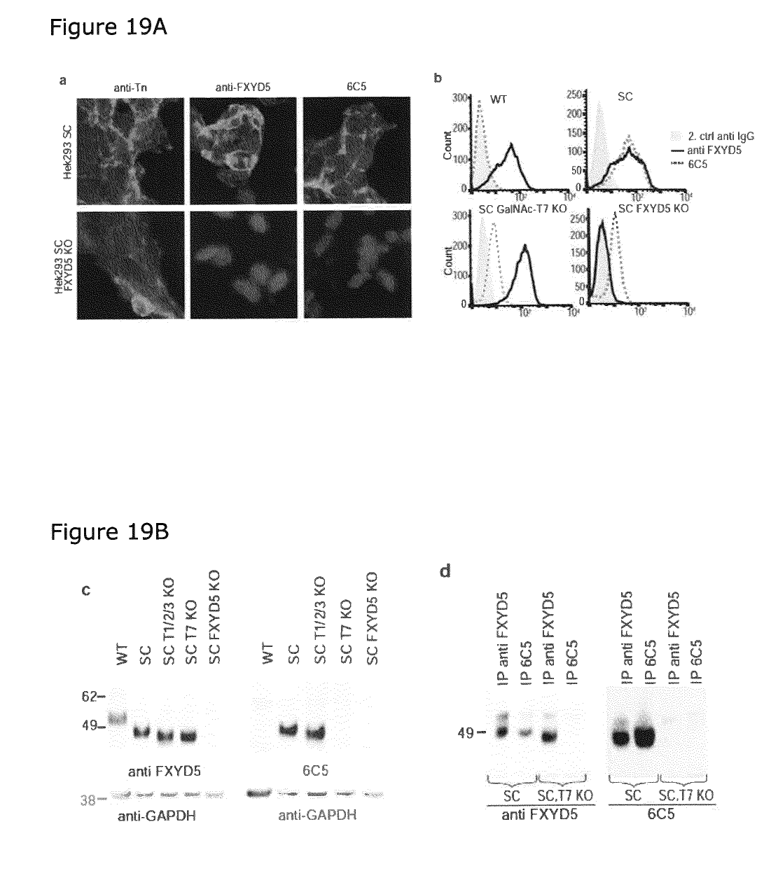

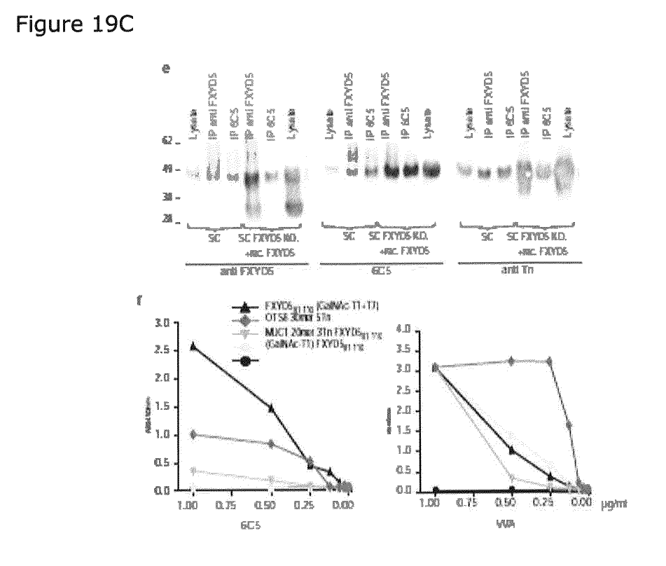

The mab 6C5 antigen epitope is dependent on GALNT7 expression—Since 6C5 was strongly positive on HEK293 SC cells we screened a panel of HEK293 cells in which GALN-Ts known to be expressed were knocked out individually or in combination (FIG. 19). While KO of the most abundant GALNTs, GALNT1/T2/T3 individually or in a triple KO, had no effect on 6C5 staining, KO of GALNT7 almost abolished 6C5 binding. The finding was confirmed by Western blot where the 6C5 ˜50 kDa antigen was clearly stained in lysates from HEK293 SC and SC GALNT1/T2/T3 triple KO while absent in the SC GALNT7 KO as well as WT cells.

Mab 6C5 reacts with a Tn-glycopeptide epitope on FXYD5 dependent on GALNT7—To further identify the O-glycoprotein and epitope recognized by mAb 6C5 we screened a panel of engineered isogenic HEK293-SC with different repertoire of GALNTs, and surprisingly found that reactivity was not dependent on the most abundant and broadly active GALNTs (GALNT1/T2/T3), while reactivity was almost abolished in HEK293-SC without GALNT7. This finding was confirmed by Western blot analysis where the ˜50 kDa band was detected in lysates from all HEK293-SCs except in cells without GALNT7 as well as in HEK293WT cells with elongated O-glycans (FIG. 19B). The molecular weight of 50 kDa pointed to the FXYD5 glycoprotein, we previously identified O-glycosites on (Steentoft et al. EMBO J 2013). To test whether the 6C5 epitope was located in FXYD5 we generated a FXYD5 KO in HEK293 SC background using CRISPR/CAS9. KO was confirmed by sequencing and an anti-FXYD5 mAb (NCC-M53)54, 55 using ICC and flow cytometry. While FXYD5 KO completely abolished 6C5 staining on ICC a faint binding could still be observed using FACS most likely representing cross-reactivity to the high concentration of the Tn-hapten present on SCs. No band was observed on Western blot of lysates from the SC FXYD5 KO with either the anti-FXYD5 mAb or 6C5, confirming that the 6C5 antigen was indeed FXYD5. Interestingly FXYD5 expression was unchanged upon GALNT7 KO and IP with 6C5 or anti-FXYD5 of either SC or SC GALNT7 KO lysates confirmed that the epitope of 6C5 is a GALNT7 specific glycopeptide on FXYD5 (FIG. 19B). Expression of a full length FXYD5 construct in the KO background reconstituted the 50 kDa band recognized by 6C5 (FIG. 19C). Surprisingly overexpression resulted in two primary bands seen by the anti-FXYD5 mAb but only one using 6C5. VVA staining confirmed that the lower band represented unglycosylated FXYD5 not recognized by 6C5 or VVA.

To narrow down the epitope of mAb 6C5 a 30 mer peptide covering amino acid 81-110 of FXYD5 (TDGPLVTDPETHKSTKAAHPTDDTTTLSER) was obtained and in vitro glycosylated with GalNacT1, T2 and T3 alone or in combination with GalNacT7. The glycopeptides were analysed by MALDI-TOF. GalNacT7 has been proposed to require neighboring GalNac residues in order to function and we observed no glycosylation of the peptide with GalNac-T7 alone. GalNacT2 only glycosylated one site, independent of GalNacT7, and GalNacT3 was unreactive (not shown). Glycosylation with GalNacT1 (FXYD5₈₁₋₁₁₀GalNacT1) or GalNacT1and GalNacT7 (FXYD5₈₁₋₁₁₀GalNac-T1+T7) resulted in a heterogeneous mixture with 0-8 O-GalNAc sites making it difficult to perceive a possible GalNacT7 specific site in the obtained MS spectrum. We therefore tested 6C5 reactivity on the peptide, the two glycopeptides as well as two Tn-glycopeptide controls in ELISA (FIG. 19C). While 6C5 did not bind to the unglycosylated FXYD5₈₁₋₁₁₀ peptide or the peptide glycosylated with GalNacT1 alone the mAb showed strong reactivity towards FXYD5₈₁₋₁₁₀GalNacT1+T7. FXYD5₈₁₋₁₁₀GalNacT1 and FXYD5₈₁₋₁₁₀GalNacT1+T7 exhibited similar reactivity with VVA.

The presented versatile strategy for discovery and generation of mAbs targeting cancer-specific truncated O-glycopeptide epitopes employs glycoengineered cancer cell lines displaying homogenous truncated O-glycans and relevant repertoires of GALN-Ts. The wide discovery potential of the strategy was illustrated by using as examples engineered breast, ovarian and pancreatic cancer cell lines for the generation of three novel mAbs. The mAb 6C5 was characterized in detail and shown to exhibit a high degree of cancer specific reactivity and be directed to a Tn-glycopeptide epitope in dysadherin (FXYD5), a known cancer-associated cell membrane glycoprotein. Moreover, we show that the epitope for 6C5 requires expression of the GALNT7 isoform and a distinct O-glycosylation pattern.

Methods

Cell line engineering—Human cancer cell lines were engineered as described in above Examples by ZFNs, TALENs, or CRISPR/Cas9 with KO of COSMC (SC) and/or KI of ST6GALNACT1 to express homogenous Tn and/or STn. KO of GALNTs and FXYD5 in HEK293 cells were made using CRISPR/Cas9. For FXYD5 KO a gRNA targeting 5′-TCGTTGGCCTGATTCTCCCC-3′ was selected and KO clones were identified using fragment analysis and KO confirmed by DNA sequencing using the following primers, 5′-GCCAGAGGTTTTTGCTCAGG-3′ and 5′-CAGGACAACGTTCACACGG-3′.

Immunogen preparation—Immunogens were prepared as follows; Whole cells (10 mio cells) were harvested by trypsin, washed in PBS, fixed in ice-cold 1% glutaraldehyde for 10 min, washed in PBS and used for immunization. VVA enrichment was performed passing either culture media or Triton x-100 cell extracts (pellets from 4× T175 lysed in 1% Triton-x-100 in lectin buffer (20 mM Tris-HCl pH 7.4, 150 mM NaCl, 1 M Urea, 1 mM CaCl₂, MgCl₂, MnCl₂ and ZnCl₂.) and protease inhibitor) over 500 μl VVA coupled agarose beads pre-equilibrated with lectin buffer containing 0.1% Triton x-100. The beads were subsequently washed and eluted with 0.4 M GalNAc as previously described (Schjoldager PNAS 2012) and eluted glycoproteins used for immunization. Membrane fractions were isolated by a one-step ultracentrifugation (100000×g 1 h) of total cell lysates (20 mM Tris-HCl pH 7.4, 250 mM Sucrose and protease inhibitor). The pellet was re-dissolved in PBS and used for immunization. Exosomes were purified by a two-step centrifugation protocol (11600 RPM and 38600 RPM) of serum free (48 h) cell culture medium. The obtained pellet was re-dissolved in PBS and used for immunization.

Lysis of cell pellets (8× T175 flask MDA-MB-231 SC or 2× T175 flasks OVCAR-3 SC confluent cells) were made in 1% Triton X-100 in lectin buffer (20 mM Tris-HCl pH 7.4, 150 mM NaCl, 1 M Urea, 1 mM CaCl₂, MgCl₂, MnCl₂ and ZnCl₂) and protease inhibitor (Complete, EDTA-free (Roche)). The lysates were diluted with lectin buffer to a final concentration of 0.1% Triton X-100 and the OVCAR-3 SC lysate neuraminidase treated 1.5 h, 37° C. with 0.01 U/mL neuraminidase (C. perfringens, type VI (Sigma)). Samples were passed over 500 μl VVA coupled agarose beads (Vector Laboratories) pre-equilibrated with lectin buffer containing 0.1% Triton X-100. The beads were subsequently washed and eluted with 2×1 ml 0.4 M GalNAc in 20 mM Tris-HCl pH 7.4, 150 mM NaCl and 0.1% triton x-100. Glycoprotein enrichment was confirmed by western blot by VVA detection and eluted glycoproteins were used for immunization.

Microvesicles were purified from 500 ml of serum free (48 h) cell culture medium from T3M4 SC by five centrifugation steps (10′ 150×g, 30′ 300×g, 30′ 850×g, 30′ 10,000×g, 1 h 100,000×g all at 4° C.). The obtained pellet was re-dissolved in PBS and used for immunization.

Immunogen preparation—Immunogens were prepared as follows; Whole cells (10 mio cells) were harvested by trypsin, washed in PBS, fixed in ice-cold 1% glutaraldehyde for 10 min, washed in PBS and used for immunization. VVA enrichment was performed passing either culture media or Triton x-100 cell extracts (pellets from 4× T175 lysed in 1% Triton-x-100 in lectin buffer (20 mM Tris-HCl pH 7.4, 150 mM NaCl, 1 M Urea, 1 mM CaCl₂, MgCl₂, MnCl₂ and ZnCl₂.) and protease inhibitor) over 500 μl VVA coupled agarose beads pre-equilibrated with lectin buffer containing 0.1% Triton x-100. The beads were subsequently washed and eluted with 0.4 M GalNAc as previously described (Schjoldager 2012) and eluted glycoproteins used for immunization. Membrane fractions were isolated by a one-step ultracentrifugation (100000×g 1 h) of total cell lysates (20 mM Tris-HCl pH 7.4, 250 mM Sucrose and protease inhibitor). The pellet was re-dissolved in PBS and used for immunization. Exosomes were purified by a two-step centrifugation protocol (11600 RPM and 38600 RPM) of serum free (48 h) cell culture medium. The obtained pellet was re-dissolved in PBS and used for immunization.

Lysis of cell pellets (8× T175 flask MDA-MB-231 SC or 2× T175 flasks OVCAR-3 SC confluent cells) were made in 1% Triton X-100 in lectin buffer (20 mM Tris-HCl pH 7.4, 150 mM NaCl, 1 M Urea, 1 mM CaCl₂, MgCl₂, MnCl₂ and ZnCl₂) and protease inhibitor (Complete, EDTA-free (Roche)). The lysates were diluted with lectin buffer to a final concentration of 0.1% Triton X-100 and the OVCAR-3 SC lysate neuraminidase treated 1.5 h, 37° C. with 0.01 U/mL neuraminidase (C. perfringens, type VI (Sigma)). Samples were passed over 500 μl VVA coupled agarose beads (Vector Laboratories) pre-equilibrated with lectin buffer containing 0.1% Triton X-100. The beads were subsequently washed and eluted with 2×1 ml 0.4 M GalNAc in 20 mM Tris-HCl pH 7.4, 150 mM NaCl and 0.1% triton x-100. Glycoprotein enrichment was confirmed by western blot by VVA detection and eluted glycoproteins were used for immunization.

Microvesicles were purified from 500 ml of serum free (48 h) cell culture medium from T3M4 SC by five centrifugation steps (10′ 150×g, 30′ 300×g, 30′ 850×g, 30′ 10,000×g, 1 h 100,000×g all at 4° C.). The obtained pellet was re-dissolved in PBS and used for immunization.

Immunization Protocol

Female Balb/c mice were injected subcutaneously or intraperitoneally with 100 μl immunogen in a total volume of 200 μl (1:1 mix with Freunds adjuvant, Sigma). Mice received three immunizations 2-3 weeks apart and finally a boost intraperitoneally 3 days before fusion. Blood samples were obtained by eye or tail bleeding one week after third immunization

Balb/c mice were immunized with a single intraperitoneal injection of 20-40 μg protein of VVA-enriched glycoproteins (MDA-MB-231 SC and OVCAR-3 SC) or with a single subcutaneous injection of microvesicle fraction (T3M4) in a total volume of 200 μl (1:1 mix with Freunds adjuvant) three times, three weeks apart and finally an intraperitoneal boost without adjuvant. Three days after the 4th immunization splenocytes from one mouse were fused with NS1 myeloma cells. Hybridoma supernatants were screened by immunocytochemistry on trypsinated and acetone fixed cells46 after 10-12 days of culture. Hybridomas producing Abs with significant reactivity to SC and not to WT cells were subjected to at least two limiting dilutions. Three mAb producing clones were finally selected for further characterization, 6C5 (MDA-MB-231 SC), 1D5 (OVCAR-3 SC) and 5D10 (T3M4 SC) and all determined to secrete IgG1.

Monoclonal antibodies were generated as previously described (Vester-Christensen 2013) from Balb/c mice immunized with human cancer cells engineered to only display Tn O-glycans or cell membrane extracts hereof. Screening was based on immunocytochemistry on acetone fixed slides with engineered cells and corresponding wt cells and immunohistology with human cancer tissues as well as ELISA assays and Western Blot. Selection was based on reactivity pattern similar to total sera of the same mice

For immunohistochemistry formalin fixed paraffin embedded TMAs (Biomax) were dewaxed, rehydrated and subjected to antigen retrieval by microwave treatment (5 min at 600 w and 15 min at 300 w) at pH 6 (citrate buffer). Sections subjected to sialidase treatment were pretreated with 0.1 U/ml neuraminidase in 0.1M sodium acetate buffer (pH 5.5) for 2 hrs at 37° C. Sections were blocked with 10% calf serum, incubated overnight at 4° C. with primary antibodies, rinsed and incubated with FITC-conjugated rabbit anti-mouse immunoglobulins (DAKO) for 45 min. Slides were mounted in Vectashield (Vector Laboratories, Inc).

For immunocytochemistry cells were seeded on sterile coverslips coated with type I collagen (0.4 mg/ml) and cultivated for 2-3 days. The cells were then washed in cold PBS and fixed in cold 4% PFA for 10 min. After washing in cold PBS, the primary antibody (1 μg/ml (NCC-M53), 0.5 μg/ml VVA-biotin, 10 or 100× diluted hybridoma supernatant (6C5), undiluted hybridoma supernatant (5F4, 1E3, 4C4, 8B8)) was added ON 4° C. After washing the secondary antibody was added (anti-Mouse Ig-FITC, 1:400 dilution in 2.5% BSA PBS or 1:2000 strepdavidin-Alexa488 (Life technologies)) and incubated for 45 min, washed and mounted with ProLong® Gold Antifade Mountant with DAPI (Life Technologies).

For immunohistochemistry formalin fixed paraffin embedded TMAs (Biomax) were heated at 60° C. 30 min, dewaxed, rehydrated and subjected to antigen retrieval by microwave treatment at pH 6 (citrate buffer). Sections were blocked with 10% calf serum, incubated overnight at 4° C. with primary antibody (undiluted supernatant), rinsed and incubated with anti-Mouse Ig-FITC, 1:200 dilution in 2.5% BSA/PBS and incubated for 45 min. Slides were washed and mounted in Vectashield (Vector Laboratories, Inc). All images were acquired using Zeiss Axioscope 2 plus with an AxioCam MRc (40×).

For FACS analysis cells were harvested by trypsin, washed in PBS, blocked in FACS buffer (2% Fetal bovine serum in PBS) and antibody added 45 min on ice (1 μg/ml (MC53), 1:100× diluted hybridoma supernatant (6C5)). Cells were washed in FACS solution, incubated 35 min on ice with anti-Mouse Ig-FITC, washed and analyzed on instrument.

Cell lysis and subsequent immunoprecipitation (IP) was performed following a modified protocol61. Cells were lysed in 1× High salt lysis buffer (10 mM, Tris-HCL, 420 mM NaCl, 0.1% NP-40), and protease inhibitor cocktail (cOmplete, EDTA free (Roche)), subjected to 3× freeze-thaw cycles and sonication. The lysate was centrifuged,13000 rpm 10 min, and the protein concentration was measured by 280 nm absorption on nanodrop. For IP 800 μg of lysate was incubated with 1 μg of antibody (or 50 μl hybridoma supernatant) and incubated for 4 h 4° C. 15 μl of Dynabeads™ Protein G (Invitrogen) were washed 3× in low salt lysis buffer (10 mM Tris-HCL, 100 mM NaCl, 0.1% NP-40), added to the lysate-Ab mix and incubated ON 4° C. Beads were washed with 4× low salt lysis buffer and eluted in 60 μl 0.5 M ammonium hydroxide or Novex NuPAGE LDS Sample Buffer with 20 mM DTT.

For Western blot, samples were mixed to a final concentration of 1× Novex NuPAGE LDS Sample Buffer and 20 mM DTT. After denaturation at 96° C. for 10 min the samples were loaded into a NuPAGE Bis-Tris 4-12% gel (Invitrogen) and electrophoresis was carried out at 200 V for 35 min. The proteins were transferred onto a nitrocellulose membrane at 30 V for 90 min and the membrane blocked in 5% skimmed milk or 1% polyvinylpyrrolidone (for VVA detection) in TBS-T. The membrane was incubated with primary antibody (1 μg/ml (MC53, VVA-biotin), 0.1 μg/ml (anti-GAPDH, FL-335 Santa cruise biotechnology), 10× diluted hybridoma supernatant (6C5)) in blocking buffer at 4° C. overnight, washed and incubated with the secondary layer at room temperature for 1 hour (Rabbit Anti-Mouse Ig-HRP, Goat Anti-Rabbit Ig-HRP or Streptavidin-HRP (Dako, 1:4000 dilution) and developed using the Thermo Scientific Pierce ECL Western Blotting Substrate kit.

FXYD5 recombinant protein and glycopeptides—Full length FXYD5 with a C-terminal Myc-tag was cloned into the PTT5 vector. HEK293 SC FXYD5 KO cells were transfected with 1 μg of DNA using lipofectamin® 3000 and cells were harvested after 48 h. Cell lysis, IP and western blot were performed as described above. A 30-mer peptide (TDGPLVTDPETHKSTKAAHPTDDTTTLSER) was purchased (SynPeptide) covering the investigated glycosylation site on FXYD5 and subjected to in vitro glycosylation using recombinant glycosyltransferases expressed as soluble secreted truncated proteins in insect cells and purified. Glycosylation of the peptide was performed in a reaction mixture (0.4 mg peptide/mL) containing 25 mM cacodylate buffer (pH 7.4), 10 mM MnCl2, 0.25% Triton X-100, 12 μg/mL GalNAc-T and 2 mM UDP-GalNAc. The reactions were incubated at 37° C., and glycopeptide development was monitored by MALDI-TOF MS (Bruker, Autoflex).

For ELISA assays, 100 μg of peptide was glycosylated with either GalNacT1 alone or in combination with GalNacT7, acidified and purified by UHPLC (Thermo, Ultimate™ 3000) on a C18 column (Phenomenex, Jupiter, 5 μm, 300 Å, 250 mm). 96-well plates (MaxiSorp, Nunc) were coated overnight with peptide or glycopeptides diluted to 50 μl/well in coating buffer (Na2CO3-buffer pH 9.6) at 4° C. ON. Plates were blocked with 150 μl/well PLI-P-buffer (PO4-buffer pH 7.4, Na/K, 1% Triton-X, 1% BSA) for 1 hour at room temperature and incubated 1 h with the primary layer (0.5 μg/ml, VVA-biotin or 6C5). After 1 hour of incubation with secondary layer (1:4000 Streptavidin-HRP or anti-IgG-HRP) the plates where developed with TMB+ chromogen (Dako) and stopped with 0.5 M H2SO4 and read at 450 nm. All washing steps were performed with PBS-T (PBS-buffer pH 7.4, 0.05% Tween-20).

### LIST OF REFERENCES

Amado M, Almeida R, Schwientek T, Clausen H (1999) Identification and characterization of large galactosyltranserase gene families: galactosyltransferases for all functions. Biochim. Biophys. Acta, 1473:35-53

Arthur, C. M., Cummings, R. D., and Stowell, S. R. (2014) Using glycan microarrays to understand immunity. Current opinion in chemical biology 18, 55-61

Bennett E P, Mandel U, Clausen H, Gerken T A, Fritz T A, Tabak L A. (2012) Control of Mucin-Type O-Glycosylation—A Classification of the Polypeptide GalNAc-transferase Gene Family. Glycobiology. 22(6):736-56

Blixt, O., Head, S., Mondala, T., Scanlan, C., Huflejt, M. E., Alvarez, R., Bryan, M. C., Fazio, F., Calarese, D., Stevens, J., Razi, N., Stevens, D. J., Skehel, J. J., van Die, I., Burton, D. R., Wilson, I. A., Cummings, R., Bovin, N., Wong, C. H., and Paulson, J. C. (2004) Printed covalent glycan array for ligand profiling of diverse glycan binding proteins. Proceedings of the National Academy of Sciences of the United States of America 101, 17033-17038

Bohm, E., Seyfried, B. K., Dockal, M., Graninger, M., Hasslacher, M., Neurath, M., Konetschny, C., Matthiessen, P., Mitterer, A., and Scheiflinger, F. (2015) Differences in N-glycosylation of recombinant human coagulation factor VII derived from BHK, CHO, and HEK293 cells. BMC biotechnology 15, 87

Colley K. J. (1997) Golgi localization of glycosyltransferases: more questions than answers. Glycobiology 7, 1-13

Duda K, Lonowski L A, Kofoed-Nielsen M, Ibarra A, Delay C M, Kang Q, Yang Z, Pruett-Miller S M, Bennett E P, Wandall H H, Davis G D, Hansen S H, Frodin M. Nucleic Acids Res. 2014 June

Durocher Y, Perret S, Kamen A (2002) High-level and high-throughput recombinant protein production by transient transfection of suspension-growing human 293-ebnal cells. Nucleic acids res. 30(2):e9

El Mai N, Donadio-Andrei S, Iss C, Calabro V, Ronin C (2013) Engineering a human-like glycosylation to produce therapeutic glycoproteins based on 6-linked sialylation in CHO cells. Methods Mol Biol 988:19-29

Geissner, A., Anish, C., and Seeberger, P. H. (2014) Glycan arrays as tools for infectious disease research. Current opinion in chemical biology 18, 38-45

Hansen, L., Lind-Thomsen, A., Joshi, H. J., Pedersen, N. B., Have, C. T., Kong, Y., Wang, S., Sparso, T., Grarup, N., Vester-Christensen, M. B., Schjoldager, K., Freeze, H. H., Hansen, T., Pedersen, O., Henrissat, B., Mandel, U., Clausen, H., Wandall, H. H., and Bennett, E. P. (2015) A glycogene mutation map for discovery of diseases of glycosylation. Glycobiology 25, 211-224

Ilver, D., Johansson, P., Miller-Podraza, H., Nyholm, P. G., Teneberg, S., and Karlsson, K. A. (2003) Bacterium-host protein-carbohydrate interactions. Methods in enzymology 363, 134-157

Jacewicz, M., Clausen, H., Nudelman, E., Donohue-Rolfe, A., and Keusch, G. T. (1986) Pathogenesis of shigella diarrhea. XI. Isolation of a shigella toxin-binding glycolipid from rabbit jejunum and HeLa cells and its identification as globotriaosylceramide. The Journal of experimental medicine 163, 1391-1404

Ju T, Lanneau G S, Gautam T, Wang Y, Xia B, Stowell S R, et al. (2008) Human tumor antigens Tn and sialyl Tn arise from mutations in Cosmc. Cancer Res 15 donadi (68):1636-1646

Kornfeld S., Kornfeld R. (1985) Assembly of asparagine-linked oligosaccharides. Annu. Rev. Biochem., 54: 631-644

Lairson L L, Henrissat B, Davies G J, Withers S G. (2008) Glycosyltransferases: Structures, functions, and mechanisms, Annu Rev Biochem 77: 521-555

Lee K, Jin X, Zhang K, Copertino L, Andrews L, Baker-Malcolm J, Geagan L, Oiu H, Seiger K, Barngrover D, McPherson J M, Edmunds T. (2003) A biochemical and pharmacological comparison of enzyme replacement therapies for the glycolipid storage disorder Fabry disease. Glycobiology 13(4): 305-313

Magnani, J. L., Nilsson, B., Brockhaus, M., Zopf, D., Steplewski, Z., Koprowski, H., and Ginsburg, V. (1982) A monoclonal antibody-defined antigen associated with gastrointestinal cancer is a ganglioside containing sialylated lacto-N-fucopentaose II. The Journal of biological chemistry 257, 14365-14369

Malphettes L, Freyvert Y, Chang J, Liu P-Q, Chan E, Miller J C, Zhou Z, Nguyen T, Tsai C, Snowden A W, et al. (2010) Highly efficient deletion of FUT8 in CHO cell lines using zinc-finger nucleases yields cells that produce completely nonfucosylated antibodies, Biotechnol Bioeng 106: 774-783

Mandel U, Hassan H, Therkildsen M H, Rygaard J, Jakobsen M H, Juhl B R, Dabelsteen E, Clausen H. (1999) Expression of polypeptide GalNAc-transferases in stratified epithelia and squamous cell carcinomas: Immunohistological evaluation using monoclonal antibodies to three members of the GalNAc-transferase family, Glycobiology 9: 43-52

Marcos, N. T., Pinho, S., Grandela, C., Cruz, A., Samyn-Petit, B., Harduin-Lepers, A., Almeida, R., Silva, F., Morais, V., Costa, J., Kihlberg, J., Clausen, H., and Reis, C. A. (2004) Cancer Res. 64, 7050-7057

Marcos N T, Bennett E P, Gomes J, Magalhaes A, Gomes C, David L, Dar I, Jeanneau C, DeFrees S, Krustrup D, Vogel L K, Kure E H, Burchell J, Taylor-Papadimitriou J, Clausen H, Mandel U, Reis C A (2011) ST6GalNAc-I controls expression of sialyl-Tn antigen in gastrointestinal tissues. Front Biosci 3:1443-55

Maresca M, Lin V G, Guo N, Yang Y. (2013) Obligate ligation-gated recombination (ObLiGaRe): custom-designed nuclease-mediated targeted integration through nonhomologous end joining. Genome Res, 23: 539-546

Narimatsu H. (2006) Human glycogene cloning: Focus on beta 3-glycosyltransferase and beta 4-glycosyltransferase families, Curr Opin Struct Biol 16: 567-575

Ohtsubo, K. and Marth, J. D. (2006) Glycosylation in cellular mechanisms of health and disease Cell 126, 855-867

Padler-Karavani, V., Song, X., Yu, H., Hurtado-Ziola, N., Huang, S., Muthana, S., Chokhawala, H. A., Cheng, J., Verhagen, A., Langereis, M. A., Kleene, R., Schachner, M., de Groot, R. J., Lasanajak, Y., Matsuda, H., Schwab, R., Chen, X., Smith, D. F., Cummings, R. D., and Varki, A. (2012) Cross-comparison of protein recognition of sialic acid diversity on two novel sialoglycan microarrays. The Journal of biological chemistry 287, 22593-22608

Palma, A. S., Feizi, T., Childs, R. A., Chai, W., and Liu, Y. (2014) The neoglycolipid (NGL)-based oligosaccharide microarray system poised to decipher the meta-glycome. Current opinion in chemical biology 18, 87-94

Patnaik, S. K. & Stanley, P. (2006) Lectin-resistant CHO glycosylation mutants. Methods Enzymol. 416, 159-182

Paulson, J. C., Blixt, O., and Collins, B. E. (2006) Sweet spots in functional glycomics. Nature chemical biology 2, 238-248

Rillahan, C. D., and Paulson, J. C. (2011) Glycan microarrays for decoding the glycome. Annual review of biochemistry 80, 797-823

Ronda, C., Pedersen, L. E., Hansen, H. G., Kallehauge, T. B., Betenbaugh, M. J., Nielsen, A. T., and Kildegaard, H. F. (2014) Accelerating genome editing in CHO cells using CRISPR Cas9 and CRISPy, a web-based target finding tool Biotechnol. Bioeng. 111, 1604-1616

Schachter H. (2014) Complex N-glycans: the story of the “yellow brick road”. Glycoconjugate journal 31(1): 1-5

Schietinger, A., Philip, M., Yoshida, B. A., Azadi, P., Liu, H., Meredith, S. C., and Schreiber, H. (2006) A mutant chaperone converts a wild-type protein into a tumor-specific antigen. Science 314, 304-308

Sewell R, Bäckström M, Dalziel M, Gschmeissner S, Karlsson H, Noll T, et al. (2006) The ST6GalNAc-I sialyltransferase localizes throughout the Golgi and is responsible for the synthesis of the tumor-associated sialyl-Tn O-glycan in human breast cancer. J Biol Chem 281:3586-3594

Sharon, N., and Lis, H. (2004) History of lectins: from hemagglutinins to biological recognition molecules. Glycobiology 14, 53R-62R

Singh P K, Behrens M E, Eggers J P, Cerny R L, Bailey J M, Shanmugam K, Gendler S J, Bennett E P, Hollingsworth M A. (2008) Phosphorylation of MUC1 by Met modulates interaction with p53 and MMP1 expression. J Biol Chem. 283:26985-26995

Steentoft C, Bennett E P, Schjoldager K T, Vakhrushev S Y, Wandall H H, Clausen H. (2014) Precision genome editing—a small revolution for glycobiology. Glycobiology 24:663-80

Steentoft, C., Vakhrushev, S. Y., Joshi, H. J., Kong, Y., Vester-Christensen, M. B., Schjoldager, K. T., Lavrsen, K., Dabelsteen, S., Pedersen, N. B., Marcos-Silva, L., Gupta, R., Paul Bennett, E., Mandel, U., Brunak, S., Wandall, H. H., Levery, S. B., and Clausen, H. (2013) Precision mapping of the human O-GalNAc glycoproteome through SimpleCell technology. The EMBO journal 32, 1478-1488

Steentoft C, Vakhrushev S Y, Vester-Christensen M B, Schjoldager K T, Kong Y, Bennett E P, Mandel U, Wandall H, Levery S B, Clausen H. (2011) Mining the O-glycoproteome using zinc-finger nuclease-glycoengineered SimpleCell lines. Nat Methods. 8(11):977-82

Stevens, J., Blixt, O., Tumpey, T. M., Taubenberger, J. K., Paulson, J. C., and Wilson, I. A. (2006) Structure and receptor specificity of the hemagglutinin from an H5N1 influenza virus. Science 312, 404-410

Tang, P. W., and Feizi, T. (1987) Neoglycolipid micro-immunoassays applied to the oligosaccharides of human milk galactosyltransferase detect blood-group related antigens on both O- and N-linked chains. Carbohydrate research 161, 133-143

Tarp M A, Clausen H. (2008) Mucin-type O-glycosylation and its potential use in drug and vaccine development. Biochim Biophys Acta 1780(3): 546-63

Tarp, M. A., Sorensen, A. L., Mandel, U., Paulsen, H., Burchell, J., Taylor-Papadimitriou, J., and Clausen, H. (2007) Identification of a novel cancer-specific immunodominant glycopeptide epitope in the MUC1 tandem repeat. Glycobiology 17, 197-209

Taylor, M. E., and Drickamer, K. (2014) Convergent and divergent mechanisms of sugar recognition across kingdoms. Current opinion in structural biology 28, 14-22

Tsuji S, Datta A K, Paulson J C. (1996) Systematic nomenclature for sialyltransferases. Glycobiology 6(7):v-vii.

Umana P, Jean-Mairet J, Moudry R, Amstutz H, Bailey J E. (1999) Engineered glycoforms of an antineuroblastoma IgG1 with optimized antibody-dependent cellular cytotoxic activity. Nat Biotechno1.17:176-180

Vester-Christensen M B, Bennett E P, Clausen H, Mandel U. (2013). Generation of monoclonal antibodies to native active human glycosyltransferases. Methods Mol Biol 1022:403-420

Walsh, G. (2014) Biopharmaceutical benchmark 2014, Nat. Biotechnol. 32, 992-1000.

Wandall H H, Blixt O, Tarp M A, Pedersen J W, Bennett E P, Mandel U, et al. (2010) Cancer biomarkers defined by autoantibody signatures to aberrant O-glycopeptide epitopes. Cancer research 70(4):1306-13

Wands, A. M., Fujita, A., McCombs, J. E., Cervin, J., Dedic, B., Rodriguez, A. C., Nischan, N., Bond, M. R., Mettlen, M., Trudgian, D. C., Lemoff, A., Quiding-Jarbrink, M., Gustaysson, B., Steentoft, C., Clausen, H., Mirzaei, H., Teneberg, S., Yrlid, U., and Kohler, J. J. (2015) Fucosylation and protein glycosylation create functional receptors for cholera toxin. eLife 4

Xu, X., Nagarajan, H., Lewis, N. E., Pan, S., Cai, Z., Liu, X., Chen, W., Xie, M., Wang, W., Hammond, S., Andersen, M. R., Neff, N., Passarelli, B., Koh, W., Fan, H. C., Wang, J., Gui, Y., Lee, K. H., Betenbaugh, M. J., Quake, S. R., Famili, I., Palsson, B. O., and Wang, J. (2011) The genomic sequence of the Chinese hamster ovary (CHO)-K1 cell line. Nature biotechnology 29, 735-741

Yamane-Ohnuki N, Kinoshita S, Inoue-Urakubo M, Kusunoki M, Iida S, Nakano R, Wakitani M, Niwa R, Sakurada M, Uchida K, Shitara K, Satoh M (2004) Establishment of FUT8 knockout Chinese hamster ovary cells: an ideal host cell line for producing completely defucosylated antibodies with enhanced antibody-dependent cellular cytotoxicity. Biotechnol Bioeng 87:614-622

Yang, Z., Wang, S., Halim, A., Schulz, M. A., Frodin, M., Rahman, S. H., Vester-Christensen, M. B., Behrens, C., Kristensen, C., Vakhrushev, S. Y., Bennett, E. P., Wandall, H. H., and Clausen, H. (2015) Engineered CHO cells for production of diverse, homogeneous glycoproteins. Nature biotechnology 33, 842-844

Yu, X., and Blanchard, H. (2014) Carbohydrate recognition by rotaviruses. Journal of structural and functional genomics 15, 101-106

1. A plurality of isogenic mammalian cells, wherein one or more endogenous glycogenes have been inactivated and/or wherein one or more exogenous glycogene have been introduced independently in individual cells of said plurality of mammalian cells.

2.-14. (canceled)

15. The plurality of isogenic mammalian cells of claim 1, furthermore encoding an exogenous protein of interest or induced to overexpress an endogenous protein of interest.

16.-18. (canceled)

19. The plurality of isogenic mammalian cells of claim 15, in which the protein of interest is a lysosomal enzyme expressed to comprise one or more posttranslational modifications independently selected from:
a) with α2,3NeuAc capping,
b) without α2,3NeuAc capping,
c) with α2,6NeuAc capping,
d) without α2,6NeuAc capping,
e) without LacDiNac structure,
f) high Mannose6phosphate,
g) low Mannose6phosphate,
h) without bisecting glycoforms; and
i) with high mannose.

20. The plurality of isogenic mammalian cells of claim 1, wherein said one or more endogenous glycogene inactivated and/or exogenous glycogene introduced independently in individual cells of said plurality of mammalian cells is selected from the list of GNPTAB, GNPTG, NAGPA, ALG3/6/8/9/10/12s, Mannosidases (MAN1A1, MAN1A2, MAN1B1, MAN1C1, MAN2A1, MAN2A2), MOGS, GANAB plus MGAT1/2 and Sialyl transferases.

21. The plurality of isogenic mammalian cells of claim 1 wherein said one or more endogenous glycogene inactivated is GNPTAB, such as in order to increase sialic acids.

22. The plurality of isogenic mammalian cells of claim 19, wherein said lysosomal enzyme has obtained increased mannose-6-phosphate (M6P) tagging of N-glycans and/or has obtained changed site occupancy of M6P, such as by knocking out a gene selected from ALG3, ALG8, NAGPA.

23. The plurality of isogenic mammalian cells of claim 19, wherein said lysosomal enzyme has obtained increased high mannose structures, such as by knocking out a gene selected from MGAT1 and/or GNPTAB and/or MOGS.

24.-39. (canceled)

40. The plurality of isogenic mammalian cells of claim 1, wherein one or more of said cells has an inactivation and/or introduction of one or more glycogene selected from the list consisting of glycogenes associated with subset of O-Mannose type glycoproteins (listed in Table 5 under group 1 genes for O-Glycans), such as POMT1 and/or POMT2 and/or TMTC1 and/or TMTC2 and/or TMTC3 and/or TMTC4 (Group 1).

41.-43. (canceled)

44. The plurality of isogenic mammalian cells of claim 1, wherein one or more of said cells has an inactivation and/or introduction of one or more glycogene selected from the list consisting of MGAT1 (N-Glycans), COSMC (O-GalNac), B4GALT7 (Glycosaminoglycans, GAG), B4GALT5/6 (Glycosphingolipids), POMGNT1 (O-Man) (Group 2).

45. (canceled)

46. The plurality of isogenic mammalian cells of claim 1, wherein one or more of said cells has an inactivation and/or introduction of one or more glycogene selected from the list consisting of MGAT2/3/4A/4B/4C/4D/5/5B, MAN1A1, MAN1A2, MAN1B1, MAN1C1, MAN2A1, MAN2A2, MOGS, GANAB, B3GALT1/T2/T4/T5, B3GALNT1/T2, B3GNT2/T3/T4/T6/T7/T8/T9, B4GALT1/T2/T3/T4, B4GALNT1/T2/T3/T4, GCNT1/T2/T3/T4/T6/T7, B3GAT1/T2, B4GAT1, LARGE, GYLT1B (LARGE2), ABO, A4GNT, XXYLT1, EXT1/2, EXTL1/3, CHPF/2, CHSY1/3, and CSGALNACT1/T2 (Group 3).

47. The plurality of isogenic mammalian cells of claim 1, wherein one or more of said cells has an inactivation and/or introduction of one or more glycogene selected from the list consisting of glycogenes associated with genes involved in N and O-glycan and glycolipid capping (sialylation), such as ST3GAL1/2/3/4/5/6 (α2,3NeuAc capping/sialylation) and/or ST6GAL1/2 (α2,6NeuAc capping/sialylation) and/or ST8SIA1/2/3/4/5/6 (capping by poly-sialylation) and/or ST6GALNAC1/2/3/4/5/6 (α2,6NeuAc capping/sialylation) (Group 4).

48. The plurality of isogenic mammalian cells of claim 1, wherein one or more of said cells has an inactivation and/or introduction of one or more glycogene selected from the list consisting of FUT1/2/3/4/5/6/7/8/9/10/11, ST3GAL1/2/3/4/5/6, ST6GAL1/2, ST6GALNAC1/2/3/4/5/6, and ST8SIA1/2/3/4/5/6 (Group 4).

49. The plurality of isogenic mammalian cells of claim 1, wherein one or more of said cells has an inactivation and/or introduction of one or more glycogene selected from the list consisting of DSE, DSEL, CHST11/T12/T13/T14/T15, UST, NDST1/T2/T3/T4, GLCE, HS2ST1, HS3ST1/T2/T3A1/T3B1/T4/T5/T6, HS6ST1/T2/T3, SULF1/2, HPSE, CHST1/T2/T3/T4/T5/T6/T7/T8/T9/T10, GAL3ST1/T2/T3/T4, CHST8/T9/T10, CASD1, FAM20B, POMK, GNPTAB (Group 5).

50. The plurality of isogenic mammalian cells of claim 1, wherein one or more of said cells are HEK293 cells that has an introduction of one or more glycogene selected from the list of A3GALT2, A4GNT, ABO, ALG1L2, B3GALNT1, B3GALT2, B3GNT6, B4GALNT2, FUT5, FUT7, FUT9, GALNT15, GALNT5, GALNT9, GALNTL5, GALNTL6, GALNT19/WBSCR17, GCNT3, GCNT4, GCNT7, GLT1D1, GLT6D1, HAS1, MGAT4C, MGAT4D, ST6GAL2, ST6GALNAC1, ST8SIA1, ST8SIA3, ST8SIA4, CHST2, GAL3ST3, HS3ST1, HS3ST4, HS3ST5, NDST3 (Table 6).

51.-52. (canceled)

53. A glycome display library comprising the plurality of isogenic mammalian cells of claim 1.

54. (canceled)

55. Use of the glycome display library of claim 53 for the display of a plurality of different glycans on the surface of or after being released from said mammalian cells.

56. Use of the glycome display library of claim 53 for probing interactions of a glycan-binding entity, such as a glycan-binding-protein (GBP) with glycans presented by said mammalian cells.

57.-64. (canceled)

65. A mammalian cell capable of expressing a gene encoding a polypeptide of interest, wherein the polypeptide of interest is expressed comprising one or more of the posttranslational modification patterns:
i) homogenous mono-antennary or biantennary N-glycans, and
a) with α2,3NeuAc capping,
b) without α2,3NeuAc capping,
c) with α2,6NeuAc capping,
d) without α2,6NeuAc capping,
e) without LacDiNac structure, or
f) with M6P.

66. (canceled)

67. A method for identifying glycoprotein glycovariants with improved drug properties comprising:
a) producing a plurality of different glycoforms of said glycoprotein by expressing the glycoprotein in a plurality of different isogenic mammalian cells, each of said isogenic mammalian cells comprising different glycosylation capacities due to their having one or more endogenous glycogene that has been inactivated and/or one or more exogenous glycogene that has been introduced;
b) determining the activity of the different glyco-forms in comparison with a reference glycoprotein in suitable bioassay; and
c) selecting the glycoform with the higher/highest/optimal activity.

68. The method of claim 67, wherein said one or more endogenous glycogene inactivated and/or exogenous glycogene introduced in said isogenic mammalian cells is selected from the list of GNPTAB, GNPTG, NAGPA, ALG3/6/8/9/10/12s, Mannosidases (MAN1A1, MAN1A2, MAN1B1, MAN1C1, MAN2A1, MAN2A2), MOGS, GANAB plus MGAT1/2 and Sialyl transferases.
